SEQUENCE LISTING (940 sequences)

SEQ ID NO: 1 - protein, 20 aa, synthetic construct
  VARIANT 1..20: note = This region may encompass 1-20 G repeating units.
GGGGGGGGGGGGGGGGGGGG

SEQ ID NO: 2 - protein, 40 aa, synthetic construct
  VARIANT 1..40: note = This region may encompass 1-20 GS repeating units.
GSGSGSGSGSGSGSGSGSGSGSGSGSGSGSGSGSGSGSGS

SEQ ID NO: 3 - protein, 60 aa, synthetic construct
  VARIANT 1..60: note = This region may encompass 1-20 GGS repeating units.
GGSGGSGGSGGSGGSGGSGGSGGSGGSGGSGGSGGSGGSGGSGGSGGSGGSGGSGGSGGS

SEQ ID NO: 4 - protein, 80 aa, synthetic construct
  VARIANT 1..80: note = This region may encompass 1-20 GGGS repeating units.
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 5 - protein, 100 aa, synthetic construct
  VARIANT 1..100: note = This region may encompass 1-20 GGGGS repeating units.
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 6 - protein, 10 aa, synthetic construct
  VARIANT 1..10: note = This region may encompass 1-10 G repeating units.
GGGGGGGGGG

SEQ ID NO: 7 - protein, 20 aa, synthetic construct
  VARIANT 1..20: note = This region may encompass 1-10 GS repeating units.
GSGSGSGSGSGSGSGSGSGS

SEQ ID NO: 8 - protein, 30 aa, synthetic construct
  VARIANT 1..30: note = This region may encompass 1-10 GGS repeating units.
GGSGGSGGSGGSGGSGGSGGSGGSGGSGGS

SEQ ID NO: 9 - protein, 40 aa, synthetic construct
  VARIANT 1..40: note = This region may encompass 1-10 GGGS repeating units.
GGGSGGGSGGGSGGGSGGGSGGGSGGGSGGGSGGGSGGGS

SEQ ID NO: 10 - protein, 50 aa, synthetic construct
  VARIANT 1..50: note = This region may encompass 1-10 GGGGS repeating units.
GGGGSGGGGSGGGGSGGGGSGGGGSGGGGSGGGGSGGGGSGGGGSGGGGS

SEQ ID NO: 11 - protein, 5 aa, synthetic construct
  VARIANT 1..5: note = This region may encompass 1-5 G repeating units.
GGGGG

SEQ ID NO: 12 - protein, 10 aa, synthetic construct
  VARIANT 1..10: note = This region may encompass 1-5 GS repeating units.
GSGSGSGSGS

SEQ ID NO: 13 - protein, 15 aa, synthetic construct
  VARIANT 1..15: note = This region may encompass 1-5 GGS repeating units.
GGSGGSGGSGGSGGS

SEQ ID NO: 14 - protein, 20 aa, synthetic construct
  VARIANT 1..20: note = This region may encompass 1-5 GGGS repeating units.
GGGSGGGSGGGSGGGSGGGS

SEQ ID NO: 15 - protein, 25 aa, synthetic construct
  VARIANT 1..25: note = This region may encompass 1-5 GGGGS repeating units
GGGGSGGGGSGGGGSGGGGSGGGGS

SEQ ID NO: 16 - protein, 5 aa, synthetic construct
GGGGS

SEQ ID NO: 17 - protein, 10 aa, synthetic construct
GGGGSGGGGS

SEQ ID NO: 18 - protein, 15 aa, synthetic construct
GGGGSGGGGSGGGGS

SEQ ID NO: 19 - protein, 20 aa, synthetic construct
GGGGSGGGGSGGGGSGGGGS

SEQ ID NO: 20-21 - skipped (2 entries)

SEQ ID NO: 22 - protein, 5 aa, synthetic construct
GGGGG

SEQ ID NO: 23 - skipped

SEQ ID NO: 24 - protein, 4 aa, synthetic construct
GGGS

SEQ ID NO: 25 - protein, 7 aa, synthetic construct
GGGGSGG

SEQ ID NO: 26 - protein, 6 aa, synthetic construct
GGSGGS

SEQ ID NO: 27 - protein, 5 aa, synthetic construct
GGGGG

SEQ ID NO: 28 - protein, 20 aa, synthetic construct
GSGSGSGSGSGSGSGSGSGS

SEQ ID NO: 29 - protein, 5 aa, synthetic construct
  VARIANT 1..5: note = This region may encompass 1-5 G repeating units.
GGGGG

SEQ ID NO: 30 - protein, 4 aa, synthetic construct
  VARIANT 1..4: note = This region may encompass 1-4 G repeating units.
GGGG

SEQ ID NO: 31 - protein, 20 aa, synthetic construct
  VARIANT 1..20: note = This region may encompass 1-20 P repeating units.
PPPPPPPPPPPPPPPPPPPP

SEQ ID NO: 32 - protein, 40 aa, synthetic construct
  VARIANT 1..40: note = This region may encompass 1-20 PA repeating units.
PAPAPAPAPAPAPAPAPAPAPAPAPAPAPAPAPAPAPAPA

SEQ ID NO: 33 - protein, 10 aa, synthetic construct
  VARIANT 1..10: note = This region may encompass 1-10 P repeating units.
PPPPPPPPPP

SEQ ID NO: 34 - protein, 20 aa, synthetic construct
  VARIANT 1..20: note = This region may ecompass 1-10 PA repeating units.
PAPAPAPAPAPAPAPAPAPA

SEQ ID NO: 35 - protein, 5 aa, synthetic construct
  VARIANT 1..5: note = This region may encompass 1-5 P repeating units.
PPPPP

SEQ ID NO: 36 - protein, 10 aa, synthetic construct
  VARIANT 1..10: note = This region may encompass 1-5 PA repeating units.
PAPAPAPAPA

SEQ ID NO: 37 - protein, 10 aa, synthetic construct
PAPAPAPAPA

SEQ ID NO: 38 - protein, 12 aa, synthetic construct
PAPAPAPAPAPA

SEQ ID NO: 39 - protein, 14 aa, synthetic construct
PAPAPAPAPAPAPA

SEQ ID NO: 40 - protein, 16 aa, synthetic construct
PAPAPAPAPAPAPAPA

SEQ ID NO: 41 - protein, 18 aa, synthetic construct
PAPAPAPAPAPAPAPAPA

SEQ ID NO: 42 - protein, 20 aa, synthetic construct
PAPAPAPAPAPAPAPAPAPA

SEQ ID NO: 43 - protein, 20 aa, synthetic construct
YPCWLARVGELCDLDSGDVH

SEQ ID NO: 44 - protein, 20 aa, synthetic construct
APCWLARVGELCDLDSGDVH

SEQ ID NO: 45 - protein, 20 aa, synthetic construct
APCALARVGELCDLDSGDVH

SEQ ID NO: 46 - protein, 20 aa, synthetic construct
APCALAAVGELCDLDSGDVH

SEQ ID NO: 47 - protein, 20 aa, synthetic construct
APCALAAVGALCDLDSGDVH

SEQ ID NO: 48 - protein, 20 aa, synthetic construct
APCALAAVGALCDLASGDVH

SEQ ID NO: 49 - protein, 20 aa, synthetic construct
APCALAAVGALCDLAAGDVH

SEQ ID NO: 50 - protein, 14 aa, synthetic construct
YWCWMAQVGELCDL

SEQ ID NO: 51 - protein, 14 aa, synthetic construct
YHCWMAQVGELCDL

SEQ ID NO: 52 - protein, 14 aa, synthetic construct
YHCWMGQVGELCDL

SEQ ID NO: 53 - protein, 14 aa, synthetic construct
YHCWMGQMGELCDL

SEQ ID NO: 54 - protein, 14 aa, synthetic construct
YHCWMGQMGELCEL

SEQ ID NO: 55 - protein, 14 aa, synthetic construct
YHCWMGQMGELCEM

SEQ ID NO: 56 - protein, 20 aa, synthetic construct
MGFYPCWTAQLGELCDLSVD

SEQ ID NO: 57 - protein, 19 aa, synthetic construct
GFYPCWTAQLGELCDLSVD

SEQ ID NO: 58 - protein, 18 aa, synthetic construct
FYPCWTAQLGELCDLSVD

SEQ ID NO: 59 - protein, 17 aa, synthetic construct
YPCWTAQLGELCDLSVD

SEQ ID NO: 60 - protein, 19 aa, synthetic construct
MGFYPCWTAQLGELCDLSV

SEQ ID NO: 61 - protein, 18 aa, synthetic construct
MGFYPCWTAQLGELCDLS

SEQ ID NO: 62 - protein, 17 aa, synthetic construct
MGFYPCWTAQLGELCDL

SEQ ID NO: 63 - protein, 18 aa, synthetic construct
GFYPCWTAQLGELCDLSV

SEQ ID NO: 64 - protein, 15 aa, synthetic construct
FYPCWTAQLGELCDL

SEQ ID NO: 65 - protein, 14 aa, synthetic construct
KYCGFAQLGELCVL

SEQ ID NO: 66 - protein, 15 aa, synthetic construct
GKYCGFAQLGELCVL

SEQ ID NO: 67 - protein, 16 aa, synthetic construct
GGKYCGFAQLGELCVL

SEQ ID NO: 68 - protein, 17 aa, synthetic construct
GGGKYCGFAQLGELCVL

SEQ ID NO: 69 - protein, 15 aa, synthetic construct
KYCGFAQLGELCVLG

SEQ ID NO: 70 - protein, 16 aa, synthetic construct
KYCGFAQLGELCVLGG

SEQ ID NO: 71 - protein, 17 aa, synthetic construct
KYCGFAQLGELCVLGGG

SEQ ID NO: 72 - protein, 16 aa, synthetic construct
GKYCGFAQLGELCVLG

SEQ ID NO: 73 - protein, 17 aa, synthetic construct
GGKYCGFAQLGELCVLG

SEQ ID NO: 74-78 - skipped (5 entries)

SEQ ID NO: 79 - protein, 20 aa, synthetic construct
QPCWLAQVGDLCDLLWPGPL

SEQ ID NO: 80 - protein, 16 aa, synthetic construct
WLPCWIARLGDLCDLE

SEQ ID NO: 81 - protein, 16 aa, synthetic construct
WYPCWMALLGELCDQE

SEQ ID NO: 82 - protein, 16 aa, synthetic construct
WYPCYRARLGELCDLD

SEQ ID NO: 83 - protein, 22 aa, synthetic construct
WQREWRWFPCWMAKLGDMCDLD

SEQ ID NO: 84 - protein, 22 aa, synthetic construct
QDEAVEWFPCWMARLGELCDLE

SEQ ID NO: 85 - protein, 16 aa, synthetic construct
YYPCWMARLGELCDLD

SEQ ID NO: 86 - protein, 22 aa, synthetic construct
SVVVNNWLPCWMAQLGELCDLD

SEQ ID NO: 87 - protein, 16 aa, synthetic construct
WYPCWLAQLGDLCDLD

SEQ ID NO: 88 - protein, 22 aa, synthetic construct
VMSPTRWLPCWIAKLGELCDLE

SEQ ID NO: 89 - protein, 16 aa, synthetic construct
WFPCWMAQLGQLCDLE

SEQ ID NO: 90 - protein, 22 aa, synthetic construct
WRPCWRAYLGELCDLEAMPRAT

SEQ ID NO: 91 - protein, 22 aa, synthetic construct
IRSCSPCWSADVGELCDLECEW

SEQ ID NO: 92 - protein, 22 aa, synthetic construct
SGHWYPCWMARLGELCDMEERA

SEQ ID NO: 93 - protein, 22 aa, synthetic construct
WYPCWMAQLGELCDLQTMGYSH

SEQ ID NO: 94 - protein, 22 aa, synthetic construct
AGDWLPCWMAELGELCDLEGPT

SEQ ID NO: 95 - protein, 22 aa, synthetic construct
WLPCWIASLGELCDLDTGKRQG

SEQ ID NO: 96 - protein, 22 aa, synthetic construct
WLPCWMAHLGQLCDLDLPGKSM

SEQ ID NO: 97 - protein, 22 aa, synthetic construct
EGVFFPCWIARLGELCDLDHGL

SEQ ID NO: 98 - protein, 22 aa, synthetic construct
TGRWKPCWMAGLHELCDLEGFR

SEQ ID NO: 99 - protein, 22 aa, synthetic construct
RKHFYPCWMAQLGELCDLEGMP

SEQ ID NO: 100 - protein, 22 aa, synthetic construct
DIGYYPCWMAQVGDLCDLDDEK

SEQ ID NO: 101 - protein, 22 aa, synthetic construct
DSDWWPCWMAQLGELCDLEDAR

SEQ ID NO: 102 - protein, 22 aa, synthetic construct
GERWKPCWIAQLGELCDLDFNW

SEQ ID NO: 103 - protein, 22 aa, synthetic construct
WWPCWMAQLGEMCDLEYPYVPG

SEQ ID NO: 104 - protein, 22 aa, synthetic construct
QTKLEGWYPCWMAQLGELCDLD

SEQ ID NO: 105 - protein, 22 aa, synthetic construct
WGRKEQWLPCWKAQLGELCDLE

SEQ ID NO: 106 - protein, 22 aa, synthetic construct
VPRANAWHPCWMAQLGELCDLE

SEQ ID NO: 107 - protein, 22 aa, synthetic construct
GRQQKGWYPCWLAQLGELCDME

SEQ ID NO: 108 - protein, 22 aa, synthetic construct
WLNRHLFNPCWMARLGELCDLE

SEQ ID NO: 109 - protein, 22 aa, synthetic construct
AQVRREWYPCWMAQLGELCDLT

SEQ ID NO: 110 - protein, 22 aa, synthetic construct
ETEQMSWYPCWVAQLWELCDLD

SEQ ID NO: 111 - protein, 21 aa, synthetic construct
WLPCWLAKLGELCDLEWLPCW

SEQ ID NO: 112 - protein, 22 aa, synthetic construct
ERRPDTWFPCWRALVGELCDLE

SEQ ID NO: 113 - protein, 22 aa, synthetic construct
WGRNRSWYPCWMAQLGELCDLE

SEQ ID NO: 114 - protein, 22 aa, synthetic construct
QDRRSPWYPCWMAKLGELCDLA

SEQ ID NO: 115 - protein, 24 aa, synthetic construct
TRRWYPCYLAKLGELCDLFEGGTR

SEQ ID NO: 116 - protein, 24 aa, synthetic construct
SEQWWPCWIARLGELCDLDRELSE

SEQ ID NO: 117 - protein, 25 aa, synthetic construct
WYPCWVAQLGEICDLEMTGPDSWYP

SEQ ID NO: 118 - protein, 22 aa, synthetic construct
QDGWLPCWMAQLGELCDLEYKR

SEQ ID NO: 119 - protein, 22 aa, synthetic construct
NRRWYPCWMAQLGELCDLDSRP

SEQ ID NO: 120 - protein, 22 aa, synthetic construct
FYPCWMAHLGELCDLDGDTDSM

SEQ ID NO: 121 - protein, 22 aa, synthetic construct
KSNFFPCWIAQLGQLCDLEPET

SEQ ID NO: 122 - protein, 22 aa, synthetic construct
FYPCWMANLGELCDLDFLRELN

SEQ ID NO: 123 - protein, 22 aa, synthetic construct
HASWLPCWLAQLGELCDLEPNP

SEQ ID NO: 124 - protein, 22 aa, synthetic construct
NGAWYPCWMAQVGELCDLEERW

SEQ ID NO: 125 - protein, 22 aa, synthetic construct
WRRWYPCWVAQVGELCDLEIEA

SEQ ID NO: 126 - protein, 22 aa, synthetic construct
RQAWYPCWMAQLGELCDLEAEL

SEQ ID NO: 127 - protein, 22 aa, synthetic construct
RQRWYPCWMARLGELCDLDEPT

SEQ ID NO: 128 - protein, 22 aa, synthetic construct
NNSREGWFPCWLAKLGDLCDLD

SEQ ID NO: 129 - protein, 16 aa, synthetic construct
YYPCWMAQLGELCDLE

SEQ ID NO: 130 - protein, 16 aa, synthetic construct
WYPCWLAQLGELCDLD

SEQ ID NO: 131 - protein, 22 aa, synthetic construct
SWHAETWYPCWLAQVGELCDLD

SEQ ID NO: 132 - protein, 22 aa, synthetic construct
KMHKAVWLPCWMAQVGELCDLE

SEQ ID NO: 133 - protein, 22 aa, synthetic construct
DVLGDRWYPCWIAKLGELCDLD

SEQ ID NO: 134 - protein, 16 aa, synthetic construct
WYPCWMAQLGELCDLD

SEQ ID NO: 135 - protein, 22 aa, synthetic construct
KLQSWRWYPCWMAQLGELCDLD

SEQ ID NO: 136 - protein, 22 aa, synthetic construct
NEPEGGFYPCWLAQLGELCDLH

SEQ ID NO: 137 - protein, 16 aa, synthetic construct
WYPCWMARLGELCDLE

SEQ ID NO: 138 - protein, 22 aa, synthetic construct
FYPCWTALLGELCDLEPGPPAM

SEQ ID NO: 139 - protein, 22 aa, synthetic construct
WGTTWRWYPCWMAQLGELCDLE

SEQ ID NO: 140 - protein, 22 aa, synthetic construct
AKGWDTWKPCWLANLGELCDLE

SEQ ID NO: 141 - protein, 22 aa, synthetic construct
RDESAGYYPCWIAQLGELCDLE

SEQ ID NO: 142 - protein, 16 aa, synthetic construct
WYPCWIAKLGELCDLE

SEQ ID NO: 143 - protein, 16 aa, synthetic construct
WYPCWIAQLGELCDLD

SEQ ID NO: 144 - protein, 16 aa, synthetic construct
WYPCWLAKLGELCDLD

SEQ ID NO: 145 - protein, 22 aa, synthetic construct
QGPVRLWYPCWMAQLGELCDLD

SEQ ID NO: 146 - protein, 16 aa, synthetic construct
WYPCWMAQPGELCDVD

SEQ ID NO: 147 - protein, 16 aa, synthetic construct
WHPCWIAQLGELCDLE

SEQ ID NO: 148 - protein, 16 aa, synthetic construct
WYPCWIAQLGELCDLE

SEQ ID NO: 149 - protein, 22 aa, synthetic construct
VRPMGVWYPCWIAQLGELCDLV

SEQ ID NO: 150 - protein, 22 aa, synthetic construct
VPRWYPCWIAQLGELCDLDSDD

SEQ ID NO: 151 - protein, 22 aa, synthetic construct
YRGWLPCWRAKLGDLCDLGQPM

SEQ ID NO: 152 - protein, 22 aa, synthetic construct
GEAWYPCWLARLGELCDMDPRV

SEQ ID NO: 153 - protein, 22 aa, synthetic construct
WYPCWMAQLGELCDLDESTRLT

SEQ ID NO: 154 - protein, 22 aa, synthetic construct
IGSWWPCWMAQLGELCDLEPEL

SEQ ID NO: 155 - protein, 22 aa, synthetic construct
GTTWYPCWLAQLGELCDLDVLE

SEQ ID NO: 156 - protein, 22 aa, synthetic construct
WWPCWMAQLGDLCDLEETSGGT

SEQ ID NO: 157 - protein, 22 aa, synthetic construct
WYPCWMAQLGELCDLGPTESNL

SEQ ID NO: 158 - protein, 22 aa, synthetic construct
WYPCWMANLGELCDLEYPSWAQ

SEQ ID NO: 159 - protein, 22 aa, synthetic construct
RGMCYPCWFARLGELCDLECDQ

SEQ ID NO: 160 - protein, 22 aa, synthetic construct
WYPCWMAQLGELCDLDAGARHL

SEQ ID NO: 161 - protein, 22 aa, synthetic construct
KSGWYPCWMAKLGELCDLEAQP

SEQ ID NO: 162 - protein, 22 aa, synthetic construct
GPRFYPCWIAQLGELCDLEDMG

SEQ ID NO: 163 - protein, 22 aa, synthetic construct
RVTWYPCWMAQLGELCDLEESV

SEQ ID NO: 164 - protein, 22 aa, synthetic construct
WLPCWMAQLGDLCDLEQYVPLP

SEQ ID NO: 165 - protein, 22 aa, synthetic construct
YLPCWMAHLGELCDLDSPLKAR

SEQ ID NO: 166 - protein, 22 aa, synthetic construct
WYPCWMAQLGELCDLDDHWPAM

SEQ ID NO: 167 - protein, 22 aa, synthetic construct
WYPCWRAQLGELCDLDPPIAVE

SEQ ID NO: 168 - protein, 22 aa, synthetic construct
WYPCWMANLGELCDLEAERSPV

SEQ ID NO: 169 - protein, 22 aa, synthetic construct
RDQYYPCWMAQLGELCDLDEVF

SEQ ID NO: 170 - protein, 22 aa, synthetic construct
WYPCWMAQLGDLCDLEKPVTER

SEQ ID NO: 171 - protein, 22 aa, synthetic construct
WYPCWIARLGELCDLETSGGFP

SEQ ID NO: 172 - protein, 21 aa, synthetic construct
SGHCYPCWLAGLGELCDLNCG

SEQ ID NO: 173-177 - skipped (5 entries)

SEQ ID NO: 178 - protein, 21 aa, synthetic construct
VRAWYPCWIARLGELCDLEVD

SEQ ID NO: 179 - protein, 16 aa, synthetic construct
WFPCWMAQLGEVCDLD

SEQ ID NO: 180 - protein, 16 aa, synthetic construct
WYPCHMAKLRELCDLD

SEQ ID NO: 181 - protein, 16 aa, synthetic construct
SLPCWMAQLGDLCELH

SEQ ID NO: 182 - protein, 16 aa, synthetic construct
SNPCWMAQLWELCDLE

SEQ ID NO: 183 - protein, 16 aa, synthetic construct
YKPCWIAQLGELCELE

SEQ ID NO: 184 - protein, 16 aa, synthetic construct
WQPCWMAQLGELCDLE

SEQ ID NO: 185 - protein, 16 aa, synthetic construct
WKPCWLAQLGDLCEME

SEQ ID NO: 186 - protein, 16 aa, synthetic construct
WLPCWMAKLGDLCELE

SEQ ID NO: 187 - protein, 16 aa, synthetic construct
WLPCWMAQLGELCVME

SEQ ID NO: 188 - protein, 16 aa, synthetic construct
LHPCWMAQLGELCDLE

SEQ ID NO: 189 - protein, 16 aa, synthetic construct
SLPCWMAQLGELCDLV

SEQ ID NO: 190 - protein, 16 aa, synthetic construct
WKPCWMAGLGELCELD

SEQ ID NO: 191 - protein, 16 aa, synthetic construct
WWPCSSAELGEICDFD

SEQ ID NO: 192 - protein, 16 aa, synthetic construct
WQPCWRAKLGELCDLE

SEQ ID NO: 193 - protein, 16 aa, synthetic construct
WSPCWIATLGELCDLD

SEQ ID NO: 194 - protein, 16 aa, synthetic construct
WNPCWIAQLGDLCDMV

SEQ ID NO: 195 - protein, 16 aa, synthetic construct
WLPCWLAHLGDICDLQ

SEQ ID NO: 196 - protein, 22 aa, synthetic construct
WLSPPSYLPCWMAQLGELCDLV

SEQ ID NO: 197 - protein, 22 aa, synthetic construct
WDVHAIRNPCWLAKLGDLCDLD

SEQ ID NO: 198 - protein, 22 aa, synthetic construct
GAGVLHRWPCWMAKLGDLCDLD

SEQ ID NO: 199 - protein, 22 aa, synthetic construct
DTGRDGWKPCWMALLGELCELE

SEQ ID NO: 200 - protein, 22 aa, synthetic construct
EQGMLGYFPCWKALLGDVCDLD

SEQ ID NO: 201 - protein, 22 aa, synthetic construct
WRVTASLQPCWMAQLGELCDLN

SEQ ID NO: 202 - protein, 22 aa, synthetic construct
GQVVETSLPCWEAQLGELCVLD

SEQ ID NO: 203 - protein, 22 aa, synthetic construct
TVGQFEWYPCSTAQLGELCDLD

SEQ ID NO: 204 - protein, 22 aa, synthetic construct
ALVGGTFYPCYVAHLGELCDIE

SEQ ID NO: 205 - protein, 22 aa, synthetic construct
IDRADGWKPCWIAQVGELCVLE

SEQ ID NO: 206 - protein, 22 aa, synthetic construct
YRRERVEFPCWLAQLGELCDKE

SEQ ID NO: 207 - protein, 22 aa, synthetic construct
AVSHGNWLPCYIAQLGELCDLD

SEQ ID NO: 208 - protein, 22 aa, synthetic construct
IPKGESWFPCWMAAMGELCDLE

SEQ ID NO: 209 - protein, 22 aa, synthetic construct
WYPCWIAQLGEVCDLEKQTGSV

SEQ ID NO: 210 - protein, 22 aa, synthetic construct
WYPCWMAHLGDVCDLESFGQTE

SEQ ID NO: 211 - protein, 22 aa, synthetic construct
YKPCQMAQLGELCDLDVDNKAE

SEQ ID NO: 212 - protein, 22 aa, synthetic construct
FKPCWIANLGELCDMDDERSSE

SEQ ID NO: 213 - protein, 22 aa, synthetic construct
WKPCWMARLGELCDIEDTKVNA

SEQ ID NO: 214 - protein, 22 aa, synthetic construct
SLPCWIARLGELCDLDGYDGEE

SEQ ID NO: 215 - protein, 22 aa, synthetic construct
FYPCWKARLGELCELEELRGYY

SEQ ID NO: 216 - protein, 22 aa, synthetic construct
WKPCWIADLGELCDLAPAWHEY

SEQ ID NO: 217 - protein, 22 aa, synthetic construct
RLPCWRAQLGDLCELDWGLDMG

SEQ ID NO: 218 - protein, 22 aa, synthetic construct
SKPCWMAQLGELCDLDVWNLQM

SEQ ID NO: 219 - protein, 22 aa, synthetic construct
GYPCWLAQLGDYCDLDAGAPSW

SEQ ID NO: 220 - protein, 22 aa, synthetic construct
SWPCWMAQLGDLCDLDGSAGAS

SEQ ID NO: 221 - protein, 22 aa, synthetic construct
WKPCWLAQLGELCDLERPSTTS

SEQ ID NO: 222 - protein, 22 aa, synthetic construct
WYSCGKAQLGELCDLDVESQPG

SEQ ID NO: 223 - protein, 22 aa, synthetic construct
YVPCYMARLGELCELEANRPGQ

SEQ ID NO: 224 - protein, 22 aa, synthetic construct
SKPCWLAQLGDLCDFDWTAADH

SEQ ID NO: 225 - protein, 22 aa, synthetic construct
WFPCWMAQLGDLCELEPDSVPA

SEQ ID NO: 226 - protein, 22 aa, synthetic construct
WTPCWIAHLGDLCDLEPQDDTD

SEQ ID NO: 227 - protein, 22 aa, synthetic construct
WKPCFIASLGELCDLDQGSVEV

SEQ ID NO: 228 - protein, 22 aa, synthetic construct
WKPCWMAALGELCDLERSVGKV

SEQ ID NO: 229 - protein, 22 aa, synthetic construct
WKPCWRAQLGELCDLELGPSER

SEQ ID NO: 230 - protein, 22 aa, synthetic construct
FFPCWMGQLGDLCDLEVRSMQK

SEQ ID NO: 231 - protein, 22 aa, synthetic construct
YWPCSMASLGELCDLEWQGRLP

SEQ ID NO: 232 - protein, 22 aa, synthetic construct
WYPCYMASLGELCDLQSSISPR

SEQ ID NO: 233 - protein, 22 aa, synthetic construct
WYPCWMAQLGELCDHEWPSYGA

SEQ ID NO: 234 - protein, 22 aa, synthetic construct
MGSWLPCWMAQLGDLCDVEGGM

SEQ ID NO: 235 - protein, 22 aa, synthetic construct
QKGFLPCWRAQLGQLCDMESQY

SEQ ID NO: 236 - protein, 22 aa, synthetic construct
GSGWQPCWMADLGELCDLDNEK

SEQ ID NO: 237 - protein, 22 aa, synthetic construct
WRRWYPCWMAQLGELCDLDQWT

SEQ ID NO: 238 - protein, 22 aa, synthetic construct
RRSWYPCRIAQLGELCDLDPRV

SEQ ID NO: 239 - protein, 22 aa, synthetic construct
AYRIYPCWKAQLGELCDLDNAD

SEQ ID NO: 240 - protein, 22 aa, synthetic construct
QRNSFPCWLAQLGDLCDLGDWA

SEQ ID NO: 241 - protein, 22 aa, synthetic construct
QPAWLPCWLAQLGELCDLGTGA

SEQ ID NO: 242 - protein, 22 aa, synthetic construct
SRFWQPCWMAQLGELCHLDPQM

SEQ ID NO: 243 - protein, 22 aa, synthetic construct
YLNFNPCWTAQLGELCDLASGE

SEQ ID NO: 244 - protein, 22 aa, synthetic construct
STGWYPCWIAEFGELCDLVKPH

SEQ ID NO: 245 - protein, 22 aa, synthetic construct
AHWSQPCWTAQLGELCDLDMGD

SEQ ID NO: 246 - protein, 22 aa, synthetic construct
HPVRYPCWVAQLGELCDLENGN

SEQ ID NO: 247 - protein, 22 aa, synthetic construct
QTGSYPCWIAHLGELCDLEGSA

SEQ ID NO: 248 - protein, 21 aa, synthetic construct
TGWWYPCWMAQLGELCDLQQT

SEQ ID NO: 249 - protein, 19 aa, synthetic construct
DLWQPCWMARLGELCDLKG

SEQ ID NO: 250 - protein, 20 aa, synthetic construct
MGTGWQNYCRYAQLGELCLL

SEQ ID NO: 251 - protein, 16 aa, synthetic construct
WYPCGVAQPGDLCDLE

SEQ ID NO: 252 - protein, 22 aa, synthetic construct
MLGEWLCEMDQLGYLCYLDHGD

SEQ ID NO: 253 - protein, 20 aa, synthetic construct
WDGWECGMDHDGWVCEFWGE

SEQ ID NO: 254-336 - skipped (83 entries)

SEQ ID NO: 337 - protein, 22 aa, synthetic construct
WYPCWIAQLGELCDLDAKGQRR

SEQ ID NO: 338 - protein, 22 aa, synthetic construct
TNNFYPCWLAKLGDLCDFDDLN

SEQ ID NO: 339 - protein, 22 aa, synthetic construct
WYPCWIARVGELCDLEEGPVNR

SEQ ID NO: 340 - protein, 21 aa, synthetic construct
AVEFYPCWLARIGELCDLVEP

SEQ ID NO: 341 - protein, 16 aa, synthetic construct
WYPCWIAHLGELCDLE

SEQ ID NO: 342 - protein, 16 aa, synthetic construct
WYPCWIARVGELCDME

SEQ ID NO: 343 - protein, 21 aa, synthetic construct
RREWYPCWIAQVGELCDLPLI

SEQ ID NO: 344 - protein, 22 aa, synthetic construct
RWPWYPCEIARIGELCDLEQAN

SEQ ID NO: 345 - protein, 22 aa, synthetic construct
FGAFYPCWKAQLGELCDLEPVT

SEQ ID NO: 346 - protein, 22 aa, synthetic construct
HGRWFPCWMAQVGDLCDLEHSN

SEQ ID NO: 347 - protein, 22 aa, synthetic construct
WYPCWLAKLGELCDLDRAEALP

SEQ ID NO: 348 - protein, 20 aa, synthetic construct
VWFPCWFAQLGDLCDLDQDP

SEQ ID NO: 349 - protein, 22 aa, synthetic construct
ETLGSVWYPCWIASIGELCDLD

SEQ ID NO: 350 - protein, 22 aa, synthetic construct
WYSCWIAQLGELCDLDDMGDRV

SEQ ID NO: 351 - protein, 16 aa, synthetic construct
WFPCWLAQLGELCDKE

SEQ ID NO: 352 - protein, 16 aa, synthetic construct
WYPCWIAQLGELCDLV

SEQ ID NO: 353 - protein, 22 aa, synthetic construct
SHLWFPCWMAQLGELCDLEGGP

SEQ ID NO: 354 - protein, 21 aa, synthetic construct
RRWLPCWMAHVGELCDLELGN

SEQ ID NO: 355 - protein, 21 aa, synthetic construct
YWCWFARVGELCDLDDGGVPS

SEQ ID NO: 356 - protein, 16 aa, synthetic construct
WYSCWIAQIGELCDLE

SEQ ID NO: 357 - protein, 22 aa, synthetic construct
MNPVGTFYPCWIAKLGELCDLQ

SEQ ID NO: 358 - protein, 22 aa, synthetic construct
HGGVIGWYPCWMAKVGELCDLD

SEQ ID NO: 359 - protein, 22 aa, synthetic construct
WYPCSIALLGELCDLESVHEKS

SEQ ID NO: 360 - protein, 22 aa, synthetic construct
EQRNRQWWPCWLARLGDLCDLD

SEQ ID NO: 361 - protein, 16 aa, synthetic construct
WYPCWLAQLGELCDLD

SEQ ID NO: 362 - protein, 16 aa, synthetic construct
WLPCWMAHLGDLCDLE

SEQ ID NO: 363 - protein, 22 aa, synthetic construct
RPSWAPCWLAQVGELCDLDHPE

SEQ ID NO: 364 - protein, 22 aa, synthetic construct
ESKEPAFWPCWMAQLGDLCDLE

SEQ ID NO: 365 - protein, 22 aa, synthetic construct
RSLRESWFPCWMAKLGDLCDLE

SEQ ID NO: 366 - protein, 16 aa, synthetic construct
WHPCWLARVGDLCDLD

SEQ ID NO: 367 - protein, 22 aa, synthetic construct
SRSAGEYYPCWLAQLGELCDLA

SEQ ID NO: 368 - protein, 22 aa, synthetic construct
RQVPKQWYPCWMAALGELCDLE

SEQ ID NO: 369 - protein, 16 aa, synthetic construct
WYPCWLAHLGELCDLE

SEQ ID NO: 370 - protein, 21 aa, synthetic construct
QVMRLWYPCWMAQLGELCDLE

SEQ ID NO: 371 - protein, 22 aa, synthetic construct
WYPCWMAQLGELCDLDEAPVQP

SEQ ID NO: 372 - protein, 22 aa, synthetic construct
RDIWWPCWVAQLGELCDLDDPQ

SEQ ID NO: 373 - protein, 22 aa, synthetic construct
SATWYPCFLANLGELCDLEQEN

SEQ ID NO: 374 - protein, 22 aa, synthetic construct
YAEWYPCWMARVGEVCDLEVTP

SEQ ID NO: 375 - protein, 22 aa, synthetic construct
RSGDKAFFPCWLAQLGDLCDLD

SEQ ID NO: 376 - protein, 16 aa, synthetic construct
WYPCWMAQLGELCDMD

SEQ ID NO: 377 - protein, 21 aa, synthetic construct
RVSYPCWLARLGELCDMDLEE

SEQ ID NO: 378 - protein, 22 aa, synthetic construct
TESWYPCWLANLGDLCDLEWSA

SEQ ID NO: 379 - protein, 16 aa, synthetic construct
WLPCWMADVGDLCDLD

SEQ ID NO: 380 - protein, 16 aa, synthetic construct
WHPCWMARLGELCDLD

SEQ ID NO: 381 - protein, 22 aa, synthetic construct
QGTKWHWNPCWMAQLGELCDLD

SEQ ID NO: 382 - protein, 22 aa, synthetic construct
KNGPKSWYPCWMAQVGDLCDLD

SEQ ID NO: 383 - protein, 22 aa, synthetic construct
SGTGPAWYPCFLASLGQLCDLE

SEQ ID NO: 384 - protein, 16 aa, synthetic construct
WYPCWMARMGELCDLE

SEQ ID NO: 385 - protein, 16 aa, synthetic construct
WLPCWRAQLGQLCDLD

SEQ ID NO: 386 - protein, 16 aa, synthetic construct
LFPCWLAQLGELCDLE

SEQ ID NO: 387 - protein, 16 aa, synthetic construct
RYPCWIAQLGELCDLD

SEQ ID NO: 388 - protein, 16 aa, synthetic construct
WHPCWIAHLGELCDLE

SEQ ID NO: 389 - protein, 16 aa, synthetic construct
MYPCWIAHLGELCDLD

SEQ ID NO: 390 - protein, 16 aa, synthetic construct
WYPCSIASLGELCDLE

SEQ ID NO: 391 - protein, 16 aa, synthetic construct
WLPCYMAQLGDLCDLE

SEQ ID NO: 392 - protein, 16 aa, synthetic construct
WHPCWMAQVGEVCDLD

SEQ ID NO: 393 - protein, 16 aa, synthetic construct
WYPCWLASLGEVCDLE

SEQ ID NO: 394 - protein, 16 aa, synthetic construct
WWPCSIARLGQLCDLD

SEQ ID NO: 395 - protein, 16 aa, synthetic construct
WYPCWLAHLGELCDLA

SEQ ID NO: 396 - protein, 16 aa, synthetic construct
WYPCWLAQLGELCDAE

SEQ ID NO: 397 - protein, 16 aa, synthetic construct
WKPCWMALLGELCDLE

SEQ ID NO: 398 - protein, 16 aa, synthetic construct
RYPCWRAKLGELCDLD

SEQ ID NO: 399 - protein, 22 aa, synthetic construct
EEQSRGFLPCWMALLGELCDLD

SEQ ID NO: 400 - protein, 22 aa, synthetic construct
LGSKRQWYPCWVAHLGELCDLE

SEQ ID NO: 401 - protein, 22 aa, synthetic construct
ESEGRGWYPCWNALLGELCDLE

SEQ ID NO: 402 - protein, 22 aa, synthetic construct
RWTQAQWYPCWLAQLGELCDLE

SEQ ID NO: 403 - protein, 21 aa, synthetic construct
LHAGRWNPCWLAQLGELCDLE

SEQ ID NO: 404 - protein, 21 aa, synthetic construct
LSSKGWYPCWKARLGDLCDLE

SEQ ID NO: 405 - protein, 22 aa, synthetic construct
DMFTHRWYPCSMAKLGELCDLE

SEQ ID NO: 406 - protein, 22 aa, synthetic construct
MTDRAFWNPCWVARLGELCDLD

SEQ ID NO: 407 - protein, 22 aa, synthetic construct
NVTYTQWFPCWLARLGELCDLV

SEQ ID NO: 408 - protein, 21 aa, synthetic construct
VRTRIWYPCWSAQLGELCDLD

SEQ ID NO: 409 - protein, 21 aa, synthetic construct
AMARRYLPCWIAKLGELCELD

SEQ ID NO: 410 - protein, 22 aa, synthetic construct
ARGEYRWFPCWMARLGELCDLE

SEQ ID NO: 411 - protein, 22 aa, synthetic construct
YLERSRWYPCFIAQLGELCDLE

SEQ ID NO: 412 - protein, 22 aa, synthetic construct
FRVSRDWFPCWMAQLGEVCDLE

SEQ ID NO: 413 - protein, 22 aa, synthetic construct
IERAWEWRPCWLASVGELCDLE

SEQ ID NO: 414 - protein, 21 aa, synthetic construct
VASERFYPCWIARLGELCDVE

SEQ ID NO: 415 - protein, 22 aa, synthetic construct
WYPCWIAKLGEVCDLDQGTTRQ

SEQ ID NO: 416 - protein, 22 aa, synthetic construct
WYPCWLAHLGELCDLDWKGRND

SEQ ID NO: 417 - protein, 22 aa, synthetic construct
WYPCWRAQLGELCDLVDLGSHL

SEQ ID NO: 418 - protein, 22 aa, synthetic construct
WSPCWMASLGDLCDLEETRQTE

SEQ ID NO: 419 - protein, 22 aa, synthetic construct
WTPCWIAQLGELCDLEGRHGTV

SEQ ID NO: 420 - protein, 21 aa, synthetic construct
LPCWIAQLGDLCDLEPEPSPE

SEQ ID NO: 421 - protein, 22 aa, synthetic construct
FYPCWAAHLGDLCDLEYQEAGP

SEQ ID NO: 422 - protein, 22 aa, synthetic construct
WLPCWLAPLGDLCDMDMSAVMN

SEQ ID NO: 423 - protein, 22 aa, synthetic construct
WRPCWMAHLGDLCDLEMANENP

SEQ ID NO: 424 - protein, 22 aa, synthetic construct
WYPCWLAQLGEVCDLDDGGGVF

SEQ ID NO: 425 - protein, 22 aa, synthetic construct
WWPCWLAQLGELCDLEVNGSLI

SEQ ID NO: 426 - protein, 22 aa, synthetic construct
TEMWYPCWMAYQGELCDLDMTY

SEQ ID NO: 427 - protein, 22 aa, synthetic construct
ARTWWPCWRAKLGELCDLVVPE

SEQ ID NO: 428 - protein, 22 aa, synthetic construct
HQGFYSCRLARLGELCDLDTGW

SEQ ID NO: 429 - protein, 22 aa, synthetic construct
VDEFYPCSMAGLGELCDLERQN

SEQ ID NO: 430 - protein, 22 aa, synthetic construct
AWDWYPCSVAALGEICDLDIQD

SEQ ID NO: 431 - protein, 22 aa, synthetic construct
RPPWYPCWMARLGEVCDMDIML

SEQ ID NO: 432 - protein, 22 aa, synthetic construct
SQRWYPCWVAHLGELCDLEGVV

SEQ ID NO: 433 - protein, 22 aa, synthetic construct
KGSWQPCWFAKLGELCDLHPTS

SEQ ID NO: 434 - protein, 22 aa, synthetic construct
QTWYLPCWMAKLGELCDLGERD

SEQ ID NO: 435 - protein, 22 aa, synthetic construct
EPRWYPCWMAQMGELCDMEMSD

SEQ ID NO: 436 - protein, 22 aa, synthetic construct
WGGRYWCWMAKLGDLCDLEDEW

SEQ ID NO: 437 - protein, 22 aa, synthetic construct
WWPCWIAQVGELCDLDGPGRPT

SEQ ID NO: 438 - protein, 19 aa, synthetic construct
RLVYDCLFAQVGDLCEVIS

SEQ ID NO: 439 - protein, 20 aa, synthetic construct
WRILWMQQCWRSHVVNQCAL

SEQ ID NO: 440 - protein, 22 aa, synthetic construct
WYPCWIAQVGELCDLDEVSHGR

SEQ ID NO: 441 - protein, 22 aa, synthetic construct
TGEWWPCWVAEVGELCDLERGP

SEQ ID NO: 442 - protein, 20 aa, synthetic construct
ARTQGWYDCLFAQVGELCDL

SEQ ID NO: 443 - protein, 22 aa, synthetic construct
FHPCWRALLGELCDLETALGPS

SEQ ID NO: 444 - protein, 20 aa, synthetic construct
LQIRKLWACRIDLVGPFCLL

SEQ ID NO: 445 - protein, 20 aa, synthetic construct
AEYSGRYDCYIAKVGELCDI

SEQ ID NO: 446 - protein, 20 aa, synthetic construct
SWRFLWQDCGRAHVGELCDL

SEQ ID NO: 447 - protein, 22 aa, synthetic construct
NRWWHPCWMARVGELCDLEPDA

SEQ ID NO: 448 - protein, 22 aa, synthetic construct
WWPCWVAKLGELCDLEGDASRV

SEQ ID NO: 449 - protein, 22 aa, synthetic construct
WYPCEFAQLGELCDLLPFPYPA

SEQ ID NO: 450 - protein, 20 aa, synthetic construct
SYMHDCFMAQVGDLCDRFIS

SEQ ID NO: 451 - protein, 22 aa, synthetic construct
WWPCWIAQVGELCDLEEESRES

SEQ ID NO: 452 - protein, 22 aa, synthetic construct
KWAWNPCYIARLGELCDLVEPE

SEQ ID NO: 453 - protein, 22 aa, synthetic construct
WWPCWIADLGELCDLEGPPRGR

SEQ ID NO: 454 - protein, 20 aa, synthetic construct
PTLITWYDCLFAEVGELCDM

SEQ ID NO: 455 - protein, 20 aa, synthetic construct
EISNWFLDCMFADVGDLCDL

SEQ ID NO: 456 - protein, 22 aa, synthetic construct
AQVWYPCWLAKVGELCDLDQWN

SEQ ID NO: 457 - protein, 22 aa, synthetic construct
FGGKMDWYPCWIANLGELCDLK

SEQ ID NO: 458 - protein, 22 aa, synthetic construct
WFPCWMAKVGDLCDVDEHQDPS

SEQ ID NO: 459 - protein, 22 aa, synthetic construct
MGDSSSWFPCWMAQLGELCDME

SEQ ID NO: 460 - protein, 22 aa, synthetic construct
MFRYYPCWIASIGELCDLEWGV

SEQ ID NO: 461 - protein, 22 aa, synthetic construct
ERRWYPCWLASVGELCDLDMGD

SEQ ID NO: 462 - protein, 16 aa, synthetic construct
WYPCWVAQLGELCDLE

SEQ ID NO: 463 - protein, 22 aa, synthetic construct
RWDYWPCYIAQVGELCDLEVYE

SEQ ID NO: 464 - protein, 22 aa, synthetic construct
SLAHRSWYPCWLAQVGELCDLD

SEQ ID NO: 465 - protein, 22 aa, synthetic construct
QNASKGWYPCWIAHVGELCDWD

SEQ ID NO: 466 - protein, 21 aa, synthetic construct
HRWYPCWLAHLGELCDLDPMS

SEQ ID NO: 467 - protein, 16 aa, synthetic construct
FYPCWIAFVGELCDLE

SEQ ID NO: 468 - protein, 20 aa, synthetic construct
EGHWYPCWIAQLGELCDLDW

SEQ ID NO: 469 - protein, 22 aa, synthetic construct
WTGWSAFYPCSIANLGELCDLD

SEQ ID NO: 470 - protein, 22 aa, synthetic construct
WEKLQNWYPCWIAQMGELCDLE

SEQ ID NO: 471 - protein, 22 aa, synthetic construct
TNGVLDWWPCWMAQVGELCDLD

SEQ ID NO: 472 - protein, 16 aa, synthetic construct
WYPCWVAKLGELCDLE

SEQ ID NO: 473 - protein, 19 aa, synthetic construct
AYYPCELAQLGELCDLYNI

SEQ ID NO: 474 - protein, 16 aa, synthetic construct
WYPCWMAHLGELCDLE

SEQ ID NO: 475 - protein, 21 aa, synthetic construct
NDHTAWWPCYFAQVGDLCDLV

SEQ ID NO: 476 - protein, 22 aa, synthetic construct
WWPCEIAQIGELCDLEWVRHAE

SEQ ID NO: 477 - protein, 22 aa, synthetic construct
WWPCDFAQIGELCDLGPRFTGE

SEQ ID NO: 478 - protein, 22 aa, synthetic construct
RDWWLPCEFALIGELCDLERSW

SEQ ID NO: 479 - protein, 20 aa, synthetic construct
MRTTFWYDCYIAQVGELCDF

SEQ ID NO: 480 - protein, 22 aa, synthetic construct
SWHAETWYPCWLAQVGELCDLD

SEQ ID NO: 481 - protein, 20 aa, synthetic construct
EWFHDCFLAKVGDLCDLFLW

SEQ ID NO: 482 - protein, 22 aa, synthetic construct
SGKTQMWNPCYVAKVGELCDLV

SEQ ID NO: 483 - protein, 22 aa, synthetic construct
DKAGPNFYPCWLAHVGELCDQA

SEQ ID NO: 484 - protein, 22 aa, synthetic construct
AGFRGRWWPCEYAQVGELCDLE

SEQ ID NO: 485 - protein, 22 aa, synthetic construct
WFPCWLAKVGELCDRDDLAGPS

SEQ ID NO: 486 - protein, 16 aa, synthetic construct
WWPCEWARIGELCDLE

SEQ ID NO: 487 - protein, 20 aa, synthetic construct
KGSSWFPCYFAQVGDLCDLY

SEQ ID NO: 488 - protein, 16 aa, synthetic construct
WYPCWLAQVGELCDRE

SEQ ID NO: 489 - protein, 21 aa, synthetic construct
RGVYFPCWLAKVGDLCDSDEF

SEQ ID NO: 490 - protein, 22 aa, synthetic construct
RAWWWPCELAQVGELCDLEPSS

SEQ ID NO: 491 - protein, 16 aa, synthetic construct
WYPCWLAKVGELCDQE

SEQ ID NO: 492 - protein, 20 aa, synthetic construct
RYVPDCLKAQVGDLCDFFAW

SEQ ID NO: 493 - protein, 16 aa, synthetic construct
WWPCYLAQIGELCDLV

SEQ ID NO: 494 - protein, 16 aa, synthetic construct
WYPCWMAKVGELCDME

SEQ ID NO: 495 - protein, 22 aa, synthetic construct
QITDSGWYPCWVAKVGELCDMD

SEQ ID NO: 496 - protein, 22 aa, synthetic construct
YRWWYPCDIAQVGELCDLDYLL

SEQ ID NO: 497 - protein, 20 aa, synthetic construct
CYMHDCFMAQVGDLCDRFIS

SEQ ID NO: 498 - protein, 16 aa, synthetic construct
WLPCWIAKIGDLCDLD

SEQ ID NO: 499 - protein, 22 aa, synthetic construct
SRVWHPCWLARVGELCDLEVSD

SEQ ID NO: 500 - protein, 22 aa, synthetic construct
WEHEFTWYPCWIAQVGELCDMD

SEQ ID NO: 501 - protein, 22 aa, synthetic construct
HRGWVGWYPCEYALPGQLCDLE

SEQ ID NO: 502 - protein, 21 aa, synthetic construct
WYPCWLAQLGELCDQDWDTPS

SEQ ID NO: 503 - protein, 22 aa, synthetic construct
RVRRHSWWPCEIAVVGELCDLE

SEQ ID NO: 504 - protein, 21 aa, synthetic construct
DGWWPCWIAQVGELCDLEDPV

SEQ ID NO: 505 - protein, 20 aa, synthetic construct
LPFQDCYIAQVGELCDLPGT

SEQ ID NO: 506 - protein, 20 aa, synthetic construct
RWMFDCLFARVGELCDIRPW

SEQ ID NO: 507 - protein, 20 aa, synthetic construct
GGYYDCLIAEVGELCDMPGQ

SEQ ID NO: 508 - protein, 20 aa, synthetic construct
VVCYACDIAHVGELCDLTCR

SEQ ID NO: 509 - protein, 20 aa, synthetic construct
TPWYDCYIANVGDLCDFASA

SEQ ID NO: 510 - protein, 20 aa, synthetic construct
LESLDCFFARIGDLCEIWDV

SEQ ID NO: 511 - protein, 20 aa, synthetic construct
WQIFDCYLAQVGELCDLQDT

SEQ ID NO: 512 - protein, 20 aa, synthetic construct
GRYPDCYIAHVGELCEFYDG

SEQ ID NO: 513 - protein, 20 aa, synthetic construct
FGDDFCRFIPLFEMCTTDVE

SEQ ID NO: 514 - protein, 20 aa, synthetic construct
LVYYDCYMAQVGELCDLPSL

SEQ ID NO: 515 - protein, 20 aa, synthetic construct
VSRYDCYIAKVGELCDFFEF

SEQ ID NO: 516 - protein, 20 aa, synthetic construct
VTVQDCYFARVGDLCDLFSP

SEQ ID NO: 517 - protein, 20 aa, synthetic construct
WEWYDCLMAQVGELCDFEGN

SEQ ID NO: 518 - protein, 20 aa, synthetic construct
WAFYDCRNAQVGDFCDLWEF

SEQ ID NO: 519 - protein, 20 aa, synthetic construct
SMDQDCYFAQVGELCVLFNQ

SEQ ID NO: 520 - protein, 20 aa, synthetic construct
GGYYDCLIAEVGELCDIYGR

SEQ ID NO: 521 - protein, 20 aa, synthetic construct
SNWHDCLFAQVGELCDLPGS

SEQ ID NO: 522 - protein, 14 aa, synthetic construct
YDCYIAQVGELCDI

SEQ ID NO: 523 - protein, 20 aa, synthetic construct
SWLSDLQDCYIAQVGDLCQI

SEQ ID NO: 524 - protein, 20 aa, synthetic construct
RLASDWWDCYIAKVGELCDF

SEQ ID NO: 525 - protein, 20 aa, synthetic construct
RLLRDCFLAKVGDLCELFVW

SEQ ID NO: 526 - protein, 20 aa, synthetic construct
QWFHNCFLARVGDTCDLFLW

SEQ ID NO: 527 - protein, 20 aa, synthetic construct
ELLVDCFKVKVGELCDLFFG

SEQ ID NO: 528 - protein, 20 aa, synthetic construct
RYVHDCFIAQVGDLCDLFLH

SEQ ID NO: 529 - protein, 20 aa, synthetic construct
KWVHDCFLAKVGDVCDLFVV

SEQ ID NO: 530 - protein, 20 aa, synthetic construct
RSLVDCFLVKVGDLCDFFNW

SEQ ID NO: 531 - protein, 20 aa, synthetic construct
RYLYDCFLALVGDLCVKFHQ

SEQ ID NO: 532 - protein, 20 aa, synthetic construct
AHFYDCFWAKAGELCDLWPS

SEQ ID NO: 533 - protein, 20 aa, synthetic construct
KWFHDCFLAKVGDLCDLFLW

SEQ ID NO: 534 - protein, 22 aa, synthetic construct
WGKLVRDCFLAKVGDLCDLFLW

SEQ ID NO: 535 - protein, 20 aa, synthetic construct
THVHDCFLAKVGDLCDLFIV

SEQ ID NO: 536 - protein, 20 aa, synthetic construct
HWVRDCFLAKVGELCDLFLW

SEQ ID NO: 537 - protein, 20 aa, synthetic construct
HGILDCYFAKVGELCELFDW

SEQ ID NO: 538 - protein, 20 aa, synthetic construct
QFVKDCFLAQVGDLCELFLW

SEQ ID NO: 539 - protein, 20 aa, synthetic construct
DWLPDCYFANVGDLCSLFGS

SEQ ID NO: 540 - protein, 20 aa, synthetic construct
HWFLDCFLANVGDLCDFFGN

SEQ ID NO: 541 - protein, 20 aa, synthetic construct
NWLPDCLFANVGELCDIFPW

SEQ ID NO: 542 - protein, 20 aa, synthetic construct
EIFKDCLFANVGELCEIFPS

SEQ ID NO: 543 - protein, 20 aa, synthetic construct
NWFHDCFLARVGDLCDLFLD

SEQ ID NO: 544 - protein, 20 aa, synthetic construct
YSFKDCYFAKVGELCELFLW

SEQ ID NO: 545 - protein, 20 aa, synthetic construct
EFFHDCYVARVGELCDLFGW

SEQ ID NO: 546 - protein, 16 aa, synthetic construct
SYLCWLDHWGVICEED

SEQ ID NO: 547 - protein, 16 aa, synthetic construct
WYTCMMDWLGVHCELE

SEQ ID NO: 548 - protein, 22 aa, synthetic construct
QMVWWDCWSTQEGPVCELNWTA

SEQ ID NO: 549 - protein, 20 aa, synthetic construct
YWCSVWQLGSVCEMNNEETQ

SEQ ID NO: 550 - protein, 22 aa, synthetic construct
WTCWLTQLGYDCNLDVVDQSLG

SEQ ID NO: 551 - protein, 22 aa, synthetic construct
DGSWYTCWFTQLGEWCEQDDAK

SEQ ID NO: 552 - protein, 22 aa, synthetic construct
EFWGWQCWQEPLGWSCDLEWMD

SEQ ID NO: 553 - protein, 16 aa, synthetic construct
WYPCWIARVGELCDLE

SEQ ID NO: 554 - protein, 16 aa, synthetic construct
WYPCWLAQVGELCDLD

SEQ ID NO: 555 - protein, 16 aa, synthetic construct
LVDCFKVKVGELCDLF

SEQ ID NO: 556 - protein, 16 aa, synthetic construct
VHDCFIAQVGDLCDLF

SEQ ID NO: 557 - protein, 14 aa, synthetic construct
WPCWAALGELCDLD

SEQ ID NO: 558 - protein, 13 aa, synthetic construct
WPCWALGELCDLD

SEQ ID NO: 559 - protein, 12 aa, synthetic construct
WPCWLGELCDLD

SEQ ID NO: 560 - protein, 12 aa, synthetic construct
WYPCWGELCDLD

SEQ ID NO: 561-567 - skipped (7 entries)

SEQ ID NO: 568 - protein, 20 aa, synthetic construct
AFFHDCFFAKAGDLCDFFDD

SEQ ID NO: 569 - protein, 20 aa, synthetic construct
DFFHDCFFAKVGDLCDFFFG

SEQ ID NO: 570 - protein, 20 aa, synthetic construct
EGFHDCFFAKVGDLCDIFGH

SEQ ID NO: 571 - protein, 20 aa, synthetic construct
EHFHDCFFAKVGDLCDIFGN

SEQ ID NO: 572 - protein, 20 aa, synthetic construct
EHFHDCFFAKVGDLCDKFGQ

SEQ ID NO: 573 - protein, 20 aa, synthetic construct
HIFHDCFIAKVGDLCDLFHS

SEQ ID NO: 574 - protein, 20 aa, synthetic construct
HLFHDCFKAKVGDLCDLFIS

SEQ ID NO: 575 - protein, 20 aa, synthetic construct
HLFKDCFLAKVGDLCDLFLS

SEQ ID NO: 576 - protein, 22 aa, synthetic construct
WGKSIKDCFLAKVGDLCDLFLV

SEQ ID NO: 577 - protein, 20 aa, synthetic construct
KSLKDCFLAKVGDLCDLFLV

SEQ ID NO: 578 - protein, 20 aa, synthetic construct
KWLLDCFLALVGDLCDLFLW

SEQ ID NO: 579 - protein, 20 aa, synthetic construct
NWLLDCFLANVGDLCDLFLW

SEQ ID NO: 580 - protein, 20 aa, synthetic construct
NWLPDCFLANVGELCDLFLW

SEQ ID NO: 581 - protein, 20 aa, synthetic construct
QWLPDCFLANVGELCDLFLW

SEQ ID NO: 582 - protein, 20 aa, synthetic construct
QWVRDCLLANVGELCELFLW

SEQ ID NO: 583 - protein, 20 aa, synthetic construct
RWVRDCLLAQVGELCELFNW

SEQ ID NO: 584 - protein, 20 aa, synthetic construct
RWVVDCYLAQVGELCELFPW

SEQ ID NO: 585 - protein, 20 aa, synthetic construct
RWVVDCYLARVGELCELFPW

SEQ ID NO: 586 - protein, 20 aa, synthetic construct
TYVYDCYVVRVGETCSLFPW

SEQ ID NO: 587 - protein, 20 aa, synthetic construct
YYVYNCYWVRVGEVCVLWVW

SEQ ID NO: 588 - protein, 18 aa, synthetic construct
GGYDCRIAQVGELCDLGG

SEQ ID NO: 589 - protein, 23 aa, synthetic construct
GVQYKKCWMAQLGDLCELDPSGG

SEQ ID NO: 590 - protein, 20 aa, synthetic construct
GGYPCWMAQLGELCDLGGRR

SEQ ID NO: 591 - protein, 20 aa, synthetic construct
GGWYPCWMAQLGELCDLDGG

SEQ ID NO: 592 - protein, 22 aa, synthetic construct
GGWYPCWMAQLGELCDLDGGRR

SEQ ID NO: 593 - protein, 26 aa, synthetic construct
GGYPCHMAQLGELCDLWSWGDIGGRR

SEQ ID NO: 594 - protein, 24 aa, synthetic construct
FYPCWTALLGELCDLEPGPPAMGG

SEQ ID NO: 595 - protein, 24 aa, synthetic construct
WRRWYPCWVAQVGELCDLEIEAGG

SEQ ID NO: 596 - protein, 24 aa, synthetic construct
RQRWYPCWMARLGELCDLDEPTGG

SEQ ID NO: 597 - protein, 24 aa, synthetic construct
WYPCWMAQLGDLCDLEKPVTERGG

SEQ ID NO: 598 - protein, 18 aa, synthetic construct
GGWYPCWMAQLGELCDLD

SEQ ID NO: 599 - protein, 26 aa, synthetic construct
GGFYPCWTALLGELCDLEPGPPAMGG

SEQ ID NO: 600 - protein, 26 aa, synthetic construct
GGWGTTWRWYPCWMAQLGELCDLEGG

SEQ ID NO: 601 - protein, 25 aa, synthetic construct
GGDVLGDRWYPCWIAKGELCDLDGG

SEQ ID NO: 602 - protein, 19 aa, synthetic construct
GGWPCWIAQLGELCDLDGG

SEQ ID NO: 603 - protein, 20 aa, synthetic construct
GGWYPCWLAKLGELCDLDGG

SEQ ID NO: 604 - protein, 26 aa, synthetic construct
GGWYPCWMAQLGDLCDLEKPVTERGG

SEQ ID NO: 605 - protein, 26 aa, synthetic construct
GGWRRWYPCWVAQVGELCDLEIEAGG

SEQ ID NO: 606 - protein, 26 aa, synthetic construct
GGRQRWYPCWMARLGELCDLDEPTGG

SEQ ID NO: 607 - protein, 24 aa, synthetic construct
GGVQYKKCWMAQLGDLCELDPSGG

SEQ ID NO: 608 - protein, 24 aa, synthetic construct
GGYPCHMAQLGELCDLWSWGDIGG

SEQ ID NO: 609 - protein, 24 aa, synthetic construct
GGWYPCWIARVGELCDLEEGPVNR

SEQ ID NO: 610 - protein, 23 aa, synthetic construct
GGAVEFYPCWLARIGELCDLVEP

SEQ ID NO: 611 - protein, 18 aa, synthetic construct
GGWYPCWIARVGELCDME

SEQ ID NO: 612 - protein, 25 aa, synthetic construct
GGEWFHDCFLAKVGDLCDLFLGGEW

SEQ ID NO: 613 - protein, 22 aa, synthetic construct
GGRYVHDCFIAQVGDLCDLFLH

SEQ ID NO: 614 - protein, 22 aa, synthetic construct
GGRSLVDCFLVKVGDLCDFFNW

SEQ ID NO: 615 - protein, 18 aa, synthetic construct
GGWYPCWIARVGELCDLE

SEQ ID NO: 616 - protein, 18 aa, synthetic construct
GGWYPCWLAQVGELCDLD

SEQ ID NO: 617 - protein, 18 aa, synthetic construct
GGLVDCFKVKVGELCDLF

SEQ ID NO: 618 - protein, 18 aa, synthetic construct
GGWYSCWMAQLGELCDLD

SEQ ID NO: 619 - protein, 20 aa, synthetic construct
VQYKKCWMAQLGDLCELDPS

SEQ ID NO: 620 - protein, 20 aa, synthetic construct
YPCHMAQLGELCDLWSWGDI

SEQ ID NO: 621 - protein, 14 aa, synthetic construct
YPCWMAQLGELCDL

SEQ ID NO: 622 - skipped

SEQ ID NO: 623 - protein, 22 aa, synthetic construct
WGTTWRWYPCWMAQLGELCDLE

SEQ ID NO: 624 - protein, 22 aa, synthetic construct
FYPCWTALLGELCDLEPGPPAM

SEQ ID NO: 625 - protein, 22 aa, synthetic construct
WRRWYPCWVAQVGELCDLEIEA

SEQ ID NO: 626 - protein, 22 aa, synthetic construct
RQRWYPCWMARLGELCDLDEPT

SEQ ID NO: 627 - protein, 22 aa, synthetic construct
DVLGDRWYPCWIAKLGELCDLD

SEQ ID NO: 628 - protein, 22 aa, synthetic construct
WYPCWMAQLGDLCDLEKPVTER

SEQ ID NO: 629 - protein, 16 aa, synthetic construct
WYPCWIAQLGELCDLD

SEQ ID NO: 630 - protein, 16 aa, synthetic construct
WYPCWLAKLGELCDLD

SEQ ID NO: 631-636 - skipped (6 entries)

SEQ ID NO: 637 - protein, 14 aa, synthetic construct
IECDTSYGVYICWQ

SEQ ID NO: 638 - protein, 14 aa, synthetic construct
IECEEWRGVELCWQ

SEQ ID NO: 639 - protein, 20 aa, synthetic construct
PEGREVVVCRDWYGVELCWQ

SEQ ID NO: 640 - protein, 20 aa, synthetic construct
IWGRTVVECQDWEGVELCWQ

SEQ ID NO: 641 - protein, 20 aa, synthetic construct
LALRKEVVCQEYYGVELCWI

SEQ ID NO: 642 - protein, 20 aa, synthetic construct
HEAREVVVCQDWYGVELCWQ

SEQ ID NO: 643 - protein, 20 aa, synthetic construct
MVNREVVVCEDWYGVELCWQ

SEQ ID NO: 644 - protein, 20 aa, synthetic construct
TANQTVVECQVWGGVELCWQ

SEQ ID NO: 645 - protein, 14 aa, synthetic construct
VECQEWGGVELCWC

SEQ ID NO: 646 - protein, 15 aa, synthetic construct
DVECVDWGGVELCWH

SEQ ID NO: 647 - protein, 14 aa, synthetic construct
IVCEEWRGVELCWL

SEQ ID NO: 648 - protein, 20 aa, synthetic construct
DFERSYVVCQDWDGVELCWI

SEQ ID NO: 649 - protein, 20 aa, synthetic construct
AHSRQEVVCEEWYGVELCWI

SEQ ID NO: 650 - protein, 20 aa, synthetic construct
SAPERWVECEDWQGVELCWV

SEQ ID NO: 651 - protein, 20 aa, synthetic construct
YSRELYVQCEDWEGVELCWI

SEQ ID NO: 652 - protein, 14 aa, synthetic construct
VVCQDWEGVELCWQ

SEQ ID NO: 653 - protein, 15 aa, synthetic construct
DVVCQNWEGVDLCWH

SEQ ID NO: 654 - protein, 20 aa, synthetic construct
SAGRQEVVCQDWNGVELCWI

SEQ ID NO: 655 - protein, 20 aa, synthetic construct
GQGREVVVCHDWYGVELCWQ

SEQ ID NO: 656 - protein, 20 aa, synthetic construct
DWRRSVVECQDWYGVELCWQ

SEQ ID NO: 657 - protein, 15 aa, synthetic construct
DVVCQNWDGVDLCWH

SEQ ID NO: 658 - protein, 20 aa, synthetic construct
TLGRTVVECQDWGGVELCWQ

SEQ ID NO: 659 - protein, 20 aa, synthetic construct
RLLNSVVECLDWEGVELCWQ

SEQ ID NO: 660 - protein, 14 aa, synthetic construct
IVCEDWRGVELCWI

SEQ ID NO: 661 - protein, 14 aa, synthetic construct
VVCQEWEGVELCWC

SEQ ID NO: 662 - protein, 20 aa, synthetic construct
GDRPKEVVCEDWKGVELCWI

SEQ ID NO: 663 - protein, 20 aa, synthetic construct
ERPRSFIECQEWEGVELCWL

SEQ ID NO: 664 - protein, 20 aa, synthetic construct
EGSTTTIECEEWAGVELCWL

SEQ ID NO: 665 - protein, 20 aa, synthetic construct
ANQNTVVECQDWHGVELCWQ

SEQ ID NO: 666 - protein, 20 aa, synthetic construct
RSDDEVVVCQEWEGVELCWQ

SEQ ID NO: 667 - protein, 14 aa, synthetic construct
IECEEWAGVELCWL

SEQ ID NO: 668 - protein, 20 aa, synthetic construct
TWNMSELECQDWNGVEICWH

SEQ ID NO: 669 - protein, 20 aa, synthetic construct
GNDDSYIVCEEWKGVELCWI

SEQ ID NO: 670 - protein, 20 aa, synthetic construct
FAHHGVVECQEWYGVELCWQ

SEQ ID NO: 671 - protein, 20 aa, synthetic construct
LNRSVWIECEEYEGVELCWL

SEQ ID NO: 672 - protein, 20 aa, synthetic construct
WSKKAEVVCEEWGGVEFCWI

SEQ ID NO: 673 - protein, 20 aa, synthetic construct
RSNQTVVECQDWEGVELCWQ

SEQ ID NO: 674 - protein, 20 aa, synthetic construct
VVCQEWEGVELCWYAGECMQ

SEQ ID NO: 675 - protein, 20 aa, synthetic construct
ILCQEFEGVELCWLEESLAE

SEQ ID NO: 676 - protein, 20 aa, synthetic construct
KSQVECQDWEGVELCWVVSE

SEQ ID NO: 677 - protein, 20 aa, synthetic construct
KITVECQDWDGVELCWPTWI

SEQ ID NO: 678 - protein, 20 aa, synthetic construct
RPQIECQEWQGVELCWTREE

SEQ ID NO: 679 - protein, 20 aa, synthetic construct
VSCQEWDGVELCWVDGDLAA

SEQ ID NO: 680 - protein, 20 aa, synthetic construct
IMCQEWDGVELCWLERDKAN

SEQ ID NO: 681 - protein, 20 aa, synthetic construct
GLEIACEDWYGVELCWLRRA

SEQ ID NO: 682 - protein, 24 aa, synthetic construct
GYGVLCQEWQGVELCWPVQREAGV

SEQ ID NO: 683 - protein, 20 aa, synthetic construct
PYGVVCQDWAGVELCWVENR

SEQ ID NO: 684 - protein, 20 aa, synthetic construct
KLTVECQDWDGVELCWVGVE

SEQ ID NO: 685 - protein, 20 aa, synthetic construct
INCQTWNGVELCWVDEGLYQ

SEQ ID NO: 686 - protein, 20 aa, synthetic construct
VVCQEWEGVELCWVEPPLLP

SEQ ID NO: 687 - protein, 20 aa, synthetic construct
RVQVECEDWNGVELCWPVRV

SEQ ID NO: 688 - protein, 20 aa, synthetic construct
DRQVVCEEWDGVELCWIEES

SEQ ID NO: 689 - protein, 20 aa, synthetic construct
KTTVACQDWGGVELCWVERV

SEQ ID NO: 690 - protein, 20 aa, synthetic construct
RPEVVCQEWEGVELCWISPL

SEQ ID NO: 691 - protein, 20 aa, synthetic construct
RLGVECQEWEGVDLCWISAF

SEQ ID NO: 692 - protein, 20 aa, synthetic construct
KPVVVCEEWQGVELCWLEIQ

SEQ ID NO: 693 - protein, 20 aa, synthetic construct
VVCEVFQGVELCWCENEEFT

SEQ ID NO: 694 - protein, 20 aa, synthetic construct
TDEVSCQEWEGVELCWIERQ

SEQ ID NO: 695 - protein, 20 aa, synthetic construct
PVEVRCQEWEGVELCWVVGI

SEQ ID NO: 696 - protein, 20 aa, synthetic construct
GPEVVCEEFNRVELCWVEYN

SEQ ID NO: 697 - protein, 20 aa, synthetic construct
KYIVECQEWGGVELCWPEMV

SEQ ID NO: 698 - protein, 20 aa, synthetic construct
VTCQEYEGVELCWTVGCAYS

SEQ ID NO: 699 - protein, 21 aa, synthetic construct
VVCQEWEGVELCWQTGPGAHA

SEQ ID NO: 700 - protein, 20 aa, synthetic construct
IVCEEYNGVELCWVETSVKP

SEQ ID NO: 701 - protein, 20 aa, synthetic construct
EQQVVCQEWNGVELCWIEAG

SEQ ID NO: 702 - protein, 20 aa, synthetic construct
QLGVECQNWRGVELCWVSEI

SEQ ID NO: 703 - protein, 20 aa, synthetic construct
TAEVVCQEWDGVELCWIEVL

SEQ ID NO: 704 - protein, 20 aa, synthetic construct
SPSIVCEEWAGVELCWVDYS

SEQ ID NO: 705 - protein, 20 aa, synthetic construct
AVCQDWYGVELCWCMQDILD

SEQ ID NO: 706 - protein, 20 aa, synthetic construct
VECEEWGGVELCWLADEVMW

SEQ ID NO: 707 - protein, 20 aa, synthetic construct
HSTVICQDWDGVELCWIEND

SEQ ID NO: 708 - protein, 20 aa, synthetic construct
KKIVVCQDWGGVELCWTEDD

SEQ ID NO: 709 - protein, 20 aa, synthetic construct
SVEVVCEEWHGVELCWPVFI

SEQ ID NO: 710 - protein, 20 aa, synthetic construct
RWAVSCQDWQGIELCWPEWD

SEQ ID NO: 711 - protein, 20 aa, synthetic construct
RTGVECQDWHGVELCWPVWE

SEQ ID NO: 712 - protein, 20 aa, synthetic construct
GYGVVCEDFRGVELCWLERK

SEQ ID NO: 713 - protein, 17 aa, synthetic construct
RTEVECEDWEGVELCWL

SEQ ID NO: 714 - protein, 19 aa, synthetic construct
ILCEEWQGVELCWLEGGGS

SEQ ID NO: 715 - protein, 16 aa, synthetic construct
VGIECEEWAGVELCWL

SEQ ID NO: 716 - protein, 14 aa, synthetic construct
IECEEWAGVELCWL

SEQ ID NO: 717-722 - skipped (6 entries)

SEQ ID NO: 723 - protein, 15 aa, synthetic construct
SLLKCYNASTCASVF

SEQ ID NO: 724 - protein, 12 aa, synthetic construct
CGMAIGDLCMWT

SEQ ID NO: 725 - protein, 12 aa, synthetic construct
RWGDVGDLLMPL

SEQ ID NO: 726 - protein, 14 aa, synthetic construct
RSCYYKRPRLWCSE

SEQ ID NO: 727 - protein, 16 aa, synthetic construct
DCSMWEGVELCWGGRR

SEQ ID NO: 728 - protein, 20 aa, synthetic construct
GGVVCQDWEGVELCWQGGRR

SEQ ID NO: 729 - protein, 18 aa, synthetic construct
GGVMCERWQGVELCWLGG

SEQ ID NO: 730 - protein, 24 aa, synthetic construct
GGRTGVECQDWHGVELCWPVWEGG

SEQ ID NO: 731 - protein, 20 aa, synthetic construct
GGVGIECEEWAGVELCWLGG

SEQ ID NO: 732 - protein, 24 aa, synthetic construct
GGTWNMSELECQDWNGVEICWHGG

SEQ ID NO: 733 - protein, 21 aa, synthetic construct
GGRTEVECEDWEGVELCWLGG

SEQ ID NO: 734 - protein, 24 aa, synthetic construct
GGWSKKAEVVCEEWGGVEFCWIGG

SEQ ID NO: 735 - protein, 20 aa, synthetic construct
WSKKAEVVCEEWGGVEFCWI

SEQ ID NO: 736 - protein, 20 aa, synthetic construct
YSRELYVQCEDWEGVELCWI

SEQ ID NO: 737 - protein, 16 aa, synthetic construct
GGDCSMWEGVELCWGG

SEQ ID NO: 738 - protein, 18 aa, synthetic construct
GGVMCERWQGVELCWLGG

SEQ ID NO: 739 - protein, 20 aa, synthetic construct
GGVGIECEEWAGVELCWLGG

SEQ ID NO: 740 - protein, 24 aa, synthetic construct
GGTWNMSELECQDWNGVEICWHGG

SEQ ID NO: 741 - protein, 21 aa, synthetic construct
GGRTEVECEDWEGVELCWLGG

SEQ ID NO: 742 - protein, 24 aa, synthetic construct
GGRTGVECQDWHGVELCWPVWEGG

SEQ ID NO: 743 - protein, 20 aa, synthetic construct
GGVVCQDWEGVABULCWQGG

SEQ ID NO: 744 - protein, 20 aa, synthetic construct
GGVVCQDWEGVALBLCWQGG

SEQ ID NO: 745 - protein, 19 aa, synthetic construct
GGVVCQDWEGVDALCWQGG

SEQ ID NO: 746 - protein, 18 aa, synthetic construct
GGVVCQDWEGVSLCWQGG

SEQ ID NO: 747 - protein, 18 aa, synthetic construct
GGVVCQDWEGVGLCWQGG

SEQ ID NO: 748 - protein, 19 aa, synthetic construct
GGVVCQDWEGVELCWQPPA

SEQ ID NO: 749 - protein, 20 aa, synthetic construct
GGVVCQDWEGVELCWQGPPA

SEQ ID NO: 750 - protein, 16 aa, synthetic construct
WHPCWIAQLGELCDLE

SEQ ID NO: 751 - protein, 18 aa, synthetic construct
GWHPCWIAQLGELCDLEG

SEQ ID NO: 752 - protein, 20 aa, synthetic construct
GGWHPCWIAQLGELCDLEGG

SEQ ID NO: 753 - protein, 22 aa, synthetic construct
GGGWHPCWIAQLGELCDLEGGG

SEQ ID NO: 754 - skipped

SEQ ID NO: 755 - protein, 19 aa, synthetic construct
GWYPCWMAQLGELCDLDGG

SEQ ID NO: 756 - protein, 18 aa, synthetic construct
WYPCWMAQLGELCDLDGG

SEQ ID NO: 757 - protein, 17 aa, synthetic construct
YPCWMAQLGELCDLDGG

SEQ ID NO: 758 - protein, 16 aa, synthetic construct
PCWMAQLGELCDLDGG

SEQ ID NO: 759 - protein, 15 aa, synthetic construct
CWMAQLGELCDLDGG

SEQ ID NO: 760 - protein, 19 aa, synthetic construct
GGWYPCWMAQLGELCDLDG

SEQ ID NO: 761 - protein, 18 aa, synthetic construct
GGWYPCWMAQLGELCDLD

SEQ ID NO: 762 - protein, 17 aa, synthetic construct
GGWYPCWMAQLGELCDL

SEQ ID NO: 763 - protein, 16 aa, synthetic construct
GGWYPCWMAQLGELCD

SEQ ID NO: 764 - protein, 15 aa, synthetic construct
GGWYPCWMAQLGELC

SEQ ID NO: 765 - protein, 18 aa, synthetic construct
GWYPCWMAQLGELCDLDG

SEQ ID NO: 766 - protein, 17 aa, synthetic construct
WYPCWMAQLGELCDLDG

SEQ ID NO: 767 - skipped

SEQ ID NO: 768 - protein, 14 aa, synthetic construct
YPCWMAQLGELCDL

SEQ ID NO: 769 - protein, 9 aa, synthetic construct
PCWMAQLGE

SEQ ID NO: 770 - protein, 22 aa, synthetic construct
GGELLVDCFKVKVGELCDLFFG

SEQ ID NO: 771 - protein, 21 aa, synthetic construct
GGELLVDCFKVKVGELCDLFF

SEQ ID NO: 772 - protein, 20 aa, synthetic construct
GGELLVDCFKVKVGELCDLF

SEQ ID NO: 773 - protein, 19 aa, synthetic construct
GGELLVDCFKVKVGELCDL

SEQ ID NO: 774 - protein, 18 aa, synthetic construct
GGELLVDCFKVKVGELCD

SEQ ID NO: 775 - protein, 17 aa, synthetic construct
GGELLVDCFKVKVGELC

SEQ ID NO: 776 - protein, 21 aa, synthetic construct
GELLVDCFKVKVGELCDLFFG

SEQ ID NO: 777 - protein, 20 aa, synthetic construct
ELLVDCFKVKVGELCDLFFG

SEQ ID NO: 778 - protein, 19 aa, synthetic construct
LLVDCFKVKVGELCDLFFG

SEQ ID NO: 779 - protein, 18 aa, synthetic construct
LVDCFKVKVGELCDLFFG

SEQ ID NO: 780 - protein, 17 aa, synthetic construct
VDCFKVKVGELCDLFFG

SEQ ID NO: 781 - protein, 16 aa, synthetic construct
DCFKVKVGELCDLFFG

SEQ ID NO: 782 - protein, 18 aa, synthetic construct
ELLVDCFKVKVGELCDLF

SEQ ID NO: 783 - protein, 15 aa, synthetic construct
LVDCFKVKVGELCDL

SEQ ID NO: 784 - protein, 13 aa, synthetic construct
VDCFKVKVGELCD

SEQ ID NO: 785 - protein, 10 aa, synthetic construct
CFKVKVGELC

SEQ ID NO: 786 - protein, 18 aa, synthetic construct
GGVVCQDWEGVELCWQGG

SEQ ID NO: 787 - protein, 17 aa, synthetic construct
GVVCQDWEGVELCWQGG

SEQ ID NO: 788 - protein, 16 aa, synthetic construct
VVCQDWEGVELCWQGG

SEQ ID NO: 789 - protein, 15 aa, synthetic construct
VCQDWEGVELCWQGG

SEQ ID NO: 790 - protein, 14 aa, synthetic construct
CQDWEGVELCWQGG

SEQ ID NO: 791 - protein, 17 aa, synthetic construct
GGVVCQDWEGVELCWQG

SEQ ID NO: 792 - protein, 16 aa, synthetic construct
GGVVCQDWEGVELCWQ

SEQ ID NO: 793 - protein, 15 aa, synthetic construct
GGVVCQDWEGVELCW

SEQ ID NO: 794 - protein, 14 aa, synthetic construct
GGVVCQDWEGVELC

SEQ ID NO: 795 - protein, 16 aa, synthetic construct
GVVCQDWEGVELCWQG

SEQ ID NO: 796 - skipped

SEQ ID NO: 797 - protein, 13 aa, synthetic construct
VCQDWEGVELCWQ

SEQ ID NO: 798 - protein, 12 aa, synthetic construct
VCQDWEGVELCW

SEQ ID NO: 799 - protein, 11 aa, synthetic construct
CQDWEGVELCW

SEQ ID NO: 800 - protein, 10 aa, synthetic construct
CQDWEGVELC

SEQ ID NO: 801 - protein, 41 aa, synthetic construct
GGWYPCWMAQLGELCDLDGGGGSGGVVCQDWEGVELCWQGG

SEQ ID NO: 802 - protein, 37 aa, synthetic construct
WYPCWMAQLGELCDLDGGGGSGGVVCQDWEGVELCWQ

SEQ ID NO: 803 - protein, 35 aa, synthetic construct
  VARIANT 19: note = This region may encompass (G)n, (GS)n, (GGS)n, (GGGS)n, (GGGGS)n or a combination of any of the foregoing where n is an integer from 1 to 5.
WYPCWMAQLGELCDLDGGXGGVVCQDWEGVELCWQ

SEQ ID NO: 804 - protein, 31 aa, synthetic construct
  VARIANT 17: note = This region may encompass (G)n, (GS)n, (GGS)n, (GGGS)n, (GGGGS)n, or a combination of any of the foregoing, where n is an integer from 1 to 5.
WYPCWMAQLGELCDLDXVVCQDWEGVELCWQ

SEQ ID NO: 805-808 - skipped (4 entries)

SEQ ID NO: 809 - protein, 39 aa, synthetic construct
GWYPCWMAQLGELCDLDGGGGSGGVVCQDWEGVELCWQG

SEQ ID NO: 810 - protein, 40 aa, synthetic construct
GGWYPCWMAQLGELCDLDGGGGSGGVVCQDWEGVELCWQG

SEQ ID NO: 811 - protein, 40 aa, synthetic construct
GWYPCWMAQLGELCDLDGGGGSGGVVCQDWEGVELCWQGG

SEQ ID NO: 812 - protein, 33 aa, synthetic construct
  VARIANT 1: note = This region may encompass (G)n where n is an integer from 1 to 5, such as G, GG, GGG, GGGG, or, GGGGG.
  VARIANT 18: note = This region may encompass (G)m, (GS)n, (GGS)n, (GGGS)n, (GGGGS)n, or a combination of any of the foregoing, where n is an integer from 1 to 5.
  VARIANT 33: note = This region may encompass 1-5 G repeating units.
XWYPCWMAQLGELCDLDXVVCQDWEGVELCWQX

SEQ ID NO: 813 - protein, 47 aa, synthetic construct
GGWYPCWIARVGELCDLEEGPVNRGGGGSGGVVCQDWEGVELCWQGG

SEQ ID NO: 814 - protein, 46 aa, synthetic construct
GGAVEFYPCWLARIGELCDLVEPGGGGSGGVVCQDWEGVELCWQGG

SEQ ID NO: 815 - protein, 41 aa, synthetic construct
GGWYPCWIARVGELCDMEGGGGSGGVVCQDWEGVELCWQGG

SEQ ID NO: 816 - protein, 45 aa, synthetic construct
GGEWFHDCFLAKVGDLCDLFLWGGGGSGGVVCQDWEGVELCWQGG

SEQ ID NO: 817 - protein, 45 aa, synthetic construct
GGRYVHDCFIAQVGDLCDLFLHGGGGSGGVVCQDWEGVELCWQGG

SEQ ID NO: 818 - protein, 45 aa, synthetic construct
GGRSLVDCFLVKVGDLCDFFNWGGGGSGGVVCQDWEGVELCWQGG

SEQ ID NO: 819 - protein, 41 aa, synthetic construct
GGWYPCWIARVGELCDLEGGGGSGGVVCQDWEGVELCWQGG

SEQ ID NO: 820 - protein, 41 aa, synthetic construct
GGWYPCWLAQVGELCDLDGGGGSGGVVCQDWEGVELCWQGG

SEQ ID NO: 821 - protein, 52 aa, synthetic construct
GGWYPCWIARVGELCDLEEGPVNRGGGGSGGGGSGGVVCQDWEGVELCWQGG

SEQ ID NO: 822 - protein, 57 aa, synthetic construct
GGWYPCWIARVGELCDLEEGPVNRGGGGSGGGGSGGGGSGGVVCQDWEGVELCWQGG

SEQ ID NO: 823 - protein, 50 aa, synthetic construct
GGRYVHDCFIAQVGDLCDLFLHGGGGSGGGGSGGVVCQDWEGVELCWQGG

SEQ ID NO: 824 - protein, 55 aa, synthetic construct
GGRYVHDCFIAQVGDLCDLFLHGGGGSGGGGSGGGGSGGVVCQDWEGVELCWQGG

SEQ ID NO: 825 - protein, 60 aa, synthetic construct
GGRYVHDCFIAQVGDLCDLFLHGGGGSGGGGSGGGGSGGGGSGGVVCQDWEGVELCWQGG

SEQ ID NO: 826 - protein, 41 aa, synthetic construct
GGLVDCFKVKVGELCDLFGGGGSGGVVCQDWEGVELCWQGG

SEQ ID NO: 827 - protein, 45 aa, synthetic construct; identical to SEQ ID NO: 817

SEQ ID NO: 828 - protein, 41 aa, synthetic construct
GGWYSCWMAQLGELCDLDGGGGSGGVVCQDWEGVELCWQGG

SEQ ID NO: 829 - protein, 41 aa, synthetic construct
ACWYPCWMAQLGELCDLDGGGGSGGVVCQDWEGVELCWQOH

SEQ ID NO: 830 - protein, 41 aa, synthetic construct
ACWYPCWMAQLGELCDLDGGGGSGGVVCQDWEGVALCWQOH

SEQ ID NO: 831 - protein, 43 aa, synthetic construct
ACWYPCWABUAQLGELCDLDGGGGSGGVVCQDWEGVELCWQOH

SEQ ID NO: 832 - protein, 43 aa, synthetic construct
ACWYPCWABUAQLGELCDLDGGGGSGGVVCQDWEGVALCWQOH

SEQ ID NO: 833 - protein, 41 aa, synthetic construct; identical to SEQ ID NO: 829

SEQ ID NO: 834 - protein, 49 aa, synthetic construct
ACGGELLVDCFKVKVGELCDLFFGGGGGSGGVVCQDWEGVELCWQGGOH

SEQ ID NO: 835 - protein, 49 aa, synthetic construct
ACGGRYVHDCFIAQVGDLCDLFLHGGGGSGGVVCQDWEGVELCWQGGOH

SEQ ID NO: 836 - protein, 49 aa, synthetic construct
ACGGKWVHDCFLAKVGDVCDLFVVGGGGSGGVVCQDWEGVELCWQGGOH

SEQ ID NO: 837 - protein, 49 aa, synthetic construct
ACGGRSLVDCFLVKVGDLCDFFNWGGGGSGGVVCQDWEGVELCWQGGOH

SEQ ID NO: 838 - protein, 49 aa, synthetic construct
ACGGEWFHDCFLAKVGDLCDLFLWGGGGSGGVVCQDWEGVELCWQGGOH

SEQ ID NO: 839 - protein, 50 aa, synthetic construct
ACGGGELLVDCFKVKVGELCDLFFGGGGGSGGVVCQDWEGVELCWQGGOH

SEQ ID NO: 840 - protein, 50 aa, synthetic construct
ACGGGRYVHDCFIAQVGDLCDLFLHGGGGSGGVVCQDWEGVELCWQGGOH

SEQ ID NO: 841 - protein, 50 aa, synthetic construct
ACGGGKWVHDCFLAKVGDVCDLFVVGGGGSGGVVCQDWEGVELCWQGGOH

SEQ ID NO: 842 - protein, 50 aa, synthetic construct
ACGGGRSLVDCFLVKVGDLCDFFNWGGGGSGGVVCQDWEGVELCWQGGOH

SEQ ID NO: 843 - protein, 50 aa, synthetic construct
ACGGGEWFHDCFLAKVGDLCDLFLWGGGGSGGVVCQDWEGVELCWQGGOH

SEQ ID NO: 844 - protein, 56 aa, synthetic construct
GGGELLVDCFKVKVGELCDLFFGPAPAPAPAPAPAPAPAGVVCQDWEGVELCWQGG

SEQ ID NO: 845 - protein, 55 aa, synthetic construct
GGELLVDCFKVKVGELCDLFFGPAPAPAPAPAPAPAPAGVVCQDWEGVELCWQGG

SEQ ID NO: 846 - protein, 51 aa, synthetic construct
ELLVDCFKVKVGELCDLFFGPAPAPAPAPAPAPAPAGVVCQDWEGVELCWQ

SEQ ID NO: 847 - protein, 40 aa, synthetic construct
  VARIANT 23: note = This region may encompass (G)n, (GS)n, (GGS)n, (GGGS)n, (GGGGS)n, or a combination of any of the foregoing, where n is an integer from 1 to 5.
GGELLVDCFKVKVGELCDLFFGXGVVCQDWEGVELCWQGG

SEQ ID NO: 848 - protein, 36 aa, synthetic construct
  VARIANT 21: note = This region may encompass (G)n, (GS)n, (GGS)n, (GGGS)n, (GGGGS)n, or a combination of any of the foregoing, where n is an integer from 1 to 5.
ELLVDCFKVKVGELCDLFFGXGVVCQDWEGVELCWQ

SEQ ID NO: 849 - protein, 38 aa, synthetic construct
  VARIANT 1: note = This region may encompass 1-5 G repeating units, such as G, GG, GGG, GGGG, or GGGGG.
  VARIANT 22: note = This region may encompass (G)n, (GS)n, (GGS)n, (GGGS)n, (GGGGS)n, or a combination of any of the foregoing, where n is an integer from 1 to 5.
  VARIANT 38: note = This region may encompass 1-5 G repeating units.
XELLVDCFKVKVGELCDLFFGXGVVCQDWEGVELCWQX

SEQ ID NO: 850 - protein, 53 aa, synthetic construct
  VARIANT 1..53: note = This region may encompass 1-5 G repeating units such as G, GG, GGG, GGGG, or GGGGG.
XELLVDCFKVKVGELCDLFFGPAPAPAPAPAPAPAPAGVVCQDWEGVELCWQX

SEQ ID NO: 851 - protein, 60 aa, synthetic construct
ACGGGELLVDCFKVKVGELCDLFFGPAPAPAPAPAPAPAPAGVVCQDWEGVELCWQGGOH

SEQ ID NO: 852 - protein, 59 aa, synthetic construct
ACGGELLVDCFKVKVGELCDLFFGPAPAPAPAPAPAPAPAGVVCQDWEGVELCWQGGOH

SEQ ID NO: 853 - protein, 43 aa, synthetic construct
VHRIPWCTLDPGGLQCAWLRQMGGGGSGGVVCQDWEGVELCWQ

SEQ ID NO: 854 - protein, 41 aa, synthetic construct
VHRIPWCTLDPGGLQCAWLRQGGGGSGGVVCQDEGVELCWQ

SEQ ID NO: 855 - protein, 41 aa, synthetic construct
VHRIPWCTLDPGGLQCAWLRGGGGSGGVVCQDWEGVELCWQ

SEQ ID NO: 856 - protein, 42 aa, synthetic construct
HRIPWCTLDPGGLQCAWLRQMGGGGSGGVVCQDWEGVELCWQ

SEQ ID NO: 857 - protein, 41 aa, synthetic construct
HRIPWCTLDPGGLQCAWLRQGGGGSGGVVCQDWEGVELCWQ

SEQ ID NO: 858 - protein, 40 aa, synthetic construct
HRIPWCTLDPGGLQCAWLRGGGGSGGVVCQDWEGVELCWQ

SEQ ID NO: 859 - protein, 41 aa, synthetic construct
RIPWCTLDPGGLQCAWLRQMGGGGSGGVVCQDWEGVELCWQ

SEQ ID NO: 860 - protein, 40 aa, synthetic construct
RIPWCTLDPGGLQCAWLRQGGGGSGGVVCQDWEGVELCWQ

SEQ ID NO: 861 - protein, 39 aa, synthetic construct
RIPWCTLDPGGLQCAWLRGGGGSGGVVCQDWEGVELCWQ

SEQ ID NO: 862 - protein, 40 aa, synthetic construct
IPWCTLDPGGLQCAWLRQMGGGGSGGVVCQDWEGVELCWQ

SEQ ID NO: 863 - protein, 39 aa, synthetic construct
IPWCTLDPGGLQCAWLRQGGGGSGGVVCQDWEGVELCWQ

SEQ ID NO: 864 - protein, 38 aa, synthetic construct
IPWCTLDPGGLQCAWLRGGGGSGGVVCQDWEGVELCWQ

SEQ ID NO: 865 - protein, 41 aa, synthetic construct
IEGRGGQCIHWDIETLLSCVGGGGSGGVVCQDWEGVELCWQ

SEQ ID NO: 866 - protein, 43 aa, synthetic construct
IEGRGGVPWCTLDPGSLQCAWFGGGGSGGVVCQDWEGVELCWQ

SEQ ID NO: 867 - protein, 43 aa, synthetic construct
IEGRGGRYECADLPGGLHCEFRGGGGSGGVVCQDWEGVELCWQ

SEQ ID NO: 868 - protein, 43 aa, synthetic construct
RHFDDIIPWCTLDPGSLQCAYLGGGGSGGVVCQDWEGVELCWQ

SEQ ID NO: 869 - protein, 46 aa, synthetic construct
VHRIPWCTLDPGGLQCAWLRQMGGGGSGGVVCQDWEGVELCWQPPA

SEQ ID NO: 870 - protein, 45 aa, synthetic construct
VHRIPWCTLDPGGLQCAWLRQGGGGSGGVVCQDWEGVELCWQPPA

SEQ ID NO: 871 - protein, 44 aa, synthetic construct
VHRIPWCTLDPGGLQCAWLRGGGGSGGVVCQDWEGVELCWQPPA

SEQ ID NO: 872 - protein, 46 aa, synthetic construct
GWGIPWCTLDPGSLQCAWLGKHGGGGSGGVVCQDWEGVELCWQPPA

SEQ ID NO: 873 - protein, 46 aa, synthetic construct
VHRIPWCTLDPGGLQCAWLRQGGGGGSGGVVCQDWEGVELCWQPPA

SEQ ID NO: 874 - protein, 46 aa, synthetic construct
VHRIPWCTLDPGGLQCAWLRGGGGGGSGGVVCQDWEGVELCWQPPA

SEQ ID NO: 875 - protein, 44 aa, synthetic construct
VHRIPWCTLDPGGLQCAWLRQGGGGSGGVVCQDWEGVELCWQGG

SEQ ID NO: 876 - protein, 43 aa, synthetic construct
VHRIPWCTLDPGGLQCAWLRGGGGSGGVVCQDWEGVELCWQGG

SEQ ID NO: 877 - protein, 45 aa, synthetic construct
GWGIPWCTLDPGSLQCAWLGKHGGGGSGGVVCQDWEGVELCWQGG

SEQ ID NO: 878 - protein, 41 aa, synthetic construct
VHRIPWCTLDPGGLQCAWLRQMPAGVVCQDWEGVELCWQGG

SEQ ID NO: 879 - skipped

SEQ ID NO: 880 - protein, 43 aa, synthetic construct
HLGVPWCTLDPGSIQCAWLAKHGGGGSGGVVCQDWEGVELCWQ

SEQ ID NO: 881 - protein, 43 aa, synthetic construct
VVCQDWEGVELCWQGGGGSGGRHFDDIIPWCTLDPGSLQCAYL

SEQ ID NO: 882 - protein, 43 aa, synthetic construct
VVCQDWEGVELCWQGGGGSGGHLGVPWCTLDPGSIQCAWLAKH

SEQ ID NO: 883 - protein, 35 aa, synthetic construct
HCKHWDLESLLLCVGGGGSGGVVCQDWEGVELCWQ

SEQ ID NO: 884 - protein, 41 aa, synthetic construct
QCVHWDLDTLFGCIREQLELGGGGSGGVVCQDWEGVELCWQ

SEQ ID NO: 885 - protein, 41 aa, synthetic construct
VVCQDWEGVELCWQGGGGSGGQCVHWDLDTLFGCIREQLEL

SEQ ID NO: 886 - protein, 43 aa, synthetic construct
IRSCLWQPGALHCTWWAEEEPVGGGGSGGVVCQDWEGVELCWQ

SEQ ID NO: 887 - protein, 43 aa, synthetic construct
VVCQDWEGVELCWQGGGGSGGIRSCLWQPGALHCTWWAEEEPV

SEQ ID NO: 888 - protein, 37 aa, synthetic construct
IPWCLLDPGGLQCVWLGGGGSGGVVCQDWEGVELCWQ

SEQ ID NO: 889 - protein, 37 aa, synthetic construct
VVCQDWEGVELCWQGGGGSGGIPWCLLDPGGLQCVWL

SEQ ID NO: 890 - protein, 44 aa, synthetic construct
VHRIPWCTLDPGGLQCAWLRQMGGGGSGGVVCQDWEGVELCWQG

SEQ ID NO: 891 - protein, 45 aa, synthetic construct
VHRIPWCTLDPGGLQCAWLRQMGGGGSGGVVCQDWEGVELCWQGG

SEQ ID NO: 892 - protein, 46 aa, synthetic construct
GVHRIPWCTLDPGGLQCAWLRQMGGGGSGGVVCQDWEGVELCWQGG

SEQ ID NO: 893 - protein, 47 aa, synthetic construct
GGVHRIPWCTLDPGGLQCAWLRQMGGGGSGGVVCQDWEGVELCWQGG

SEQ ID NO: 894 - protein, 41 aa, synthetic construct; identical to SEQ ID NO: 855

SEQ ID NO: 895 - protein, 42 aa, synthetic construct
WGIPWCTLDPGSLQCAWLGKHGGGGSGGVVCQDWEGVELCWQ

SEQ ID NO: 896 - protein, 42 aa, synthetic construct
VHRIPWCTLDPGGLQCAWLRQGGGGSGGVVCQDWEGVELCWQ

SEQ ID NO: 897 - protein, 42 aa, synthetic construct
VHRIPWCTLDPGGLQCAWLRMGGGGSGGVVCQDWEGVELCWQ

SEQ ID NO: 898 - protein, 43 aa, synthetic construct
VHRIPWCTLDPGGLQCAWIRQMGGGGSGGVVCQDWEGVELCWQ

SEQ ID NO: 899 - protein, 43 aa, synthetic construct
VHRIPWCTLDPGGLQCAWVRQMGGGGSGGVVCQDWEGVELCWQ

SEQ ID NO: 900 - protein, 43 aa, synthetic construct
VHRIPWCTLDPGGLQCAWARQMGGGGSGGVVCQDWEGVELCWQ

SEQ ID NO: 901 - protein, 45 aa, synthetic construct
GVHRIPWCTLDPGGLQCAWLRQMGGGGSGGVVCQDWEGVELCWQG

SEQ ID NO: 902 - protein, 41 aa, synthetic construct; identical to SEQ ID NO: 854

SEQ ID NO: 903 - protein, 41 aa, synthetic construct; identical to SEQ ID NO: 855

SEQ ID NO: 904 - protein, 42 aa, synthetic construct
VHRIPWCTLDPGGLQCAWLGKHGGGGSGGVVCQWEGVELCWQ

SEQ ID NO: 905 - protein, 42 aa, synthetic construct; identical to SEQ ID NO: 897

SEQ ID NO: 906 - protein, 43 aa, synthetic construct
GWGIPWCTLDPGGLQCAWLRQMGGGGSGGVVCQDWEGVELCWQ

SEQ ID NO: 907 - protein, 42 aa, synthetic construct
GWGIPWCTLDPGGLQCAWLRQGGGGSGGVVCQDWEGVELCWQ

SEQ ID NO: 908 - protein, 41 aa, synthetic construct
GWGIPWCTLDPGGLQCAWLRGGGGSGGVVCQDWEGVELCWQ

SEQ ID NO: 909 - protein, 42 aa, synthetic construct
GWGIPWCTLDPGGLQCAWLGKHGGGGSGGVVCQWEGVELCWQ

SEQ ID NO: 910 - protein, 42 aa, synthetic construct
GWGIPWCTLDPGGLQCAWLRMGGGGSGGVVCQDWEGVELCWQ

SEQ ID NO: 911 - protein, 40 aa, synthetic construct; identical to SEQ ID NO: 862

SEQ ID NO: 912 - protein, 39 aa, synthetic construct; identical to SEQ ID NO: 863

SEQ ID NO: 913 - protein, 38 aa, synthetic construct; identical to SEQ ID NO: 864

SEQ ID NO: 914 - protein, 39 aa, synthetic construct
IPWCTLDPGGLQCAWLGKHGGGGSGGVVCQWEGVELCWQ

SEQ ID NO: 915 - protein, 39 aa, synthetic construct
IPWCTLDPGGLQCAWLRMGGGGSGGVVCQDWEGVELCWQ

SEQ ID NO: 916 - protein, 37 aa, synthetic construct
  VARIANT 23: note = This region may encompass (G)n, (GS)n, (GGS)n, (GGGS)n, (GGGGS)n, or a combination of any of the foregoing, where n is an integer from 1 to 5.
VHRIPWCTLDPGGLQCAWLRQMXVVCQDWEGVELCWQ

SEQ ID NO: 917 - protein, 36 aa, synthetic construct
  VARIANT 22: note = This region may encompass (G)n, (GS)n, (GGS)n, (GGGS)n, (GGGGS)n, or a combination of any of the foregoing, where n is an integer from 1 to 5.
VHRIPWCTLDPGGLQCAWLRQXVVCQDWEGVELCWQ

SEQ ID NO: 918 - protein, 35 aa, synthetic construct
  VARIANT 21: note = This region may encompass (G)n, (GS)n, (GGS)n, (GGGS)n, (GGGGS)n, or a combination of any of the foregoingm, where n is an integer from 1 to 5.
VHRIPWCTLDPGGLQCAWLRXVVCQDWEGVELCWQ

SEQ ID NO: 919 - protein, 37 aa, synthetic construct
  VARIANT 23: note = This region may encompass (G)n, (GS)n, (GGS)n, (GGGS)n, (GGGGS)n, or a combination of any of the foregoing, where n is 1-5.
VHRIPWCTLDPGGLQCAWLGKHXVVCQDWEGVELCWQ

SEQ ID NO: 920 - protein, 36 aa, synthetic construct
  VARIANT 22: note = This region may encompass (G)n, (GS)n, (GGS)n, (GGGS)n, (GGGGS)n, or a combination of any of the foregoing, where n is 1-5.
VHRIPWCTLDPGGLQCAWLRMXVVCQDWEGVELCWQ

SEQ ID NO: 921 - protein, 37 aa, synthetic construct
  VARIANT 23: note = This region may encompass (G)n, (GS)n, (GGS)n, (GGGS)n, (GGGGS)n, or a combination of any of the foregoing, where n is 1-5.
GWGIPWCTLDPGGLQCAWLRQMXVVCQDWEGVELCWQ

SEQ ID NO: 922 - protein, 36 aa, synthetic construct
  VARIANT 22: note = This region may encompass (G)n, (GS)n, (GGS)n, (GGGS)n, (GGGGS)n or a combination of any of the foregoing, where n is 1-5.
GWGIPWCTLDPGGLQCAWLRQXVVCQDWEGVELCWQ

SEQ ID NO: 923 - protein, 35 aa, synthetic construct
  VARIANT 21: note = This region may encompass (G)n, (GS)n, (GGS)n, (GGGS)n, (GGGGS)n or a combination of any of the foregoiong, where n is 1-5.
GWGIPWCTLDPGGLQCAWLRXVVCQDWEGVELCWQ

SEQ ID NO: 924 - protein, 37 aa, synthetic construct
  VARIANT 23: note = This region may encompass (G)n, (GS)n, (GGS)n, (GGGS)n, (GGGGS)n, or a combination of any of the foregoing, where n is 1-5.
GWGIPWCTLDPGGLQCAWLGKHXVVCQDWEGVELCWQ

SEQ ID NO: 925 - protein, 36 aa, synthetic construct
  VARIANT 22: note = This region may encompass (G)n, (GS)n, (GGS)m, (GGGS)n, (GGGGS)n, GGGGS)n or a combination of any of the foregoing, where n is 1-5.
GWGIPWCTLDPGGLQCAWLRMXVVCQDWEGVELCWQ

SEQ ID NO: 926 - protein, 34 aa, synthetic construct
  VARIANT 20: note = This region may encompass (G)n, (GS)n, (GGS)n, (GGGS)n, (GGGGS)n, or a combination of any of the foregoing, where n is 1-5.
IPWCTLDPGGLQCAWLRQMXVVCQDWEGVELCWQ

SEQ ID NO: 927 - protein, 33 aa, synthetic construct
  VARIANT 19: note = This region may encompass (G)n, (GS)n, (GGS)n, (GGGS)n, (GGGGS)n or a combination of any of the foregoing, where n is 1-5.
IPWCTLDPGGLQCAWLRQXVVCQDWEGVELCWQ

SEQ ID NO: 928 - protein, 32 aa, synthetic construct
  VARIANT 18: note = This region may encompass (G)n, (GS)n, (GGS)n, (GGGS)n, (GGGGS)n, or a combination of any of the foregoing, where n is 1-5.
IPWCTLDPGGLQCAWLRXVVCQDWEGVELCWQ

SEQ ID NO: 929 - protein, 34 aa, synthetic construct
  VARIANT 20: note = This region may encompass (G)n, (GS)n, (GGS)n, (GGGS)n, (GGGGS)n, or a combination of any of the foregoing, where n is 1-5.
IPWCTLDPGGLQCAWLGKHXVVCQDWEGVELCWQ

SEQ ID NO: 930 - protein, 33 aa, synthetic construct
  VARIANT 19: note = This region may encompass (G)n, (GS)n, (GGS)n, (GGGS)n, (GGGGS)n, or a combination of any of the foregoing, where n is 1-5.
IPWCTLDPGGLQCAWLRMXVVCQDWEGVELCWQ

SEQ ID NO: 931 - protein, 14 aa, synthetic construct
RSCYYKRPRLWCSE

SEQ ID NO: 932 - protein, 14 aa, synthetic construct
YDCRIAQVGELCDL

SEQ ID NO: 933 - protein, 14 aa, synthetic construct
NMCLVGDYWPSCQI

SEQ ID NO: 934 - protein, 12 aa, synthetic construct
RWGDVGDLLMPL

SEQ ID NO: 935 - protein, 12 aa, synthetic construct
DCSMWEGVELCW

SEQ ID NO: 936 - protein, 14 aa, synthetic construct
KVCEMWGGVLLCWN

SEQ ID NO: 937 - protein, 14 aa, synthetic construct
RTCTEWENVVLCWV

SEQ ID NO: 938 - protein, 14 aa, synthetic construct
MCWLEWGEWVGSCL

SEQ ID NO: 939 - protein, 12 aa, synthetic construct
DLSDLSTFWLSQ

SEQ ID NO: 940 - protein, 14 aa, synthetic construct
CPSMLQGPERTWVC

(19) United States
(12) Patent Application Publication
(10) Pub. No.: US 2024/0101959 A1
(43) Pub. Date: Mar. 28, 2024

(54) USE OF IL-2R-BETA-GAMMA AGONIST PEPTIDES IN CELL MANUFACTURING

(71) Applicant: MEDIKINE, INC., MENLO PARK, CA (US)

(72) Inventors: WILLIAM J. DOWER, MENLO PARK, CA (US); MICHAEL C. NEEDELS, MENLO PARK, CA (US); RONALD W. BARRETT, SARATOGA, CA (US); ALICE V. BAKKER, CUPERTINO, CA (US); STEVEN E. CWIRLA, MENLO PARK, CA (US); SUNIL BAJAD, FREMONT, CA (US); INKYUNG PARK, MORGAN HILL, CA (US)

(21) Appl. No.: 18/462,315

(22) Filed: Sep. 6, 2023

Related U.S. Application Data
(60) Provisional application No. 63/403,996, filed on Sep. 6, 2022.

(51) Int. Cl. C12N 5/0783; C07K 14/55

(52) U.S. Cl. CPC C12N 5/0646; C07K 14/55; C12N 5/0636; C07K 2319/00; C12N 2501/2302

(57) ABSTRACT

The use of IL-2Rβγc agonist peptides in cell manufacturing is disclosed. The IL-2Rβγc agonist peptides can facilitate the selective growth of immune cell populations. Culture media comprising the IL-2Rβγc agonist peptides, methods of expanding a target immune cell population, and methods of manufacturing a target immune cell population are disclosed. The enriched immune cell populations can be used in immunotherapy.

This application claims the benefit under 35 U.S.C. § 119(e) of U.S. Provisional Application No. 63/403,996 filed on Sep. 6, 2022, which is incorporated by reference in its entirety.

### FIELD

The present disclosure relates to the use of IL-2Rβγc agonist peptides in cell manufacturing. The IL-2Rβγc agonist peptides can facilitate the selective growth of immune cell populations. The enriched immune cell populations are useful in immunotherapy.

This application claims the benefit under 35 U.S.C. § 119(e) of U.S. Provisional Application No. 63/403,996 filed on Sep. 6, 2022, which is incorporated by reference in its entirety.

### SEQUENCE LISTING

The Sequence Listing associated with this application is filed in electronic format by EFS-Web and is incorporated by reference in its entirety. The XML document in WIPO ST.26 format is named 62AJ-001510US-374590 Sequence Listing Sep-6-2023 XML and is 750 kilobytes in size.

### BACKGROUND

Recombinant cytokines can be used to stimulate immune cell proliferation.

The use of recombinant cytokines to stimulate immune cell proliferation has a number of drawbacks. Recombinant cytokines can have a wide range of activity depending on the source and batch and therefore the activity must be measured at the time of use and custom dilutions made to accommodate accurate and reproducible cell manufacturing. Recombinant cytokines are unstable as liquid formulations and therefore are typically provided as lyophilizates that must be reconstituted before use. The process for manufacturing GMP-grade recombinant cytokines is expensive. The purity of recombinant cytokines can be compromised by contamination with endotoxins and host cell proteins for bacterial systems, and by adventitious agents for mammalian and insect recombinant systems. Also, quality control procedures required to ensure product quality can be time-consuming and expensive.

Aspects of the present invention are directed to the use of small peptides as stimulants of T cell proliferation. The small peptides can replace the corresponding recombinant cytokines used in the manufacture of immune cells and cell therapy products.

IL-2Rβγc peptides that function as IL-2R agonists are disclosed in U.S. Application Publication No. 2021/0198336 A1, which is incorporated by reference in its entirety.

### SUMMARY

According to the present disclosure culture media for expanding a target immune cell population comprise a first stimulant of proliferation for the target immune cell population, wherein the first stimulant comprises an IL-2Rβγc agonist peptide.

According to the present disclosure methods of expanding a target immune cell population of an initial immune cell population comprise incubating an initial immune cell population in a culture medium according to the present disclosure to provide an expanded target immune cell population.

According to the present disclosure, methods of manufacturing an immune cell population, comprise activating a target immune cell population of a population of primary cells to provide an activated target immune cell population; and expanding the activated target immune cell population, wherein expanding comprises incubating the activated target immune cell population in the presence of an IL-2Rβγc agonist peptide to provide an expanded target immune cell population.

According to the present disclosure, an enriched population of immune cells is prepared using the culture medium according to the present disclosure.

According to the present disclosure, an enriched population of immune cells is prepared using a method of expanding an immune cell population according to the present disclosure.

According to the present disclosure, an enriched population of immune cells is prepared using a method of manufacturing an immune cell population according to the present disclosure.

According to the present disclosure, pharmaceutical compositions comprise an enriched population of immune cells according to the present disclosure.

According to the present disclosure, methods of treating a disease in a patient comprise administering to a patient in need of such treatment a therapeutically effective amount of a pharmaceutical composition according to the present disclosure.

According to the present disclosure, articles of manufacture comprise a culture medium according to the present disclosure.

According to the present disclosure, articles of manufacture comprise an enriched population of immune cells according to the present disclosure.

According to the present disclosure, articles of manufacture comprise a pharmaceutical composition according to the present disclosure.

According to the present disclosure, immobilized IL-2Rβγc agonist peptides comprise an IL-2Rβγc agonist peptides bound to a substrate.

### BRIEF DESCRIPTION OF THE DRAWINGS

The drawings described herein are for illustration purposes only. The drawings are not intended to limit the scope of the present disclosure.

FIGS. 1A-1D show STAT5 phosphorylation in resting PBMCs by an IL-2Rβγc agonist peptide (SEQ ID NO: 851) and by an IL-7Rαγc agonist peptide (SEQ ID NO: 891).

FIGS. 2A-2D show STAT5 phosphorylation in activated PBMCs by an IL-2Rβγc agonist peptide (SEQ ID NO: 851) and by an IL-7Rαγc agonist peptide (SEQ ID NO: 891).

FIGS. 3A-3E show the cell count of CD8 memory cell subpopulations following incubation of resting PBMCs for four weeks with an IL-2Rβγc agonist peptide (SEQ ID NO: 852), with an IL-7Rαγc agonist peptide (SEQ ID NO: 891), or without an agonist peptide.

FIGS. 4A-4E show the cell count of CD8 memory cell subpopulations following incubation of activated PBMCs for four weeks with an IL-2Rβγc agonist peptide (SEQ ID NO: 852), with an IL-7Rαγc agonist peptide (SEQ ID NO: 891), or without an agonist peptide.

FIGS. 5A-5B show NK subpopulations in resting NK cells following incubation with or without an IL-2Rβγc agonist peptide (SEQ ID NO: 852) for twenty-one (21) days.

FIGS. 6A-6B show NK subpopulations in resting NK cells following incubation with or without an IL-7Rαγc agonist peptide (SEQ ID NO: 891) for twenty-one (21) days.

FIGS. 7A-7C show NK subpopulations following incubation of resting NK cells without an agonist peptide, with an IL-2Rβγc agonist peptide (SEQ ID NO: 852), or with an IL-7Rαγc agonist peptide (SEQ ID NO: 891).

FIGS. 8A-8C show NK subpopulations following incubation of activated NK cells without an agonist peptide, with an IL-2Rβγc agonist peptide (SEQ ID NO: 852), or with an IL-7Rαγc agonist peptide (SEQ ID NO: 891).

FIG. 9A shows activation of NK-92 cells following exposure to IL-2 or an IL-2Rβγc agonist peptide having SEQ ID NO: 801 (Ac).

FIG. 9B shows proliferation of NK cells in human PBMCs following exposure to different concentrations of IL-2 or an IL-2Rβγc agonist peptide having SEQ ID NO: 801 (Ac).

### DETAILED DESCRIPTION

A dash (“—”) that is not between two letters or symbols is used to indicate a point of attachment for a moiety or substituent. For example, —CONH₂ is bonded through the carbon atom and —X¹—X²— denotes amino acids X¹ and X² covalently bound together through a single bond.

“Alkyl” refers to a saturated, branched, or straight-chain, monovalent hydrocarbon radical derived by the removal of one hydrogen atom from a single carbon atom of a parent alkane, alkene, or alkyne. Examples of alkyl groups include methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like. The term “alkyl” is specifically intended to include groups having any degree or level of saturation, i.e., groups having exclusively carbon-carbon single bonds, groups having one or more carbon-carbon double bonds, groups having one or more carbon-carbon triple bonds, and groups having combinations of carbon-carbon single, double, and triple bonds. Where a specific level of saturation is intended, the terms alkanyl, alkenyl, and alkynyl are used. In certain embodiments, an alkyl group is C₁₋₆ alkyl, C₁₋₅ alkyl, C₁₋₄ alkyl, C₁₋₃ alkyl, and in certain embodiments, ethyl or methyl.

“Cycloalkyl” refers to a saturated or partially unsaturated cyclic alkyl radical. In certain embodiments, a cycloalkyl group is C₃₋₆ cycloalkyl, C₃₋₅ cycloalkyl, C₅₋₆ cycloalkyl, cyclopropyl, cyclopentyl, and in certain embodiments, cyclohexyl. In certain embodiments, cycloalkyl is selected from cyclopropyl, cyclobutyl, cyclopentyl, and cyclohexyl.

“Heterocycloalkyl” by itself or as part of another substituent refers to a saturated or unsaturated cyclic alkyl radical in which one or more carbon atoms (and certain associated hydrogen atoms) are independently replaced with the same or a different heteroatom; or to a parent aromatic ring system in which one or more carbon atoms (and certain associated hydrogen atoms) are independently replaced with the same or a different heteroatom such that the ring system violates the Hückel-rule. Examples of heteroatoms to replace the carbon atom(s) include N, P, O, S, and Si. Examples of heterocycloalkyl groups include groups derived from epoxides, azirines, thiiranes, imidazolidine, morpholine, piperazine, piperidine, pyrazolidine, pyrrolidine, and quinuclidine. In certain embodiments, heterocycloalkyl is C₅ heterocycloalkyl and is selected from pyrrolidinyl, tetrahydrofuranyl, tetrahydrothiophenyl, imidazolidinyl, oxazolidinyl, thiazolidinyl, doxolanyl, and dithiolanyl. In certain embodiments, heterocycloalkyl is C₆ heterocycloalkyl and is selected from piperidinyl, tetrahydropyranyl, piperizinyl, oxazinyl, dithianyl, and dioxanyl. A heterocycloalkyl group can be C₃₋₆ heterocycloalkyl, C₃₋₅ heterocycloalkyl, C₅₋₆ heterocycloalkyl, and in certain embodiments, C₅ heterocycloalkyl or C₆ heterocycloalkyl. A heteroatomic group can be selected from —O—, —S—, —NH—, —N(—CH₃)—, —SO—, and —SO₂—, in certain embodiments, the heteroatomic group is selected from —O— and —NH—, and in certain embodiments the heteroatomic group is —O— or —NH—.

“Binding affinity” refers to the strength of the binding interaction between a single biomolecule and its ligand/binding partner. Binding affinity is expressed as the IC₅₀. For example, the binding affinity of a compound such as an IL-2Rβγc agonist peptide refers to the IC₅₀ as determined using, for example, a method described in the examples.

“Bond” or “bonded” refers to covalent bonding involving the sharing of electrons to form electron pairs between atoms.

“Direct binding” refers to the binding interaction between a single biomolecule and its binding partner such as, for example, the interaction of an IL-2Rβ ligand and the hu-IL-2Rb subunit or the interaction of an IL-2Rβγc agonist peptide and IL-2R. Direct binding can be determined using phage ELISA assays.

“Agonist” refers to a biologically active ligand which binds to its complementary biologically active receptor or receptor subunit(s) and activates the receptor to cause a biological response mediated by the receptor, or to enhance a preexisting biological activity mediated by the receptor.

“Partial agonist” refers to a compound that provides a level of activation, that is, for example, less than 75% of maximum activation, less than 50%, less than 25%, less than 10%, or less than 1% of the maximum activation. For example, a partial IL-2R agonist exhibits a level of activation that is less than the level of activation provided by IL-2. For example, a partial IL-2R agonist exhibits a level of activation that is less than the level of activation provided by IL-2 and a partial IL-2R agonist exhibits a level of activation that is less than the level of activation provided by IL-2.

“Antagonist” refers to a biologically active ligand or compound that binds to its complementary receptor or subunit(s) and blocks or reduces a biological response of the receptor. For example, an IL-2R antagonist binds to IL-2R with an IC₅₀ of less than 100 μM and inhibits functional activity of IL-2 as determined, for example, using any of the functional assays disclosed in the examples. For example, an IL-2R antagonist binds to IL-2R with an IC₅₀ of less than 100 μM and inhibits the functional activity of IL-2 as determined, for example, using a functional assay such as any of the functional assays disclosed in the examples.

Amino acid residues are abbreviated as follows: alanine is Ala or A; arginine is Arg or R; asparagine is Asn or N; aspartic acid is Asp or D; cysteine is Cys or C; glutamic acid is Glu or E; glutamine is Gln or Q; glycine is Gly or G; histidine is His or H; isoleucine is Ile or I; leucine is Leu or L; lysine is Lys or K; methionine is Met or M; phenylalanine is Phe or F; proline is Pro or P; serine is Ser or S; threonine is Thr or T; tryptophan is Trp or W; tyrosine is Tyr or Y; and valine is Val or V.

“Non-natural amino acids” include, for example, β-amino acids, homo-amino acids, proline and pyruvic acid derivatives, histidine derivatives with alkyl or heteroatom moieties bonded to the imidazole ring, amino acids with pyridine-containing side chains, 3-substituted alanine derivatives, glycine derivatives, ring-substituted phenylalanine, and tyrosine derivatives, and N-methyl amino acids.

Amino acids having a large hydrophobic side chain include, for example, isoleucine (I), leucine (L), methionine (M), valine (V), phenylalanine (F), tyrosine (Y), and tryptophan (W).

Amino acids having a small hydrophobic side chain include, for example, alanine (A), glycine (G), proline (P), serine (S), and threonine (T).

Amino acids having a basic side chain include, for example, arginine (R), lysine (K), and histidine (H).

Amino acids having an acidic side chain include, for example, aspartate (D) and glutamate (E).

Amino acids having a polar/neutral side chain include histidine (H), asparagine (N), glutamine (Q), serine (S), threonine (T), and tyrosine (Y).

Amino acids having an aromatic side chain include, for example, phenylalanine (F), histidine (H), tryptophan (W), and tyrosine (Y).

Amino acids having a hydroxyl side chain include, for example, serine (S), threonine (T), and tyrosine (Y).

“Conservative amino acid substitution” means that amino acids within each of the following groups can be substituted with another amino acid within the group such as, for example, amino acids having a small hydrophobic side chain comprising alanine (A), glycine (G), proline (P), serine (S), and threonine (T); amino acids having a hydroxyl-containing side chain comprising serine (S), threonine (T), and tyrosine (Y); amino acids having an acidic side chain comprising aspartate (D) and glutamate (E); amino acids comprising a polar-neutral side chain comprising histidine (H), asparagine (N), glutamine (Q), serine (S), threonine (T), and tyrosine (Y); amino acids having a basic side chain comprising arginine (R), lysine (K), and histidine (H); amino acids having a large hydrophobic side chain comprising isoleucine (I), leucine (L), methionine (M), valine (V), phenylalanine (F), tyrosine (Y), and tryptophan (W); and amino acids having an aromatic side chain comprising phenylalanine (F), histidine (H), tryptophan (W), and tyrosine (Y).

“PEG,” “polyethylene glycol” and “poly(ethylene glycol)” refer to any suitable nonpeptidic water-soluble poly(ethylene oxide). PEGs can comprise a structure —(OCH₂CH₂)ₙ— where n is, for example, an integer from 1 to 4,000. A PEG can also include moieties such as —CH₂CH₂—O(CH₂CH₂O)ₙ—CH₂CH₂— and/or —(OCH₂CH₂)ₙO—, depending upon whether or not the terminal oxygens have been displaced, e.g., during a synthetic transformation. A PEG can be capped with a suitable end group. At least 50% of the repeating subunits of a PEG can have the structure —CH₂CH₂—. A PEG can have any suitable molecular weight, structure, and/or geometry such as branched, linear, forked, or multifunctional.

A “physiologically cleavable” or “hydrolyzable” or “degradable” bond is a bond that reacts with water (i.e., is hydrolyzed) under physiological conditions. The tendency of a bond to hydrolyze in water will depend not only on the general type of linkage connecting two central atoms but also on the substituents bonded to these central atoms. Suitable hydrolytically unstable or weak linkages include, for example, carboxylate ester, phosphate ester, anhydrides, acetals, ketals, acyloxyalkyl ether, imines, orthoesters, peptides, and oligonucleotides.

An “enzymatically degradable linkage” refers to a chemical linkage that can be degraded or cleaved by one or more enzymes.

A “hydrolytically stable” linkage or bond refers to a chemical bond, such as a covalent bond, that is substantially stable in water such that the chemical bond does not undergo hydrolysis under physiological conditions to any appreciable extent over an extended period of time. Examples of hydrolytically stable linkages include, but are not limited to, the following: carbon-carbon bonds (e.g., in aliphatic chains), ethers, amides, urethanes, and the like. Generally, a hydrolytically stable linkage is one that exhibits a rate of hydrolysis of less than about 1% to 2% per day under physiological conditions such as in human blood.

An “IL-2Rα ligand” refers to a peptide capable of binding to the IL-2Rα subunit of a mammalian IL-2 receptor, such as a hu-IL-2 receptor, with an IC₅₀ less than 100 μM.

An “IL-2Rβ ligand” refers to a peptide capable of binding to the IL-2Rβ subunit of a mammalian IL-2 receptor, such as the hu-IL-2 receptor, with an IC₅₀ less than 100 μM.

An “IL-2Rβ ligand” or an “IL-2Rβ ligand provided by the present disclosure” includes, for example, a peptide having an amino acid sequence of any one of SEQ ID NO: 74-172, 173-253, 330-560, 561-587, and 588-608; a truncated amino acid sequence based on SEQ ID NO: 74-172, 173-253, 330-560, 561-587, and 588-608; an amino acid sequence having SEQ ID NO: 74-172, 173-253, 330-560, 561-587, and 588-608 and flanking amino acids; an amino acid sequence based on SEQ ID NO: 74-172, 173-253, 330-560, 561-587, and 588-608 having one or more conservative or non-conservative amino acid substitutions, an amino acid sequence having greater than 60% sequence similarity to any one of SEQ ID NO: 74-172, 173-253, 330-560, 561-587, and 588-608; or a combination of any of the foregoing.

An “IL-2Rγc ligand” or “Rγc ligand” refers to a peptide capable of binding to the IL-2Rγc subunit of a mammalian IL-2 receptor, such as the hu-IL-2 receptor, and the IL-7Rγc subunit of a mammalian IL-7 receptor such as the hu-IL-7 receptor, with an IC₅₀ less than 100 μM.

An “IL-2Rγc ligand”, an “IL-2Rγc ligand provided by the present disclosure”, an “Rγc ligand”, and an “Rγc ligand provided by the present disclosure” includes, for example, a peptide having an amino acid sequence of any one of SEQ ID NO: 631-715 and 723-749, a truncated amino acid sequence based on SEQ ID NO: 631-715 and 723-749, an amino acid sequence having SEQ ID NO: 631-715 and 723-749 and flanking amino acids, an amino acid sequence based on SEQ ID NO: 631-715, 723-749 having one or more conservative or non-conservative amino acid substitutions, an amino acid sequence having greater than 60% sequence similarity to any one of SEQ ID NO: 631-715 and 723-749, or a combination of any of the foregoing. An IL-2Rγc ligand is the same as an IL-7Rγc ligand and can be referred to as a Rγc ligand.

A ligand comprising an amino acid sequence having SEQ ID NO: refers to a ligand having the amino acid sequence identified by the SEQ ID NO:, a truncated amino acid sequence based on the SEQ ID NO:, an amino acid sequence having the SEQ ID NO: and flanking amino acids, an amino acid sequence based on the SEQ ID NO: having one or more conservative or non-conservative amino acid substitutions, an amino acid sequence having greater than 60% sequence similarity to the amino acid sequence identified by the SEQ ID NO:, or a combination of any of the foregoing.

The “hu-IL-2Rβ subunit” refers to the human (Homo sapiens) interleukin-2 receptor subunit β precursor NCBI Reference Sequence NP_000689.1.

The “hu-IL-2Rγc subunit” refers to the human (Homo sapiens) interleukin-2 receptor subunit γ precursor NCBI Reference Sequence NP_000197.1.

The “cyano-IL-2Rβ subunit” refers to the cynomolgus monkey interleukin-2 receptor subunit β precursor NCBI Reference Sequence NP_001244989.1.

The “cyano-IL-2Rγc subunit” refers to the cynomolgus monkey interleukin-2 receptor subunit α precursor NCBI Reference Sequence XP_005593949.

An “IL-7Rα ligand” refers to a peptide capable of binding to the IL-7Rα subunit of a mammalian IL-7 receptor, such as the hu-IL-7 receptor, with an IC₅₀ of less than 100 μM.

The “hu-IL-7Rα subunit” refers to the human (Homo sapiens) interleukin-7 receptor subunit α precursor NCBI Reference Sequence NP_002176.2.

An “IL-2Rβγc agonist peptide” or IL-2Rβγc agonist peptide provided by the present disclosure” includes, for example, at least one IL-2Rβ ligand provided by the present disclosure and at least one IL-2Rγc ligand provided by the present disclosure. An IL-2Rβγc agonist peptide is capable of activating hu-IL-2R with an EC50 less than 100 μM, less than 10 μM, less than 1 μM, less than 0.1 μM, or less than 0.01 μM, wherein the EC50 is determined using ELISA competition assays.

An “IL-2Rβγc agonist peptide” comprises one or more IL-2Rβ ligands and one or more IL-2Rγc ligands. The one or more IL-2Rβ ligands and one or more IL-2Rγc ligands can be bound to an IL-2Rβγc linker. An IL-2Rβγc agonist peptide can comprise two or more IL-2Rb ligands linked to two or more IL-2Rγc ligands, or an IL-2Rβγc agonist peptide can comprise a single ligand that simultaneously binds to both the IL-2R□ subunit and the IL-2Rγc subunit. An IL-2Rβγc peptide agonist can comprise an IL-2Rβγc peptide agonist bound to one or more IL-2Rβγc peptide agonists. An IL-2Rβγc agonist peptide is binds to both the IL-2Rβ subunit and to the IL-2Rγc subunit of IL-2R such as hu-IL-2R with an IC₅₀ less than 100 μM, less than 10 μM, less than 1 μM, or less than 0.1 μM. An IL-2Rβγc agonist peptide can bind to IL-2R such as hu-IL-2R with an IC₅₀, for example, of less than 100 μM such as less than 10 μM, less than 1 μM, less than 0.1 μM, or less than 0.01 μM.

An “IL-7Rαγc agonist peptide” refers to a compound consisting of or comprising one or more IL-7Rα ligands and one or more Rγc ligands. The one or more IL-7Rα ligands and one or more Rγc ligands can be bound to an IL-7Rαγc linker. An IL-7Rαγc agonist peptide can comprise an IL-7Rαγc agonist peptide comprising two or more IL-7Rαγc agonist peptides, or an IL-7Rαγc agonist peptide can comprise a single ligand that simultaneously binds to both the hu-IL-7Rα subunit and the hu-IL-7Rγc subunit. An IL-7Rαγc agonist peptide is capable of binding to the hu-IL-7Rα subunit and to the hu-IL-7Rγc subunit of hu-IL-7R with an IC₅₀ of less than 100 μM. An IL-7Rαγc agonist peptide is capable of binding to hu-IL-7R with an IC₅₀ of less than 100 μM such as less than 10 μM, less than 1 μM, less than 0.1 μM, or less than 0.01 μM.

Bioisosteres are atoms or molecules that fit the broadest definition for isosteres. The concept of bioisosterism is based on the concept that single atoms, groups, moieties, or whole molecules, which have chemical and physical similarities produce similar biological effects. A bioisostere of a parent compound can still be recognized and accepted by its appropriate target, but its functions will be altered as compared to the parent molecule. Parameters influenced by bioisosteric replacements include, for example, size, conformation, inductive and mesomeric effects, polarizability, capacity for electrostatic interactions, charge distribution, H-bond formation capacity, pKa (acidity), solubility, hydrophobicity, lipophilicity, hydrophilicity, polarity, potency, selectivity, reactivity, or chemical and metabolic stability, ADME (absorption, distribution, metabolism, and excretion). Although common in pharmaceuticals, carboxyl groups or carboxylic acid functional groups (—CO₂H) in a parent molecule may be replaced with a suitable surrogate or (bio)isostere to overcome chemical or biological shortcomings while retaining the desired attributes of the parent molecule bearing one or more carboxyl groups or carboxylic acid functional groups (—CO₂H). IL-2Rβγc agonist peptides include bioisosteres of the IL-2Rβγc agonist peptides provided by the present disclosure.

“Isostere” or “isostere replacement” refers to any amino acid or other analog moiety having physiochemical and/or structural properties similar to a specified amino acid. An “isostere” or “suitable isostere” of an amino acid is another amino acid of the same class, wherein amino acids belong to the following classes based on the propensity of the side chain to be in contact with a polar solvent like water: hydrophobic (low propensity to be in contact with water), polar or charged (energetically favorable contact with water). Examples of charged amino acid residues include lysine (+), arginine (+), aspartate (−), and glutamate (−). Examples of polar amino acids include serine, threonine, asparagine, glutamine, histidine, and tyrosine. Illustrative hydrophobic amino acids include alanine, valine, leucine, isoleucine, proline, phenylalanine, tryptophan, cysteine, and methionine. The amino acid glycine does not have a side chain and is difficult to assign to one of the above classes. However, glycine is often found at the surface of proteins, often within loops, providing high flexibility to these regions, and an isostere may have a similar feature. Proline has the opposite effect, providing rigidity to the protein structure by imposing certain torsion angles on the segment of the polypeptide chain. An isostere can be a derivative of an amino acid, e.g., a derivative having one or more modified side chains as compared to the reference amino acid. IL-2Rβγc agonist peptides include isosteres of the IL-2Rβγc agonist peptides provided by the present disclosure. IL-7Rαγc agonist peptides include isosteres of the IL-7Rαγc agonist peptides provided by the present disclosure

“Cyclized” refers to a reaction in which one part of a peptide or polypeptide molecule becomes linked to another part of the peptide or polypeptide molecule to form a closed ring, such as by forming a disulfide bridge or other similar bond, e.g., a lactam bond. Peptide monomer compounds or monomer subunits of peptide dimer compounds described herein can be cyclized via an intramolecular bond between two amino acid residues present in the peptide monomer or monomer subunit. For example, an IL-2Rβγc agonist peptide can include cysteines that are bound together through disulfide bonds and thereby is a cyclized IL-2Rβγc agonist peptide. For example, an IL-7Rαγc agonist peptide can include cysteines that are bound together through disulfide bonds and thereby is a cyclized IL-7Rαγc agonist peptide.

“Patient” refers to a mammal, for example, a human.

“Peptide” refers to amino acids joined together through amide bonds. A peptide can comprise, for example, less than 200 amino acids, less than 100 amino acids, less than 50 amino acids, less than 40 amino acids, less than 30 amino acids, or less than 20 amino acids. A peptide can comprise naturally occurring amino acids, non-naturally occurring amino acids, or a combination thereof.

In addition to peptides consisting only of naturally occurring amino acids, peptidomimetics or peptide analogs are also provided. Peptide mimetics that are structurally similar to therapeutically useful peptides may be used to produce an equivalent or enhanced therapeutic or prophylactic effect. Generally, peptidomimetics are structurally similar to a paradigm peptide, for example, a peptide that has a biological or pharmacological activity, such as a naturally-occurring receptor-binding peptide, but have one or more peptide linkages optionally replaced by a linkage such as —CH₂—NH—, —CH₂—S—, —CH₂—CH₂—, —CH═CH— (cis and trans), —COCH₂—, —CH(OH)CH₂—, and —CH₂SO—, by methods known in the art.

Substitution of one or more amino acids of a consensus sequence with a D-amino acid of the same type, such as D-lysine in place of L-lysine, may be used to generate more stable peptides. In addition, constrained peptides comprising a consensus sequence or a substantially identical consensus sequence variation may be generated by methods known in the art; for example, by adding internal cysteine residues capable of forming intramolecular disulfide bridges which cyclize the peptide.

Synthetic or non-naturally occurring amino acids refer to amino acids that do not naturally occur in vivo but which, nevertheless, can be incorporated into the peptide ligands provided by the present disclosure. Suitable examples of synthetic amino acids include the D-α-amino acids of naturally occurring L-α-amino acid as well as non-naturally occurring D- and L-α-amino acids represented by the formula H₂NCHR⁵COOH where R⁵ is C₁₋₆ alkyl, C₃₋₈ cycloalkyl, C₃₋₈ heterocycloalkyl; an aromatic residue of from 6 to 10 carbon atoms optionally having from 1 to 3 substituents on the aromatic nucleus selected from hydroxyl, lower alkoxy, amino, and carboxyl; -alkylene-Y where alkylene is an alkylene group of from 1 to 7 carbon atoms and Y is selected from a hydroxyl, amino, cycloalkyl, and cycloalkenyl having from 3 to 7 carbon atoms; aryl of from 6 to 10 carbon atoms, such as from 1 to 3 substituents on the aromatic nucleus selected hydroxyl, lower alkoxy, amino and carboxyl; heterocyclic of from 3 to 7 carbon atoms and 1 to 2 heteroatoms selected from oxygen, sulfur, and nitrogen; —C(O)R² where R² is selected from hydrogen, hydroxy, lower alkyl, lower alkoxy, and —NR³R⁴ where each of R³ and R⁴ is independently selected from hydrogen and lower alkyl; —S(O)ₙR⁶ where n is 1 or 2 and R⁶ is C₁₋₆ alkyl, and with the proviso that R⁶ does not define a side chain of a naturally occurring amino acid.

Examples of other synthetic amino acids include amino acids in which the amino group is separated from the carboxyl group by more than one carbon atom such as β-alanine and γ-aminobutyric acid.

Examples of suitable synthetic amino acids include the D-amino acids of naturally occurring L-amino acids, L-1-naphthyl-alanine, L-2-naphthylalanine, L-cyclohexylalanine, L-2-amino isobutyric acid, the sulfoxide and sulfone derivatives of methionine, i.e., HOOC—(H₂NCH)CH₂CH₂—S(O)₁R, where n and R are as defined above as well as the lower alkoxy derivative of methionine, i.e., HOOC—(H₂NCH)CH₂CH₂OR where R is as defined above.

“N-terminus” refers to the end of a peptide or polypeptide, such as an N-terminus of an IL-2Rβγc agonist peptide, an IL-2Rβ ligand, or an IL-2Rγc ligand, that bears an amino group in contrast to the carboxyl end bearing a carboxylic acid group.

“C-terminus” refers to the end of a peptide or polypeptide, such as a C-terminus of an IL-2Rβγc agonist peptide, an IL-2Rβ ligand or an IL-2Rγc ligand that bears a carboxylic acid group in contrast to the amino terminus bearing an amino group. In certain synthetic peptides, the N-terminus does not bear an amino group and/or the C-terminus does not bear a carboxyl group. In such cases the nomenclature refers to the direction of the peptide backbone.

“Pharmaceutically acceptable” refers to approved or approvable by a regulatory agency of the Federal or a state government or listed in the U.S. Pharmacopoeia or other generally recognized pharmacopeia for use in animals, and more particularly in humans.

“Pharmaceutically acceptable salt” refers to a salt of a compound, which possesses a desired pharmacological activity of the parent compound. Such salts include acid addition salts, formed with inorganic acids and one or more protonate-able functional groups such as primary, secondary, or tertiary amines within the parent compound. Examples of inorganic acids include hydrochloric acid, hydrobromic acid, sulfuric acid, nitric acid, and phosphoric acid. A salt can be formed with organic acids such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, or muconic acid. A salt can be formed when one or more acidic protons present in the parent compound are replaced by a metal ion, e.g., an alkali metal ion, an alkaline earth ion, or an aluminum ion, or combinations thereof; or coordinates with an organic base such as ethanolamine, diethanolamine, triethanolamine, or N-methylglucamine. A pharmaceutically acceptable salt can be a hydrochloride salt. A pharmaceutically acceptable salt can be a sodium salt. A compound can have two or more ionizable groups, and a pharmaceutically acceptable salt can comprise one or more counterions, such as a bi-salt, for example, a dihydrochloride salt.

“Pharmaceutically acceptable salt” refers to hydrates and other solvates, as well as salts in crystalline or non-crystalline form. Where a particular pharmaceutically acceptable salt is disclosed, it is understood that the particular salt (e.g., a hydrochloride salt) is an example of a salt and that other salts may be formed using techniques known to one of skill in the art. Additionally, a pharmaceutically acceptable salt to the corresponding compound, free base and/or free acid, using techniques generally known in the art. See also: Stahl and Wermuth, C. G. (Editors), Handbook of Pharmaceutical Salts, Wiley-VCH, Weinheim, Germany, 2008.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to a composition comprising an IL-2Rβγc agonist peptide or a pharmaceutically acceptable salt thereof and at least one pharmaceutically acceptable vehicle or excipient with which the IL-2Rβγc agonist peptide or a pharmaceutically acceptable salt thereof is administered to a patient.

“Preventing” or “prevention” refers to a reduction in risk of acquiring a disease or disorder such as, for example causing at least one of the clinical symptoms of the disease not to develop in a patient that may be exposed to or predisposed to the disease but does not yet experience or display symptoms of the disease. In some embodiments, “preventing” or “prevention” refers to reducing symptoms of the disease by taking the compound in a preventative fashion.

“Therapeutically effective amount” refers to the amount of a compound that, when administered to a patient for treating a disease, or at least one of the clinical symptoms of a disease, is sufficient to treat the disease or symptom thereof. A “therapeutically effective amount” may vary depending, for example, on the compound, the disease and/or symptoms of the disease, severity of the disease and/or symptoms of the disease or disorder, the age, weight, and/or health of the patient to be treated, and the judgment of a prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

“Treating” or “treatment” of a disease refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter or manifestation that may or may not be discernible to the patient. In certain embodiments, “treating” or “treatment” refers to delaying the onset of the disease or at least one or more symptoms thereof in a patient who may be exposed to or predisposed to a disease or disorder even though that patient does not yet experience or display symptoms of the disease.

“Tregs” or “Treg cells” refer to regulatory T-cells. Regulatory T-cells are a class of T-cells that suppress the activity of other immune cells and are defined using flow cytometry by the cell marker phenotypes CD4+/CD25+/FOXP3+, CD4+CD25+CD127lo, or CD4+/CD25+/FOXP3+/CD127lo. Because FOXP3 is an intracellular protein and requires cell fixation and permeabilization for staining, the cell surface phenotype CD4+CD25+CD127lo− can be used for defining live Tregs. Tregs also include various Treg subclasses, such as tTregs (thymus-derived) and pTregs (peripherally derived, differentiated from naive T-cells in the periphery). Tregs play a critical role in the induction and maintenance of peripheral self-tolerance to antigens, including those expressed by tumors.

“CD4+T cells” are a type of lymphocyte that functions to coordinate the immune response by stimulating other immune cells such as macrophages, B lymphocytes (B cells), and CD8+ T lymphocytes (CD8+T cells) to fight infection. CD4+T cells recognize peptides presented on MHC Class II molecules, which are found on antigen-presenting cells.

As with CD4+ T cells, “CD8+(cytotoxic) T-cells” are generated in the thymus and express the T-cell receptor. Cytotoxic T-cells express a dimeric co-receptor, CD8, which typically comprises one CD8a and one CD8β chain. CD8+T-cells recognize peptides presented by MHC Class 1 molecules found on most nucleated cells. The CD8 heterodimer binds to a conservative portion of MHC Class 1 during T-cell/antigen presenting cell interactions. CD8+T-cells (cytotoxic T lymphocytes, or CTLs) are important for immune defense against intracellular pathogens, including viruses and bacteria, and for tumor surveillance.

“NK (natural killer) cells” are lymphocytes of the innate immune system and are classified as group I innate lymphocytes (ILCs). NK cells respond to a wide variety of pathological challenges, including by killing virally infected cells and detecting and controlling early signs of cancer.

IL-7 mediates a variety of responses in lymphocytes and other immune cell types. Assays for such responses include stimulation of pSTAT5, cell proliferation or markers of proliferation such as Ki67, change in immune cell type ratios, and stimulation of the levels of effector proteins.

“Functional activation of cells” refers to a cytokine-mediated response in cells such as, for example, T-cells and NK cells. Assays for functional activation of cells include stimulation of pSTAT5, cell proliferation or markers of proliferation (such as Ki67), change in immune cell type ratios, and stimulation of the levels of effector proteins.

“Effector cells” refers to a population of lymphocytes that mediate the helper (CD4+ cells) and cytotoxic (CD8+ and NK cells) effects. Effector cells include effector T-cells such as CD4+ helper T-cells, CD8+ cytotoxic T-cells, NK cells, lymphokine-activated killer (LAK) cells, and macrophages/monocytes.

“Naïve T-cells” refer to T-cells that have differentiated in bone marrow and undergone the positive and negative processes of central selection in the thymus. Naïve T-cells include naïve forms of helper T cells, CD4+ T-cells) and naïve cytotoxic T-cells (CD8+ T-cells). Naive T-cells are commonly characterized by the surface expression of L-selectin (CD62L) and C—C chemokine receptor type 7 (CCR7) and the expression of IL-7R (CD127) and the absence of the activation markers CD25, CD44, and CD69.

An “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. Immune cells can comprise T cells or NK cells.

“T-cells” can include, for example, CD4+ helper T-cells CD8+ T-cytotoxic T cell; memory T-cells such as (i) stem memory Tscm cells which express CCR7, CD45RA, CD62L (L-selectin), CD2, CD28, IL-7Ra, CD95, IL-2R, CXCR3, and LFA-1; (ii) central memory Tcm cells that express CD62L, CCR7 and CD45RO+ and secrete IL-2, but not IFN g or IL-4, and (iii) effector memory Tem cells that do not express L-selectin or CCR7 but express CD45RO and produce effector cytokines like IFNg and IL-4; (iv) terminal differentiated effector memory Temra cells that re-express CD45RA but CCR7−; regulatory T-cells such as Tregs, suppressor T cells, or CD4+CD25+ regulatory T cells; natural killer T cells (NKT); and γδT cells. T cells found within tumors are referred to as tumor infiltrating lymphocytes or TILs. A “naïve” T cell refers to a mature T cell that remains immunologically undifferentiated. Following positive and negative selection in the thymus, T cells emerge as either CD4+ or CD8+ naive T cells. In their naive state, T cells express L-selectin (CD62L), IL-7 receptor a (IL-7Ra), and CD132, but do not express CD25, CD44, CD69, or CD45RO. As used herein, “immature” can also refer to a T cell which exhibits a phenotype characteristic of either a naive T cell or an immature T cell, such as a Tscm cell or a Tcm cell. For example, an immature T cell can express one or more of L-selectin (CD62L), IL-7Ra, CD132, CCR7, CD45RA, CD45RO, CD27, CD28, CD95, CXCR3, and LFA-1. Naive or immature T cells can be contrasted with terminal differentiated effector T cells, such as Teff cells.

“Memory T-cells” are a subset of T lymphocytes including both CD4+ and CD8+. The primary function of memory T-cells is rapid augmented immune response after reactivation of those cells by reintroduction of a relevant antigen or pathogen into the body. Memory T cells refers to T cells that have previously encountered and responded to a cognate antigen (e.g., in vivo, in vitro, or ex vivo) or which have been stimulated in vitro or ex vivo with an antibody, such as, for example, with an anti-CD3 antibody. Immune cells having a “memory-like” phenotype upon secondary exposure, such memory T cells can mount a faster and stronger immune response than during the primary exposure. Memory T cells can comprise central memory T cells (Tcm cells), effector memory T cells (Tem cells), tissue resident memory T cells (Trm cells), stem cell-like memory T cells (Tscm cells), or any combination thereof.

“Stem cell-like memory T cells,” “T memory stem cells,” or “Tscm cells” refers to memory T cells that express CD95, CD45RA, CCR7, and CD62L and are endowed with the stem cell-like ability to self-renew and the multipotent capacity to reconstitute the entire spectrum of memory and effector T cell subsets.

“Central memory T cells” or “Tcm cells” refers to memory T cells that express CD45RO, CCR7, and CD62L. Central memory T cells are generally found within the lymph nodes and in the peripheral circulation.

“Effector memory T cells” or “Tem cells” refers to memory T cells that express CD45RO but lack expression of CCR7 and CD62L. Because effector memory T cells lack lymph node-homing receptors such as CCR7 and CD62L, these cells are typically found in the peripheral circulation and in non-lymphoid tissue.

“Terminally differentiated memory T cells” or “Temra cells” refer to memory T cells expressing CD45RA but lack expression of CCR7 and CD62L.

“Tissue resident memory T cells” or “TRM cells” refer to memory T cells that do not circulate and remain resident in peripheral tissue, such as skin, lung, and the gastrointestinal tract. Tissue-resident memory T cells can also be effector memory T cells.

“Naive T cells” or “Tn cells” refer to T cells that express CD45RA, CCR7, and CD62L, but do not express CD95. The interaction between a Tn cell and an antigen-presenting cell (APC) induces differentiation of the Tn cell towards an activated Teff cell and an immune response.

“Cytokine” refers to small, secreted proteins released by cells that have a specific effect on the interactions and communications between cells. Examples of cytokines include interleukins such as interleukin (IL)-1, IL-2, IL-4, IL-7, IL-9, IL-13, IL-15, IL-3, IL-5, IL-6, IL-11, IL-10, IL-20, IL-14, IL-16, IL-17, IL-21 and IL-23, interferons such as IFN; e.g., IFNa, IFN b, and IFNg), tumor necrosis factors (TNF), and transforming growth factors (TGF).

“Antigen binding moiety” refers to a polypeptide molecule that specifically binds to an antigenic determinant. An antigen binding moiety can direct, for example, the entity to which it is bonded, such as a cytokine or a second antigen binding moiety, to a target site, for example, to a specific type of tumor cell or tumor stroma bearing the antigenic determinant. Antigen binding moieties include antibodies and fragments thereof. Examples of antigen binding moieties include an antigen binding domain of an antibody comprising an antibody heavy chain variable region and an antibody light chain variable region. An antigen binding moiety can include antibody constant regions. Useful heavy chain constant regions can include any of the five isotypes: α, δ, ε, γ, or μ. Useful light chain constant regions can include any of the two isotypes K and A.

“Antibody” in the broadest sense encompasses various antibody structures including, for example, monoclonal antibodies, polyclonal antibodies, multi-specific antibodies such as bispecific antibodies, and antibody fragments that exhibit a desired antigen binding activity. The term “antibody” can be abbreviated as “ab” or “Ab” such as in the expression Fab or anti-phage Ab. Antibody refers to a polypeptide that includes canonical immunoglobulin sequence elements sufficient to confer specific binding to a particular target antigen. Intact antibodies as produced in nature are approximately 150 kD tetrameric agents comprising two identical heavy chain polypeptides (about 50 kD each) and two identical light chain polypeptides (about 25 kD each) that associate with each other into what is commonly referred to as a Y-shaped structure. Each heavy chain comprises at least four domains with each about 110 amino acids long, an amino-terminal variable (VH) domain located at the tips of the Y structure, followed by three constant domains: CHI, CH2, and the carboxy-terminal CH3 located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another switch.

“Full-length antibody,” “intact antibody,” and “whole antibody” refer to an antibody having a structure substantially similar to a native antibody structure or having heavy chains that contain both Fab and an Fc region.

“Antibody fragment” refers to a molecule other than an intact antibody that comprises a portion of an intact antibody that binds the antigen to which the intact antibody binds. Examples of antibody fragments include Fv, Fab, Fab′, Fab′-SH, F(ab′)2, diabodies, linear antibodies, single-chain antibody molecules such as scFv, and multi-specific antibodies formed from antibody fragments. Diabodies are antibody fragments with two antigen-binding sites that may be bivalent or bispecific. Antibody fragments can be made by various techniques, including but not limited to proteolytic digestion of an intact antibody as well as production by recombinant host cells such as E. coli or phage.

“Fab” or “Fab region” refers to a polypeptide that comprises the VH, CHI, VL, and CL immunoglobulin domains, generally on two different polypeptide chains such as VH-CHI on one chain and VL-CL on the other. Fab may refer to this region in isolation or this region in the context of a bispecific antibody. In the context of a Fab, the Fab comprises an Fv region in addition to the CHI and CL domains.

“Fv” or “Fv fragment” or “Fv region” refers to a polypeptide that comprises the VL and VH domains of an ABD. Fv regions can be formatted as both Fabs (generally two different polypeptides that also include the constant regions) and scFvs, where the VL and VH domains are combined (generally with a linker as discussed) to form an scFv.

“Single chain Fv” or “scFv” refers to a variable heavy domain covalently bonded to a variable light domain, generally using a scFv linker as discussed herein, to form a scFv or scFv domain. A scFv domain can be in either orientation with the VL domain at the N- or C-terminus of the polypeptide, and conversely for the VH domain.

“Effector function” refers to a biochemical event that results from the interaction of an antibody Fc region with an Fc receptor or ligand. Effector functions include, for example, antibody-dependent cellular toxicity (ADCC), antibody-dependent cellular phagocytosis (ADCP), and complement-dependent cytotoxicity (CDC).

“Fc” or “Fc region” or “Fc chain” refers to polypeptide comprising the constant region of an antibody, in some instances, excluding all or a portion of the first constant region immunoglobulin domain (e.g., CHI) or a portion thereof, and in some cases, further excluding all or a portion of the hinge. Thus, an Fc can refer to the last two constant region immunoglobulin domains (e.g., CH₂ and CH₃) of lgA, IgD, and IgG, the last three constant region immunoglobulin domains of IgE and IgM, and optionally, all or a portion of the flexible hinge N-terminal to these domains. For IgA and IgM, Fc may include the J chain. For IgG, the Fc chain comprises immunoglobulin domains CH₂ and CH₃ (Cy2 and Cy3), and optionally all or a portion of the hinge region between CHI (Cyl) and CH₂ (Cy2). Although the boundaries of the Fc region may vary, the hu-IgG heavy chain Fc region is usually defined to include residues E216, C226, or A231 to its carboxyl-terminus, wherein the numbering is according to the EU index as in Kabat. An amino acid modification can be made to the Fc region, for example to alter binding to one or more FcyR or to the FcRn. In EU numbering for hu-IgG1, the CH2-CH3 domain comprises amino acids 231 to 447, and the hinge is 216 to 230. Thus, the definition of Fc chain includes both amino acids 231-447 (CH2-CH3) or 216-447 (hinge-CH2-CH3), or fragments thereof. An Fc fragment can contain fewer amino acids from either or both of the N- and C-termini that retains the ability to form a dimer with another Fc chain or Fc fragment as can be detected using standard methods, generally based on size (e.g., non-denaturing chromatography, size exclusion chromatography, etc.). Hu-IgG Fc chains are of particular use and can be the Fc chain from hu-IgGl, hu-IgG2, or hu-IgG4.

“Heavy constant region” refers to the CH1-hinge-CH2-CH3 portion of an antibody or fragments thereof, excluding the variable heavy domain; in EU numbering of hu-IgGl, such as amino acids 118-447.

“Heavy chain constant region fragment” refers to a heavy chain constant region that contains fewer amino acids from either or both of the N- and C-termini that retains the ability to form a dimer with another heavy chain constant region.

“Immunoglobulin” refers to a protein having the structure of a naturally occurring antibody. For example, immunoglobulins of the IgG class are heterotetrameric glycoproteins of about 150,000 Da, composed of two light chains and two heavy chains that are bonded together through disulfide bonds. From N- to C-terminus, each heavy chain has a variable region (VH), also called a variable heavy domain or a heavy chain variable domain, followed by three constant domains (CHI, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five classes, called a (IgA), Ii (IgD), E (IgE), y (IgG), or μ (IgM), some of which may be further divided into subclasses, e.g., γ1 (IgG1), γ2 (IgG2), γ3 (IgG3), γ4 (gG4), α1 (IgA1) and α2 (IgA2). The light chain of an immunoglobulin may be assigned to one of two types, kappa (k) or lambda (L), based on the amino acid sequence of its constant domain. An immunoglobulin essentially consists of two Fab molecules and an Fc chain, linked via the immunoglobulin hinge region.

“Linker” refers to a moiety that binds one compound to another compound. Linkers can include IL-2Rβγc linkers and tandem IL-2Rβγc linkers. A linker can be a chemical linker. A linker can be an amino acid or peptide linker. For example, linkers provided by the present disclosure can facilitate the ability of an IL-2Rβγc agonist peptide to interact with IL-2R, to bind to IL-2R with low IC₅₀, and/or to activate IL-2R. A linker can comprise a peptide or a non-peptide. Non-peptide linkers include those containing, for example, a triazole moiety derived from a Cu(I)-catalyzed reaction of alkyne and azide functionalities. An IL-2Rβγc linker refers to a moiety that binds at least one IL-2R ligand such as an IL-2Rβ ligand and/or an IL-2Rγc ligand to another IL-2R ligand. For example, a ligand linker can bind an IL-2Rβ ligand to an Rγc ligand. A linker can bind to another IL-2R ligand which can be the same IL-2Rβγc ligand or a different IL-2Rβγc ligand. A linker can also bind to one or more additional moieties that provide a desired physiological function. For example, a construct linker can bind an IL-2Rβγc peptide agonist or an IL-7Rαγc agonist to a construct partner such as an antibody or an antibody fragment. A linker can be divalent or multivalent. A linker can be hydrolytically stable or may include a physiologically hydrolyzable or enzymatically degradable or cleavable linkage. A linker can bind IL-2R ligands to form dimers, trimers, or higher order multi-ligand peptides (heteromers) and compounds.

A “flexible linker” refers to a peptidyl linker comprising flexible amino acids such as glycine and serine. A flexible linker can comprise, for example, from 1 to 100 amino acids such as from 1 to 50, from 1 to 40, from 1 to 30, from 1 to 20, from 1 to 10, or from 1 to 5 amino acids, where each amino acid is independently selected from glycine and serine. Examples of flexible linkers include (G)ₙ (SEQ ID NO: 1), (GS)ₙ (SEQ ID NO: 2), (GGS)ₙ (SEQ ID NO: 3), (GGGS)ₙ (SEQ ID NO: 4), or (GGGGS)ₙ (SEQ ID NO: 5) where n can be an integer from 1 to 20; (G)ₙ (SEQ ID NO: 6), (GS)ₙ (SEQ ID NO: 7), (GGS)ₙ (SEQ ID NO: 8), (GGGS)ₙ (SEQ ID NO: 9), or (GGGGS)ₙ (SEQ ID NO: 10) where n can be an integer from 1 to 10; or (G)ₙ (SEQ ID NO: 11), (GS)ₙ (SEQ ID NO: 12), (GGS)ₙ (SEQ ID NO: 13), (GGGS)ₙ (SEQ ID NO: 14), or (GGGGS)ₙ (SEQ ID NO: 15) where n can be an integer from 1 to 5. A flexible linker can have the amino acid sequence, for example, (GGGGS) (SEQ ID NO: 16), (GGGGS)2 (SEQ ID NO: 17), (GGGGS)3 (SEQ ID NO: 18), (GGGGS)4 (SEQ ID NO: 19), (GG) (SEQ ID NO: 20), (GGG) (SEQ ID NO: 21), (GGGGG) (SEQ ID NO: 22), (GGS) (SEQ ID NO: 23), (GGGS) (SEQ ID NO: 24), (GGGGSGG) (SEQ ID NO: 25), (GGS)2 (SEQ ID NO: 26), (G)5 (SEQ ID NO: 27), or (GS)10 (SEQ ID NO: 28).

A “rigid linker” refers to a peptidyl linker that is proline rich and can include other amino acids such as alanine, lysine, and/or glutamic acid. A rigid linker can comprise, for example, from 1 to 100 amino acids such as from 1 to 50, from 1 to 40, from 1 to 30, from 1 to 20, from 1 to 10, or from 1 to 5 amino acids, where each amino acid is independently selected from proline, alanine, lysine, and glutamic acid. A rigid linker can comprise, for example, from 1 to 100 amino acids such as from 1 to 50, from 1 to 40, from 1 to 30, from 1 to 20, from 1 to 10, or from 1 to 5 amino acids, where each amino acid is independently selected from proline and alanine. A rigid linker can have the sequence (P)ₙ (SEQ ID NO: 31) or (PA)ₙ (SEQ ID NO: 32), where n is an integer from 1 to 20. A rigid linker can have the sequence (P)ₙ (SEQ ID NO: 33) or (PA)ₙ (SEQ ID NO: 34), where n is an integer from 1 to 10. A rigid linker can have the sequence (P)ₙ (SEQ ID NO: 35) or (PA)ₙ (SEQ ID NO: 36), where n is an integer from 1 to 5. A rigid linker can have the sequence (PA)₅ (SEQ ID NO: 37), (PA)₆ (SEQ ID NO: 38), (PA)₇ (SEQ ID NO: 39), (PA)₈ (SEQ ID NO: 40), (PA)₉ (SEQ ID NO: 41), or (PA)₁₀ (SEQ ID NO: 42).

“Percent (%) sequence similarity” is determined by comparing the number of amino acids that are the same in a subject ligand and a reference ligand. A ligand provided by the present disclosure can comprise, for example, greater than 60% sequence similarity, greater than 70%, greater than 80%, or greater than 90% sequence similarity to a reference ligand. For example, based on a reference ligand having SEQ ID NO: 43, ligands having SEQ ID NO: 44-49, have either 1, 2, 3, 4, or 5 amino acids in which an amino acid of the reference ligand has been substituted or replaced with the amino acid, alanine. Ligands having SEQ ID NO: 44-49 are characterized by a 95%, 90%, 85%, 80%, 75%, or 70% sequence similarity, respectively, to the reference ligand having SEQ ID NO: 43.

| | |
|---|---|
| SEQ ID NO: 43 | Y P C W L A R V G E L C D L D S G D V H |
| SEQ ID NO: 44 | A P C W L A R V G E L C D L D S G D V H |
| SEQ ID NO: 45 | A P C A L A R V G E L C D L D S G D V H |
| SEQ ID NO: 46 | A P C A L A A V G E L C D L D S G D V H |
| SEQ ID NO: 47 | A P C A L A A V G A L C D L D S G D V H |
| SEQ ID NO: 48 | A P C A L A A V G A L C D L A S G D V H |
| SEQ ID NO: 49 | A P C A L A A V G A L C D L A A G D V H |

For example, an IL-2Rβγc agonist peptide can comprise an amino acid sequence in which, for example, from 1 to 10 amino acids or from 1 to 5 amino acids of a reference amino acid sequence are substituted with another amino acid. For example, an IL-2Rβ ligand or an Rγc ligand provided by the present disclosure can have an amino acid sequence in which, for example, from 1 to 10 amino acids or from 1 to 5 amino acids of a reference amino acid sequence are substituted with another amino acid.

For example, a peptide derived from a reference peptide can have from 1 to 10 amino acid substitutions, from 1 to 5 amino acid substitutions, from 1 to 4, from 1 to 3, or from 1 to 2 amino acid substitutions. For example, a peptide derived from a reference peptide can have 1 amino acid substitution, 2 amino acid substitutions, 3 amino acid substitutions, 4 amino acid substitutions, or 5 amino acid substitutions. For example, an IL-2Rβ ligand or an Rγc ligand derived from a reference IL-2Rβ ligand or Rγc ligand can have from 1 to 10 amino acid substitutions, from 1 to 5 amino acid substitutions, from 1 to 4, from 1 to 3, or from 1 to 2 amino acid substitutions. For example, an IL-2Rβ ligand or Rγc ligand derived from a reference IL-2Rβ ligand or Rγc ligand can have 1 amino acid substitution, 2 amino acid substitutions, 3 amino acid substitutions, 4 amino acid substitutions, or 5 amino acid substitutions.

An amino acid substitution can be independent of other amino acid substitutions.

Each amino acid substitution can independently be a conservative amino acid substitution or a non-conservative amino acid substitution.

For example, a reference peptide can have the amino acid sequence of SEQ ID NO: 50. Peptides having SEQ ID NO: 51-55 represent substituted peptides in which the reference peptide having SEQ ID NO: 50 has been substituted with from 1 to 5 conservative amino acid substitutions, respectively.

| | |
|---|---|
| SEQ ID NO: 50 | Y H C W M A Q V G E L C D L |
| SEQ ID NO: 51 | Y H C W M A Q V G E L C D L |
| SEQ ID NO: 52 | Y H C W M G Q V G E L C D L |
| SEQ ID NO: 53 | Y H C W M G Q M G E L C D L |
| SEQ ID NO: 54 | Y H C W M G Q M G E L C E L |
| SEQ ID NO: 55 | Y H C W M G Q M G E L C E M |

An IL-2Rβγc agonist peptide can have, for example, greater than 80%, greater than 85%, greater than 90%, greater than 95%, greater than 96%, greater than 98%, or greater than 99% sequence similarity to an IL-2Rβγc agonist peptide identified by a specific SEQ ID NO:.

A peptide such as an IL-2Rβ ligand, an IL-2Rγc ligand, and an IL-2Rβγc agonist peptide can comprise a truncated peptide. A “truncated peptide” refers to a peptide in which, for example, from 1 to 10 or from 1 to 5 amino acids have independently been removed from the N-terminus, the C-terminus, or from both the N-terminus and the C-terminus of the corresponding reference peptide. A truncated peptide derived from the corresponding reference peptide can independently have, for example from 1 to 5 amino acids, such as from 1 to 4 amino acids, from 1 to 3 amino acids, or from 1 to 2 amino acids independently removed from the N-terminus, the C-terminus, or from both the N-terminus and the C-terminus of the reference peptide. A truncated peptide derived from the corresponding ligand can independently have, for example, 1 amino acid, 2 amino acids, 3 amino acids, 4 amino acids, or 5 amino acids removed from the N-terminus, the C-terminus, or from both the N-terminus and the C-terminus of the reference peptide.

For example, a reference peptide can have the amino acid sequence of SEQ ID NO: 56. Examples of truncated peptides derived from the reference peptide having SEQ ID NO: 56 include truncated peptides having an amino acid sequence of SEQ ID NO: 57-64.

| | |
|---|---|
| SEQ ID NO: 56 | M G F Y P C W T A Q L G E L C D L S V D |
| SEQ ID NO: 57 | G F Y P C W T A Q L G E L C D L S V D |
| SEQ ID NO: 58 | F Y P C W T A Q L G E L C D L S V D |
| SEQ ID NO: 59 | Y P C W T A Q L G E L C D L S V D |
| SEQ ID NO: 60 | M G F Y P C W T A Q L G E L C D L S V |
| SEQ ID NO: 61 | M G F Y P C W T A Q L G E L C D L S |
| SEQ ID NO: 62 | M G F Y P C W T A Q L G E L C D L |
| SEQ ID NO: 63 | G F Y P C W T A Q L G E L C D L S V |
| SEQ ID NO: 64 | F Y P C W T A Q L G E L C D L |

The truncated peptides having SEQ ID NO: 57-59 have from 1 to 3 amino acids removed from the N-terminus of the reference peptide, respectively; truncated peptides having SEQ ID NO: 60 to 62 have from 1 to 3 amino acids removed from the C-terminus of the reference peptide, respectively; and truncated peptides having SEQ ID NO: 63 and 64 have amino acids removed from both the N-terminus and from the C-terminus of the reference peptide.

As another example, a reference peptide can comprise an amino acid sequence of Formula (A):

-X¹-X²-C-X³-X⁴-X⁵-X⁶-X⁷-X⁸-X⁹-X¹⁰-C-X¹¹-X¹²- (A)

where each —X— independently represents an amino acid. Amino acid sequences of Formula (A1)-(A5) represent examples of truncated peptides derived from the reference peptide comprising the amino acid sequence of Formula (A):

-X²-C-X³-X⁴-X⁵-X⁶-X⁷-X⁸-X⁹-X¹⁰-C-X¹¹-X¹²- (A1)

-C-X³-X⁴-X⁵-X⁶-X⁷-X⁸-X⁹-X¹⁰-C-X¹¹-X¹²- (A2)

-C-X³-X⁴-X⁵-X⁶-X⁷-X⁸-X⁹-X¹⁰-C- (A3)

-X³-X⁴-X⁵-X⁶-X⁷-X⁸-X⁹-X¹⁰-X¹¹- (A4)

-X³-X⁴-X⁵-X⁶-X⁷-X⁸-X⁹-X¹⁰- (A5)

A peptide can comprise, for example, an amino acid sequence in which from 1 to 3 flanking amino acids such as glycines are independently bonded to the N-terminus, to the C-terminus, or to both the N-terminus and to the C-terminus of a reference peptide.

For example, a reference peptide can have SEQ ID NO: 65. Peptides shaving SEQ ID NO: 66-68 have from 1 to 3 glycines bonded to the N-terminus of the reference peptide, respectively; peptides having SEQ ID NO: 69-71 have from 1 to 3 glycines bonded to the C-terminus of the reference peptide, respectively; and peptides having SEQ ID NO: 72 and 73 have 1 or 2 glycines bonded to both the N-terminus and to the C-terminus of the reference peptide.

| | |
|---|---|
| SEQ ID NO: 65 | K Y C G F A Q L G E L C V L |
| SEQ ID NO: 66 | G K Y C G F A Q L G E L C V L |
| SEQ ID NO: 67 | G G K Y C G F A Q L G E L C V L |
| SEQ ID NO: 68 | G G G K Y C G F A Q L G E L C V L |
| SEQ ID NO: 69 | K Y C G F A Q L G E L C V L G |
| SEQ ID NO: 70 | K Y C G F A Q L G E L C V L G G |
| SEQ ID NO: 71 | K Y C G F A Q L G E L C V L G G G |
| SEQ ID NO: 72 | G K Y C G F A Q L G E L C V L G |
| SEQ ID NO: 73 | G G K Y C G F A Q L G E L C V L G |

A peptide can comprise, for example, one or more flanking amino acids such as, for example, flanking glycine groups on the N-terminus and/or the C-terminus of the respective peptide. For example, a ligand can comprise one or more flanking amino acids having an amino acid sequence of any one of SEQ ID NO: 1-42.

“IL-2Rβγc agonist peptide” refers to a peptide comprising at least one IL-2Rβ ligand and at least one IL-2Rγc ligand provided by the present disclosure. An IL-2Rβγc agonist peptide can bind to both the IL-2Rβ subunit and to the IL-2Rγc subunit with an IC₅₀, for example, less than 100 μM, less than 10 μM, less than 1 μM, less than 0.1 μM, or less than 0.01 μM.

IL-2R, the IL-2Rβ subunit, and the IL-2Rγc subunit refer to mammalian IL-2R, the IL-2Rβ subunit, and the IL-2Rγc subunit, respectively, such as hu-IL-2R, the hu-IL-2Rβ subunit, and the hu-IL-2Rγc subunit, respectively.

The expression “at least one” refers to “one or more.” For example, the expression “at least one” can refer to from 1 to 10, from 1 to 8, from 1 to 6, from 1 to 5, from 1 to 4, from 1 to 3, or from 1 to 2. For example, the expression “at least one” can refer to 1, 2, 3, 4, 5, 6, 7, 8, 9, or 10.

“Concentration of the target immune cell population” refers to the number of cells per milliliter of blood or culture medium, where the number of cells can be determined using flow cytometry.

“Target immune cell population” refers to a population or subpopulation of immune cells having a desired phenotype. A target immune cell population can comprise a single subpopulation of immune cells. A target immune cell population can comprise two or more subpopulations of immune cells where each of the subpopulations of immune cells has a different phenotype.

“IL-2Rβγc agonist peptide” refers to a peptide that binds to the hu-IL-2R with an IC₅₀ less than 100 μM, as determined using phage display ELISA assays. An IL-2Rβγc agonist peptide can exhibit an EC50 for STAT5 phosphorylation in hu-PBMCs and in IL-2Rβ expressing TF-1 (TF-1β) cells of less than 100 μM, less than 10 μM, less than 1 μM, or less than 0.1 μM. An IL-2Rβγc agonist peptide can activate the STAT5 phosphorylation pathway, the AKT phosphorylation pathway, and the ERK1/2 phosphorylation pathway in CD4+ and CD8+ cells. An IL-2Rβγc can comprise, for example, an amino acid sequence of any one of SEQ ID NO: 330-560, 561-587, 609-618, or 770-785.

“IL-7Rαγc agonist peptide” refers to a peptide comprising at least one IL-7Rα ligand and at least one Rγc ligand provided by the present disclosure. An IL-7Rαγc agonist peptide can bind to both the IL-7Rα subunit and to the IL-7Rγc subunit with an IC₅₀, for example, less than 100 μM, less than 10 μM, less than 1 μM, less than 0.1 μM, or less than 0.01 μM.

“IL-15Rβγc agonist peptide” refers to a peptide that binds to the hu-IL-15R with an IC₅₀ less than 100 μM, as determined using phage display ELISA assays. An IL-2Rβγc agonist peptide can exhibit an EC50 for STAT5 phosphorylation in hu-PBMCs and in IL-2Rβ expressing TF-1 (TF-113) cells of less than 100 μM, less than 10 μM, less than 1 μM, or less than 0.1 μM. An IL-2Rβγc agonist peptide can activate the STAT5 phosphorylation pathway, the AKT phosphorylation pathway, and the ERK1/2 phosphorylation pathway in CD4+ and CD8+ cells.

“Anti-CD3 monoclonal antibody” refers to a monoclonal antibody that binds to the CD3 antigen, a human glycoprotein that is primarily expressed on T cells, certain NKT cells, and thymocytes during T cell differentiation. Binding of an anti-CD3 monoclonal antibody to CD3 stimulates T cell activation. Anti-CD3 monoclonal antibodies can be used to expand T cells in culture.

“Anti-CD28 monoclonal antibody” refers to a monoclonal antibody that binds to the CD80 and CD86 antigen. CD28 is expressed on most T cell lineages, NK cell subsets, and plasma cells. CD28 binding induces T cell activation, IL-2 synthesis, and prevents cell death. In vitro, CD28 binding to T cells provides a costimulatory signal required for T cell activation and proliferation.

An “enriched immune cell population provided by the present disclosure” refers to a population of immune cells, a subpopulation of immune cells, or a combination of subpopulations of immune cells that are produced using a culture medium provided by the present disclosure, an immobilized IL-2Rβγc agonist peptide, a method of enrichment provided by the present disclosure, a method of manufacturing provided by the present disclosure or a combination of any of the foregoing. An enriched immune cell population comprises a population of immune having a higher concentration of a target immune cell population than the initial concentration of the target immune cell population before enrichment.

Reference is now made in detail to certain embodiments of compounds, compositions, and methods. The disclosed embodiments are not intended to be limiting of the claims. On the contrary, the claims are intended to cover all alternatives, modifications, and equivalents.

Interleukin-2 (IL-2) plays a crucial role in regulating immune responses and maintaining peripheral self-tolerance by having both immuno-stimulatory and immuno-regulatory functions. IL-2 acts primarily as a T-cell growth factor and is essential for the proliferation and survival of T-cells as well as for the generation of effector and memory T-cells. IL-2 is a four α-helical bundle cytokine that belongs to a family of structurally related cytokines that includes IL-4, IL-7, IL-9, IL-15, and IL-21. IL-2 is produced by activated CD4+ T-cells in response to antigen stimulation and can also be produced by CD8+ T-cells and innate immune cells such as activated dendritic cells (DCs) and natural killer (NK) cells.

IL-2 binds to various forms of the IL-2 receptor (IL-2R), notably the monomeric, dimeric, and trimeric forms. Monomeric IL-2R consists of the membrane-associated IL-2Rα (CD25) chain, which also exists in a soluble form; however, IL-2Rα is not capable of inducing signaling events. The trimeric IL-2R consists of IL-2Rα (CD25), IL-2Rβ (CD122), and IL-2Rγc (CD132), also known as the common γ-chain (γc) and is shared by all members of the IL-2 cytokine family. Dimeric IL-2R comprises the IL-2Rβ and IL-2Rγc subunits. In contrast to monomeric IL-2R, activation of both dimeric IL-2R and trimeric IL-2R leads to a downstream signaling cascade upon IL-2 binding. IL-2 binds with high affinity to trimeric IL-2R but with low to moderate affinity to dimeric IL-2R, varying with the sensitivity of the cell to IL-2. Additionally, IL-2 can bind to IL-2Rα expressed on the surface of activated dendritic cells for trans presentation to neighboring cells, including antigen-specific naïve T-cells and NK cells that express both the IL-2Rβ and IL-2Rγc subunits. This trans presentation of IL-2 has been shown to facilitate initial high-affinity IL-2 signaling, required early in the immune response to prime naïve T-cells to produce IL-2.

During activation, IL-2 is first captured by IL-2Rα, inducing a conformational change to IL-2 and increasing its affinity for IL-2Rβ. Association of IL-2 with the IL-2Rβγc subunits induces the dimerization of the signaling motifs in the cytoplasmic tails of IL-2Rβ and IL-2Rγc, leading to the phosphorylation/activation of the Janus kinases, JAK1 and JAK3, which in turn exert kinase activity on key tyrosine residues in the tail of the IL-2Rβ subunit.

Downstream signaling occurs via three major pathways, the JAK-STAT pathway, the phosphoinositide 3-kinase (PI3K)-AKT pathway, and the mitogen-activated protein kinase (MARK) pathway. These pathways result in the transcription of target genes that contribute to IL-2-dependent biological actions through the recruitment of the adaptor protein SHC and the transcription factor STAT5. Target genes of IL-2 signaling include cyclin D2, bcl-2, fasL, cd25 (encoding IL-2Rα), socs1-2, and the IL-2 silencing gene prdm1, which encodes for the transcription factor, BLIMP1. The production of the negative regulator of IL-2 BLIMP1 maintains the balance between effector T-cells and Treg cells, which is important for immune homeostasis.

IL-2 plays a dual role in T-cell activation by stimulating the proliferation and differentiation of T-cells as well as by maintaining and expanding the population of immuno-suppressive Treg cells. Naïve CD4+ and CD8+ T-cells express dimeric IL-2R, requiring a high concentration of IL-2 to induce their initial proliferation. Once activated, these T-cells express the high-affinity trimeric IL-2R, driving the differentiation of the T-cells into either effector (Teff) or memory cells. This differentiation depends on the strength and duration of the IL-2 signal.

During the primary expansion of CD8+ T-cells in the presence of low-to-moderate levels of IL-2, a subset of CD8+ T-cells differentiates into memory T-cells. The cells do this by downregulating CD25 and upregulating CD127 (IL-7R) and CD62L (L-selectin), which are receptors associated with secondary responses upon re-infection. During an acute infection, sustained high levels of IL-2 lead to a rapid upregulation of CD25 and the differentiation of CD8+ cells into cytotoxic effector cells. The up-regulation induces an IL-2-driven expression of the death receptor FAS and FASL, causing activation-induced cell death (AICD) upon pathogen clearance. For CD4+ T-cells, the activation of STAT5 signaling by IL-2 influences their differentiation into multiple helper T-cell populations, including Th1, Th2, and Th17 by regulating the expression of the appropriate receptors, cytokines, and chemokines for each response.

Homeostatic or background levels of IL-2 are important for the survival and function of Treg cells by maintaining the expression of FOXP3 and CD25. Treg cells naturally occur in the thymus and are activated upon contact with self-peptides. Additionally, Treg cells can be generated by stimulation of conventional CD4+ T-cells upon interaction with antigens in peripheral lymphoid organs. Because Treg cells do not produce IL-2, Treg cells depend on IL-2-producing cells such as conventional T-cells. Due to their high expression of IL-2Rα (CD25), Tregs can consume and limit the systemic concentration of IL-2, ensuring regulation of the immune balance. In the absence of IL-2, the ratio of Treg cells to effector cells decreases, leading to enhanced susceptibility to autoimmune and inflammatory disorders. The unique activation of Treg cells at low levels of IL-2, which does not activate CD4+ or CD8+ T-cells, has allowed for the development of IL-2 as a promising therapeutic in autoimmune and inflammatory diseases.

The production of IL-2 from both the innate and adaptive arms of the immune system highlights the importance of this cytokine in the early stages of infection, as well as in the secondary adaptive immune response. Furthermore, the dual functions of IL-2 in both protective immunity and immune tolerance allow IL-2 to be a potential therapeutic in seemingly contrasting therapies, as both an immune stimulant and as an immune suppressor, for cancer and autoimmune disease, respectively.

The present disclosure is directed to IL-2Rβγc agonist peptides, culture media comprising the IL-2Rβγc agonist peptides, and to methods of expanding a selected or target immune cell population and methods of manufacturing a selected immune cell population using IL-2Rβγc agonist peptides.

An IL-2Rβγc agonist peptide refers to a peptide comprising an IL-2Rβ ligand and an IL-2Rγc ligand, wherein the IL-2Rβγc agonist peptide has an EC50 for STAT5 phosphorylation of IL-2Rβ expressing TF-1 (TF-113) cells of less than 100 μM, less than 10 μM, less than 1 μM, less than 100 μM, less than 10 pM, or less than 1 pM.

IL-2Rβγc agonist peptides comprise an IL-2Rβ ligand, an IL-2Rγc ligand, and an IL-2Rβγc linker coupling the IL-2Rβ and IL-2Rγc ligands. An IL-2Rβγc agonist peptide can be an IL-2R agonist or a partial IL-2R agonist. Because the IL-2Rβγc agonist peptides do not bind to IL-2Rα, the IL-2Rβγc agonist peptides do not preferentially activate Tregs, have the potential for lower receptor-mediated clearance, and can exhibit decreased toxicity.

Tandem IL-2Rβγc agonist peptides provided by the present disclosure comprise two or more IL-2Rβγc agonist peptides coupled together by one or more linkers.

An IL-2Rβγc agonist peptide can comprise an IL-2Rβ ligand. Examples of suitable IL-2Rβ ligands are disclosed, for example, in U.S. Application Publication No. 2020/0040034 A1, which is incorporated by reference in its entirety.

An IL-2Rβ ligand such as an IL-2Rβ provided by the present disclosure can bind to the hu-IL-2Rβ subunit with an IC₅₀, for example, of less than 100 μM, less than 10 μM, less than 1 μM, less than 0.1 μM, or less than 0.01 μM.

An IL-2Rβ ligand can bind to the hu-IL-2Rβ subunit with an IC₅₀, for example, from 1 pM to 100 μM, from 10 pM to 10 μM, from 100 pM to 1 μM, from 0.001 μM to 1 μM, or from 0.01 μM to 1 μM.

An IL-2Rβ ligand provided by the present disclosure can bind to a mammalian IL-2Rβ subunit with an IC₅₀ of less than 100 μM, less than 10 μM, less than 1 μM, less than 0.1 μM, or less than 0.01 μM.

An IL-2Rβ ligand can bind to a mammalian IL-2Rβ subunit with an IC₅₀, for example, from 1 pM to 100 μM, from 10 pM to 10 μM, from 100 pM to 1 μM, from 0.001 μM to 1 μM, or from 0.01 μM to 1 μM.

An IL-2Rβ ligand can bind to each of the hu-IL-2Rβ subunit and to a mammalian IL-2Rγc subunit with an IC₅₀, for example, of less than 100 μM, less than 10 μM, less than 1 μM, less than 0.1 μM, or less than 0.01 μM.

An IL-2Rβ ligand can comprise an amino acid sequence of Formula (1) (SEQ ID NO: 74), Formula (1a) (SEQ ID NO: 75), Formula (1b) (SEQ ID NO: 76), Formula (1c) (SEQ ID NO: 77), or Formula (1d) (SEQ ID NO: 78):

-X²¹¹-X²¹²-X²¹³-X²¹⁴-C-X²¹⁵-X²¹⁶-X²¹⁷-X²¹⁸-X²¹⁹-X²²⁰-X²²¹-X²²²-C-X²²³-X²²⁴-X²²⁵- (1)

-X²¹²-X²¹³-X²¹⁴-C-X²¹⁵-X²¹⁶-X²¹⁷-X²¹⁸-X²¹⁹-X²²⁰-X²²¹-X²²²-C-X²²³-X²²⁴ (1a)

-X²¹³-X²¹⁴-C-X²¹⁵-X²¹⁶-X²¹⁷-X²¹⁸-X²¹⁹-X²²⁰-X²²¹-X²²²-C-X²²³-X²²⁴ (1b)

-X²¹⁴-C-X²¹⁵-X²¹⁶-X²¹⁷-X²¹⁸-X²¹⁹-X²²⁰-X²²¹-X²²²-C-X²²³- (1c)

-C-X²¹⁵-X²¹⁶-X²¹⁷-X²¹⁸-X²¹⁹-X²²⁰-X²²¹-X²²²-C- (1d)

wherein each of X²¹¹-X²²⁵ is independently selected from an amino acid.

In IL-2Rβ ligands of Formula (1), (1a), (1b), (1c), and/or (1d), X²¹¹ can be selected from an amino acid; X²¹² can be selected from an amino acid comprising an aromatic side chain; X²¹³ can be selected from an amino acid comprising a large hydrophobic side chain or an aromatic side chain; X²¹⁴ can be P; X²¹⁵ can be selected from an amino acid comprising an aromatic side chain; X²¹⁶ can be selected from an amino acid comprising a large hydrophobic side chain; X²¹⁷ can be A; X²¹⁸ can be selected from an amino acid comprising a basic side chain or a polar/neutral side chain; X²¹⁹ can be selected from an amino acid comprising a large hydrophobic side chain; X²²⁰ can be G; X²²¹ can be selected from an amino acid comprising an acidic side chain or a polar/neutral side chain; X²²² can be L; X²²³ can be D; X²²⁴ can be selected from an amino acid comprising a large hydrophobic side chain; and X²²⁵ can be selected from an amino acid comprising an acidic side chain.

In IL-2Rβ ligands of Formula (1), (1a), (1b), (1c), and/or (1d), X²¹¹ can be selected from an amino acid; X²¹² can be selected from F, H, W, and Y; X²¹³ can be selected from F, H, I, L, M, V, W, and Y; X²¹⁴ can be P; X²¹⁵ can be selected from F, H, W, and Y; X²¹⁶ can be selected from F, I, L, M, V, W, and Y; X²¹⁷ can be A; X²¹⁸ can be selected from K, R, H, N, Q, S, T, and Y; X²¹⁹ can be selected from F, I, L, M, V, W, and Y; X²²⁰ can be G; X₂₂₁ can be selected from D, E, H, N, Q, S, T, and Y; X²²² can be L; X₂₂₃ can be D; X²²⁴ can be selected from F, I, L, M, V, W, and Y; and X₂₂₅ can be selected from D and E.

In IL-2Rβ ligands of Formula (1), X²¹¹ can be selected from an amino acid.

In IL-2Rβ ligands of Formula (1), X²¹¹ can be selected from H, K, and R.

In IL-2Rβ ligands of Formula (1), X²¹¹ can be selected from H and R.

In IL-2Rβ ligands of Formula (1)-(1a), X²¹² can be selected from F, H, W, and Y.

In IL-2Rβ ligands of Formula (1)-(1a), X²¹² can be W.

In IL-2Rβ ligands of Formula (1)-(1b), X²¹³ can be selected from F, H, I, L, M, V, W, and Y.

In IL-2Rβ ligands of Formula (1)-(1b), X²¹³ can be L.

In IL-2Rβ ligands of Formula (1)-(1b), X²¹³ can be Y.

In IL-2Rβ ligands of Formula (1)-(1c), X²¹⁴ can be P.

In IL-2Rβ ligands of Formula (1)-(1d), X²¹⁵ can be selected from F, H, W, and Y.

In IL-2Rβ ligands of Formula (1)-(1d), X²¹⁵ can be W.

In IL-2Rβ ligands of Formula (1)-(1d), X²¹⁶ can be selected from F, I, L, M, V, W, and Y.

In IL-2Rβ ligands of Formula (1)-(1d), X²¹⁶ can be M.

In IL-2Rβ ligands of Formula (1)-(1d), X²¹⁷ can be A.

In IL-2Rβ ligands of Formula (1)-(1d), X²¹⁸ can be selected from K, R, H, N, Q, S, T, and Y.

In IL-2Rβ ligands of Formula (1)-(1d), X²¹⁸ can be selected from K and R.

In IL-2Rβ ligands of Formula (1)-(1d), X²¹⁸ can be Q.

In IL-2Rβ ligands of Formula (1)-(1d), X²¹⁹ can be selected from F, I, L, M, V, W, and Y.

In IL-2Rβ ligands of Formula (1)-(1d), X²¹⁹ can be L.

In IL-2Rβ ligands of Formula (1)-(1d), X²²⁰ can be G.

In IL-2Rβ ligands of Formula (1)-(1d), X²²¹ can be selected from D, E, H, N, Q, S, T, and Y.

In IL-2Rβ ligands of Formula (1)-(1d), X²²¹ can be E.

In IL-2Rβ ligands of Formula (1)-(1d), X²²² can be L.

In IL-2Rβ ligands of Formula (1)-(1c), X²²³ can be D.

In IL-2Rβ ligands of Formula (1)-(1b), X²²⁴ can be selected from F, I, L, M, V, W, and Y.

In IL-2Rβ ligands of Formula (1)-(1b), X²²⁴ can be L.

In IL-2Rβ ligands of Formula (1)-(1a), X²²⁵ can be selected from D and E.

In IL-2Rβ ligands of Formula (1), (1a), (1b), (1c), and/or (1d), X²¹¹ can be selected from H, K, and R; X²¹² can be W; X²¹³ can be Y; X²¹⁴ can be P; X²¹⁵ can be W; X²¹⁶ can be M; X²¹⁷ can be A; X²¹⁸ can be selected N and Q; X²¹⁹ can be selected from L and V; X²²⁰ can be G; X²²¹ can be selected from E, D, and Q; X²²² can be L; X²²³ can be D; X²²⁴ can be selected from L and M; and X²²⁵ can be selected from D and E.

In IL-2Rβ ligands of Formula (1), (1a), (1b), (1c), and/or (1d), X²¹¹ can be selected from A, D, E, G, H, L, M, N, Q, R, S, T, and V; X²¹² can be selected from C, F, W, and Y; X²¹³ can be selected from F, H, K, L, N, Q, R, S, W, and Y; X²¹⁴ can be P; X²¹⁵ can be selected from W and Y; X²¹⁶ can be selected from F, I, K, L, M, R, S, T, and V; X²¹⁷ can be A; X²¹⁸ can be selected from D, E, G, H, K, L, N, Q, R, S, and Y; X²¹⁹ can be selected from L, P, and V; X²²⁰ can be selected from G, H, and W; X²²¹ can be selected from D, E, and Q; X²²² can be selected from L and M; X²²³ can be D; X²²⁴ can be selected from L, M, Q, and V; and X²²⁵ can be selected from A, D, E, F, G, H, L, N, Q, T, and V.

In IL-2Rβ ligands of Formula (1), (1a), (1b), (1c), and/or (1d), X²¹¹ can be selected from H an R; X²¹² can be selected from F and W; X²¹³ can be selected from F, L, W, and Y; X²¹⁴ can be P; X²¹⁵ can be selected from W and Y; X²¹⁶ can be selected from F, I, L, M, and V; X²¹⁷ can be A; X²¹⁸ can be selected D, E, H, K, N, Q, and R; X²¹⁹ can be selected from L and V; X²²⁰ can be G; X²²¹ can be selected from D, E, and Q; X²²² can be selected from L and M; X²²³ can be D; X²²⁴ can be selected L, M, and V; and X²²⁵ can be selected from D and E.

In IL-2Rβ ligands of Formula (1), (1a), (1b), (1c), and/or (1d), X²¹¹ can be selected from H and R; X²¹² can be W; X²¹³ can be Y; X²¹⁴ can be P; X²¹⁵ can be W; X²¹⁶ can be M; X²¹⁷ can be A; X²¹⁸ can be Q; X²¹⁹ can be L; X²²⁰ can be G; X²²¹ can be Q; X²²² can be L; X²²³ can be D; X²²⁴ can be L; and X²²⁵ can be selected from D and E.

In IL-2Rβ ligands of Formula (1), (1a), (1b), (1c), and/or (1d), X²¹¹ can be selected from H and R; X²¹² can be W; X²¹³ can be L; X²¹⁴ can be P; X²¹⁵ can be W; X²¹⁶ can be M; X²¹⁷ can be A; X²¹⁸ can be Q; X²¹⁹ can be L; X²²⁰ can be G; X²²¹ can be Q; X²²² can be L; X²²³ can be D; X²²⁴ can be L; and X²²⁵ can be selected from D and E.

In IL-2Rβ ligands of Formula (1), (1a), (1b), (1c), and/or (1d), X²¹¹ can be selected from H and R; X²¹² can be W; X²¹³ can be Y; X²¹⁴ can be P; X²¹⁵ can be W; X²¹⁶ can be M; X²¹⁷ can be A; X²¹⁸ can be selected from K and R; X²¹⁹ can be L; X²²⁰ can be G; X²²¹ can be Q; X²²² can be L; X²²³ can be D; X²²⁴ can be L; and X²²⁵ can be selected from D and E.

An IL-2Rβ ligand can comprise an amino acid sequence selected from any one of SEQ ID NO: 79-172.

| | |
|---|---|
| SEQ ID NO: 79 | Q P C W L A Q V G D L C D L L W P G P L |
| SEQ ID NO: 80 | W L P C W I A R L G D L C D L E |
| SEQ ID NO: 81 | W Y P C W M A L L G E L C D Q E |
| SEQ ID NO: 82 | W Y P C Y R A R L G E L C D L D |
| SEQ ID NO: 83 | W Q R E W R W F P C W M A K L G D M C D L D |
| SEQ ID NO: 84 | Q D E A V E W F P C W M A R L G E L C D L E |
| SEQ ID NO: 85 | Y Y P C W M A R L G E L C D L D |
| SEQ ID NO: 86 | S V V V N N W L P C W M A Q L G E L C D L D |
| SEQ ID NO: 87 | W Y P C W L A Q L G D L C D L D |
| SEQ ID NO: 88 | V M S P T R W L P C W I A K L G E L C D L E |
| SEQ ID NO: 89 | W F P C W M A Q L G Q L C D L E |
| SEQ ID NO: 90 | W R P C W R A Y L G E L C D L E A M P R A T |
| SEQ ID NO: 91 | I R S C S P C W S A D V G E L C D L E C E W |
| SEQ ID NO: 92 | S G H W Y P C W M A R L G E L C D M E E R A |
| SEQ ID NO: 93 | W Y P C W M A Q L G E L C D L Q T M G Y S H |
| SEQ ID NO: 94 | A G D W L P C W M A E L G E L C D L E G P T |
| SEQ ID NO: 95 | W L P C W I A S L G E L C D L D T G K R Q G |
| SEQ ID NO: 96 | W L P C W M A H L G Q L C D L D L P G K S M |
| SEQ ID NO: 97 | E G V F F P C W I A R L G E L C D L D H G L |
| SEQ ID NO: 98 | T G R W K P C W M A G L H E L C D L E G F R |
| SEQ ID NO: 99 | R K H F Y P C W M A Q L G E L C D L E G M P |
| SEQ ID NO: 100 | D I G Y Y P C W M A Q V G D L C D L D D E K |
| SEQ ID NO: 101 | D S D W W P C W M A Q L G E L C D L E D A R |
| SEQ ID NO: 102 | G E R W K P C W I A Q L G E L C D L D F N W |
| SEQ ID NO: 103 | W W P C W M A Q L G E M C D L E Y P Y V P G |
| SEQ ID NO: 104 | Q T K L E G W Y P C W M A Q L G E L C D L D |
| SEQ ID NO: 105 | W G R K E Q W L P C W K A Q L G E L C D L E |
| SEQ ID NO: 106 | V P R A N A W H P C W M A Q L G E L C D L E |
| SEQ ID NO: 107 | G R Q Q K G W Y P C W L A Q L G E L C D M E |
| SEQ ID NO: 108 | W L N R H L F N P C W M A R L G E L C D L E |
| SEQ ID NO: 109 | A Q V R R E W Y P C W M A Q L G E L C D L T |
| SEQ ID NO: 110 | E T E Q M S W Y P C W V A Q L W E L C D L D |
| SEQ ID NO: 111 | W L P C W L A K L G E L C D L E W L P C W |
| SEQ ID NO: 112 | E R R P D T W F P C W R A L V G E L C D L E |
| SEQ ID NO: 113 | W G R N R S W Y P C W M A Q L G E L C D L E |
| SEQ ID NO: 114 | Q D R R S P W Y P C W M A K L G E L C D L A |
| SEQ ID NO: 115 | T R R W Y P C Y L A K L G E L C D L F E G G T R |
| SEQ ID NO: 116 | S E Q W W P C W I A R L G E L C D L D R E L S E |
| SEQ ID NO: 117 | W Y P C W V A Q L G E I C D L E M T G P D S W Y P |
| SEQ ID NO: 118 | Q D G W L P C W M A Q L G E L C D L E Y K R |
| SEQ ID NO: 119 | N R R W Y P C W M A Q L G E L C D L D S R P |
| SEQ ID NO: 120 | F Y P C W M A H L G E L C D L D D D T D S M |
| SEQ ID NO: 121 | K S N F F P C W I A Q L G Q L C D L E P E T |
| SEQ ID NO: 122 | F Y P C W M A N L G E L C D L D F L R E L N |
| SEQ ID NO: 123 | H A S W L P C W L A Q L G E L C D L E P N P |
| SEQ ID NO: 124 | N G A W Y P C W M A Q V G E L C D L E E R W |
| SEQ ID NO: 125 | W R R W Y P C W V A Q V G E L C D L E I E A |
| SEQ ID NO: 126 | R Q A W Y P C W M A Q L G E L C D L E A E L |
| SEQ ID NO: 127 | R Q R W Y P C W M A R L G E L C D L D E P T |
| SEQ ID NO: 128 | N N S R E G W F P C W L A K L G D L C D L D |
| SEQ ID NO: 129 | Y Y P C W M A Q L G E L C D L E |
| SEQ ID NO: 130 | W Y P C W L A Q L G E L C D L D |
| SEQ ID NO: 131 | S W H A E T W Y P C W L A Q V G E L C D L D |
| SEQ ID NO: 132 | K M H K A V W L P C W M A Q V G E L C D L E |
| SEQ ID NO: 133 | D V L G D R W Y P C W I A K L G E L C D L D |
| SEQ ID NO: 134 | W Y P C W M A Q L G E L C D L D |
| SEQ ID NO: 135 | K L Q S W R W Y P C W M A Q L G E L C D L D |
| SEQ ID NO: 136 | N E P E G G F Y P C W L A Q L G E L C D L H |
| SEQ ID NO: 137 | W Y P C W M A R L G E L C D L E |
| SEQ ID NO: 138 | F Y P C W T A L L G E L C D L E P G P P A M |
| SEQ ID NO: 139 | W G T T W R W Y P C W M A Q L G E L C D L E |
| SEQ ID NO: 140 | A K G W D T W K P C W L A N L G E L C D L E |
| SEQ ID NO: 141 | R D E S A G Y Y P C W I A Q L G E L C D L E |
| SEQ ID NO: 142 | W Y P C W I A K L G E L C D L E |
| SEQ ID NO: 143 | W Y P C W I A Q L G E L C D L D |
| SEQ ID NO: 144 | W Y P C W L A K L G E L C D L D |
| SEQ ID NO: 145 | Q G P V R L W Y P C W M A Q L G E L C D L D |
| SEQ ID NO: 146 | W Y P C W M A Q P G E L C D V D |
| SEQ ID NO: 147 | W H P C W I A Q L G E L C D L E |
| SEQ ID NO: 148 | W Y P C W I A Q L G E L C D L E |
| SEQ ID NO: 149 | V R P M G V W Y P C W I A Q L G E L C D L V |
| SEQ ID NO: 150 | V P R W Y P C W I A Q L G E L C D L D S D D |
| SEQ ID NO: 151 | Y R G W L P C W R A K L G D L C D L G Q P M |
| SEQ ID NO: 152 | G E A W Y P C W L A R L G E L C D M D P R V |
| SEQ ID NO: 153 | W Y P C W M A Q L G E L C D L D E S T R L T |
| SEQ ID NO: 154 | I G S W W P C W M A Q L G E L C D L E P E L |
| SEQ ID NO: 155 | G T T W Y P C W L A Q L G E L C D L D V L E |
| SEQ ID NO: 156 | W W P C W M A Q L G D L C D L E E T S G G T |
| SEQ ID NO: 157 | W Y P C W M A Q L G E L C D L G P T E S N L |
| SEQ ID NO: 158 | W Y P C W M A N L G E L C D L E Y P S W A Q |
| SEQ ID NO: 159 | R G M C Y P C W F A R L G E L C D L E C D Q |
| SEQ ID NO: 160 | W Y P C W M A Q L G E L C D L D A G A R H L |
| SEQ ID NO: 161 | K S G W Y P C W M A K L G E L C D L E A Q P |
| SEQ ID NO: 162 | G P R F Y P C W I A Q L G E L C D L E D M G |
| SEQ ID NO: 163 | R V T W Y P C W M A Q L G E L C D L E E S V |
| SEQ ID NO: 164 | W L P C W M A Q L G D L C D L E Q Y V P L P |
| SEQ ID NO: 165 | Y L P C W M A H L G E L C D L D S P L K A R |
| SEQ ID NO: 166 | W Y P C W M A Q L G E L C D L D D H W P A M |
| SEQ ID NO: 167 | W Y P C W R A Q L G E L C D L D P P I A V E |
| SEQ ID NO: 168 | W Y P C W M A N L G E L C D L E A E R S P V |
| SEQ ID NO: 169 | R D Q Y Y P C W M A Q L G E L C D L D E V F |
| SEQ ID NO: 170 | W Y P C W M A Q L G D L C D L E K P V T E R |
| SEQ ID NO: 171 | W Y P C W I A R L G E L C D L E T S G G F P |
| SEQ ID NO: 172 | S G H C Y P C W L A G L G E L C D L N C G |

An IL-2Rβ ligand can comprise an amino acid sequence selected from any one of SEQ ID NO: 74-133, 652, and 135-172, wherein the amino acid sequence can be terminated with one or more amino acids on the N-terminus, on the C-terminus, or on both the N- and C-termini. For example, an amino acid sequence can include terminal glycines and/or serines. For example, an IL-2Rβ ligand can comprise a -G-G- moiety (SEQ ID NO: 20) on the N-terminus, on the C-terminus, or on both the N- and C-termini.

An IL-2Rβ ligand can comprise an amino acid sequence selected from any one of SEQ ID NO: 74-172, wherein each amino acid independently comprises one or more of the following conservative substitutions: amino acids having a small hydrophobic side chain comprising alanine (A), glycine (G), proline (P), serine (S), or threonine (T); amino acids having a hydroxyl-containing side chain comprising serine (S), threonine (T), or tyrosine (Y); amino acids having an acidic side chain comprising aspartate (D) or glutamate (E); amino acids having a polar-neutral side chain comprising histidine (H), asparagine (N), glutamine (Q), serine (S), threonine (T), or tyrosine (Y); amino acids having a basic side chain comprising arginine (R), lysine (K), or histidine (H); and amino acids having a large hydrophobic side chain comprising isoleucine (I), leucine (L), methionine (M), valine (V), phenylalanine (F), tyrosine (Y), or tryptophan (W); and amino acids having an aromatic side chain comprising phenylalanine (F), histidine (H), tryptophan (W), or tyrosine (Y).

An IL-2Rβ ligand can comprise an amino acid sequence selected from any one of SEQ ID NO: 74-172 having from 1 to 5 amino acid substitutions wherein each substitution is independently selected from a conservative amino acid substitution and a non-conservative amino acid substitution.

IL-2Rβ ligands of SEQ ID NO: 79-172 bind to the hu-IL-2Rβ subunit with an IC₅₀ of less than 100 μM as determined using phage ELISA competition assays. Certain of the IL-2Rβ ligands of SEQ ID NO: 74-78 bind to the hu-IL-2Rβ subunit with an IC₅₀ of less than 1 μM as determined using phage ELISA competition assays.

IL-2Rβ ligands of SEQ ID NO: 79-172 bind to the hu-IL-2Rβ subunit with an IC₅₀ of less than 100 μM as determined using phage ELISA competition assays. Certain of the IL-2Rβ ligands of SEQ ID NO: 74-78 bind to the hu-IL-2Rβ subunit with an IC₅₀ of less than 1 μM as determined using phage ELISA competition assays.

An IL-2Rβ ligand can comprise an amino acid sequence similarity greater than 60%, greater than 70%, greater than 75%, greater than 80%, greater than 85%, greater than 90%, or greater than 95% to an amino acid sequence of any one of SEQ ID NO: 74-172.

An IL-2Rβ ligand provided by the present disclosure can comprise a truncated amino acid sequence of any one of SEQ ID NO: 74-172

An IL-2Rβ ligand provided by the present disclosure can comprise an amino acid sequence selected from any one of SEQ ID NO: 74-172 or a truncated amino acid sequence of any one of SEQ ID NO: 74-172, wherein the amino acid sequence can independently comprise from 1 to 4 glycines (G) (SEQ ID NO: 30) on the N-terminus, on the C-terminus, or on both the N- and C-termini.

An IL-2Rβ ligand provided by the present disclosure can comprise an amino acid sequence selected from any one of SEQ ID NO: 74-172 or a truncated amino acid sequence of any one of SEQ ID NO: 74-172, wherein the amino acid sequence comprises one or more amino acid substitutions such as from 1 to 5 amino acid substitutions. Each amino acid substitution can independently be selected from a conservative amino acid substitution or a non-conservative amino acid substitution.

An IL-2Rβ ligand can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 74-172 or to a truncated amino acid sequence of any one of SEQ ID NO: 74-172.

Certain of the IL-2Rβ ligands of any one of SEQ ID NO: 79-172 bind to the hu-IL-2Rβ subunit with an IC₅₀ of less than 10 μM as determined using phage ELISA competition assays.

Certain of the IL-2Rβ ligand of any one of SEQ ID NO: 74-172, a truncated IL-2Rβ ligand of any one of SEQ ID NO: 74-172, or a substituted IL-2Rβ ligand of any one of SEQ ID NO: 74-172 can bind to the hu-IL-2Rβ subunit with an IC₅₀ of less than 100 μM or less than 10 μM as determined using phage ELISA competition assays.

An IL-2Rβ ligand can comprise an amino acid sequence of Formula (2) (SEQ ID NO: 173), an amino acid sequence of Formula (2a) (SEQ ID NO: 174), an amino acid sequence of Formula (2b) (SEQ ID NO: 175), an amino acid sequence of Formula (2c) (SEQ ID NO: 176), or an amino acid sequence of Formula (2d) (SEQ ID NO: 177):

-X¹-X²-X³-C-X⁴-X⁵-X⁶-X⁷-X⁸-X⁹-X¹⁰-X¹¹-C-X¹²-X¹³-X¹⁴- (2)

-X²-X³-C-X⁴-X⁵-X⁶-X⁷-X⁸-X⁹-X¹⁰-X¹¹-C-X¹²-X¹³- (2a)

-X³-C-X⁴-X⁵-X⁶-X⁷-X⁸-X⁹-X¹⁰-X¹¹-C-X¹²- (2b)

-C-X⁴-X⁵-X⁶-X⁷-X⁸-X⁹-X¹⁰-X¹¹-C- (2c)

-X⁴-X⁵-X⁶-X⁷-X⁸-X⁹-X¹⁰-X¹¹- (2d)

wherein,
X¹ can be selected from E, F, G, I, L, R, S, W, and Y;
X² can be selected from F, H, K, L, N, Q, S, T, V, W, and Y;
X³ can be selected from E, G, L, P, and S;
X⁴ can be selected from E, F, G, H, Q, R, S, W, and Y;
X⁵ can be selected from E, I, K, L, M, N, R, S, T, and V;
X⁶ can be selected from A, D, G, and Y;
X⁷ can be selected from A, C, D, E, G, H, K, L, N, Q, R, S, and T;
X⁸ can be selected from D, F, L, M, P, R, and V;
X⁹ can be selected from G, R W, and Y;
X¹⁰ can be selected from A, D, E, Q, W, and Y;
X¹¹ can be selected from I, L, Q, V, and Y;
X¹² can be selected from D, E, G, H, V, and Y;
X¹³ can be selected from D, F, H, I, K, L, M, and V; and
X¹⁴ can be selected from A, D, E, G, H, K, L, N, Q, V, and W.

In an IL-2Rβ ligand of Formula (2), X¹ can be selected from S, W, and Y.

In an IL-2Rβ ligand of Formula (2), X¹ can be W.

In an IL-2Rβ ligand of Formula (2)-(2a), X² can be selected from K, L, W, and Y.

In an IL-2Rβ ligand of Formula (2)-(2b), X³ can be P.

In an IL-2Rβ ligand of Formula (2)-(2d), X⁴ can be W.

In an IL-2Rβ ligand of Formula (2)-(2d), X⁵ can be selected from I, L, and M

In an IL-2Rβ ligand of Formula (2)-(2d), X⁶ can be A.

In an IL-2Rβ ligand of Formula (2)-(2d), X⁷ can be Q.

In an IL-2Rβ ligand of Formula (2)-(2d), X⁸ can be L

In an IL-2Rβ ligand of Formula (2)-(2d), X⁹ can be G

In an IL-2Rβ ligand of Formula (2)-(2d), X¹⁰ can be selected from D and E.

In an IL-2Rβ ligand of Formula (2)-(2d), X¹¹ can be L.

In an IL-2Rβ ligand of Formula (2)-(2b), X¹² can be selected from D and E.

In an IL-2Rβ ligand of Formula (2)-(2a), X¹³ can be L.

In an IL-2Rβ ligand of Formula (2), X¹⁴ can be selected from D and E.

In an IL-2Rβ ligand of Formula (2)-(2d), the IL-2Rβ ligand can be defined by any combination of X¹ to X¹⁴ as defined in the immediately preceding sixteen (16) paragraphs.

In an IL-2Rβ ligand of Formula (2)-(2d),
X¹ can be selected from S, W, and Y;
X² can be selected from K, L, W, and Y;
X³ can be P;
X⁴ can be W;
X⁵ can be selected from I, L, and M;
X⁶ can be A;
X⁷ can be Q;
X⁸ can be L;
X⁹ can be G;
X¹⁰ can be selected from D and E;
X¹¹ can be L;
X¹² can be selected from D and E;
X¹³ can be L; and
X¹⁴ can be selected from D and E.

In an IL-2Rβ ligand of Formula (2)-(2d),
X¹ can be W;
X² can be selected from K, L, W, and Y;
X³ can be P;
X⁴ can be W;
X⁵ can be selected from I, L, and M;
X⁶ can be A;
X⁷ can be Q;
X⁸ can be L;
X⁹ can be G;
X¹⁰ can be selected from D and E;
X¹¹ can be L;
X¹² can be selected from D and E;
X¹³ can be L; and
X¹⁴ can be selected from D and E.

An IL-2Rβ ligand can comprise the amino acid sequence of any one of SEQ ID NO: 178-253.

| | |
|---|---|
| SEQ ID NO: 178 | V R A W Y P C W I A R L G E L C D L E V D |
| SEQ ID NO: 179 | W F P C W M A Q L G E V C D L D |
| SEQ ID NO: 180 | W Y P C H M A K L R E L C D L D |
| SEQ ID NO: 181 | S L P C W M A Q L G D L C E L H |
| SEQ ID NO: 182 | S N P C W M A Q L W E L C D L E |
| SEQ ID NO: 183 | Y K P C W I A Q L G E L C E L E |
| SEQ ID NO: 184 | W Q P C W M A Q L G E L C D L E |
| SEQ ID NO: 185 | W K P C W L A Q L G D L C E M E |
| SEQ ID NO: 186 | W L P C W M A K L G D L C E L E |
| SEQ ID NO: 187 | W L P C W M A Q L G E L C V M E |
| SEQ ID NO: 188 | L H P C W M A Q L G E L C D L E |
| SEQ ID NO: 189 | S L P C W M A Q L G E L C D L V |
| SEQ ID NO: 190 | W K P C W M A G L G E L C E L D |
| SEQ ID NO: 191 | W W P C S S A E L G E I C D F D |
| SEQ ID NO: 192 | W Q P C W R A K L G E L C D L E |
| SEQ ID NO: 193 | W S P C W I A T L G E L C D L D |
| SEQ ID NO: 194 | W N P C W I A Q L G D L C D M V |
| SEQ ID NO: 195 | W L P C W L A H L G D I C D L Q |
| SEQ ID NO: 196 | W L S P P S Y L P C W M A Q L G E L C D L V |
| SEQ ID NO: 197 | W D V H A I R N P C W L A K L G D L C D L D |
| SEQ ID NO: 198 | G A G V L H R W P C W M A K L G D L C D L D |
| SEQ ID NO: 199 | D T G R D G W K P C W M A L L G E L C E L E |
| SEQ ID NO: 200 | E Q G M L G Y F P C W K A L L G D V C D L D |
| SEQ ID NO: 201 | W R V T A S L Q P C W M A Q L G E L C D L N |
| SEQ ID NO: 202 | G Q V V E T S L P C W E A Q L G E L C V L D |
| SEQ ID NO: 203 | T V G Q F E W Y P C S T A Q L G E L C D L D |
| SEQ ID NO: 204 | A L V G G T F Y P C Y V A H L G E L C D I E |
| SEQ ID NO: 205 | I D R A D G W K P C W I A Q V G E L C V L E |
| SEQ ID NO: 206 | Y R R E R V E F P C W L A Q L G E L C D K E |
| SEQ ID NO: 207 | A V S H G N W L P C Y I A Q L G E L C D L D |
| SEQ ID NO: 208 | I P K G E S W F P C W M A A M G E L C D L E |
| SEQ ID NO: 209 | W Y P C W I A Q L G E V C D L E K Q T G S V |
| SEQ ID NO: 210 | W Y P C W M A H L G D V C D L E S F G Q T E |
| SEQ ID NO: 211 | Y K P C Q M A Q L G E L C D L D V D N K A E |
| SEQ ID NO: 212 | F K P C W I A N L G E L C D M D D E R S S E |
| SEQ ID NO: 213 | W K P C W M A R L G E L C D I E D T K V N A |
| SEQ ID NO: 214 | S L P C W I A R L G E L C D L D G Y D G E E |
| SEQ ID NO: 215 | F Y P C W K A R L G E L C E L E E L R G Y Y |
| SEQ ID NO: 216 | W K P C W I A D L G E L C D L A P A W H E Y |
| SEQ ID NO: 217 | R L P C W R A Q L G D L C E L D W G L D M G |
| SEQ ID NO: 218 | S K P C W M A Q L G E L C D L D V W N L Q M |
| SEQ ID NO: 219 | G Y P C W L A Q L G D Y C D L D A G A P S W |
| SEQ ID NO: 220 | S W P C W M A Q L G D L C D L D G S A G A S |
| SEQ ID NO: 221 | W K P C W L A Q L G E L C D L E R P S T T S |
| SEQ ID NO: 222 | W Y S C G K A Q L G E L C D L D V E S Q P G |
| SEQ ID NO: 223 | Y V P C Y M A R L G E L C E L E A N R P G Q |
| SEQ ID NO: 224 | S K P C W L A Q L G D L C D F D W T A A D H |
| SEQ ID NO: 225 | W F P C W M A Q L G D L C E L E P D S V P A |
| SEQ ID NO: 226 | W T P C W I A H L G D L C D L E P Q D D T D |
| SEQ ID NO: 227 | W K P C F I A S L G E L C D L D Q G S V E V |
| SEQ ID NO: 228 | W K P C W M A A L G E L C D L E R S V G K V |
| SEQ ID NO: 229 | W K P C W R A Q L G E L C D L E L G P S E R |
| SEQ ID NO: 230 | F F P C W M G Q L G D L C D L E V R S M Q K |
| SEQ ID NO: 231 | Y W P C S M A S L G E L C D L E W Q G R L P |
| SEQ ID NO: 232 | W Y P C Y M A S L G E L C D L Q S S I S P R |
| SEQ ID NO: 233 | W Y P C W M A Q L G E L C D H E W P S Y G A |
| SEQ ID NO: 234 | M G S W L P C W M A Q L G D L C D V E G G M |
| SEQ ID NO: 235 | Q K G F L P C W R A Q L G Q L C D M E S Q Y |
| SEQ ID NO: 236 | G S G W Q P C W M A D L G E L C D L D N E K |
| SEQ ID NO: 237 | W R R W Y P C W M A Q L G E L C D L D Q W T |
| SEQ ID NO: 238 | R R S W Y P C R I A Q L G E L C D L D P R V |
| SEQ ID NO: 239 | A Y R I Y P C W K A Q L G E L C D L D N A D |
| SEQ ID NO: 240 | Q R N S F P C W L A Q L G D L C D L G D W A |
| SEQ ID NO: 241 | Q P A W L P C W L A Q L G E L C D L G T G A |
| SEQ ID NO: 242 | S R F W Q P C W M A Q L G E L C H L D P Q M |
| SEQ ID NO: 243 | Y L N F N P C W T A Q L G E L C D L A S G E |
| SEQ ID NO: 244 | S T G W Y P C W I A E F G E L C D L V K P H |
| SEQ ID NO: 245 | A H W S Q P C W T A Q L G E L C D L D M G D |
| SEQ ID NO: 246 | H P V R Y P C W V A Q L G E L C D L E N G N |
| SEQ ID NO: 247 | Q T G S Y P C W I A H L G E L C D L E G S A |
| SEQ ID NO: 248 | T G W W Y P C W M A Q L G E L C D L Q Q T |
| SEQ ID NO: 249 | D L W Q P C W M A R L G E L C D L K G |
| SEQ ID NO: 250 | M G T G W Q N Y C R Y A Q L G E L C L L |
| SEQ ID NO: 251 | W Y P C G V A Q P G D L C D L E |
| SEQ ID NO: 252 | M L G E W L C E M D Q L G Y L C Y L D H G D |
| SEQ ID NO: 253 | W D G W E C G M D H D G W V C E F W G E |

An IL-2Rβ ligand provided by the present disclosure can comprise a truncated amino acid sequence of any one of SEQ ID NO: 173-253.

An IL-2Rβ ligand provided by the present disclosure can comprise an amino acid sequence selected from any one of SEQ ID NO: 173-253, or a truncated amino acid sequence of any one of SEQ ID NO: 173-253, wherein the amino acid sequence can independently comprise from 1 to 4 glycines (SEQ ID NO: 30) on the N-terminus, on the C-terminus, or on both the N- and C-termini.

An IL-2Rβ ligand provided by the present disclosure can comprise an amino acid sequence selected from any one of SEQ ID NO: 173-253, or a truncated amino acid sequence of any one of SEQ ID NO: 173-253, wherein the amino acid sequence comprises one or more amino acid substitutions such as from 1 to 5 amino acid substitutions. Each amino acid substitutions can independently be selected from a conservative amino acid substitution or a non-conservative amino acid substitution.

An IL-2Rβ ligand can comprise an amino acid sequence selected from any one of SEQ ID NO: 74-172 having from 1 to 5 amino acid substitutions wherein each amino acid substitution is independently selected from a conservative amino acid substitution and a non-conservative amino acid substitution.

An IL-2Rβ ligand can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 173-253 or to a truncated amino acid sequence of any one of SEQ ID NO: 173-253.

An IL-2Rβ ligand of any one of SEQ ID NO: 173-253, a truncated IL-2Rβ ligand of any one of SEQ ID NO: 173-253, or a substituted IL-2Rβ ligand of any one of SEQ ID NO: 173-253 can bind to the hu-IL-2Rβ subunit with an IC₅₀ of less than 100 μM or less than 10 μM and to the cyno-IL-2Rβ subunit with an IC₅₀ of less than 100 μM or less than 10 μM as determined using phage ELISA competition assays.

Certain of the IL-2Rβ ligands of any one of SEQ ID NO: 173-177 bind to the hu-IL-2Rβ subunit with an IC₅₀ of less than 100 μM as determined using phage ELISA competition assays.

An IL-2Rβ ligand of any one of SEQ ID NO: 178-253 binds to the hu-IL-2Rβ subunit with an IC₅₀ of less than 100 μM as determined using phage ELISA competition assays.

An IL-2Rβ ligand can comprise the amino acid sequence of Formula (30 (SEQ ID NO: 330), an amino acid sequence of Formula (3) (SEQ ID NO: 331), an amino acid sequence of Formula (3a) (SEQ ID NO: 332), an amino acid sequence of Formula (3b) (SEQ ID NO: 333), an amino acid sequence of Formula (3c) (SEQ ID NO: 334), an amino acid sequence of Formula (3d) (SEQ ID NO: 335), or an amino acid sequence of Formula (3e) (SEQ ID NO: 336:

-X⁰-X¹-X²-X³-X⁴-C-X⁵-X⁶-X⁷-X⁸-X⁹-X¹⁰-X¹¹-X¹²-C-X¹³-X¹⁴-X¹⁵-X¹⁶-X¹⁷- (3f)

-X¹-X²-X³-X⁴-C-X⁵-X⁶-X⁷-X⁸-X⁹-X¹⁰-X¹¹-X¹²C-X¹³-X¹⁴-X¹⁵-X¹⁶-X¹⁷- (3)

-X²-X³-X⁴-C-X⁵-X⁶-X⁷-X⁸-X⁹-X¹⁰-X¹¹-X¹²-C-X¹³-X¹⁴-X¹⁵- (3a)

-X³-X⁴-C-X⁵-X⁶-X⁷-X⁸-X⁹-X¹⁰-X¹¹-X¹²C-X¹³-X¹⁴- (3b)

-X⁴-C-X⁵-X⁶-X⁷-X⁸-X⁹-X¹⁰-X¹¹-X¹²-C-X¹³- (3c)

-C-X⁵-X⁶-X⁷-X⁸-X⁹-X¹⁰-X¹¹-X¹²-C- (3d)

-X⁵-X⁶-X⁷-X⁸-X⁹-X¹⁰-X¹¹-X¹²- (3e)

wherein,
X⁰ can be selected from an amino acid;
X¹ can be selected from A, D, E, F, G, H, I, K, L, M, N, P, Q, R, S, T, V, W, and Y;
X² can be selected from C, D, F, G, I, L, M, R, S, V, W, and Y;
X³ can be selected from A, D, F, H, K, L, N, P, Q, R, S, T, V, W, and Y;
X⁴ can be selected from A, D, F, L, N, P, Q, S, T, and W;
X⁵ can be selected from D, E, F, G, L, M, Q, R, S, W, and Y;
X⁶ can be selected from A, F, I, K, L, M, N, Q, R, S, V, W, and Y;
X⁷ can be selected from A, D, E, I, S, T, V, and W;
X⁸ can be selected from A, E, F, G, H, K, L, N, P, Q, R, S, V, W, and Y;
X⁹ can be selected from A, E, I, L, M, P, Q V, and W;
X¹⁰ can be selected from F, G, and V;
X¹¹ can be selected from D, E, N, P, Q, S, V, W, and Y;
X¹² can be selected from D, F, H, I, L, M, Q, S, T, V, and W;
X¹³ can be selected from A, D, E, L, N, Q, S, T, and V;
X¹⁴ can be selected from A, E, F, I, K, L, M, Q, R S, T V, and W;
X¹⁵ can be selected from A, D, E, F, G, I, K, L, N, P, Q, R, T, V, W, and Y;
X¹⁶ can be selected from A, C, D, E, F, G, H, I, L, M, N, P, Q, R, S, T, V, W, and Y; and
X¹⁷ can be selected from A, D, E, F, G, H, I, K, L, M N, P, Q, R, S, T, V, W, and Y.

In an IL-2Rβ ligand of Formula (3)-(3e), X¹ can be selected from A, D, E, G, R, S, T, V, and W.

In an IL-2Rβ ligand of Formula (3), X¹ can be selected from G, R, and W.

In an IL-2Rβ ligand of Formula (3)-(3a), X² can be selected from F, L, S, V, W, and Y

In an IL-2Rβ ligand of Formula (3)-(3a), X² can be selected from F and W.

In an IL-2Rβ ligand of Formula (3)-(3b), X³ can be selected from F, H, K, L, N, Q, W, and Y.

In an IL-2Rβ ligand of Formula (3)-(3b), X³ can be selected from F, H, L, W, and Y.

In an IL-2Rβ ligand of Formula (3)-(3c), X⁴ can be selected from D and P.

In an IL-2Rβ ligand of Formula (3)-(3e), X⁵ can be selected from F, L, S, W, and Y.

In an IL-2Rβ ligand of Formula (3)-(3e), X⁵ can be selected from F, W, and Y.

In an IL-2Rβ ligand of Formula (3)-(3e), X⁶ can be selected from F, I, K, L, M, R, and V.

In an IL-2Rβ ligand of Formula (3)-(3e), X⁶ can be selected from I, L, and M.

In an IL-2Rβ ligand of Formula (3)-(3e), X⁷ can be A.

In an IL-2Rβ ligand of Formula (3)-(3e), X⁸ can be selected from H, K, L, Q, R, and S.

In an IL-2Rβ ligand of Formula (3)-(3e), X⁸ can be Q.

In an IL-2Rβ ligand of Formula (3)-(3e), X⁹ can be selected from I, L, and V.

In an IL-2Rβ ligand of Formula (3)-(3e), X⁹ can be selected from L and V.

In an IL-2Rβ ligand of Formula (3)-(3e), X¹⁰ can be G.

In an IL-2Rβ ligand of Formula (3)-(3e), X¹¹ can be selected from D and E.

In an IL-2Rβ ligand of Formula (3)-(3e), X¹¹ can be E.

In an IL-2Rβ ligand of Formula (3)-(3e), X¹² can be selected from L and V.

In an IL-2Rβ ligand of Formula (3)-(3e), X¹² can be L.

In an IL-2Rβ ligand of Formula (3)-(3c), X¹³ can be selected from D and E.

In an IL-2Rβ ligand of Formula (3)-(3c), X¹³ can be D.

In an IL-2Rβ ligand of Formula (3)-(3b), X¹⁴ can be selected from F, I, L, and M.

In an IL-2Rβ ligand of Formula (3)-(3b), X¹⁴ can be L.

In an IL-2Rβ ligand of Formula (3)-(3a), X¹⁵ can be selected from D, E, F, G, and V.

In an IL-2Rβ ligand of Formula (3)-(3a), X¹⁵ can be selected from D, E, F, and G.

In an IL-2Rβ ligand of Formula (3), X¹⁶ can be selected from D, E, G, K, P, V, and W.

In an IL-2Rβ ligand of Formula (3), X¹⁶ can be G.

In an IL-2Rβ ligand of Formula (3), X¹⁷ can be selected from A, E, G, P, Q, S, T, V, and W.

In an IL-2Rβ ligand of Formula (3), X¹⁷ can be G.

In an IL-2Rβ ligand of Formula (3)-(3e), the IL-2Rβ ligand can be defined by any combination of X¹ to X¹⁷ as defined in the immediately preceding thirty one (31) paragraphs.

In an IL-2Rβ ligand of Formula (3)-(3e),
X¹ can be selected from A, D, E, G, R, S, T, V, and W;
X² can be selected from F, L, S, V, W, and Y;
X³ can be selected from F, H, K, L, N, Q, W, and Y;
X⁴ can be selected from D and P;
X⁵ can be selected from F, L, S, W, and Y;
X⁶ can be selected from F, I, K, L, M, R, and V;
X⁷ can be A;
X⁸ can be selected from H, K, L, Q, R, and S;
X⁹ can be selected from I, L, and V;
X¹⁰ can be G;
X¹¹ can be selected from D and E;
X¹² can be selected from L and V;
X¹³ can be selected from D and E;
X¹⁴ can be selected from F, I, L, and M;
X¹⁵ can be selected from D, E, F, G, and V;
X¹⁶ can be selected from D, E, G, K, P, V, and W; and
X¹⁷ can be selected from A, E, G, P, Q, S, T, V, and W.

In an IL-2Rβ ligand of Formula (3)-(3e),
X¹ can be selected from G, R, and W;
X² can be selected from F and W;
X³ can be selected from F, H, L, W, and Y;
X⁴ can be selected from D and P;
X⁵ can be selected from F, W, and Y;
X⁶ can be selected from I, L, and M;
X⁷ can be A;
X⁸ can be Q;
X⁹ can be selected from L and V;
X¹⁰ can be G;
X¹¹ can be E;
X¹² can be L;
X¹³ can be D;
X¹⁴ can be L;
X¹⁵ can be selected from D, E, F, and G;
X¹⁶ can be G; and
X¹⁷ can be G.

An IL-2Rβ ligand can comprise the amino acid sequence of any one of SEQ ID NO: 337-560.

| | |
|---|---|
| SEQ ID NO: 337 | W Y P C W I A Q L G E L C D L D A K G Q R R |
| SEQ ID NO: 338 | T N N F Y P C W L A K L G D L C D F D D L N |
| SEQ ID NO: 339 | W Y P C W I A R V G E L C D L E E G P V N R |
| SEQ ID NO: 340 | A V E F Y P C W L A R I G E L C D L V E P |
| SEQ ID NO: 341 | W Y P C W I A H L G E L C D L E |
| SEQ ID NO: 342 | W Y P C W I A R V G E L C D M E |
| SEQ ID NO: 343 | R R E W Y P C W I A Q V G E L C D L P L I |
| SEQ ID NO: 344 | R W P W Y P C E I A R I G E L C D L E Q A N |
| SEQ ID NO: 345 | F G A F Y P C W K A Q L G E L C D L E P V T |
| SEQ ID NO: 346 | H G R W F P C W M A Q V G D L C D L E H S N |
| SEQ ID NO: 347 | W Y P C W L A K L G E L C D L D R A E A L P |
| SEQ ID NO: 348 | V W F P C W F A Q L G D L C D L D Q D P |
| SEQ ID NO: 349 | E T L G S V W Y P C W I A S I G E L C D L D |
| SEQ ID NO: 350 | W Y S C W I A Q L G E L C D L D D M G D R V |
| SEQ ID NO: 351 | W F P C W L A Q L G E L C D K E |
| SEQ ID NO: 352 | W Y P C W I A Q L G E L C D L V |
| SEQ ID NO: 353 | S H L W F P C W M A Q L G E L C D L E G G P |
| SEQ ID NO: 354 | R R W L P C W M A H V G E L C D L E L G N |
| SEQ ID NO: 355 | Y W C W F A R V G E L C D L D D G G V P S |
| SEQ ID NO: 356 | W Y S C W I A Q I G E L C D L E |
| SEQ ID NO: 357 | M N P V G T F Y P C W I A K L G E L C D L Q |
| SEQ ID NO: 358 | H G G V I G W Y P C W M A K V G E L C D L D |
| SEQ ID NO: 359 | W Y P C S I A L L G E L C D L E S V H E K S |
| SEQ ID NO: 360 | E Q R N R Q W W P C W L A R L G D L C D L D |
| SEQ ID NO: 361 | W Y P C W L A Q L G E L C D L D |
| SEQ ID NO: 362 | W L P C W M A H L G D L C D L E |
| SEQ ID NO: 363 | R P S W A P C W L A Q V G E L C D L D H P E |
| SEQ ID NO: 364 | E S K E P A F W P C W M A Q L G D L C D L E |
| SEQ ID NO: 365 | R S L R E S W F P C W M A K L G D L C D L E |
| SEQ ID NO: 366 | W H P C W L A R V G D L C D L D |
| SEQ ID NO: 367 | S R S A G E Y Y P C W L A Q L G E L C D L A |
| SEQ ID NO: 368 | R Q V P K Q W Y P C W M A A L G E L C D L E |
| SEQ ID NO: 369 | W Y P C W L A H L G E L C D L E |
| SEQ ID NO: 370 | Q V M R L W Y P C W M A Q L G E L C D L E |
| SEQ ID NO: 371 | W Y P C W M A Q L G E L C D L D E A P V Q P |
| SEQ ID NO: 372 | R D I W W P C W V A Q L G E L C D L D D P Q |
| SEQ ID NO: 373 | S A T W Y P C F L A N L G E L C D L E Q E N |
| SEQ ID NO: 374 | Y A E W Y P C W M A R V G E V C D L E V T P |
| SEQ ID NO: 375 | R S G D K A F F P C W L A Q L G D L C D L D |
| SEQ ID NO: 376 | W Y P C W M A Q L G E L C D M D |
| SEQ ID NO: 377 | R V S Y P C W L A R L G E L C D M D L E E |
| SEQ ID NO: 378 | T E S W Y P C W L A N L G D L C D L E W S A |
| SEQ ID NO: 379 | W L P C W M A D V G D L C D L D |
| SEQ ID NO: 380 | W H P C W M A R L G E L C D L D |
| SEQ ID NO: 381 | Q G T K W H W N P C W M A Q L G E L C D L D |
| SEQ ID NO: 382 | K N G P K S W Y P C W M A Q V G D L C D L D |
| SEQ ID NO: 383 | S G T G P A W Y P C F L A S L G Q L C D L E |
| SEQ ID NO: 384 | W Y P C W M A R M G E L C D L E |
| SEQ ID NO: 385 | W L P C W R A Q L G Q L C D L D |
| SEQ ID NO: 386 | L F P C W L A Q L G E L C D L E |
| SEQ ID NO: 387 | R Y P C W I A Q L G E L C D L D |
| SEQ ID NO: 388 | W H P C W I A H L G E L C D L E |
| SEQ ID NO: 389 | M Y P C W I A H L G E L C D L D |
| SEQ ID NO: 390 | W Y P C S I A S L G E L C D L E |
| SEQ ID NO: 391 | W L P C Y M A Q L G D L C D L E |
| SEQ ID NO: 392 | W H Y C W M A Q V G E V C D L D |
| SEQ ID NO: 393 | W Y P C W L A S L G E V C D L E |
| SEQ ID NO: 394 | W W P C S I A R L G Q L C D L D |
| SEQ ID NO: 395 | W Y P C W L A H L G E L C D L A |
| SEQ ID NO: 396 | W Y P C W L A Q L G E L C D A E |
| SEQ ID NO: 397 | W K P C W M A L L G E L C D L E |
| SEQ ID NO: 398 | R Y P C W R A K L G E L C D L D |
| SEQ ID NO: 399 | E E Q S R G F L P C W M A L L G E L C D L D |
| SEQ ID NO: 400 | L G S K R Q W Y P C W V A H L G E L C D L E |
| SEQ ID NO: 401 | E S E G R G W Y P C W N A L L G E L C D L E |
| SEQ ID NO: 402 | R W T Q A Q W Y P C W L A Q L G E L C D L E |
| SEQ ID NO: 403 | L H A G R W N P C W L A Q L G E L C D L E |
| SEQ ID NO: 404 | L S S K G W Y P C W K A R L G D L C D L E |
| SEQ ID NO: 405 | D M F T H R W Y P C S M A K L G E L C D L E |
| SEQ ID NO: 406 | M T D R A F W N P C W V A R L G E L C D L D |
| SEQ ID NO: 407 | N V T Y T Q W F P C W L A R L G E L C D L V |
| SEQ ID NO: 408 | V R T R I W Y P C W S A Q L G E L C D L D |
| SEQ ID NO: 409 | A M A R R Y L P C W I A K L G E L C E L D |
| SEQ ID NO: 410 | A R G E Y R W F P C W M A R L G E L C D L E |
| SEQ ID NO: 411 | Y L E R S R W Y P C F I A Q L G E L C D L E |
| SEQ ID NO: 412 | F R V S R D W F P C W M A Q L G E V C D L E |
| SEQ ID NO: 413 | I E R A W E W R P C W L A S V G E L C D L E |
| SEQ ID NO: 414 | V A S E R F Y P C W I A R L G E L C D V E |
| SEQ ID NO: 415 | W Y P C W I A K L G E V C D L D Q G T T R Q |
| SEQ ID NO: 416 | W Y P C W L A H L G E L C D L D W K G R N D |
| SEQ ID NO: 417 | W Y P C W R A Q L G E L C D L V D L G S H L |
| SEQ ID NO: 418 | W S P C W M A S L G D L C D L E E T R Q T E |
| SEQ ID NO: 419 | W T P C W I A Q L G E L C D L E G R H G T V |
| SEQ ID NO: 420 | L P C W I A Q L G D L C D L E P E P S P E |
| SEQ ID NO: 421 | F Y P C W A A H L G D L C D L E Y Q E A G P |
| SEQ ID NO: 422 | W L P C W L A P L G D L C D M D M S A V M N |
| SEQ ID NO: 423 | W R P C W M A H L G D L C D L E M A N E N P |
| SEQ ID NO: 424 | W Y P C W L A Q L G E V C D L D D G G G V F |
| SEQ ID NO: 425 | W W P C W L A Q L G E L V D L E V N G S L I |
| SEQ ID NO: 426 | T E M W Y P C W M A Y Q G E L C D L D M T Y |
| SEQ ID NO: 427 | A R T W W P C W R A K L G E L C D L V V P E |
| SEQ ID NO: 428 | H Q G F Y S C R L A R L G E L C D L D T G W |
| SEQ ID NO: 429 | V D E F Y P C S M A G L G E L C D L E R Q N |
| SEQ ID NO: 430 | A W D W Y P C S V A A L G E I C D L D I Q D |
| SEQ ID NO: 431 | R P P W Y P C W M A R L G E V C D M D I M L |
| SEQ ID NO: 432 | S Q R W Y P C W V A H L G E L C D L E G V V |
| SEQ ID NO: 433 | K G S W Q P C W F A K L G E L C D L H P T S |
| SEQ ID NO: 434 | Q T W Y L P C W M A K L G E L C D L G E R D |
| SEQ ID NO: 435 | E P R W Y P C W M A Q M G E L C D M E M S D |
| SEQ ID NO: 436 | W G G R Y W C W M A K L G D L C D L E D E W |
| SEQ ID NO: 437 | W W P C W I A Q V G E L C D L D G P G R P T |
| SEQ ID NO: 438 | R L V Y D C L F A Q V G D L C E V I S |
| SEQ ID NO: 439 | W R I L W M Q Q C W R S H V V N Q C A L |
| SEQ ID NO: 440 | W Y P C W I A Q V G E L C D L D E V S H G R |
| SEQ ID NO: 441 | T G E W W P C W V A E V G E L C D L E R G P |
| SEQ ID NO: 442 | A R T Q G W Y D C L F A Q V G E L C D L |
| SEQ ID NO: 443 | F H P C W R A L L G E L C D L E T A L G P S |
| SEQ ID NO: 444 | L Q I R K L W A C R I D L V G P F C L L |
| SEQ ID NO: 445 | A E Y S G R Y D C Y I A K V G E L C D I |
| SEQ ID NO: 446 | S W R F L W Q D C G R A H V G E L C D L |
| SEQ ID NO: 447 | N R W W H P C W M A R V G E L C D L E P D A |
| SEQ ID NO: 448 | W W P C W V A K L G E L C D L E G D A S R V |
| SEQ ID NO: 449 | W Y P C E F A Q L G E L C D L L P F P Y P A |
| SEQ ID NO: 450 | S Y M H D C F M A Q V G D L C D R F I S |
| SEQ ID NO: 451 | W W P C W I A Q V G E L C D L E E E S R E S |
| SEQ ID NO: 452 | K W A W N P C Y I A R L G E L C D L V E P E |
| SEQ ID NO: 453 | W W P C W I A D L G E L C D L E G P P R G R |
| SEQ ID NO: 454 | P T L I T W Y D C L F A E V G E L C D M |
| SEQ ID NO: 455 | E I S N W F L D C M F A D V G D L C D L |
| SEQ ID NO: 456 | A Q V W Y P C W L A K V G E L C D L D Q W N |
| SEQ ID NO: 457 | F G G K M D W Y P C W I A N L G E L C D L K |
| SEQ ID NO: 458 | W F P C W M A K V G D L C D V D E H Q D P S |
| SEQ ID NO: 459 | M G D S S S W F P C W M A Q L G E L C D M E |
| SEQ ID NO: 460 | M F R Y Y P C W I A S I G E L C D L E W G V |
| SEQ ID NO: 461 | E R R W Y P C W L A S V G E L C D L D M G D |
| SEQ ID NO: 462 | W Y P C W V A Q L G E L C D L E |
| SEQ ID NO: 463 | R W D Y W P C Y I A Q V G E L C D L E V Y E |
| SEQ ID NO: 464 | S L A H R S W Y P C W L A Q V G E L C D L D |
| SEQ ID NO: 465 | Q N A S K G W Y P C W I A H V G E L C D W D |
| SEQ ID NO: 466 | H R W Y P C W L A H L G E L C D L D P M S |
| SEQ ID NO: 467 | F Y P C W I A F V G E L C D L E |
| SEQ ID NO: 468 | E G H W Y P C W I A Q L G E L C D L D W |
| SEQ ID NO: 469 | W T G W S A F Y P C S I A N L G E L C D L D |
| SEQ ID NO: 470 | W E K L Q N W Y P C W I A Q M G E L C D L E |
| SEQ ID NO: 471 | T N G V L D W W P C W M A Q V G E L C D L D |
| SEQ ID NO: 472 | W Y P C W V A K L G E L C D L E |
| SEQ ID NO: 473 | A Y Y P C E L A Q L G E L C D L Y N I |
| SEQ ID NO: 474 | W Y P C W M A H L G E L C D L E |
| SEQ ID NO: 475 | N D H T A W W P C Y F A Q V G D L C D L V |
| SEQ ID NO: 476 | W W P C E I A Q I G E L C D L E W V R H A E |
| SEQ ID NO: 477 | W W P C D F A Q I G E L C D L G P R F T G E |
| SEQ ID NO: 478 | R D W W L P C E F A L I G E L C D L E R S W |
| SEQ ID NO: 479 | M R T T F W Y D C Y I A Q V G E L C D F |
| SEQ ID NO: 480 | S W H A E T W Y P C W L A Q V G E L C D L D |
| SEQ ID NO: 481 | E W F H D C F L A K V G D L C D L F L W |
| SEQ ID NO: 482 | S G K T Q M W N P C Y V A K V G E L C D L V |
| SEQ ID NO: 483 | D K A G P N F Y P C W L A H V G E L C D Q A |
| SEQ ID NO: 484 | A G F R G R W W P C E Y A Q V G E L C D L E |
| SEQ ID NO: 485 | W F P C W L A K V G E L C D R D D L A G P S |
| SEQ ID NO: 486 | W W P C E W A R I G E L C D L E |
| SEQ ID NO: 487 | K G S S W F P C Y F A Q V G D L C D L Y |
| SEQ ID NO: 488 | W Y P C W L A Q V G E L C D R E |
| SEQ ID NO: 489 | R G V Y F P C W L A K V G D L C D S D E F |
| SEQ ID NO: 490 | R A W W W P C E L A Q V G E L C D L E P S S |
| SEQ ID NO: 491 | W Y P C W L A K V G E L C D Q E |
| SEQ ID NO: 492 | R Y V P D C L K A Q V G D L C D F F A W |
| SEQ ID NO: 493 | W W P C Y L A Q I G E L C D L V |
| SEQ ID NO: 494 | W Y P C W M A K V G E L C D M E |
| SEQ ID NO: 495 | Q I T D S G W Y P C W V A K V G E L C D M D |
| SEQ ID NO: 496 | Y R W W Y P C D I A Q V G E L C D L D Y L L |
| SEQ ID NO: 497 | C Y M H D C F M A Q V G D L C D R F I S |
| SEQ ID NO: 498 | W L P C W I A K I G D L C D L D |
| SEQ ID NO: 499 | S R V W H P C W L A R V G E L C D L E V S D |
| SEQ ID NO: 500 | W E H E F T W Y P C W I A Q V G E L C D M D |
| SEQ ID NO: 501 | H R G W V G W Y P C E Y A L P G Q L C D L E |
| SEQ ID NO: 502 | H Y P C W L A Q L G E L C D Q D W D T P S |
| SEQ ID NO: 503 | R V R R H S W W P C E I A V V G E L C D L E |
| SEQ ID NO: 504 | D G W W P C W I A Q V G E L C D L E D P V |
| SEQ ID NO: 505 | L P F Q D C Y I A Q V G E L C D L P G T |
| SEQ ID NO: 506 | R W M F D C L F A R V G E L C D I R P W |
| SEQ ID NO: 507 | G G Y Y D C L I A E V G E L C D M P G Q |
| SEQ ID NO: 508 | V V C Y A C D I A H V G E L C D L T C R |
| SEQ ID NO: 509 | T P W Y D C Y I A N V G D L C D F A S A |
| SEQ ID NO: 510 | L E S L D C F F A R I G D L C E I W D V |
| SEQ ID NO: 511 | W Q I F D C Y L A Q V G E L C D L Q D T |
| SEQ ID NO: 512 | G R Y P D C Y I A H V G E L C E F Y D G |
| SEQ ID NO: 513 | F G D D F C R F I P L F E M C T T D V E |
| SEQ ID NO: 514 | L V Y Y D C Y M A Q V G E L C D L P S L |
| SEQ ID NO: 515 | V S R Y D C Y I A K V G E L C D F F E F |
| SEQ ID NO: 516 | V T V Q D C Y F A R V G D L C D L F S P |
| SEQ ID NO: 517 | W E W Y D C L M A Q V G E L C D F E G N |
| SEQ ID NO: 518 | W A F Y D C R N A Q V G D F C D L W E F |
| SEQ ID NO: 519 | S M D Q D C Y F A Q V G E L C V L F N Q |
| SEQ ID NO: 520 | G G Y Y D C L I A E V G E L C D I Y G R |
| SEQ ID NO: 521 | S N W H D C L F A Q V G E L C D L P G S |
| SEQ ID NO: 522 | Y D C Y I A Q V G E L C D I |
| SEQ ID NO: 523 | S W L S D L Q D C Y I A Q V G D L C Q I |
| SEQ ID NO: 524 | R L A S D W W D C Y I A K V G E L C D F |
| SEQ ID NO: 525 | R L L R D C F L A K V G D L C E L F V W |
| SEQ ID NO: 526 | Q W F H N C F L A R V G D T C D L F L W |
| SEQ ID NO: 527 | E L L V D C F K V K V G E L C D L F F G |
| SEQ ID NO: 528 | R Y V H D C F I A Q V G D L C D L F L H |
| SEQ ID NO: 529 | K W V H D C F L A K V G D V C D L F V V |
| SEQ ID NO: 530 | R S L V D C F L V K V G D L C D F F N W |
| SEQ ID NO: 531 | R Y L Y D C F L A L V G D L C V K F H Q |
| SEQ ID NO: 532 | A H F Y D C F W A K A G E L C D L W P S |
| SEQ ID NO: 533 | K W F H D C F L A K V G D L C D L F L W |
| SEQ ID NO: 534 | W G K L V R D C F L A K V G D L C D L F L W |
| SEQ ID NO: 535 | T H V H D C F L A K V G D L C D L F I V |
| SEQ ID NO: 536 | H W V R D C F L A K V G E L C D L F L W |
| SEQ ID NO: 537 | H G I L D C Y F A K V G E L C E L F D W |
| SEQ ID NO: 538 | Q F V K D C F L A Q V G D L C E L F L W |
| SEQ ID NO: 539 | D W L P D C Y F A N V G D L C S L F G S |
| SEQ ID NO: 540 | H W F L D C F L A N V G D L C D F F G N |
| SEQ ID NO: 541 | N W L P D C L F A N V G E L C D I F P W |
| SEQ ID NO: 542 | E I F K D C L F A N V G E L C E I F P S |
| SEQ ID NO: 543 | N W F H D C F L A R V G D L C D L F L D |
| SEQ ID NO: 544 | Y S F K D C Y F A K V G E L C E L F L W |
| SEQ ID NO: 545 | E F F H D C Y V A R V G E L C D L F G W |
| SEQ ID NO: 546 | S Y L C W L D H W G V I C E E D |
| SEQ ID NO: 547 | W Y T C M M D W L G V H C E L E |
| SEQ ID NO: 548 | Q M V W W D C W S T Q E G P V C E L N W T A |
| SEQ ID NO: 549 | Y W C S V W Q L G S V C E M N N E E T Q |
| SEQ ID NO: 550 | W T C W L T Q L G Y D C N L D V V D Q S L G |
| SEQ ID NO: 551 | D G S W Y T C W F T Q L G E W C E Q D D A K |
| SEQ ID NO: 552 | E F W G W Q C W Q E P L G W S C D L E W M D |
| SEQ ID NO: 553 | W Y P C W I A R V G E L C D L E |
| SEQ ID NO: 554 | W Y P C W L A Q V G E L C D L D |
| SEQ ID NO: 555 | L V D C F K V K V G E L C D L F |
| SEQ ID NO: 556 | V H D C F I A Q V G D L C D L F |
| SEQ ID NO: 557 | W P C W A A L G E L C D L D |
| SEQ ID NO: 558 | W P C W A L G E L C D L D |
| SEQ ID NO: 559 | W P C W L G E L C D L D |
| SEQ ID NO: 560 | W Y P C W G E L C D L D |

An IL-2Rβ ligand can comprise a truncated amino acid sequence of any one of SEQ ID NO: 330-560.

An IL-2Rβ ligand can comprise an amino acid sequence selected from any one of SEQ ID NO: 330-560, or a truncated amino acid sequence of any one of SEQ ID NO: 330-560, wherein the amino acid sequence can independently comprise from 1 to 4 glycines (G) (SEQ ID NO: 30) on the N-terminus, on the C-terminus, or on both the N- and C-termini.

An IL-2Rβ ligand can comprise an amino acid sequence selected from any one of SEQ ID NO: 330-560, or a truncated amino acid sequence of any one of SEQ ID NO: 330-560, wherein the amino acid sequence comprises one or more amino acid substitutions such as from 1 to 5 amino acid substitutions. Each of the amino acid substitutions can independently be selected from conservative amino acid substitution and a non-conservative amino acid substitution.

An IL-2Rβ ligand can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 330-560 or to a truncated amino acid sequence of any one of SEQ ID NO: 330-560.

Certain of the IL-2Rβ ligand of any one of SEQ ID NO: 330-560, a truncated IL-2Rβ ligand of any one of SEQ ID NO: 330-560, or a substituted IL-2Rβ ligand of any one of SEQ ID NO: 330-560 can bind to the hu-IL-2Rβ subunit with an IC₅₀ of less than 100 μM or less than 10 μM and to the cyno-IL-2Rβ subunit with an IC₅₀ of less than 100 μM or less than 10 μM as determined using phage ELISA competition assays.

Certain of the IL-2Rβ ligands of any one of SEQ ID NO: 330-336 bind to the hu-IL-2Rβ subunit with an IC₅₀ of less than 100 μM as determined using phage ELISA competition assays.

An IL-2Rβ ligand of any one of SEQ ID NO: 337-560 binds to the hu-IL-2Rβ subunit with an IC₅₀ of less than 100 μM as determined using phage ELISA competition assays.

An IL-2Rβ ligand can comprise the amino acid sequence of Formula (4) (SEQ ID NO: 561), an amino acid sequence of Formula (4a) (SEQ ID NO: 562), an amino acid sequence of Formula (4b) (SEQ ID NO: 563), an amino acid sequence of Formula (4c) (SEQ ID NO: 564), an amino acid sequence of Formula (4d) (SEQ ID NO: 565), an amino acid sequence of Formula (4e) (SEQ ID NO: 566), or an amino acid sequence of Formula (40 (SEQ ID NO: 567):

-X¹-X²-X³-X⁴-X⁵-C-X⁶-X⁷-X⁸-X⁹-X¹⁰-X¹¹-X¹²-X¹³-C-X¹⁴-X¹⁵-X¹⁶-X¹⁷-X¹⁸- (4)

-X²-X³-X⁴-X⁵-C-X⁶-X⁷-X⁸-X⁹-X¹⁰-X¹¹-X¹²-X¹³-C-X¹⁴-X¹⁵-X¹⁶-X¹⁷- (4a)

-X³-X⁴-X⁵-C-X⁶-X⁷-X⁸-X⁹-X¹⁰-X¹¹-X¹²-X¹³-C-X¹⁴-X¹⁵-X¹⁶- (4b)

-X⁴-X⁵-C-X⁶-X⁷-X⁸-X⁹-X¹⁰-X¹¹-X¹²³-X¹³-C-X¹⁴-X¹⁵- (4c)

-X⁵-C-X⁶-X⁷-X⁸-X⁹-X¹⁰-X¹¹-X¹²-X¹³-C-X¹⁴- (4d)

-C-X⁶-X⁷-X⁸-X⁹-X¹⁰-X¹¹-X¹²-X¹³-C- (4e)

-X⁶-X⁷-X⁸-X⁹-X¹⁰-X¹¹-X¹²-X¹³- (4f)

wherein,
X¹ can be selected from A, D, E, H, K, N, Q, R, and T;
X² can be selected from F, G, H, I, L, S, W, and Y;
X³ can be selected from F, I, L, and V;
X⁴ can be selected from H, K, L, P, R, V, and Y;
X⁵ can be D;
X⁶ can be selected from F, L, and Y;
X⁷ can be selected from F, I, K, L, V, and W;
X⁸ can be selected from A and V;
X⁹ can be selected from K, L, N, Q, and R;
X¹⁰ can be selected from A and V;
X¹¹ can be G;
X¹² can be selected from D and E;
X¹³ can be selected from L, T, and V;
X¹⁴ can be selected from D, E, S, and V;
X¹⁵ can be selected from F, I, K, and L;
X¹⁶ can be selected from F and W;
X¹⁷ can be selected from D, F, G, I, L, N, P, V; and
X¹⁸ can be selected from D, G, H, N, Q, S V, and W.

In an IL-2Rβ ligand of Formula (4)-(4e), X² can be W.

In an IL-2Rβ ligand of Formula (4)-(4e), X⁶ can be F.

In an IL-2Rβ ligand of Formula (4)-(4e), X⁷ can be selected from F and L.

In an IL-2Rβ ligand of Formula (4)-(4e), X⁸ can be A.

In an IL-2Rβ ligand of Formula (4)-(4e), X⁹ can be K.

In an IL-2Rβ ligand of Formula (4)-(4e), X¹⁰ can be V.

In an IL-2Rβ ligand of Formula (4)-(4e), X¹³ can be L.

In an IL-2Rβ ligand of Formula (4)-(4e), X¹⁴ can be D.

In an IL-2Rβ ligand of Formula (4)-(4e), X¹⁵ can be L.

In an IL-2Rβ ligand of Formula (4)-(4e), X¹⁶ can be F.

In an IL-2Rβ ligand of Formula (4)-(4e), X¹⁷ can be L.

In an IL-2Rβ ligand of Formula (4)-(4e), X¹⁸ can be W.

In an IL-2Rβ ligand of Formula (4)-(4e), the IL-2Rb ligand can be defined by any combination of X¹ to X¹⁸ as defined in the immediately preceding twelve (12) paragraphs.

An IL-2Rβ ligand can comprise the amino acid sequence of any one of SEQ ID NO: 568-587.

| | |
|---|---|
| SEQ ID NO: 568 | A F F H D C F F A K A G D L C D F F D D |
| SEQ ID NO: 569 | D F F H D C F F A K V G D L C D F F F G |
| SEQ ID NO: 570 | E G F H D C F F A K V G D L C D I F G H |
| SEQ ID NO: 571 | E H F H D C F F A K V G D L C D I F G N |
| SEQ ID NO: 572 | E H F H D C F F A K V G D L C D K F G Q |
| SEQ ID NO: 573 | H I F H D C F I A K V G D L C D L F H S |
| SEQ ID NO: 574 | H L F H D C F K A K V G D L C D L F I S |
| SEQ ID NO: 575 | H L F K H C F L A K V G D L C D L F L S |
| SEQ ID NO: 576 | W G K S I K D C F L A K V G D L C D L F L V |
| SEQ ID NO: 577 | K S L K D C F L A K V G D L C D L F L V |
| SEQ ID NO: 578 | K W L L D C F L A L V G D L C D L F L W |
| SEQ ID NO: 579 | N W L L D C F L A N V G D L C D L F L W |
| SEQ ID NO: 580 | N W L P D C F L A N V G E L C D L F L W |
| SEQ ID NO: 581 | Q W L P D C F L A N V G E L C D L F L W |
| SEQ ID NO: 582 | Q W V R D C L L A N V G E L C E L F L W |
| SEQ ID NO: 583 | R W V R D C L L A Q V G E L C E L F N W |
| SEQ ID NO: 584 | R W V V G C Y L A Q V G E L C E L F P W |
| SEQ ID NO: 585 | R W V V D C Y L A R V G E L C E L F P W |
| SEQ ID NO: 586 | T Y V Y D C Y V V R V G E T C S L F P W |
| SEQ ID NO: 587 | Y Y V Y N C Y W V R V G E V C V L W V W |

An IL-2Rβ ligand can comprise a truncated amino acid sequence of any one of SEQ ID NO: 561-587.

An IL-2Rβ ligand can comprise an amino acid sequence selected from any one of SEQ ID NO: 561-587, or a truncated amino acid sequence of any one of SEQ ID NO: 561-587, wherein the amino acid sequence can independently comprise from 1 to 4 glycines (G) (SEQ ID NO: 30) on the N-terminus, on the C-terminus, or on both the N- and C-termini.

An IL-2Rβ ligand can comprise an amino acid sequence selected from any one of SEQ ID NO: 561-587, or a truncated amino acid sequence of any one of SEQ ID NO: 561-587, wherein the amino acid sequence comprises one or more amino acid substitutions such as from 1 to 5 amino acid substitutions. Each of the amino acid substitutions can independently be selected from a conservative amino acid substitution and a non-conservative amino acid substitution.

An IL-2Rβ ligand can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 561-587 or to a truncated amino acid sequence of any one of SEQ ID NO: 561-587.

An IL-2Rβ ligand of any one of SEQ ID NO: 561-587, a truncated IL-2Rβ ligand of any one of SEQ ID NO: 561-587, or a substituted IL-2Rβ ligand of any one of SEQ ID NO: 561-587 can bind to the hu-IL-2Rβ subunit with an IC₅₀ of less than 100 μM or less than 10 μM as determined using phage ELISA competition assays.

Certain of the IL-2Rβ ligands of any one of SEQ ID NO: 561-567 bind to the hu-IL-2Rβ subunit with an IC₅₀ of less than 100 μM as determined using phage ELISA competition assays.

The IL-2Rβ ligand of any one of SEQ ID NO: 568-587 bind to the hu-IL-2Rβ subunit with an IC₅₀ of less than 100 μM as determined using phage ELISA competition assays.

Certain IL-2Rβ ligands having SEQ ID NO: 173-253, 330-560, 561-587, and 609-618 bound to the hu-IL-2Rβ subunit with an IC₅₀ of less than 100 μM and to the cyno-IL-2Rβ subunit with an IC₅₀ of less than 100 μM as determined using phage ELISA competition assays.

An IL-2Rβ ligand can have the amino acid sequence of any of SEQ ID NO: 588-618:

| | |
|---|---|
| SEQ ID NO: 588 | G G Y D C R I A Q V G E L C D L G G |
| SEQ ID NO: 589 | G V Q Y K K C W M A Q L G D L C E L D P S G G |
| SEQ ID NO: 590 | G G Y P C W M A Q L G E L C D L G G R R |
| SEQ ID NO: 591 | G G W Y P C W M A Q L G E L C D L D G G |
| SEQ ID NO: 592 | G G W Y P C W M A Q L G E L C D L D G G R R |
| SEQ ID NO: 593 | G G Y P C H M A Q L G E L C D L W S W G D I G G R R |
| SEQ ID NO: 594 | F Y P C W T A L L G E L C D L E P G P P A M G G |
| SEQ ID NO: 595 | W R R W Y P C W V A Q V G E L C D L E I E A G G |
| SEQ ID NO: 596 | R Q R W Y P C W M A R L G E L C D L D E P T G G |
| SEQ ID NO: 597 | W Y P C W M A Q L G D L C D L E K P V T E R G G |
| SEQ ID NO: 598 | G G W Y P C W M A Q L G E L C D L D |
| SEQ ID NO: 590 | G G F Y P C W T A L L G E L C D L E P G P P A M G G |
| SEQ ID NO: 600 | G G W G T T W R W Y P C W M A Q L G E L C D L E G G |
| SEQ ID NO: 601 | G G D V L G D R W Y P C W I A K G E L C D L D G G |
| SEQ ID NO: 602 | G G W P C W I A Q L G E L C D L D G G |
| SEQ ID NO: 603 | G G W Y P C W L A K L G E L C D L D G G |
| SEQ ID NO: 604 | G G W Y P C W M A Q L G D L C D L E K P V T E R G G |
| SEQ ID NO: 605 | G G W R R W Y P C W V A Q V G E L C D L E I E A G G |
| SEQ ID NO: 606 | G G R Q R W Y P C W M A R L G E L C D L D E P T G G |
| SEQ ID NO: 607 | G G V Q Y K K C W M A Q L G D L C E L D P S G G |
| SEQ ID NO: 608 | G G Y P C H M A Q L G E L C D L W S W G D I G G |
| SEQ ID NO: 609 | G G W Y P C W I A R V G E L C D L E E G P V N R |
| SEQ ID NO: 610 | G G A V E F Y P C W L A R I G E L C D L V E P |
| SEQ ID NO: 611 | G G W Y P C W I A R V G E L C D M E |
| SEQ ID NO: 612 | G G E W F H D C F L A K V G D L C D L F L G G E W |
| SEQ ID NO: 613 | G G R Y V H D C F I A Q V G D L C D L F L H |
| SEQ ID NO: 614 | G G R S L V D C F L V K V G D L C D F F N W |
| SEQ ID NO: 615 | G G W Y P C W I A R V G E L C D L E |
| SEQ ID NO: 616 | G G W Y P C W L A Q V G E L C D L D |
| SEQ ID NO: 617 | G G L V D C F K V K V G E L C D L F |
| SEO ID NO. 618 | G G W Y S C W M A Q L G E L C D L D |

An IL-2Rβ ligand can be selected from SEQ ID NO: 619-621, 134, and 623-630.

| | |
|---|---|
| SEQ ID NO: 619 | V Q Y K K C W M A Q L G D L C E L D P S |
| SEQ ID NO: 620 | Y P C H M A Q L G E L C D L W S W G D I |
| SEQ ID NO: 621 | Y P C W M A Q L G E L C D L |
| SEQ ID NO: 134 | W Y P C W M A Q L G E L C D L D |
| SEQ ID NO: 623 | W G T T W R W Y P C W M A Q L G E L C D L E |
| SEQ ID NO: 624 | F Y P C W T A L L G E L C D L E P G P P A M |
| SEQ ID NO: 625 | W R R W Y P C W V A Q V G E L C D L E I E A |
| SEQ ID NO: 626 | R Q R W Y P C W M A R L G E L C D L D E P T |
| SEQ ID NO: 627 | D V L G D R W Y P C W I A K L G E L C D L D |
| SEQ ID NO: 628 | W Y P C W M A Q L G D L C D L E K P V T E R |
| SEQ ID NO: 629 | W Y P C W I A Q L G E L C D L D |
| SEQ ID NO: 630 | W Y P C W L A K L G E L C D L D |

An IL-2Rβ ligand can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 619-621, 134, and 623-630.

An IL-2Rβ ligand provided by the present disclosure can comprise a truncated amino acid sequence of any one of SEQ ID NO: 619-621, 134, and 623-630.

An IL-2Rβ ligand provided by the present disclosure can comprise an amino acid sequence selected from any one of SEQ ID NO: 619-621, 134, and 623-630, or a truncated amino acid sequence of any one of SEQ ID NO: 619-621, 134, and 623-630, wherein the amino acid sequence can independently comprise from 1 to 4 glycines (G) (SEQ ID NO: 30) on the N-terminus, on the C-terminus, or on both the N- and C-termini.

An IL-2Rβ ligand provided by the present disclosure can comprise an amino acid sequence selected from any one of SEQ ID NO: 619-621, 134, and 623-630, or a truncated amino acid sequence of any one of SEQ ID NO: 619-621, 134, and 623-630, wherein the amino acid sequence comprises one or more amino acid substitutions such as from 1 to 5 amino acid substitutions. Each of the amino acid substitutions can independently be selected from a conservative amino acid substitution and a non-conservative amino acid substitution.

An IL-2Rβ ligand can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 619-621, 134, and 623-630 or to a truncated amino acid sequence of any one of SEQ ID NO: 619-621, 134, and 623-630.

An IL-2Rβ ligand of any one of SEQ ID NO: 619-621, 134, and 623-630 bound to the hu-IL-2Rβ subunit with an IC₅₀ of less than 10 μM as determined using phage ELISA competition assays.

An IL-2Rβ ligand of any one of SEQ ID NO: 619-621, 134, and 623-630, a truncated IL-2Rβ ligand of any one of SEQ ID NO: 619-621, 134, and 623-630, or a substituted IL-2Rβ ligand of any one of SEQ ID NO: 619-621, 134, and 623-630 can bind to the hu-IL-2Rβ subunit with an IC₅₀ of less than 100 μM or less than 10 μM as determined using phage ELISA competition assays.

Certain IL-2Rβ ligands provided by the present disclosure can bind to a specific binding site on the hu-IL-2Rβ subunit that is different than the hu-IL-2Rβ binding site on the hu-IL-2Rβ subunit to which IL-2 binds.

These IL-2Rβ ligands do not compete for binding to the specific IL-2 binding site with IL-2, have no detectable binding to the hu-IL-2Rγc subunit, and bind to the hu-IL-2Rβ subunit with an IC₅₀ of less than 10 μM.

The specific binding site on the hu-IL-2Rβ subunit can be characterized by at least the following properties: (1) a group of IL-2Rβ ligands bind to a specific binding site on the hu-IL-2Rβ subunit with an IC₅₀ of less than 10 μM; (2) each IL-2Rβ ligands within the group competitively binds to the specific binding site on the hu-IL-2Rβ subunit with each of the other IL-2Rβ ligands within the group; (3) a peptide having the amino acid sequence of SEQ ID NO: 931 does not compete for binding to a specific binding site on the IL-2Rβ subunit with the peptides within the group of IL-2Rβ ligands; and (4) IL-2Rβ ligands having SEQ ID NO: 932, 933, 934, and 935 do not bind competitively with IL-2 binding to IL-2Rβ, indicating that this IL-2Rβ ligand binding site is distinct from that of IL-2.

An IL-2Rγc ligand having the amino acid sequence of SEQ ID NO: 935 does not compete for binding to the specific binding site with the group of IL-2Rβ ligands.

| | |
|---|---|
| SEQ ID NO: 931 | R S C Y Y K R P R L W C S E |
| SEQ ID NO: 932 | Y D C R I A Q V G E L C D L |
| SEQ ID NO: 933 | N M C L V G D Y W P S C Q I |
| SEQ ID NO: 934 | R W G D V G D L L M P L |
| SEQ ID NO: 935 | D C S M W E G V E L C W |

This specific binding site on the hu-IL-2Rβ subunit to which the group of IL-2Rβ ligands bind can be characterized using competitive binding assays as described.

An IL-2Rβ ligand provided by the present disclosure can be a pH-biased IL-2Rβ ligand.

pH-Biased IL-2Rβ ligands can bind to the IL-2Rβ subunit with an IC₅₀ at pH 6 that is at least 10% less than the IC₅₀ for binding to the IL-2Rβ subunit at pH 7.5.

For example, the IC₅₀ for binding of a hu-IL-2Rβ ligand at pH 6.0 can be 0.5 μM, and the IC₅₀ for binding of the same IL-2Rβ ligand at pH 7.5 can be 1 μM. A pH-biased IL-2Rβ ligand can bind to the IL-2Rβ subunit at pH 6.0 with an IC₅₀ that is at least 10% less than the IC₅₀ for binding to the hu-IL-2Rβ subunit at pH 7.5, at least 25% less, at least 50% less, at least 100% less, or at least 200% less than the IC₅₀ for binding to the hu-IL-2Rβ subunit at pH 7.5.

A pH-biased IL-2Rβ ligand can comprise, for example, from 5 to 30 amino acids.

A pH-biased IL-2Rβ ligand can bind to the hu-IL-2Rβ subunit at pH 6.0, for example, with an IC₅₀ from 1 pM to 100 μM and can bind to the hu-IL-2Rβ subunit with an IC₅₀ greater than 100 μM at pH 7.5.

A pH-biased IL-2Rβ ligand can bind to the hu-IL-2Rβ subunit at pH 6.0, for example, with an IC₅₀ from 0.1 μM to 50 μM and can bind to the hu-IL-2Rβ subunit with an IC₅₀ greater than 100 μM at pH 7.5.

A pH-biased IL-2Rβ ligand can bind to the hu-IL-2Rβ subunit at pH 6.0, for example, with an IC₅₀ of less than 100 μM and can bind to the hu-IL-2Rβ subunit, for example, with an IC₅₀ greater than 100 μM at pH 7.5.

A pH-biased IL-2Rβ ligand can bind to a mammalian IL-2Rβ subunit at pH 6.0, for example, with an IC₅₀ of less than 100 μM and can bind to the hu-IL-2Rβ subunit with an IC₅₀ greater than 100 μM at pH 7.5.

pH-biased IL-2Rβ ligands can be identified using methods based on acidic-biased affinity selection. These methods have been used to identify IL-2Rβ ligands having increased binding affinity at a pH less than pH 6.5 commensurate with a solid tumor microenvironment and having decreased binding affinity at a pH greater than pH 7.0 commensurate with that of normal tissue.

An IL-2Rγc ligand can comprise an IL-2Rγc ligand. Examples of suitable IL-2Rγc ligands are disclosed in U.S. Application Publication No. 2020/0040036 A1, which is incorporated by reference in its entirety.

An IL-2Rγc ligand such as an IL-2Rγc ligand provided by the present disclosure can bind to the hu-IL-2Rγc subunit with an IC₅₀ of less than 100 μM, less than 10 μM, less than 1 μM, less than 0.1 μM, or less than 0.01 μM.

An IL-2Rγc ligand provided by the present disclosure can bind to the hu-IL-2Rγc subunit with an IC₅₀ from 1 pM to 100 μM, from 10 pM to 10 μM, from 100 pM to 1 μM, from 0.001 μM to 1 μM, or from 0.01 μM to 1 μM.

An IL-2Rγc ligand provided by the present disclosure can bind to a mammalian IL-2Rγc subunit with an IC₅₀, for example, of less than 100 μM, less than 10 μM, less than 1 μM, less than 0.1 μM, or less than 0.01 μM.

An IL-2Rγc ligand provided by the present disclosure can bind to a mammalian IL-2Rγc subunit with an IC₅₀, for example, from 1 pM to 100 μM, from 10 pM to 10 μM, from 100 pM to 1 μM, from, 0.001 μM to 1 μM, or from 0.01 μM to 1 μM.

An IL-2Rγc ligand can comprise the amino acid sequence of Formula (5) (SEQ ID NO: 631), Formula (5a) (SEQ ID NO: 632), Formula (5b) (SEQ ID NO: 633), Formula (5c) (SEQ ID NO: 634), Formula (5d) (SEQ ID NO: 653), and/or Formula (5e) (SEQ ID NO: 636):

-X¹⁷¹-X¹⁷²-X¹⁷³-X¹⁷⁴-X¹⁷⁵-C-X¹⁷⁶-X¹⁷⁷-X¹⁷⁸-X¹⁷⁹-X¹⁸⁰-X¹⁸¹-X¹⁸²-X¹⁸³-C-X¹⁸⁴-X¹⁸⁵-X¹⁸⁶-X¹⁸⁷-X¹⁸⁸- (5)

-X¹⁷²-X¹⁷³-X¹⁷⁴-X¹⁷⁵-C-X¹⁷⁶-X¹⁷⁷-X¹⁷⁸-X¹⁷⁹-X¹⁸⁰-X¹⁸¹-X¹⁸²-X¹⁸³-C-X¹⁸⁴-X¹⁸⁵-X¹⁸⁶-X¹⁸⁷- (5A)

-X¹⁷³-X¹⁷⁴-X¹⁷⁵-C-X¹⁷⁶-X¹⁷⁷-X¹⁷⁸-X¹⁷⁹-X¹⁸⁰-X¹⁸¹-X¹⁸²-X¹⁸³-C-X¹⁸⁴-X¹⁸⁵-X¹⁸⁶- (5B)

-X¹⁷⁴-X¹⁷⁵-C-X¹⁷⁶-X¹⁷⁷-X¹⁷⁸-X¹⁷⁹-X¹⁸⁰-X¹⁸¹-X¹⁸²-X¹⁸³-C-X¹⁸⁴-X¹⁸⁵- (5C)

-X¹⁷⁵-C-X¹⁷⁶-X¹⁷⁷-X¹⁷⁸-X¹⁷⁹-X¹⁸⁰-X¹⁸¹-X¹⁸²-X¹⁸³-C-X¹⁸⁴- (5D)

-C-X¹⁷⁶-X¹⁷⁷-X¹⁷⁸-X¹⁷⁹-X¹⁸⁰-X¹⁸¹-X¹⁸²-X¹⁸³-C- (5E)

wherein each of X¹⁷¹-X¹⁸⁸ is independently selected from an amino acid.

In an IL-2Rγc ligand of Formula (5)-(5e), X¹⁷¹ can be selected from an amino acid comprising a basic side chain; X¹⁷² can be selected from an amino acid comprising a hydroxyl-containing side chain; X¹⁷³ can be selected from an amino acid comprising an acidic side chain or a large hydrophobic side chain; X¹⁷⁴ can be selected from an amino acid comprising a large hydrophobic side chain; X¹⁷⁵ can be selected from an amino acid comprising an acidic side chain or a large hydrophobic side chain; X¹⁷⁶ can be selected from an amino acid comprising an acidic side chain or a polar/neutral side chain; X¹⁷⁷ can be selected from an amino acid comprising an acidic side chain; X¹⁷⁸ can be selected from an amino acid comprising a large hydrophobic side chain or an aromatic side chain; X¹⁷⁹ can be selected from an amino acid comprising an acidic side chain or a polar/neutral side chain; X¹⁸⁰ can be G; X¹⁸¹ can be V; X¹⁸² can be E; X¹⁸³ can be L; X¹⁸⁴ can be W; X¹⁸⁵ can be selected from an amino acid comprising a large hydrophobic side chain; X¹⁸⁶ can be E; X¹⁸⁷ can be selected from an amino acid; and X¹⁸⁸ can be selected from an amino acid comprising an acidic side chain.

In an IL-2Rγc ligand of Formula (5), (5a), (5b), (5c), (5d), and/or (5e), X¹⁷¹ can be selected from H, K, and R; X¹⁷² can be selected from S, T, and Y; X¹⁷³ can be selected from D, E, F, I, L, M, V, W, and Y; X¹⁷⁴ can be selected from F, I, L, M, V, W, and Y; X¹⁷⁵ can be selected from D, E, F, I, L, M, V, W, and Y; X¹⁷⁶ can be selected from D, E, H, N, Q, S, T, and Y; X¹⁷⁷ can be selected from D and E; X¹⁷⁸ can be selected from F, H, I, L, M, V, W, and Y; X¹⁷⁹ can be selected from D, E, H, N, Q, S, T, and Y; X¹⁸⁰ can be G; X¹⁸¹ can be V; X¹⁸² can be E; X¹⁸³ can be L; X¹⁸⁴ can be W; X¹⁸⁵ can be selected from F, I, L, M, V, W, Y, H, N, Q, S, and T; X¹⁸⁶ can be E; X¹⁸⁷ can be selected from an amino acid; and X¹⁸⁸ can be selected from D and E.

In an IL-2Rγc ligand of Formula (5), (5a), (5b), (5c), (5d), and/or (5e), X¹⁷¹ can be selected from D, E, G, H, K, M, N, P, Q, R, S, and T; X¹⁷² can be selected from A, D, E, G, I, K, L, P, Q, R, S, T, V, W, and Y; X¹⁷³ can be selected from A, D, E, F, G, I, Q, S, T, V, W, and Y; X¹⁷⁴ can be selected from A, I, E, I, L, M, N, Q, R, S, T, and V; X¹⁷⁵ can be selected from A, E, I, L, M, N, Q, R, S, T, and V; X¹⁷⁶ can be selected from D, E, H, L, Q, R, and V; X¹⁷⁷ can be selected from D, E, N, T, and V; X¹⁷⁸ can be selected from F, S, W, and Y; X¹⁷⁹ can be selected from A, D, E, G, H, K, N, Q, R, and Y; X¹⁸⁰ can be selected from G and R; X¹⁸¹ can be V; X¹⁸² can be selected from D, E, and Y; X¹⁸³ can be selected from F, I, and L; X¹⁸⁴ can be W; X¹⁸⁵ can be selected from C, H, I, L, P, Q, T, V, and Y; X¹⁸⁶ can be selected from A, D, E, G, M, R, S, T, and V; X¹⁸⁷ can be selected from A, D, E, F, G, I, M, N, P, Q, R, S, T, V, W, and Y; and X¹⁸⁸ can be selected from A, C, D, E, F, G, I, K, L, N, P, Q, R, S, and V.

In an IL-2Rγc ligand of Formula (5), (5a), (5b), (5c), (5d), and/or (5e), X¹⁷¹ can be selected from H, K, and R; X¹⁷² can be selected from S, T, and Y; X¹⁷³ can be selected from D, E, F, I, and V; X¹⁷⁴ can be selected from I and V; X¹⁷⁵ can be selected from E, I, L, M, and V; X¹⁷⁶ can be selected from D, E, and Q; X¹⁷⁷ can be selected from D and E; X¹⁷⁸ can be selected from F and W; X¹⁷⁹ can be selected from D, E, N, and Q; X¹⁸⁰ can be G; X¹⁸¹ can be V; X¹⁸² can be selected from D and E; X¹⁸³ can be L; X¹⁸⁴ can be W; X¹⁸⁵ can be selected from I, L, Q, and V; X¹⁸⁶ can be selected from D and E; X¹⁸⁷ can be selected from A, D, E, F, G, I, M, N, P, Q, R, S, T, V, W, and Y; and X¹⁸⁸ can be selected from D, E, N, and Q.

In an IL-2Rγc ligand of Formula (5), (5a), (5b), (5c), (5d), and/or (5e), X¹⁷¹ can be selected from K and R; X¹⁷² can be selected from S, T, and Y; X¹⁷³ can be selected from D, E, F, I, and V; X¹⁷⁴ can be V; X¹⁷⁵ can be selected from E, L, M, and V; X¹⁷⁶ can be Q; X¹⁷⁷ can be selected from D and E; X¹⁷⁸ can be W; X¹⁷⁹ can be selected from D, E, N, and Q; X¹⁸⁰ can be G; X¹⁸¹ can be V; X¹⁸² can be E; X¹⁸³ can be L; X¹⁸⁴ can be W; X¹⁸⁵ can be selected from I, L, Q, and V; X¹⁸⁶ can be selected from D and E; X¹⁸⁷ can be selected from A, D, E, F, G, I, M, N, P, Q, R, S, T, V, W, and Y; and X¹⁸⁸ can be selected from D, E, N, and Q.

In an IL-2Rγc ligand of Formula (5), X¹⁷¹ can be selected from H, K, and R.

In an IL-2Rγc ligand of Formula (5)-(5a), X¹⁷² can be selected from S, T, and Y.

In an IL-2Rγc ligand of Formula (5)-(5b), X¹⁷³ can be selected from D, E, F, I, L, M, V, W, and Y.

In an IL-2Rγc ligand of Formula (5)-(5b), X¹⁷³ can be selected from D and E.

In an IL-2Rγc ligand of Formula (5)-(5b), X¹⁷³ can be selected from F, I, L, M, V, W, and Y.

In an IL-2Rγc ligand of Formula (5)-(5c), X¹⁷⁴ can be selected from F, I, L, M, V, W, and Y.

In an IL-2Rγc ligand of Formula (5)-(5c), X¹⁷⁴ can be V.

In an IL-2Rγc ligand of Formula (5)-(5d), X¹⁷⁵ can be selected from D, E, F, I, L, M, V, W, and Y.

In an IL-2Rγc ligand of Formula (5)-(5d), X¹⁷⁵ can be selected from D and E.

In an IL-2Rγc ligand of Formula (5)-(5d), X¹⁷⁵ can be selected from F, I, L, M, V, W, and Y.

In an IL-2Rγc ligand of Formula (5)-(5e), X¹⁷⁶ can be selected from D, E, H, N, Q, S, T, and Y.

In an IL-2Rγc ligand of Formula (5)-(5e), X¹⁷⁶ can be selected from E and Q.

In an IL-2Rγc ligand of Formula (5)-(5e), X¹⁷⁷ can be selected from D and E.

In an IL-2Rγc ligand of Formula (5)-(5e), X¹⁷⁸ can be selected from F, H, I, L, M, V, W, and Y.

In an IL-2Rγc ligand of Formula (5)-(5e), X¹⁷⁸ can be selected from F, H, W, and Y.

In an IL-2Rγc ligand of Formula (5)-(5e), X¹⁷⁸ can be W.

In an IL-2Rγc ligand of Formula (5)-(5e), X¹⁷⁹ can be selected from D, E, H, N, Q, S, T, and Y.

In an IL-2Rγc ligand of Formula (5)-(5e), X¹⁷⁹ can be selected from D, E, and Q.

In an IL-2Rγc ligand of Formula (5)-(5e), X¹⁸⁰ can be G.

In an IL-2Rγc ligand of Formula (5)-(5e), X¹⁸¹ can be V.

In an IL-2Rγc ligand of Formula (5)-(5e), X¹⁸² can be E.

In an IL-2Rγc ligand of Formula (5)-(5e), X¹⁸³ can be L.

In an IL-2Rγc ligand of Formula (5)-(5d), X¹⁸⁴ can be W.

In an IL-2Rγc ligand of Formula (5)-(5c), X¹⁸⁵ can be selected from F, I, L, M, V, W, and Y.

In an IL-2Rγc ligand of Formula (5)-(5c), X¹⁸⁵ can be L.

In an IL-2Rγc ligand of Formula (5)-(5b), X¹⁸⁶ can be E.

In an IL-2Rγc ligand of Formula (5)-(5a), X¹⁸⁷ can be selected from an amino acid.

In an IL-2Rγc ligand of Formula (5), X¹⁸⁸ can be selected from D and E.

In an IL-2Rγc ligand of Formula (5), (5a), (5b), (5c), (5d), and/or (5e), X¹⁷¹ can be selected from H, K, and R; X¹⁷² can be selected from S, T, and Y; X¹⁷³ can be selected from D, E, F, I, L, M, V, W, and Y; X¹⁷⁴ can be selected from F, I, L, M, V, W, and Y; X¹⁷⁵ can be selected from D, E, F, I, L, M, V, W, and Y; X¹⁷⁶ can be selected from D, E, H, N, Q, S, T, and Y; X¹⁷⁷ can be selected from D and E; X¹⁷⁸ can be selected from F, H, I, L, M, V, W, and Y; X¹⁷⁹ can be selected from D, E, H, N, Q, S, T, and Y; X¹⁸⁰ can be G; X¹⁸¹ can be V; X¹⁸² can be E; X¹⁸³ can be L; X¹⁸⁴ can be selected from W; X¹⁸⁵ can be selected from F, I, L, M, V, W, and Y; X¹⁸⁶ can be E; X¹⁸⁷ can be selected from an amino acid; and X¹⁸⁸ can be selected from D and E.

In an IL-2Rγc ligand of Formula (5), (5a), (5b), (5c), (5d), and/or (5e), X¹⁷¹ can be selected from H, K, and R; X¹⁷² can be selected from S, T, and Y; X¹⁷³ can be selected from D and E; X¹⁷⁴ can be V; X¹⁷⁵ can be selected from D and E; X¹⁷⁶ can be selected from E and Q; X¹⁷⁷ can be selected from D and E; X¹⁷⁸ can be selected from F, H, W, and Y; X¹⁷⁹ can be selected from D, E, and Q; X¹⁸⁰ can be G; X¹⁸¹ can be V; X¹⁸² can be E; X¹⁸³ can be L; X¹⁸⁴ can be W; X¹⁸⁵ can be selected from F, I L, M, V, W, Y, H, N, Q, S, and T; X¹⁸⁶ can be E; X¹⁸⁷ can be selected from an amino acid; and X¹⁸⁸ can be selected from D and E.

In an IL-2Rγc ligand of Formula (5), (5a), (5b), (5c), (5d), and/or (5e), X¹⁷¹ can be selected from H, K, and R; X¹⁷² can be selected from S, T, and Y; X¹⁷³ can be selected from F, I, L, M, V, W, and Y; X¹⁷⁴ can be V; X¹⁷⁵ can be selected from F, I, L, M, V, W, and Y; X¹⁷⁶ can be selected from E and Q; X¹⁷⁷ can be selected from D and E; X¹⁷⁸ can be selected from F, H, W, and Y; X¹⁷⁹ can be selected from D, E, and Q; X¹⁸⁰ can be G; X¹⁸¹ can be V; X¹⁸² can be E; X¹⁸³ can be L; X¹⁸⁴ can be W; X¹⁸⁵ can be selected from F, I, L, M, V, W, Y, H, N, Q, S, and T; X¹⁸⁶ can be E; X¹⁸⁷ can be selected from an amino acid; and X¹⁸⁸ can be selected from D and E.

In an IL-2Rγc ligand of Formula (5), (5a), (5b), (5c), (5d), and/or (5e), X¹⁷¹ can be selected from H, K, and R; X¹⁷² can be selected from S, T, and Y; X¹⁷³ can be selected from D, E, F, I, L, M, V, W, and Y; X¹⁷⁴ can be V; X¹⁷⁵ can be selected from D, E, F, I, L, M, V, W, and Y; X¹⁷⁶ can be selected from D, E, H, N, Q, S, T, and Y; X¹⁷⁶ can be selected from E and Q; X¹⁷⁷ can be selected from D and E; X¹⁷⁸ can be W; X¹⁷⁹ can be selected from D, E, and Q; X¹⁸⁰ can be G; X¹⁸¹ can be V; X¹⁸² can be E; X¹⁸³ can be L; X¹⁸⁴ can be W; X¹⁸⁵ can be selected from F, I L, M, V, W, Y, H, N, Q, S, and T; X¹⁸⁶ can be E; X¹⁸⁷ can be selected from an amino acid; and X¹⁸⁸ can be selected from D and E.

An IL-2Rγc ligand can comprise an amino acid sequence selected from any one of SEQ ID NO: 637-715.

| | |
|---|---|
| SEQ ID NO: 637 | I E C D T S Y G V Y I C W Q |
| SEQ ID NO: 638 | I E C E E W R G V E L C W Q |
| SEQ ID NO: 639 | P E G R E V V V C R D W Y G V E L C W Q |
| SEQ ID NO: 640 | I W G R T V V E C Q D W E G V E L C W Q |
| SEQ ID NO: 641 | L A L R K E V V C Q E Y Y G V E L C W I |
| SEQ ID NO: 642 | H H A R E V V V C Q D W Y G V E L C W Q |
| SEQ ID NO: 643 | M V N R E V V V C E D W Y G V E L C W Q |
| SEQ ID NO: 644 | T A N Q T V V E C Q V W G G V E L C W Q |
| SEQ ID NO: 645 | V E C Q E W G G V E L C W C |
| SEQ ID NO: 646 | D V E C V D W G G V E L C W H |
| SEQ ID NO: 647 | I V C E E W R G V E L C W L |
| SEQ ID NO: 648 | D F E R S Y V V C Q D W D G V E L C W I |
| SEQ ID NO: 649 | A H S R Q E V V C E E W Y G V E L C W I |
| SEQ ID NO: 650 | S A P E R W V E C E D W Q G V E L C W V |
| SEQ ID NO: 651 | Y S R E L Y V Q C E D W E G V E L C W I |
| SEQ ID NO: 652 | V V C Q D W E G V E L C W Q |
| SEQ ID NO: 653 | D V V C Q N W E G V D L C W H |
| SEQ ID NO: 654 | S A G R Q E V V C Q D W N G V E L C W I |
| SEQ ID NO: 655 | G Q G R E V V V C H D W Y G V E L C W Q |
| SEQ ID NO: 656 | D W R R S V V E C Q D W Y G V E L C W Q |
| SEQ ID NO: 657 | D V V C Q N W D G V D L C W H |
| SEQ ID NO: 658 | T L G R T V V E C Q D W G G V E L C W Q |
| SEQ ID NO: 659 | R L L N S V V E C L D W E G V E L C W Q |
| SEQ ID NO: 660 | I V C E D W R G V E L C W I |
| SEQ ID NO: 661 | V V C Q E W E G V E L C W C |
| SEQ ID NO: 662 | G D R P K E V V C E D W K G V E L C W I |
| SEQ ID NO: 663 | E R P R S F I E C Q E W E G V E L C W L |
| SEQ ID NO: 664 | E G S T T T I E C E E W A G V E L C W L |
| SEQ ID NO: 665 | A N Q N T V V E C Q D W H G V E L C W Q |
| SEQ ID NO: 666 | R S D D E V V V C Q E W E G V E L C W Q |
| SEQ ID NO: 667 | I E C E E W A G V E L C W L |
| SEQ ID NO: 668 | T W N M S E L E C Q D W N G V E I C W H |
| SEQ ID NO: 669 | G N D D S Y I V C E E W K G V E L C W I |
| SEQ ID NO: 670 | F A H H G V V E C Q E W Y G V E L C W Q |
| SEQ ID NO: 671 | L N R S V W I E C E E Y E G V E L C W L |
| SEQ ID NO: 672 | W S K K A E V V C E E W G G V E F C W I |
| SEQ ID NO: 673 | R S N Q T V V E C Q D W E G V E L C W Q |
| SEQ ID NO: 674 | V V C Q E W E G V E L C W Y A G E C M Q |
| SEQ ID NO: 675 | I L C Q E F E G V E L C W L E E S L A E |
| SEQ ID NO: 676 | K S Q V E C Q D W E G V E L C W V V S E |
| SEQ ID NO: 677 | K I T V E C Q D W D G V E L C W P T W I |
| SEQ ID NO: 678 | R P Q I E C Q E W Q G V E L C W T R E E |
| SEQ ID NO: 679 | V S C Q E W D G V E L C W V D G D L A A |
| SEQ ID NO: 680 | I M C Q E W D G V E L C W L E R D K A N |
| SEQ ID NO: 681 | G L E I A C E D W Y G V E L C W L R R A |
| SEQ ID NO: 682 | G Y G V L C Q E W Q G V E L C W P V Q R E A G V |
| SEQ ID NO: 683 | P Y G V V C Q D W A G V E L C W V E N R |
| SEQ ID NO: 684 | K L T V E C Q D W D G V E L C W V G V E |
| SEQ ID NO: 685 | I N C Q T W N G V E L C W V D E G L Y Q |
| SEQ ID NO: 686 | V V C Q E W E G V E L C W V E P P L L P |
| SEQ ID NO: 687 | R V Q V E C E D W N G V E L C W P V R V |
| SEQ ID NO: 688 | D R Q V V C E E W D G V E L C W I E E S |
| SEQ ID NO: 689 | K T T V A C Q D W G G V E L C W V E R V |
| SEQ ID NO: 690 | R P E V V C Q E W E G V E L C W I S P L |
| SEQ ID NO: 691 | R L G V E C Q E W E G V D L C W I S A F |
| SEQ ID NO: 692 | K P V V V C E E W Q G V E L C W L E I Q |
| SEQ ID NO: 693 | V V C E V F Q G V E L C W C E N E E F T |
| SEQ ID NO: 694 | T D E V S C Q E W E G V E L C W I E R Q |
| SEQ ID NO: 695 | P V E V R C Q E W E G V E L C W V V G I |
| SEQ ID NO: 696 | G P E V V C E E F N R V E L C W V E Y N |
| SEQ ID NO: 697 | K Y I V E C Q E W G G V E L C W P E M V |
| SEQ ID NO: 698 | V T C Q E Y E G V E L C W T V G C A Y S |
| SEQ ID NO: 698 | V V C Q E W E G V E L C W Q T G P G A H A |
| SEQ ID NO: 700 | I V C E E Y N G V E L C W V E T S V K P |
| SEQ ID NO: 701 | E Q Q V V C Q E W N G V E L C W I E A G |
| SEQ ID NO: 702 | Q L G V E C Q N W R G V E L C W V S E I |
| SEQ ID NO: 703 | T A E V V C Q E W D G V E L C W I E V L |
| SEQ ID NO: 704 | S P S I V C E E W A G V E L C W V D Y S |
| SEQ ID NO: 705 | A V C Q D W Y G V E L C W C M Q D I L D |
| SEQ ID NO: 706 | V E C E E W G G V E L C W L A D E V M W |
| SEQ ID NO: 707 | H S T V I C Q D W D G V E L C W I E N D |
| SEQ ID NO: 708 | K K I V V C Q D W G G V E L C W T E D D |
| SEQ ID NO: 709 | S V E V V C E E W H G V E L C W P V F I |
| SEQ ID NO: 710 | R W A V S C Q D W Q G I E L C W P E W D |
| SEQ ID NO: 711 | R T G V E C Q D W H G V E L C W P V W E |
| SEQ ID NO: 712 | G Y G V V C E D F R G V E L C W L E R K |
| SEQ ID NO: 713 | R T E V E C E D W E G V E L C W L |
| SEQ ID NO: 714 | I L C E E W Q G V E L C W L E G G G S |
| SEQ ID NO: 715 | V G I E C E E W A G V E L C W L |

An IL-2Rγc ligand can comprise an amino acid sequence selected from any one of SEQ ID NO: 631-715, wherein the amino acid sequence can be terminated with flanking amino acids such as -G-G- (SEQ ID NO: 20) on the N-terminus, on the C-terminus, or on both the N- and C-termini.

An IL-2Rγc ligand can comprise an amino acid sequence selected from any one of SEQ ID NO: 631-715, wherein the amino acid sequence can be terminated with one or more amino acids on the N-terminus, on the C-terminus, or on both the N- and C-termini. For example, an amino acid sequence can include terminal glycines and/or serines. For example, an IL-2Rγc ligand can comprise a -G-G- (SEQ ID NO: 20) moiety on the N-terminus, on the C-terminus, or on both the N- and C-termini.

An IL-2Rγc ligand can comprise an amino acid sequence selected from any one of SEQ ID NO: 631-715, wherein each amino acid independently comprises one or more of the following conservative substitutions: amino acids having a small hydrophobic side chain comprising alanine (A), glycine (G), proline (P), serine (S), or threonine (T); amino acids having a hydroxyl-containing side chain comprising serine (S), threonine (T), or tyrosine (Y); amino acids having an acidic side chain comprising aspartate (D) or glutamate (E); amino acids having a polar-neutral side chain comprising histidine (H), asparagine (N), glutamine (Q), serine (S), threonine (T), or tyrosine (Y); amino acids having a basic side chain comprising arginine (R), lysine (K), or histidine (H); amino acids having a large hydrophobic side chain comprising isoleucine (I), leucine (L), methionine (M), valine (V), phenylalanine (F), tyrosine (Y), or tryptophan (W); and amino acids having an aromatic side chain comprising phenylalanine (F), histidine (H), tryptophan (W), or tyrosine (Y).

Certain of the IL-2Rγc ligands having SEQ ID NO: 631-949 bind to the hu-IL-2Rγc subunit with an IC₅₀ of less than 100 μM.

IL-2Rγc ligands having SEQ ID NO: 631-715 bind to the hu-IL-2Rγc subunit with an IC₅₀ of less than 100 μM.

An IL-2Rγc ligand can comprise an amino acid sequence having an amino acid similarity greater than 60%, greater than 70%, greater than 75%, greater than 80%, greater than 85%, greater than 90%, or greater than 95% to the amino acid sequence of any one of SEQ ID NO: 631-715.

An IL-2Rγc ligand provided by the present disclosure can comprise a truncated amino acid sequence of any one of SEQ ID NO: 631-715.

An IL-2Rγc ligand provided by the present disclosure can comprise an amino acid sequence selected from any one of SEQ ID NO: 631-715, or a truncated amino acid sequence of any one of SEQ ID NO: 631-715, wherein the amino acid sequence can independently comprise from 1 to 4 glycines (G) (SEQ ID NO: 30) on the N-terminus, on the C-terminus, or on both the N- and C-termini.

An IL-2Rγc ligand provided by the present disclosure can comprise an amino acid sequence selected from any one of SEQ ID NO: 631-715, or a truncated amino acid sequence of any one of SEQ ID NO: 631-715, wherein the amino acid sequence comprises one or more amino acid substitutions such as from 1 to 5 amino acid substitutions. The amino acid substitutions can comprise conservative amino acid substitutions or non-conservative amino acid substitutions.

An IL-2Rγc ligand can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 631-715 or to a truncated amino acid sequence of any one of SEQ ID NO: 631-715.

An IL-2Rγc ligand of any one of SEQ ID NO: 37-715 bind to the hu-IL-2Rγc subunit with an IC₅₀ of less than 10 μM as determined using phage ELISA competition assays.

Certain of the IL-2Rγc ligands of any one of SEQ ID NO: 631-715, a truncated IL-2Rγc ligand of any one of SEQ ID NO: 631-715, or a substituted IL-2Rγc ligand of any one of SEQ ID NO: 631-715 can bind to the hu-IL-2Rγc subunit with an IC₅₀ of less than 100 μM or less than 10 μM as determined using phage ELISA competition assays.

An IL-2Rγc ligand can comprise an amino acid sequence of any one of SEQ ID NO: 652, 668, 711, 713, 715, 716, and 718.

| | |
|---|---|
| SEQ ID NO: 716 | I E C E E W A G V E L C W L |
| SEQ ID NO: 652 | V V C Q D W E G V E L C W Q |
| SEQ ID NO: 718 | W S K K A E V V C E E W G G V E F C W I |
| SEQ ID NO: 711 | R T G V E C Q D W H G V E L C W P V W E |
| SEQ ID NO: 715 | V G I E C E E W A G V E L C W L |
| SEQ ID NO: 668 | T W N M S EL E C Q D W N G V E I C W H |
| SEQ ID NO: 713 | R T E V E C E D W E G V E L C W L |

An IL-2Rγc ligand can comprise an amino acid sequence having an amino acid sequence similarity greater than 60%, greater than 70%, greater than 75%, greater than 80%, greater than 85%, greater than 90%, or greater than 95%, to the amino acid sequence of any one of SEQ ID NO: 652, 668, 711, 713, 715, 716, and 718.

An IL-2Rγc ligand provided by the present disclosure can comprise a truncated amino acid sequence of any one of SEQ ID NO: 652, 668, 711, 713, 715, 716, and 718.

An IL-2Rγc ligand provided by the present disclosure can comprise an amino acid sequence selected from any one of SEQ ID NO: 652, 668, 711, 713, 715, 716, and 718, or a truncated amino acid sequence of any one of SEQ ID NO: 652, 668, 711, 713, 715, 716, and 718, wherein the amino acid sequence can independently comprise from 1 to 4 glycines (G) (SEQ ID NO: 30) on the N-terminus, on the C-terminus, or on both the N- and C-termini.

An IL-2Rγc ligand provided by the present disclosure can comprise an amino acid sequence selected from any one of SEQ ID NO: 652, 668, 711, 713, 715, 716, and 718, or a truncated amino acid sequence of any one of SEQ ID NO: 652, 668, 711, 713, 715, 716, and 718, wherein the amino acid sequence comprises one or more amino acid substitutions such as from 1 to 5 amino acid substitutions. The amino acid substitutions can comprise conservative amino acid substitutions or non-conservative amino acid substitutions.

An IL-2Rγc ligand can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 652, 668, 711, 713, 715, 716, and 718 or to a truncated amino acid sequence of any one of SEQ ID NO: 652, 668, 711, 713, 715, 716, and 718.

An IL-2Rγc ligand of any one of SEQ ID NO: 652, 668, 711, 713, 715, 716, and 718 binds to the hu-IL-2Rγc subunit with an IC₅₀ of less than 10 μM as determined using phage ELISA competition assays.

An IL-2Rγc ligand of any one of SEQ ID NO: 652, 668, 711, 713, 715, 716, and 718, a truncated IL-2Rγc ligand of any one of SEQ ID NO: 652, 668, 711, 713, 715, 716, and 718, or a substituted IL-2Rγc ligand of any one of SEQ ID NO: 652, 668, 711, 713, 715, 716, and 718 can bind to the hu-IL-2Rγc subunit with an IC₅₀ of less than 100 μM or less than 10 μM as determined using phage ELISA competition assays.

An IL-2Rγc ligand can have any one of SEQ ID NO: 723-749.

| | |
|---|---|
| SEQ ID NO: 723 | S L L K C Y N A S T C A S V F |
| SEQ ID NO: 724 | C G M A I G D L C M W T |
| SEQ ID NO: 725 | R W G D V G D L L M P L |
| SEQ ID NO: 726 | R S C Y Y K R P R L W C S E |
| SEQ ID NO: 727 | D C S M W E G V E L C W G G R R |
| SEQ ID NO: 728 | G G V V C Q D W E G V E L C W Q G G R R |
| SEQ ID NO: 729 | G G V M C E R W Q G V E L C W L G G |
| SEQ ID NO: 730 | G G R T G V E C Q D W H G V E L C W P V W E G G |
| SEQ ID NO: 731 | G G V G I E C E E W A G V E L C W L G G |
| SEQ ID NO: 732 | G G T W N M S E L E C Q D W N G V E I C W H G G |
| SEQ ID NO: 733 | G G R T E V E C E D W E G V E L C W L G G |
| SEQ ID NO: 734 | G G W S K K A E V V C E E W G G V E F C W I G G |
| SEQ ID NO: 735 | W S K K A E V V C E E W G G V E F C W I |
| SEQ ID NO: 736 | Y S R E L Y V Q C E D W E G V E L C W I |
| SEQ ID NO: 737 | G G D C S M W E G V E L C W G G |
| SEQ ID NO: 738 | G G V M C E R W Q G V E L C W L G G |
| SEQ ID NO: 739 | G G V G I E C E E W A G V E L C W L G G |
| SEQ ID NO: 740 | G G T W N M S E L E C Q D W N G V E I C W H G G |
| SEQ ID NO: 741 | G G R T E V E C E D W E G V E L C W L G G |
| SEQ ID NO: 742 | G G R T G V E C Q D W H G V E L C W P V W E G G |
| SEQ ID NO: 743 | G G V V C Q D W E G V Abu L C W Q G G |
| SEQ ID NO: 744 | G G V V C Q D W E G V Alb L C W Q G G |
| SEQ ID NO: 745 | G G V V C Q D W E G V DA L C W Q G G |
| SEQ ID NO: 746 | G G V V C Q D W E G V S L C W Q G G |
| SEQ ID NO: 747 | G G V V C Q D W E G V G L C W Q G G |
| SEQ ID NO: 748 | G G V V C Q D W E G V E L C W Q P P A |
| SEQ ID NO: 749 | G G V V C Q D W E G V E L C W Q G P P A |

An IL-2Rγc ligand provided by the present disclosure can comprise a truncated amino acid sequence of any one of SEQ ID NO: 723-749.

An IL-2Rγc ligand provided by the present disclosure can comprise an amino acid sequence selected from any one of SEQ ID NO: 723-749, or a truncated amino acid sequence of any one of SEQ ID NO: 723-749, wherein the amino acid sequence can independently comprise from 1 to 4 glycines (G) (SEQ ID NO: 30) on the N-terminus, on the C-terminus, or on both the N- and C-termini.

An IL-2Rγc ligand provided by the present disclosure can comprise an amino acid sequence selected from any one of SEQ ID NO: 723-749, or a truncated amino acid sequence of any one of SEQ ID NO: 723-749, wherein the amino acid sequence comprises one or more amino acid substitutions such as from 1 to 5 amino acid substitutions. The amino acid substitutions can comprise conservative amino acid substitutions.

An IL-2Rγc ligand can comprise an amino acid sequence having an amino acid sequence similarity greater than 60%, greater than 70%, greater than 75%, greater than 80%, greater than 85%, greater than 90%, or greater than 95% to the amino acid sequence of any one of SEQ ID NO: 723-7493 or to a truncated amino acid sequence of any one of SEQ ID NO: 723-749.

An IL-2Rγc ligand of any one of SEQ ID NO: 723-749, a truncated Rγc ligand of any one of SEQ ID NO: 723-749, or a substituted Rγc ligand of any one of SEQ ID NO: 723-749 bind to the hu-Rγc subunit with an IC₅₀ of less than 100 μM or less than 10 μM as determined using phage ELISA competition assays.

An IL-2Rγc ligand of any one of SEQ ID NO: 737-749 binds to the hu-IL-2Rγc subunit with an IC₅₀ of less than 100 μM.

Certain IL-2Rγc ligands provided by the present disclosure can bind to a specific binding site on the hu-IL-2Rγc subunit that is different than the IL-2Rγc binding site on the hu-IL-2Rγc subunit to which IL-2 binds.

These IL-2Rγc ligands do not compete for binding to the specific hu-IL-2Rγc binding site with IL-2, have no detectable binding to the hu-IL-2Rβ subunit, and bind to the hu-IL-2Rγc subunit with an IC₅₀ of less than 10 μM.

The specific binding site on the hu-IL-2Rγc subunit can be characterized by at least the following properties: (1) a group of IL-2Rγc ligands bind to the specific binding site on the hu-IL-2Rγc subunit with an IC₅₀ of less than 10 μM; (2) IL-2Rγc ligands within the group competitively bind to the specific binding site on the hu-IL-2Rγc subunit with other IL-2Rγc ligands within the group; and (3) IL-2Rγc ligands within the group do not compete for binding to the specific binding site with an IL-2Rγc ligand having the amino acid sequence of SEQ ID NO: 723.

An IL-2Rβ ligand having the amino acid sequence of SEQ ID NO: 932 does not compete for binding to the binding site with the group of IL-2Rγc ligands.

The group of IL-2Rγc ligands comprises IL-2Rγc ligands having an amino acid sequence of SEQ ID NO: 935-940.

| | |
|---|---|
| SEQ ID NO: 932 | Y D C R I A Q V G E L C D L |
| SEQ ID NO: 935 | D C S M W E G V E L C W |
| SEQ ID NO: 936 | K V C E M W G G V L L C W N |
| SEQ ID NO: 937 | R T C T E W E N V V L C W V |
| SEQ ID NO: 938 | M C W L E W G E W V G S C L |
| SEQ ID NO: 939 | D L S D L S T F W L S Q |
| SEQ ID NO: 940 | C P S M L Q G P E R T W V C |

IL-2Rγc ligands within the group of IL-2Rγc ligands bind to the hu-IL-2Rγc subunit with an IC₅₀ of less than 100 μM and bind to the IL-2Rβ subunit with an IC₅₀ of greater than 100 μM.

The specific binding site of the hu-IL-2Rβ subunit for these IL-2Rγc ligands can be characterized using competitive binding assays.

An IL-2Rγc ligand provided by the present disclosure can comprise a pH-biased IL-2Rγc ligand.

pH-Biased IL-2Rγc ligands bind to the hu-IL-2Rγc subunit at pH 6 with an IC₅₀ that is less than the IC₅₀ for binding of the same IL-2Rγc ligand to the IL-2Rγc subunit at pH 7.5.

A pH-biased IL-2Rγc ligand can bind to the hu-IL-2Rγc subunit at pH 6 with an IC₅₀ that is at least at least 10% less than the IC₅₀ for binding to the hu-IL-2Rγc subunit at pH 7.5, at least 25% less, at least 50% less, at least 100% less, or at least 200% less than the IC₅₀ for binding to the hu-IL-2Rγc subunit at pH 7.5.

A pH-biased IL-2Rγc ligand can comprise, for example, from 5 to 30 amino acids.

A pH-biased IL-2Rγc ligand can bind to the hu-IL-2Rγc subunit at pH 6.0 with an IC₅₀ from 1 pM to 100 μM and can bind to the hu-IL-2Rγc subunit with an IC₅₀ greater than 100 μM at pH 7.5.

A pH-biased IL-2Rγc ligand can bind to the hu-IL-2Rγc subunit at pH 6.0 with an IC₅₀ from 0.1 μM to 50 μM and bind to the hu-IL-2Rγc subunit with an IC₅₀ greater than 100 μM at pH 7.5.

A pH-biased IL-2Rγc ligand can bind to the hu-IL-2Rγc subunit at pH 6.0 with an IC₅₀ of less than 100 μM.

A pH-biased IL-2Rγc ligand can bind to each of the hu-IL-2Rγc subunit and to the hu-IL-2Rγc subunit with an IC₅₀ of less than 100 μM and a bind to the hu-IL-2Rγc subunit with an IC₅₀ greater than 100 μM at pH 7.5.

A pH-biased IL-2Rγc ligand can bind to a mammalian IL-2Rγc subunit at pH 6.0 with an IC₅₀ of less than 100 μM and bind to the hu-IL-2Rγc subunit with an IC₅₀ greater than 100 μM at pH 7.5.

An IL-2Rβ ligand, an IL-2Rγc ligand, and an IL-2Rβγc agonist peptide may not comprise any flanking amino acids bound to the N-terminus and/or to the C-terminus of the ligand.

IL-2Rβ ligands and IL-2Rγc ligands can comprise one or more flanking amino acids bound to the N-terminus and/or to the C-terminus of the ligand.

The flanking amino acids can separate the portion of the ligand that interacts with IL-2R from other portions of a ligand, an IL-2Rβγc agonist peptide, and/or a ligand construct.

A ligand can comprise flanking amino acids such as, for example, from 1 to 20 amino acids, from 1 to 10 amino acids, such as from 1 to 8 amino acids, from 2 to 6 amino acids, or from 2 to 4 amino acids bound to the N-terminus and/or the C-terminus of the ligand.

Flanking amino acids can comprise any suitable naturally occurring or non-naturally occurring amino acids.

Flanking amino acids can be selected from serine and flexible amino acids such as serine.

A ligand can comprise flanking amino acids such as, for example, terminal glycine groups on the N-terminus and/or the C-terminus of the respective ligand. For example, a ligand can comprise flanking amino acids having an amino acid sequence of any one of SEQ ID NO: 1-42. For example, an IL-2Rβ ligand, an IL-2Rγc ligand, or an IL-2Rβγc agonist peptide can independently comprise flanking amino acids such as 1, 2, or 3 terminal glycines.

An IL-2Rβ ligand, an IL-2Rγc ligand, and an IL-2Rβγc agonist peptide can comprise, for example, from 1 to 20 amino acids, from 1 to 10 amino acids, such as from 1 to 8 amino acids, from 2 to 6 amino acids, or from 2 to 4 amino acids bound to the N-terminus and/or the C-terminus of the IL-2Rβ and IL-2Rγc ligand.

Flanking amino acids can comprise any suitable naturally occurring or non-naturally occurring amino acids.

Flanking amino acids can be selected from flexible amino acids such as glycine and serine. An IL-2Rβ ligand and/or an IL-2Rγc ligand can comprise, for example, terminal glycine groups on the N-terminus and/or the C-terminus of the respective ligand. For example, an IL-2Rβ and an IL-2Rγc ligand can comprise (G)ₙ glycine groups (SEQ ID. NO: 1) where n is from 1 to 20. For example, each of an IL-2Rβ ligand and an IL-2Rγc ligand can independently comprise 1, 2, or 3 terminal glycine groups. For example, a ligand having SEQ ID NO: 750, having the amino acid sequence -W-H-P-C-W-I-A-Q-L-G-E-L-C-D-L-E- can independently include, for example, one, two, or three glycines on both the N-terminus and the C-terminus such that the ligand can have the amino acid sequence -G-W-H-P-C-W-I-A-Q-L-G-E-L-C-D-L-E-G- (SEQ ID NO:751), -G-G-W-H-P-C-W-I-A-Q-L-G-E-L-C-D-L-E-G-G- (SEQ ID NO:752), or -G-G-G-W-H-P-C-W-I-A-Q-L-G-E-L-C-D-L-E-G-G-G- (SEQ ID NO: 753), respectively.

An IL-2Rβ ligand, an IL-2Rγc ligand, and an IL-2Rβγc agonist peptide provided by the present disclosure can comprise, for example, an amino acid substitution such as from 1 to 10 amino acid substitutions, from 1 to 8, from 1 to 6, from 1 to 4, such as 1, 2, 3, 4, 5, 6, 7, 8, 9, or 10 amino acid substitutions. An amino acid substitution can be a conservative amino acid substitution or a non-conservative amino acid substitution.

A ligand can comprise a truncated amino acid sequence.

A truncated amino acid sequence refers to an amino acid sequence that does not include one or more of the terminal amino acids of a reference amino acid sequence. For example, in a truncated peptide, one or more amino acids are removed from the N-terminus, the C-terminus, or both the N-terminus and the C-terminus of a reference amino acid sequence. Removing one or more amino acids from the N-terminus and/or the C-terminus of an amino acid sequence provided by the present disclosure can result in improved properties such as a higher binding affinity, a lower binding affinity, or greater binding selectivity. Thus, ligands such as IL-2Rβ ligands and IL-2Rγc ligands provided by the present disclosure include truncated IL-2Rβ ligands and truncated Rγc ligands.

Examples of truncated IL-2Rβ ligands based on SEQ ID NO: 591 include ligands having an amino acid sequence of any one of SEQ ID NO: 591, 755-766, 134, 768, and 769.

| | |
|---|---|
| SEQ ID NO: 591 | G G W Y P C W M A Q L G E L C D L D G G |
| SEQ ID NO: 755 | G W Y P C W M A Q L G E L C D L D G G |
| SEQ ID NO: 756 | W Y P C W M A Q L G E L C D L D G G |
| SEQ ID NO: 757 | Y P C W M A Q L G E L C D L D G G |
| SEQ ID NO: 758 | P C W M A Q L G E L C D L D G G |
| SEQ ID NO: 759 | C W M A Q L G E L C D L D G G |
| SEQ ID NO: 760 | G G W Y P C W M A Q L G E L C D L D G |
| SEQ ID NO: 761 | G G W Y P C W M A Q L G E L C D L D |
| SEQ ID NO: 762 | G G W Y P C W M A Q L G E L C D L |
| SEQ ID NO: 763 | G G W Y P C W M A Q L G E L C D |
| SEQ ID NO: 764 | G G W Y P C W M A Q L G E L C |
| SEQ ID NO: 765 | G W Y P C W M A Q L G E L C D L D G |
| SEQ ID NO: 766 | W Y P C W M A Q L G E L C D L D G |
| SEQ ID NO: 134 | W Y P C W M A Q L G E L C D L D |
| SEQ ID NO: 768 | Y P C W M A Q L G E L C D L |
| SEQ ID NO: 769 | P C W M A Q L G E |

Examples of truncated IL-2Rβ ligands based on SEQ ID NO: 770 include ligands having an amino acid sequence of any one of SEQ ID NO: 770-785.

| | |
|---|---|
| SEQ ID NO: 770 | G G E L L V D C F K V K V G E L C D L F F G |
| SEQ ID NO: 771 | G G E L L V D C F K V K V G E L C D L F F |
| SEQ ID NO: 772 | G G E L L V D C F K V K V G E L C D L F |
| SEQ ID NO: 773 | G G E L L V D C F K V K V G E L C D L |
| SEQ ID NO: 774 | G G E L L V D C F K V K V G E L C D |
| SEQ ID NO: 775 | G G E L L V D C F K V K V G E L C |
| SEQ ID NO: 776 | G E L L V D C F K V K V G E L C D L F F G |
| SEQ ID NO: 777 | E L L V D C F K V K V G E L C D L F F G |
| SEQ ID NO: 778 | L L V D C F K V K V G E L C D L F F G |
| SEQ ID NO: 779 | L V D C F K V K V G E L C D L F F G |
| SEQ ID NO: 780 | V D C F K V K V G E L C D L F F G |
| SEQ ID NO: 781 | D C F K V K V G E L C D L F F G |
| SEQ ID NO: 782 | E L L V D C F K V K V G E L C D L F |
| SEQ ID NO: 783 | L V D C F K V K V G E L C D L |
| SEQ ID NO: 784 | V D C F K V K V G E L C D |
| SEQ ID NO: 785 | C F K V K V G E L C |

Examples of truncated IL-2Rγc ligands based on SEQ ID NO: 786 include ligands having an amino acid sequence of any one of SEQ ID NO: 134, 787-795, and 797-800.

| | |
|---|---|
| SEQ ID NO: 786 | G G V V C Q D W E G V E L C W Q G G |
| SEQ ID NO: 787 | G V V C Q D W E G V E L C W Q G G |
| SEQ ID NO: 788 | V V C Q D W E G V E L C W Q G G |
| SEQ ID NO: 789 | V C Q D W E G V E L C W Q G G |
| SEQ ID NO: 790 | C Q D W E G V E L C W Q G G |
| SEQ ID NO: 791 | G G V V C Q D W E G V E L C W Q G |
| SEQ ID NO: 792 | G G V V C Q D W E G V E L C W Q |
| SEQ ID NO: 793 | G G V V C Q D W E G V E L C W |
| SEQ ID NO: 794 | G G V V C Q D W E G V E L C |
| SEQ ID NO: 795 | G V V C Q D W E G V E L C W Q G |
| SEQ ID NO: 134 | V V C Q D W E G V E L C W Q |
| SEQ ID NO: 797 | V C Q D W E G V E L C W Q |
| SEQ ID NO: 798 | V C Q D W E G V E L C W |
| SEQ ID NO: 799 | C Q D W E G V E L C W |
| SEQ ID NO: 800 | C Q D W E G V E L C |

An IL-2Rβ ligand provided by the present disclosure can have greater than 60%, greater than 70%, greater than 75%, greater than 80%, greater than 85%, greater than 90%, or greater than 95% sequence similarity to any one of SEQ ID NO: 74-172 and 330-560. An IL-2Rβ ligand provided by the present disclosure can have greater than 60%, greater than 70%, greater than 75%, greater than 80%, greater than 85%, greater than 90%, or greater than 95% sequence similarity to SEQ ID NO: 134. An IL-2Rβ ligand provided by the present disclosure can have greater than 60%, greater than 70%, greater than 75%, greater than 80%, greater than 85%, greater than 90%, or greater than 95% sequence similarity to SEQ ID NO: 527.

An IL-2Rγc ligand provided by the present disclosure can have greater than 60%, greater than 70%, greater than 75%, greater than 80%, greater than 85%, greater than 90%, or greater than 95% sequence similarity to any one of SEQ ID NO: 631-715. An IL-2Rγc ligand provided by the present disclosure can have greater than 60%, greater than 70%, greater than 75%, greater than 80%, greater than 85%, greater than 90%, or greater than 95% sequence similarity to SEQ ID NO: 652.

An IL-2Rβ ligand, an IL-2Rγc ligand, and an IL-2Rβγc agonist peptide provided by the present disclosure can comprise an acetyl terminal group on the N-terminus and a carboxamide group on the C-terminus.

An IL-2Rβγc agonist peptide can comprise an IL-2Rβ ligand and an IL-2Rγc ligand bonded to an IL-2Rβγc linker.

Each of an IL-2Rβ ligand and an IL-2Rγc ligand can independently be covalently bonded to an IL-2Rβγc linker through the N-terminus or the C-terminus of the respective ligand. For example, an IL-2Rβ ligand can be bonded to the IL-2Rβγc linker through the N-terminus and an IL-2Rγc ligand can be bonded to an IL-2Rβγc linker through the N-terminus; an IL-2Rβ ligand can be bonded to an IL-2Rβγc linker through the N-terminus and an IL-2Rγc ligand can be bonded to the IL-2Rβγc linker through the C-terminus; an IL-2Rβ ligand can be bonded to the IL-2Rβγc linker through the C-terminus and an IL-2Rγc ligand can be bonded to the IL-2Rβγc linker through the N-terminus; or an IL-2Rβ ligand can be bonded to the IL-2Rβγc linker through the C-terminus and an IL-2Rγc ligand can be bonded to the IL-2Rβγc linker through the C-terminus.

IL-2Rβγc agonist peptides having various C/N orientations of the IL-2Rβ ligand and the IL-2Rγc ligand can be synthesized using click chemistry. The triazole linkage is an example of a component of an IL-2Rβγc linker, which can comprise various chemical moieties and can have various lengths and properties.

An IL-2Rβγc linker can be configured to facilitate the binding of an IL-2Rβγc agonist peptide to the IL-2Rβ subunit and the IL-2Rγc subunit of IL-2R. For example, an IL-2Rβγc agonist peptide linker can be configured to facilitate activation of IL-2R by the IL-2Rβγc agonist peptide. For example, an IL-2Rβγc agonist peptide can be configured to induce IL-2R-mediated STAT5 phosphorylation in TF-1β and NK-92 cells.

An IL-2Rβγc linker can have a length, for example, from 2 Å to 100 Å, from 2 Å to 80 Å, from 2 Å to 60 Å, from 2 Å to 40 Å, from 2 Å to 20 Å, from 4 Å to 18 Å, from 6 Å to 16 Å, or from 8 Å to 14 Å. A linker can have a length, for example, less than 100 Å, less than 80 Å, less than 60 Å, less than 40 Å, less than 20 Å, less than 15 Å, or less than 10 Å.

An IL-2Rβγc linker can comprise a backbone having, for example, from 2 to 50 bonds, from 2 to 45 bonds, from 2 to 40 bonds, from 2 to 35 bonds, from 2 to 30 bonds, from 2 to 25 bonds, from 2 to 20 bonds, from 4 to 18 bonds, from 6 to 16 bonds, or from 8 to 14 bonds. An IL-2Rβγc linker can comprise a backbone having, for example, less than 50 bonds, less than 40 bonds, less than 30 bonds, less than 20 bonds, or less than 10 bonds.

An IL-2Rβγc linker provided by the present disclosure can comprise a peptidyl IL-2Rβγc linker or a non-peptidyl IL-2Rβγc linker.

An IL-2Rβγc linker provided by the present disclosure can comprise a peptidyl IL-2Rβγc linker.

A peptidyl ligand linker can comprise, for example, from 2 to 100 amino acids, from 2 to 80 amino acids, from 2 to 60 amino acids, from 2 to 40 amino acids, from 2 to 20 amino acids, from 5 to 10 amino acids, or from 2 to 5 amino acids. A peptidyl ligand linker can comprise, for example, less than 100 amino acids, less than 80 amino acids, less than 40 amino acids, less than 20 amino acids, less than 15 amino acids, less than 10 amino acids, or less than 5 amino acids. Amino acids forming a peptidyl IL-2Rβγc linker can comprise naturally occurring amino acids and/or non-naturally occurring amino acids.

A peptidyl IL-2Rβγc linker can comprise, for example, flexible amino acids such as glycine, serine, and/or threonine. Flexible linkers can include small, non-polar amino acids such as glycine or polar amino acids such as serine or threonine. The small size of these amino acids provides flexibility and allows for mobility of the connecting functional domains. The incorporation of serine or threonine can maintain the stability of the linker in aqueous solutions by forming hydrogen bonds with water molecules and thereby reducing unfavorable interactions between the linker and protein moieties. Amino acids such as lysine and glutamic acid can be included to improve solubility. The length of a peptidyl IL-2Rβγc linker can be selected to provide a suitable separation between the IL-2Rβ and IL-2Rγc ligands to favor a desired interaction with IL-2R such as enhancing agonist activity. Examples of flexible linkers include peptides having an amino acid sequence of any one of SEQ ID NO: 1-28.

A peptidyl IL-2Rβγc linker can be a rigid linker. Rigid linkers can be proline-rich and can include other amino acids such as alanine, lysine, and/or glutamic acid. Examples of rigid peptidyl ligand linkers include peptides having an amino acid sequence of any one of SEQ ID NO: 31-42.

An IL-2Rβγc agonist peptide comprising a peptidyl IL-2Rβγc linker can be synthesized using non-recombinant methods such as using the solid phase synthesis as described in Example 1 or can be synthesized using recombinant DNA technology.

An IL-2Rβγc linker can comprise a synthetic chemical IL-2Rβγc linker. A synthetic chemical IL-2Rβγc linker refers to a linker that is synthesized using chemical methods and can include amino acids or may not include amino acids. A synthetic chemical IL-2Rβγc linker can comprise a triazole moiety.

A synthetic chemical ligand linker can have the structure, for example, of Formula (L2)-(L13) as shown in Table 3.

TABLE 3
Examples of IL-2Rβγc synthetic chemical linkers.

| Formula | |
|---|---|
| No. | Chemical Structure |
| (L1) | 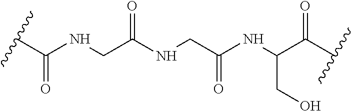 [SMILES: *C(=O)NCC(=O)NCC(=O)NC(CO)C(*)=O] |
| (L2) | 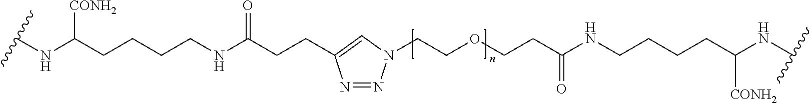 |
| | n = 2 |
| (L3) | 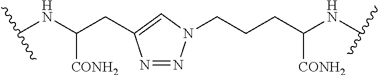 [SMILES: *NC(CCCn1cc(CC(N*)C(N)=O)nn1)C(N)=O] |
| (L4) | 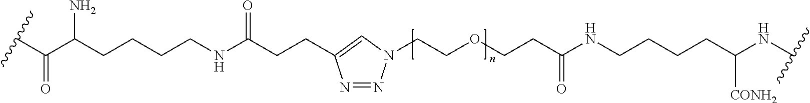 |
| | n = 2 |
| (L5) | 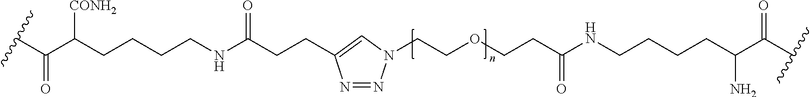 |
| | n = 2 |
| (L6) | 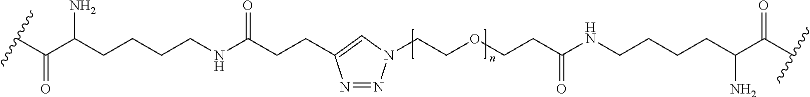 |
| | n = 2 |
| (L7) | 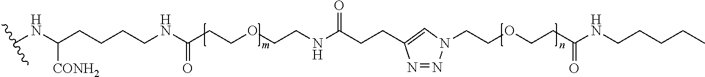 |
| | m = 4 and n = 2 |
| (L8) | 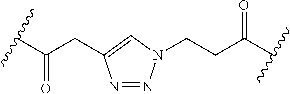 [SMILES: *C(=O)CCn1cc(CC(*)=O)nn1] |
| (L9) | 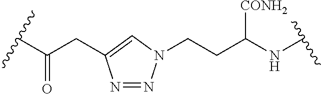 [SMILES: *NC(CCn1cc(CC(*)=O)nn1)C(N)=O] |
| (L10) | 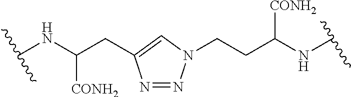 [SMILES: *NC(CCn1cc(CC(N*)C(N)=O)nn1)C(N)=O] |
| (L11) | 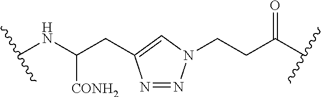 [SMILES: *NC(Cc1cn(CCC(*)=O)nn1)C(N)=O] |
| (L12) | 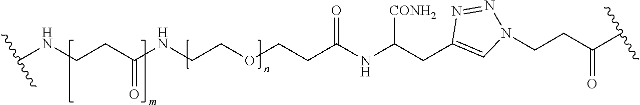 |
| | m = 2, n = 1 |
| (L13) | 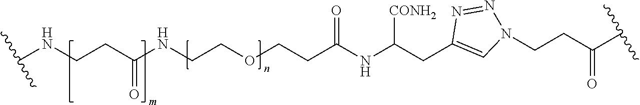 |
| | m = 2, n = 4 |
| (L14) | 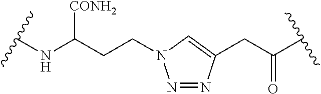 [SMILES: *NC(CCn1cc(CC(*)=O)nn1)C(N)=O] |
| (L15) | 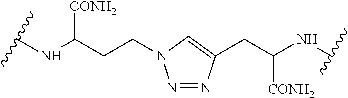 [SMILES: *NC(CCn1cc(CC(N*)C(N)=O)nn1)C(N)=O] |
| (L16) | 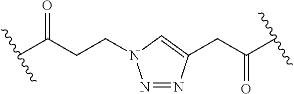 [SMILES: *C(=O)CCn1cc(CC(*)=O)nn1] |
| (L17) | 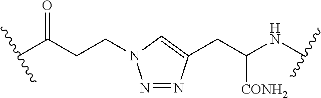 [SMILES: *NC(Cc1cn(CCC(*)=O)nn1)C(N)=O] |

In IL-2Rγc linkers (L2), (L4)-(L7), (L12), and (L13), m and/or n can be an integer, for example, from 1 to 10.

A chemical IL-2Rβγc linker can be synthesized using click chemistry to provide IL-2Rβγc agonist peptides having various C/N orientations of the IL-2Rβ and IL-2Rγc ligands. C/N orientation refers to the termini of the IL-2Rβ and IL-2Rγc ligands, respectively, which are bonded to the IL-2Rβγc linker. For example, for an IL-2Rβγc agonist peptide having a C/N orientation, the C-terminus of the IL-2Rβ ligand is bonded to the IL-2Rβγc linker, and the N-terminus of the IL-2Rγc ligand is bonded to the IL-2Rβγc linker. As another example, for an IL-2Rβγc agonist peptide having an N/C orientation, the N-terminus of the IL-2Rβ ligand is bonded to the IL-2Rβγc linker, and the C-terminus of the IL-2Rγc ligand is bonded to the IL-2Rβγc linker.

IL-2Rβ and IL-2Rγc ligands can be prepared using standard solid phase peptide synthesis and Fmoc-protected amino acids. A swollen resin can be treated with either an activated solution of Fmoc-propargyl glycine or 2-(Fmoc-NH)-azido-pentanoic acid to provide the corresponding Fmoc-protected resin. The alkyne-containing peptide and the azide-containing peptide can be configured to have, for example, a desired length, rigidity/flexibility, polarity, lipophilicity, and/or steric property. The protected resin can be subjected to repeated cycles of Fmoc-amino acid couplings with HATU activation and Fmoc removal to synthesize the respective IL-2Rβ ligand or IL-2Rγc ligand. After Fmoc removal from the final amino acid of the IL-2Rβ or IL-2Rγc ligand and acylation of terminal amine groups, the peptide ligands can be cleaved from the resin and purified.

The alkyne-containing peptide and azide-containing peptide can be reacted, for example, in the presence of CuSO₄ and a metal chelator to provide an IL-2Rβγc agonist peptide comprising a synthetic chemical IL-2Rβγc linker. The reacted alkyne-containing moiety and azide-containing moiety form the chemical ligand linker. For example, referring to Tables 3-5, an alkyne-containing moiety of Formula (AL) in Table 4 can be reacted with an azide-containing moiety of Formula (AZ) in Table 5 to provide a chemical IL-2Rβγc linker of Formula (L) in Table 3.

Using this click-chemistry method, IL-2Rβγc agonist peptides comprising IL-2Rβ and IL-2Rγc ligands having differing N-terminal and C-terminal orientations and different ligand linker lengths can be synthesized.

Examples of alkyne-containing moieties bonded to peptides are provided in Table 4, and examples of azide-containing moieties bonded to peptides are provided in Table 5.

TABLE 4
Examples of alkyne-containing moieties.

| Formula | |
|---|---|
| No. | Chemical Structure |
| (AL1) | 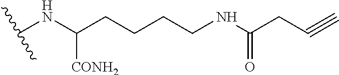 [SMILES: *NC(CCCCNC(=O)CC#C)C(N)=O] |
| (AL2) | 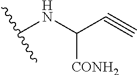 [SMILES: *NC(C#C)C(N)=O] |
| (AL3) | 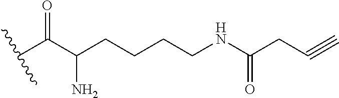 [SMILES: *C(=O)C(N)CCCCNC(=O)CC#C] |
| (AL4) | 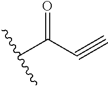 [SMILES: *C(=O)C#C] |
| (AL5) | 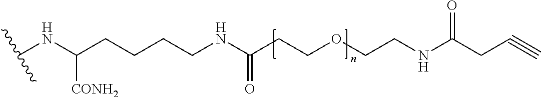 |
| | n = 4 |
| (AL6) | 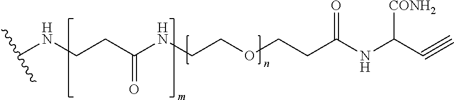 |
| | m = 2 and n = 1 |
| (AL7) | 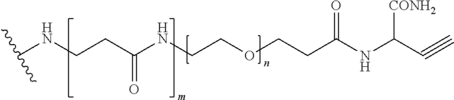 |
| | m = 2 and n = 4 |
| (AL8) | 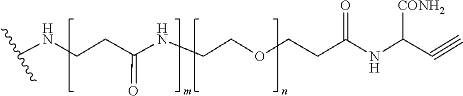 |
| | m = 1 to 10, and n = 1 to 10 |
| (AL9) | 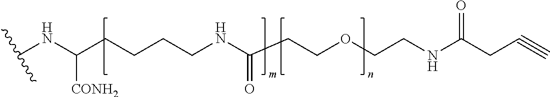 |
| | m = 1 to 10, and n = 1 to 10 |

TABLE 5
Examples of azide-containing moieties,

| Formula | |
|---|---|
| No. | Chemical Structure |
| (AZ1) | 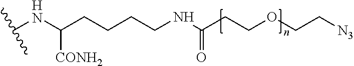 |
| | n = 2 |
| (AZ2) | 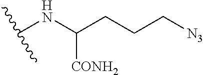 [SMILES: *NC(CCCN=[N+]=[N-])C(N)=O] |
| (AZ3) | 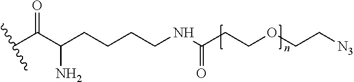 |
| | n = 2 |
| (AZ4) | 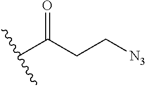 [SMILES: *C(=O)CCN=[N+]=[N-]] |
| (AZ5) | 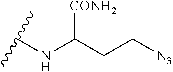 [SMILES: *NC(CCN=[N+]=[N-])C(N)=O] |

An IL-2Rβγc agonist peptide can comprise N- and/or C-terminal modifications to prevent or minimize degradation by aminopeptidases and carboxypeptidases. Examples of terminal groups include an acetyl group on the N-terminus and a carboxamide group on the C-terminus.

An IL-2Rβγc agonist peptide provided by the present disclosure can comprise, for example, a moiety having the structure of Formula (6):

-BL-L-GL- (6)

where BL comprises an IL-2Rβ ligand provided by the present disclosure, L comprises an IL-2Rβγc linker provided by the present disclosure, and GL comprises an IL-2Rγc ligand provided by the present disclosure. In a moiety of Formula (6) the carboxyl terminus of the BL ligand can be covalently bonded to the amino terminus of the IL-2Rβγc linker and the amino terminus of the GL ligand can be covalently bonded to the IL-2Rβγc linker.

A moiety of Formula (6) can be terminated in small chemical moieties and can have a molecular weight, for example, less than 12,000 Da, less than 11,000 Da, less than 10,000 Da, less than 9,000 Da, less than 8,000 Da, less than 7,000 Da, or less than 6,000 Da. An IL-2Rβγc agonist peptide can have a molecular weight, for example, from 6,000 Da to 12,000 Da, from 7,000 Da to 11,000 Da, or from 8,000 Da to 10,000 Da.

A moiety of Formula (6) can be terminated on the C-terminus and/or N-terminus in glycines such as from 1 to 10 glycines, from 1 to 5 glycines, or 1, 2, 3, 4, or 5 glycines.

In IL-2Rβγc agonist peptide of Formula (6), BL can comprise an IL-2Rβ ligand having an amino acid sequence of any one of SEQ ID NO: 74-172 and 330-560 or having an amino acid sequence having greater than 60%, greater than 70%, greater than 80%, greater than 85%, greater than 90%, or greater than 95% sequence similarity to any one of SEQ ID NO: 74-172 and 330-560; GL can comprise an IL-2Rγc ligand having an amino acid sequence of SEQ ID NO: 631-715, or having an amino acid sequence having greater than 60%, greater than 70%, greater than 80%, greater than 85%, greater than 90%, or greater than 95% sequence similarity to any one of SEQ ID NO: 631-715; and L can comprise a peptidyl IL-2Rβγc linker or a chemical IL-2Rβγc linker.

In IL-2Rβγc agonist peptide of Formula (6), either the N-terminus or the C-terminus of the IL-2Rβ ligand can be bound to the IL-2Rβγc linker, and either the N-terminus or the C-terminus of IL-2Rγc ligand can be bound to the IL-2Rβγc linker. For example, the C-terminus of the IL-2Rβ ligand (BL) can be bound to the linker (L), and the N-terminus of the IL-2Rγc ligand (GL) can be bound to the linker (L).

In IL-2Rβγc agonist peptide of Formula (6), each of the IL-2Rβ ligands and the IL-2Rγc ligands can independently comprise one or more flanking amino acids bound to the N-terminus and/or the C-terminus of the ligand. For example, both the N-terminus and the C-terminus of the IL-2Rβ ligand can comprise (G)ₙ (SEQ ID NO: 1), and both the N-terminus and the C-terminus of the IL-2Rγc ligand can comprise (G)ₙ (SEQ ID NO: 1). The flanking amino acids can be bound to the IL-2Rβγc agonist peptide linker.

IL-2Rβγc agonist peptide of Formula (6) can comprise an acetyl terminal group on the N-terminus and a carboxamide group on the C-terminus.

An IL-2Rβγc agonist peptide provided by the present disclosure can comprise the structure of Formula (6a):

-(A)ₙ-BL-(A)ₙ-L-(A)ₙ-GL-(A)ₙ- (6a)

where,
each n can independently be an integer from 0 to 10;
BL is an IL-2Rβ ligand provided by the present disclosure:
GL is an IL-2Rγc ligand provided by the present disclosure;
each A is independently selected from 1 to 5 amino acids; and
L is a peptidyl ligand linker comprising from 1 to 50 amino acids.

In an IL-2Rβγc agonist peptide of Formula (6a),
each n is independently an integer from 0 to 10;
BL is an IL-2Rβ ligand comprising:
   an amino acid sequence selected from any one of SEQ ID NO: 74-172, 173-253, 330-560, 561-587, 588-608, 609-618, and 770-785;
   an amino acid sequence having greater than 60%, greater than 70%, greater than 80%, greater than 85%, or greater than 90% sequence identity to any one of SEQ ID NO: 74-172, 588-608, 173-253, 330-560, 561-587, 588-608, 609-618, and 770-785;
   a truncated amino acid sequence of any one of SEQ ID NO: 74-172, 588-608, 173-253, 330-560, 561-587, 588-608, 609-618, and 770-785; or
   a combination of any of the foregoing;
GL is an IL-2Rγc ligand comprising:
   an amino acid sequence selected from any one of SEQ ID NO: 631-718, 723-749, 652, 786-795, and 797-800;
   an amino acid sequence having greater than 60%, greater than 70%, greater than 80%, greater than 85%, or greater than 90% sequence identity to any one of SEQ ID NO: 631-718, 723-749, 652, 786-795, and 797-800; and/or
   a truncated amino acid sequence of any one of SEQ ID NO: 631-718, 723-749, 652, 786-795, and 797-800;
each A is an independently selected amino acid; and
L is a peptidyl ligand linker comprising from 1 to 50 amino acids.

In IL-2Rβγc agonist peptides of Formula (6a), the C-terminus of the IL-2Rβ ligand can be bound to the peptidyl ligand linker, and the N-terminus of the IL-2Rγc ligand can be bonded to the peptidyl ligand linker.

In IL-2Rβγc agonist peptides of Formula (6a) each n can independently be selected from, for example, an integer from 0 to 8, from 0 to 6, from 0 to 4, or from 0 to 2. For example, n can be 0, 1, 2, or 3. (A)ₙ represents flanking amino acids.

Each A can independently be selected from a naturally occurring or non-naturally occurring amino acid. Each A can be independently selected from a flexible amino acid such as glycine or serine. Each A can be glycine.

L can comprise, for example, from 1 to 40 amino acids, from 1 to 30 amino acids, from 1 to 20 amino acids, from 1 to 10 amino acids, or from 1 to 5 amino acids. Lₐ can be selected from a peptidyl ligand linker. For example, L can be a peptidyl ligand linker having an amino acid sequence of any one of SEQ ID NO: 1-42.

IL-2Rβγc agonist peptides can comprise disulfide bonds. IL-2Rβ ligands and IL-2Rγc ligands can comprise at least two cysteines. Two cysteines of an IL-2Rβ ligand can be bonded through disulfide bonds and two cysteines of an IL-2Rγc ligand can be bonded through a disulfide bond.

In an IL-2Rβγc agonist peptide, two cysteines of the IL-2Rβ ligand can be bonded together through a disulfide bond and/or two cysteines of the IL-2Rγc ligand can be bonded together through a disulfide bond. In an IL-2Rβγc agonist peptide a cysteine of an IL-2Rβ ligand can be bonded to a cysteine of the IL-2Rγc ligand through a disulfide bond, or each of the two cysteines of an IL-2Rβ ligand can be bonded to a cysteine of the IL-2Rγc ligand. For example, in an IL-2Rβγc agonist peptide having the structure of Formula (7):

-X-C¹-X-C²-X-L-Y-C³-Y-C⁴-Y- (7)

where -X-C¹-X-C²-X-represents an amino acid sequence of an IL-2Rβ ligand having two cysteines such as any one of SEQ ID NO: 74-172 and 330-560, and where each X is independently one or more amino acids, -Y-C³-Y-C⁴-Y- represents an amino acid sequence of an IL-2Rγc ligand having two cysteines such as any one of SEQ ID NO: 631-715 and where each Y is independently one or more amino acids, and -L- is an IL-2Rβγc linker coupling the IL-2Rβ ligand and the IL-2Rγc ligand.

In an IL-2Rβγc agonist peptide of Formula (7), C¹ can be linked to C² and C³ can be linked to C⁴ through disulfide bonds; C¹ can be bonded to C³ and C² can be bonded to C⁴ through disulfide bonds, or C¹ can be bonded to C⁴ and C² can be bonded to C³ through disulfide bonds.

An IL-2Rβγc agonist peptide can comprise an IL-2Rβ ligand provided by the present disclosure such as an amino acid sequence selected from any one of SEQ ID NO: 74-172, 588-608, 173-253, 330-560, 561-587, 609-618, and 770-785, a substituted amino acid sequence of an IL-2Rβ ligand of an IL-2Rβ ligand provided by the present disclosure, an amino acid sequence of an IL-2Rβ ligand provided by the present disclosure having from 1 to 5 flanking glycines (SEQ ID NO: 29) on the N-terminus and/or the C-terminus, an amino acid sequence having greater than 60% sequence similarity to an IL-2Rβ ligand provided by the present disclosure, or a combination of any of the foregoing; and a Rγc ligand provided by the present disclosure such as an amino acid sequence selected from any one of SEQ ID NO: 631-715, 723-749, and 652, 786-795, and 797-800, a substituted amino acid sequence of an IL-2Rγc ligand provided by the present disclosure, a truncated amino acid sequence of an IL-2Rγc ligand provided by the present disclosure, an amino acid sequence an IL-2Rγc ligand provided by the present disclosure having from 1 to 5 flanking glycines (SEQ ID NO: 29) on the N-terminus and/or the C-terminus, an amino acid sequence having greater than 60% sequence similarity to an IL-2Rγc ligand provided by the present disclosure, or a combination of any of the foregoing.

An IL-2Rβγc agonist peptide provided by the present disclosure can comprise an IL-2Rβ ligand having an amino acid sequence of any one of SEQ ID NO: 134, 527, 786-788, 791,792, 795, 770, 776, and 777, or an amino acid sequence having greater than 60%, greater than 70%, greater than 75%, greater than 80%, greater than 85%, greater than 90%, or greater than 95% sequence similarity to any one of SEQ ID NO: 134, 527, 786-788, 791,792, 795, 770, 776, and 777: and an IL-2Rγc ligand having an amino acid sequence of any one of SEQ ID NO: 652, 786-795, and 797-800, or an amino acid sequence having greater than 60%, greater than 70%, greater than 75%, greater than 80%, greater than 85%, greater than 90%, or greater than 95% sequence similarity to any one of SEQ ID NO: 652, 5786-795, and 797-800.

IL-2Rβγc agonist peptide provided by the present disclosure can comprise an IL-2Rβ ligand having SEQ ID NO: 134, 527, or an amino acid sequence having greater than 60%, greater than 70%, greater than 75%, greater than 80%, greater than 85%, greater than 90%, or greater than 95 sequence similarity to SEQ ID NO: 134 or 527; and an IL-2Rγc ligand having SEQ ID NO: 652 or an amino acid sequence having greater than 60%, greater than 70%, greater than 75%, greater than 80%, greater than 85%, greater than 90%, or greater than 95% sequence similarity to SEQ ID NO: 652.

IL-2Rβγc agonist peptide provided by the present disclosure can comprise an IL-2Rβ ligand having SEQ ID NO: 134 or an amino acid sequence having greater than 60%, greater than 70%, greater than 75%, greater than 80%, greater than 85%, greater than 90%, or greater than 95% sequence similarity to SEQ ID NO: 652; and an IL-2Rγc ligand having SEQ ID NO: 652 or an amino acid sequence having greater than 60%, greater than 70%, greater than 75%, greater than 80%, greater than 85%, greater than 90%, or greater than 95% sequence similarity to SEQ ID NO: 652.

IL-2Rβγc agonist peptide provided by the present disclosure can comprise an IL-2Rβ ligand having SEQ ID NO: 527 or an amino acid sequence having greater than 60%, greater than 70%, greater than 75%, greater than 80%, greater than 85%, greater than 90%, or greater than 95% sequence similarity to SEQ ID NO: 527; and an IL-2Rγc ligand having SEQ ID NO: 652 or an amino acid sequence having greater than 60%, greater than 70%, greater than 75%, greater than 80%, greater than 85%, greater than 90%, or greater than 95% sequence similarity to SEQ ID NO: 652.

In IL-2Rβγc agonist peptides, the C-terminus of the IL-2Rβ ligand can be linked to the N-terminus of the IL-2Rγc ligand.

An IL-2Rβγc agonist peptide can comprise an IL-2Rβ ligand having a truncated amino acid sequence of SEQ ID NO: 134 and an IL-2Rγc ligand having a truncated amino acid sequence SEQ ID NO: 652.

An IL-2Rβγc agonist peptide can comprise an IL-2Rβ ligand having a truncated amino acid sequence of SEQ ID NO: 527 and an IL-2Rγc ligand having a truncated amino acid sequence SEQ ID NO: 652.

An IL-2Rβγc agonist peptide can comprise an IL-2Rβ ligand having an acid sequence of SEQ ID NO: 134 and an IL-2Rγc ligand having an amino acid sequence SEQ ID NO: 652.

An IL-2Rβγc agonist peptide can comprise an IL-2Rβ ligand having an amino acid sequence of SEQ ID NO: 527 and an IL-2Rγc ligand having an amino acid sequence SEQ ID NO: 652.

An IL-2Rβγc agonist peptide can comprise an IL-2Rβ ligand having a substituted SEQ ID NO: 134 and an IL-2Rγc ligand having a substituted SEQ ID NO: 652, wherein the substitutions can comprise from 1 to 5 conservative amino acid substitutions or non-conservative amino acid substitutions, such as from 1 to 2 conservative amino acid substitutions or non-conservative amino acid substitutions.

An IL-2Rβγc agonist peptide can comprise an IL-2Rβ ligand having a substituted SEQ ID NO: 527 and an IL-2Rγc ligand having a substituted SEQ ID NO: 652, wherein the substitutions can comprise from 1 to 5 conservative amino acid substitutions or non-conservative amino acid substitutions, such as from 1 to 2 conservative amino acid substitutions or non-conservative amino acid substitutions.

Each of the IL-2Rβ ligand and the IL-2Rγc ligand can independently comprise one or more flanking amino acids such as one or more glycines. For example, each of the N-terminus and the C-terminus of the IL-2Rβ ligand and the IL-2Rγc ligand can independently comprise from 1 to 5 glycines (SEQ ID NO: 29).

The N-terminus of the IL-2Rβ ligand can be coupled to the C-terminus of the IL-2Rγc ligand through a flexible linker comprising, for example, from 1 to 10 amino acids. The linker can be, for example, a peptidyl linker having an amino acid sequence of any one of SEQ ID NO: 1-42.

An IL-2Rβγc agonist peptide can comprise the amino acid sequence of SEQ ID NO: 801 (-GGWYPCWMAQLGELCDLDGGGGSGGVVCQDWEGVELCWQGG-), or an amino acid sequence having greater than 60%, greater than 70%, greater than 75%, greater than 80%, greater than 85%, greater than 90%, or greater than 95% sequence similarity to SEQ ID NO: 801.

An IL-2Rβγc agonist peptide can comprise the amino acid sequence of SEQ ID NO: 802 (-WYPCWMAQLGELCDLDGGGGSGGVVCQDWEGVELCWQ-) or an amino acid sequence having greater than 60%, greater than 70%, greater than 75%, greater than 80%, greater than 85%, greater than 90%, or greater than 95% sequence similarity to SEQ ID NO: 802.

An IL-2Rβγc agonist peptide can comprise the amino acid sequence of SEQ ID NO: 803 (-WYPCWMAQLGELCDLDGG-X³⁰⁰-GGVVCQDWEGVELCWQ-) or can comprise an amino acid sequence having greater than 60%, greater than 70%, greater than 80%, greater than 85%, greater than 90%, or greater than 95% amino acid sequence similarity to SEQ ID NO: 803, where X³⁰⁰ can include from 1 to 20 amino acids. For example, X³⁰⁰ can be selected from a peptidyl linker having an amino acid sequence of any one of SEQ ID NO: 1-28 and 31-42. X³⁰⁰ can be selected such that an IL-2Rβγc agonist peptide of any one of SEQ ID NO: 801-803 or an amino acid sequence having greater than 60%, greater than 70%, greater than 75%, greater than 80%, greater than 85%, greater than 90%, or greater than 95% sequence similarity to any one of SEQ ID NO: 801-803 is an agonist for IL-2R or a partial agonist of IL-2R.

An IL-2Rβγc agonist peptide can comprise the amino acid sequence of SEQ ID NO: 804 (-WYPCWMAQLGELCDLD-X³⁰⁰-VVCQDWEGVELCWQ-) or an amino acid sequence having greater than 60%, greater than 70%, greater than 80%, greater than 85%, greater than 90%, or greater than 95% amino acid sequence similarity to SEQ ID NO: 804, where X³⁰⁰ includes from 1 to 20 amino acids -X³⁰⁰ can be selected such that an IL-2Rβγc agonist peptide of SEQ ID NO: 804 or an amino acid sequence having greater than 60%, greater than 70%, greater than 75%, greater than 80%, greater than 85%, greater than 90%, or greater than 95% sequence similarity to SEQ ID NO: 804 is an agonist for IL-2R or a partial agonist of IL-2R.

In IL-2Rβγc agonist peptides of any one of SEQ ID NO: 801-804 and 809-812 or an amino acid sequence having greater than 60%, greater than 70%, greater than 75%, greater than 80%, greater than 85%, greater than 90%, or greater than 95% sequence similarity to any one of SEQ ID NO: 801-804 and 809-812, the cysteines of the IL-2Rβ ligand can be bound together through a disulfide bond, and the cysteines of the IL-2Rγc ligand can be bonded together through disulfide bonds. In certain IL-2Rβγc agonist peptides, the cysteines of the IL-2Rβ ligand can be bonded to the cysteines of the IL-2Rγc ligand.

An IL-2Rβγc agonist peptide can comprise an amino acid sequence selected from any one of SEQ ID NO: 801-804 and 809-812, a substituted amino acid sequence of any one of SEQ ID NO: 801-804 and 809-812, a truncated amino acid sequence of any one of SEQ ID NO: 801-804 and 809-812 an amino acid sequence of any one of SEQ ID NO: 801-804 and 809-812 having from 1 to 5 flanking glycines (SEQ ID NO: 29) on the N-terminus and/or the C-terminus, an amino acid sequence having greater than 60% sequence similarity to any one of SEQ ID NO: 801-804 and 809-812, or a combination of any of the foregoing.

| | |
|---|---|
| SEQ ID NO: 801 | -GGWYPCWMAQLGELCDLDGGGGSGGVVCQDWEGVELCWQGG- |
| SEQ ID NO: 802 | -WYPCWMAQLGELCDLDGGGGSGGVVCQDWEGVELCWQ- |
| SEQ ID NO: 803 | -WYPCWMAQLGELCDLDGG-X³⁰⁰-GGVVCQDWEGVELCWQ- |
| SEQ ID NO: 804 | -WYPCWMAQLGELCDLD-X³⁰⁰-VVCQDWEGVELCWQ- |
| SEQ ID NO: 809 | -GWYPCWMAQLGELCDLDGGGGSGGVVCQDWEGVELCWQG- |
| SEQ ID NO: 810 | -GGWYPCWMAQLGELCDLDGGGGSGGVVCQDWEGVELCWQG- |
| SEQ ID NO: 811 | -GWYPCWMAQLGELCDLDGGGGSGGVVCQDWEGVELCWQGG- |
| SEQ ID NO: 812 | (X³⁰¹)ₙ)-WYPCWMAQLGELCDLD-X³⁰⁰-VVCQDWEGVELCWQ-(X³⁰¹)ₙ- |

In an IL-2Rβγc agonist peptide having an amino acid sequence of any one of SEQ ID NO: 803 and 812, X³⁰⁰ can include from 1 to 20 amino acids. For example, X³⁰⁰ can be selected from having an amino acid sequence of any one of SEQ ID NO: 1-28. For example, X³⁰⁰ can be GGS (SEQ ID NO: 23) or GGGGSGG (SEQ ID NO: 25). For example, X³⁰⁰ can be selected from a peptidyl linker having an amino acid sequence of any one of SEQ ID NO: 31-42.

In IL-2Rβγc agonist peptide having SEQ ID NO: 812, each X³⁰¹ can independently comprise a flanking amino acid such as a glycine, where each n is independently an integer from 0 to 5.

An IL-2Rβγc agonist peptide having an amino acid sequence of any one of SEQ ID NO: 801-804 and 809-812, the cysteines of the IL-2Rβ ligand can be bound together through a disulfide bond, and the cysteines of the Rγc ligand can be bonded together through a disulfide bond. In certain IL-2Rβγc peptide agonists, the cysteines of the IL-2Rβ ligand can be bonded to the cysteines of the Rγc ligand.

An IL-2Rβγc agonist peptide can have greater than 60%, greater than 70%, greater than 75%, greater than 80%, greater than 85%, greater than 90%, or greater than 95% sequence similarity to any one of SEQ ID NO: 801-804 and 809-812.

An IL-2Rβγc agonist peptide can comprise an amino acid sequence selected from any one of SEQ ID NO: 813-828, a substituted amino acid sequence of any one of SEQ ID NO: 813-828, a truncated amino acid sequence of any one of SEQ ID NO: 813-828, an amino acid sequence of any one of SEQ ID NO: 813-828 having from 1 to 5 flanking glycines (SEQ ID NO: 29) on the N-terminus and/or the C-terminus, an amino acid sequence having greater than 60% sequence similarity to any one of SEQ ID NO: 813-828, or a combination of any of the foregoing.

| | |
|---|---|
| SEQ ID NO: 813 | GGWYPCWIARVGELCDLEEGPVNRGGGGSGGVVCQDWEGVELCWQGG |
| SEQ ID NO: 814 | GGAVEFYPCWLARIGELCDLVEPGGGGSGGVVCQDWEGVELCWQGG |
| SEQ ID NO: 815 | GGWYPCWIARVGELCDMEGGGGSGGVVCQDWEGVELCWQGG |
| SEQ ID NO: 816 | GGEWFHDCFLAKVGDLCDLFLWGGGGSGGVVCQDWEGVELCWQGG |
| SEQ ID NO: 817 | GGRYVHDCFIAQVGDLCDLFLHGGGGSGGVVCQDWEGVELCWQGG |
| SEQ ID NO: 818 | GGRSLVDCFLVKVGDLCDFFNWGGGGSGGVVCQDWEGVELCWQGG |
| SEQ ID NO: 819 | GGWYPCWIARVGELCDLEGGGGSGGVVCQDWEGVELCWQGG |
| SEQ ID NO: 820 | GGWYPCWLAQVGELCDLDGGGGSGGVVCQDWEGVELCWQGG |
| SEQ ID NO: 821 | GGWYPCWIARVGELCDLEEGPVNRGGGGSGGGGSGGVVCQDWEGVELCW |
| | QGG |
| SEQ ID NO: 822 | GGWYPCWIARVGELCDLEEGPVNRGGGGSGGGGSGGGGSGGVVCQDWEG |
| | VELCWQGG |
| SEQ ID NO: 823 | GGRYVHDCFIAQVGDLCDLFLHGGGGSGGGGSGGVVCQDWEGVELCWQGG |
| SEQ ID NO: 824 | GGRYVHDCFIAQVGDLCDLFLHGGGGSGGGGSGGGGSGGVVCQDWEGVEL |
| | CWQGG |
| SEQ ID NO: 825 | GGRYVHDCFIAQVGDLCDLFLHGGGGSGGGGSGGGGSGGGGSGGVVCQDW |
| | EGVELCWQGG |
| SEQ ID NO: 826 | GGLVDCFKVKVGELCDLFGGGGSGGVVCQDWEGVELCWQGG |
| SEQ ID NO: 827 | GGRYVHDCFIAQVGDLCDLFLHGGGGSGGVVCQDWEGVELCWQGG |
| SEQ ID NO: 828 | GGWYSCWMAQLGELCDLDGGGGSGGVVCQDWEGVELCWQGG |

An IL-2Rβγc agonist peptide having an amino acid sequence of any one of SEQ ID NO: 813-828, the cysteines of the IL-2Rβ ligand can be bonded together through a disulfide bond, and the cysteines of the Rγc ligand can be bonded together through a disulfide bond. In certain IL-2Rβγc agonist peptides, the cysteines of the IL-2Rβ ligand can be bonded to the cysteines of the Rγc ligand.

An IL-2Rβγc agonist peptide can have greater than 60%, greater than 70%, greater than 75%, greater than 80%, greater than 85%, greater than 90%, or greater than 95% sequence similarity to any one of SEQ ID NO: 813-828.

An IL-2Rβγc agonist peptide can comprise an amino acid sequence of any one of SEQ ID NO: 813-828 having from 1 to 10 amino acid substitutions. The amino acid substitutions can be conservative amino acid substitutions or non-conservative amino acid substitutions.

In IL-2Rβγc agonist peptide having SEQ ID NO: 813-828, the ligand linker can be another ligand linker such as any of those disclosed herein.

An IL-2Rβγc agonist peptide can have the amino acid sequence of any one of SEQ ID NO: 829-833.

| | |
|---|---|
| SEQ ID NO: 829 | Ac-WYPCWMAQLGELCDLDGGGGSGGVVCQDWEGVELCWQ-OH |
| SEQ ID NO: 830 | Ac-WYPCWMAQLGELCDLDGGGGSGGVVCQDWEGVALCWQ-OH |
| SEQ ID NO: 831 | Ac-WYPCW(Abu)AQLGELCDLDGGGGSGGVVCQDWEGVELCWQ-OH |
| SEQ ID NO: 832 | Ac-WYPCW(Abu)AQLGELCDLDGGGGSGGVVCQDWEGVALCWQ-OH |
| SEQ ID NO: 833 | Ac-WYPCWMAQLGELCDLDGGGGSGGVVCQDWEGVELCWQ-OH |

An IL-2Rβγc agonist peptide having the amino acid sequence of any one of SEQ ID NO: 829-833 can bind the hu-IL-2Rβ and hu-IL-2Rγc subunits with an IC₅₀ of less than 100 μM.

An IL-2Rβγc agonist peptide can have the amino acid sequence of any one of SEQ ID NO: 834-852.

| | |
|---|---|
| SEQ ID NO: 834 | Ac-GGELLVDCFKVKVGELCDLFFGGGGGSGGVVCQDWEGVELCWQGG-OH |
| SEQ ID NO: 835 | Ac-GGRYVHDCFIAQVGDLCDLFLHGGGGSGGVVCQDWEGVELCWQGG-OH |
| SEQ ID NO: 836 | Ac-GGKWVHDCFLAKVGDVCDLFVVGGGGSGGVVCQDWEGVELCWQGG-OH |
| SEQ ID NO: 837 | Ac-GGRSLVDCFLVKVGDLCDFFNWGGGGSGGVVCQDWEGVELCWQGG-OH |
| SEQ ID NO: 838 | Ac-GGEWFHDCFLAKVGDLCDLFLWGGGGSGGVVCQDWEGVELCWQGG-OH |
| SEQ ID NO: 839 | Ac-GGGELLVDCFKVKVGELCDLFFGGGGGSGGVVCQDWEGVELCWQGG-OH |
| SEQ ID NO: 840 | Ac-GGGRYVHDCFIAQVGDLCDLFLHGGGGSGGVVCQDWEGVELCWQGG-OH |
| SEQ ID NO: 841 | Ac-GGGKWVHDCFLAKVGDVCDLFVVGGGGSGGVVCQDWEGVELCWQGG-OH |
| SEQ ID NO: 842 | Ac-GGGRSLVDCFLVKVGDLCDFFNWGGGGSGGVVCQDWEGVELCWQGG-OH |
| SEQ ID NO: 843 | Ac-GGGEWFHDCFLAKVGDLCDLFLWGGGGSGGVVCQDWEGVELCWQGG-OH |
| SEQ ID NO: 844 | -GGGELLVDCFKVKVGELCDLFFG-(PA)8-GVVCQDWEGVELCWQGG- |
| SEQ ID NO: 845 | -GGELLVDCFKVKVGELCDLFFG-(PA)8-GVVCQDWEGVELCWQGG- |
| SEQ ID NO: 846 | -ELLVDCFKVKVGELCDLFFG-(PA)8-GVVCQDWEGVELCWQ- |
| SEQ ID NO: 847 | -GGELLVDCFKVKVGELCDLFFG-X⁴⁰⁰-GVVCQDWEGVELCWQGG- |
| SEQ ID NO: 848 | -ELLVDCFKVKVGELCDLFFG-X⁴⁰⁰-GVVCQDWEGVELCWQ- |
| SEQ ID NO: 849 | -(X⁴⁰¹)ₙ)-ELLVDCFKVKVGELCDLFFG-X⁴⁰⁰-GVVCQDWEGVELCWQ-(X⁴⁰¹)ₙ)- |
| SEQ ID NO: 850 | -(X⁴⁰¹)ₙ)-ELLVDCFKVKVGELCDLFFG-(PA)8-GVVCQDWEGVELCWQ-(X⁴⁰¹)ₙ)- |
| SEQ ID NO: 851 | Ac-GGGELLVDCFKVKVGELCDLFFG-(PA)8-GVVCQDWEGVELCWQGG-OH |
| SEQ ID NO: 852 | Ac-GGELLVDCFKVKVGELCDLFFG-(PA)8-GVVCQDWEGVELCWQGG-OH |

In an IL-2Rβγc agonist peptide having an amino acid sequence of any one of SEQ ID NO: 847-849, X⁴⁰⁰ can include from 1 to 20 amino acids. For example, X⁴⁰⁰ can be selected from having an amino acid sequence of any one of SEQ ID NO: 1-28. For example, X⁴⁰⁰ can be GGS (SEQ ID NO: 23) or GGGGSGG (SEQ ID NO: 25). For example, X⁴⁰⁰ can be selected from a peptidyl linker having an amino acid sequence of any one of SEQ ID NO: 31-39.

In an IL-2Rβγc agonist peptide having SEQ ID NO: 849 and 850 each (X⁴⁰¹)n can independently be selected from (G)n where n is an integer from 1 to 10 (SEQ ID NO: 2).

An IL-2Rβγc agonist peptide having the amino acid sequence of any one of SEQ ID NO: 834-852 can bind the hu-IL-2Rβ subunit and to the hu-IL-2Rγc subunit with an IC₅₀ of less than 100 μM.

Certain IL-2Rβγc agonist peptides having an amino acid sequence of any one of SEQ ID NO: 801-804, 809-828, and 829-852 can bind the hu-IL-2Rβ subunit, to the hu-IL-2Rγc subunit, to the cyno-IL-2Rβ subunit, and to the cyno-IL-2Rγc subunit with an IC₅₀ of less than 100 μM.

An IL-2Rβγc agonist peptide provided by the present disclosure can bind to IL-2R such as hu-IL-2R with an IC₅₀ from 1 pM to 100 μM, from 10 pM to 10 μM, from 100 pM to 1 μM, from 0.001 μM to 1 μM, or from 0.01 μM to 1 μM.

An IL-2Rβγc agonist peptide provided by the present disclosure can bind to IL-2R such as hu-IL-2R with an IC₅₀ of less than 100 μM, less than 10 μm, less than 1 μm, less than 100 pM, less than 10 pM, or less than 1 pM.

An IL-2Rβγc agonist peptide provided by the present disclosure can bind to each of the IL-2Rβ subunit and to the IL-2Rγc subunit, such as each of the hu-IL-2Rβ subunit and the hu-IL-2Rγc subunit, with an IC₅₀ from 1 pM to 100 μM, from 10 pM to 10 μM, from 100 pM to 1 μM, from 0.001 μM to 1 μM, or from 0.01 μM to 1 μM.

An IL-2Rβγc agonist peptide provided by the present disclosure can bind to each of the IL-2Rβ subunit and the IL-2Rγc subunit, such as each of the hu-IL-2Rβ subunit and to the hu-IL-2Rγc subunit with an IC₅₀ of less than 100 μm, less than 10 μm, less than 1 μm, less than 100 pM, less than 10 pM, or less than 1 pM.

An IL-2Rβγc agonist peptide provided by the present disclosure can exhibit an EC₅₀ for STAT5 phosphorylation in TF-1β cells and/or NK-92 cells, for example, of less than 100 μM, less than 10 μM, less than 1 μM, less than 100 pM, less than 10 pM, or less than 1 pM.

An IL-2Rβγc agonist peptide provided by the present disclosure can exhibit an EC₅₀ for STAT5 phosphorylation in TF-1β cells and/or NK-92 cells, for example, from 1 pM to 100 μM, from 10 pM to 10 μM, from 100 pM to 1 μM, from 0.001 μM to 1 μM, or from 0.01 μM to 1 μM.

Solid tumors exhibit metabolic differences from normal tissues. The greater reliance of solid tumors on glycolysis produces a more acidic tumor microenvironment. For example, the solid tumor microenvironment can have a pH that is from 1 pH to 2 pH units less than that of most normal tissue. This pH differential can be exploited to enhance the activity of therapeutic agents in solid tumors relative to activity in normal peripheral tissue.

Using suitable pH-selective screening methods, IL-2Rβ and IL-2Rγc ligands can be identified that bind to IL-2R at low pH with an IC₅₀ that is less than the IC₅₀ for binding at a neutral pH. For example, the IC₅₀ for binding at a pH less than 6 can be at least 10 times or at least 100 times less than the IC₅₀ for binding at a pH greater than 6. These pH-biased IL-2Rβ and/or IL-2Rγc ligands can be incorporated into an IL-2Rβγc agonist peptide to provide a pH-biased IL-2Rβγc agonist peptide.

IL-2Rβγc agonist peptides can comprise a pH-biased IL-2Rβ ligand and/or a pH-biased IL-2Rγc ligand. These pH-biased IL-2Rβγc agonist peptides can exhibit an enhanced therapeutic index with respect to increased cytotoxicity targeting solid tumors and with reduced toxicity to normal tissue.

An IL-2Rβγc agonist peptide comprising a pH-biased IL-2Rβ ligand and/or a pH-biased IL-2Rβγc peptide agonist can exhibit a pH-biased binding affinity (IC₅₀) to the IL-2Rβ subunit and/or to the IL-2Rγc subunit.

For example, a pH-biased IL-2Rβγc agonist peptide can bind to IL-2R, such as hu-IL-2R, at pH 6 with an IC₅₀ at least 10% less than the IC₅₀ for binding to IL-2R, such as hu-IL-2R, at pH 7.5, at least 25% less, at least 50% less, at least 100% less, or at least 200% less than the IC₅₀ for binding to IL-2R, such as hu-IL-2R, at pH 7.5.

A pH-biased IL-2Rβγc agonist peptide can bind to IL-2R, such as hu-IL-2R, at pH 6.0 with an IC₅₀ from 1 pM to 100 μM; and bind to IL-2R, such as hu-IL-2R, with an IC₅₀ greater than 100 μM at pH 7.5.

A pH-biased IL-2Rβγc agonist peptide can bind to IL-2Rγc, such as hu-IL-2Rγc, at pH 6.0 with an IC₅₀ from 0.1 μM to 50 μM; and bind to IL-2Rγc, such as hu-IL-2Rγc with an IC₅₀ greater than 100 μM at pH 7.5.

A pH-biased IL-2Rβγc agonist peptide can bind to IL-2Rγc, such as hu-IL-2Rβγc at pH 6.0 with an IC₅₀ of less than 100 μM; and bind to IL-2Rγc, such as hu-IL-2Rγc with an IC₅₀ greater than 100 μM at pH 7.5.

A pH-biased IL-2Rβγc agonist peptide can bind to each of the IL-2Rβ subunit and to the IL-2Rγc subunit, such as the human IL-2Rβ subunit and the IL-2Rγc subunit, with an IC₅₀ of less than 100 μM at pH 6 and bind to each of the IL-2Rβ subunit and to the IL-2Rγc subunit, such as the hu-IL-2Rβ subunit and the IL-2Rγc subunit, with an IC₅₀ greater than 100 μM at pH 7.5.

pH-Biased IL-2Rβγc agonist peptides can be useful, for example, to selectively activate cells expressing IL-2R in low pH cell environments such as in solid tumors compared to cells in neutral and high pH environments.

Using suitable pH-selective screening methods, pH-biased IL-2Rβγc agonist peptides can be identified that have a higher binding affinity (lower IC₅₀) to IL-2R at lower pH and a lower binding affinity (higher IC₅₀) at a neutral pH. IL-2Rβγc agonist peptides and IL-2Rβγc agonist peptide constructs can include one or more pH-biased IL-2Rβ ligands and/or one or more pH-biased IL-2Rγc ligands. These pH-biased IL-2Rβγc agonist peptides, and pH-biased IL-2Rβγc agonist peptide constructs can exhibit an enhanced therapeutic index reflecting increased cytotoxicity for targeting solid tumors and with reduced toxicity to normal tissue.

Similarly, IL-2Rβγc agonist peptides can be selected that exhibit enhanced IL-2R agonist activity at lower pH such as at pH 6 compared to higher pH such as at pH 7 using pH-selective functional screening methods. For example, pH-biased IL-2Rβγc agonist peptides provided by the present disclosure can provide a lower EC₅₀ for STAT5 phosphorylation in TF-1β cells and/or NK-92 cells at pH 6.0 relative to the EC₅₀ for the same IL-2Rβγc agonist peptide at pH 7.5. Such pH-biased IL-2Rβγc agonist peptides exhibit enhanced IL-2R agonist activity or partial agonist activity at lower pH.

For example, a pH-biased IL-2Rβγc agonist peptide can exhibit an EC₅₀ for STAT5 phosphorylation in TF-1β and NK-92 cells at pH 6 that is at least 10% less than the EC₅₀ for STAT5 phosphorylation in TF-1β and NK-92 cells at pH 7.5, at least 25% less, at least 50% less, at least 100% less, or at least 200% less than the EC₅₀ for STAT5 phosphorylation in TF-1β and NK-92 cells at pH 7.5.

Using pH-selective screening methods similar to those described in the examples, pH-biased IL-2Rβ ligands and/or IL-2Rγc ligands having other pH biases, such as other pH-biased functional properties or decreased IC₅₀ or EC₅₀ at pH values greater than normal healthy tissue, can be identified and incorporated into IL-2Rβγc agonist peptides to provide additional biased IL-2Rβγc agonist peptides.

An IL-2Rβγc agonist peptide provided by the present disclosure can comprise two or more IL-2Rβγc agonist peptides. The two or more IL-2Rβγc agonist peptides can be bound together to form a linear or non-linear structure. For example, a tandem IL-2Rβγc agonist peptide can have the structure of Formula (8a) or Formula (8b):

BGL-(-Lₜ₁-BGL-)ₙ₁-Lₜ₁-BGL (8a)

Lₜ₂{(-Lₜ₁-BGL-)ₙ₂-Lₜ₁-BGL}ₚ (8b)

where,
each BGL can be independently selected from an IL-2Rβγc agonist peptide; and
Lₜ₁ can be a divalent linker;
Lₜ₂ can be a p-valent linker;
n1 can be an integer from 1 to 6; and
n2 can be an integer from 0 to 6; and
p can be an integer from 3 to 8.

In IL-2Rβγc agonist peptide of Formula (8a) and (8b), each IL-2Rβγc agonist peptide can be the same.

In IL-2Rβγc agonist peptides of Formula (8a) and (8b), at least one IL-2Rβγc agonist peptide can be different than another IL-2Rβγc agonist peptide.

In IL-2Rβγc agonist peptides of Formula (8a) and (8b), each IL-2Rβγc agonist peptide can independently be bound to a linker through the N-terminus or through the C-terminus of the respective IL-2Rβγc agonist peptide.

In IL-2Rβγc agonist peptides of Formula (8a) and (8b), each of the IL-2Rβγc agonist peptides can comprise one or more flanking amino acids.

A linker, Lₜ₁ and Lₜ₂, can be a peptidyl linker and can, for example, from 1 to 50 amino acids, from 2 to 40 amino acids, or from 5 to 30 amino acids.

A linker can comprise a chemical linker such as a triazole-containing linker provided by the present disclosure.

Each divalent linker Lₜ₁ can be the same as each of the other divalent linkers, or at least one of the divalent linkers can be different than another linker.

In an IL-2Rβγc agonist peptide of Formula (8a), n can be, for example, 1, 2, 3, 4, 5, or 6.

In an IL-2Rβγc agonist peptide of Formula (8b), each n can independently be selected from 0, 1, 2, 3, 4, 5, or 6.

In an IL-2Rβγc agonist peptide of Formula (8b), p can be, for example, 3, 4, 5, 6, 7, or 8.

A p-valent linker can comprise any suitable polyfunctional chemical moiety. For example, IL-2Rβγc agonist peptides of Formula (8a) and (8b) can have a molecular weight, for example, less than 10,000 Da, less than 6,000 Da, less than 2,000 Da, less than 1,000 Da, or less than 500 Da.

An IL-2Rβγc agonist peptide provided by the present disclosure can comprise two or more IL-2Rβ ligands and two or more IL-2Rγc ligands. The two or more IL-2Rβ ligands and two or more IL-2Rγc ligands can be bonded together to form a linear or non-linear structure. For example, a IL-2Rβγc agonist peptide can have the structure of Formula (9a) or Formula (9b):

L-(-Lₜ₁-L-)ₙ₁-Lₜ₁-L (9a)

Lₜ₂{(-Lₜ₁-L-)ₙ₂-Lₜ₁-L}ₚ (9b)

where,
each L can be independently selected from an IL-2Rβ ligand and an IL-2Rγc ligand; and
Lₜ₁ can be a divalent linker;
Lₜ₂ can be a p-valent linker;
n1 can be an integer from 1 to 6;
n2 can be an integer from 0 to 6; and
p can be an integer from 3 to 8, and
wherein the IL-2Rβγc agonist peptide comprises at least one IL-2Rβ ligand and at least one IL-2Rγc ligand.

In IL-2Rβγc agonist peptide of Formula (9a) and (9b), each IL-2Rβ ligand and each IL-2Rγc ligand can be the same.

In IL-2Rβγc agonist peptides of Formula (9a) and (9b), at least one IL-2Rβ ligand can be different than another IL-2Rβ ligand and/or at least one IL-2Rγc ligand can be different than another IL-2Rγc ligand.

In IL-2Rβγc agonist peptides of Formula (9a) and (9b), each IL-2Rβ ligand and IL-2Rγc ligand can independently be bound to a linker through the N-terminus or through the C-terminus of the respective IL-2Rβγc agonist peptide.

In IL-2Rβγc agonist peptides of Formula (9a) and (9b), each IL-2Rβ ligand and IL-2Rγc ligand can independently comprise one or more flanking amino acids.

A linker, Lₜ₁ and Lₜ₂, can be a peptidyl linker and can, for example, from 1 to 50 amino acids, from 2 to 40 amino acids, or from 5 to 30 amino acids.

A linker can comprise a chemical linker such as a triazole-containing linker provided by the present disclosure.

Each divalent linker Lₜ₁ can be the same as each of the other divalent linkers, or at least one of the divalent linkers can be different than another divalent linker.

In an IL-2Rβγc agonist peptide of Formula (9a), n can be, for example, 1, 2, 3, 4, 5, or 6.

In an IL-2Rβγc agonist peptide of Formula (9b), each n can independently be selected from 0, 1, 2, 3, 4, 5, or 6.

In an IL-2Rβγc agonist peptide of Formula (9b), p can be, for example, 3, 4, 5, 6, 7, or 8.

A p-valent linker can comprise any suitable polyfunctional chemical moiety. For example, IL-2Rβγc agonist peptides of Formula (9a) and (9b) can have a molecular weight, for example, less than 10,000 Da, less than 6,000 Da, less than 2,000 Da, less than 1,000 Da, or less than 500 Da.

Aspects of the present invention include a culture medium comprising an IL-2Rβγc peptide agonist such as an IL-2Rβγc agonist peptide provided by the present disclosure.

A culture medium can comprise any suitable culture medium for expanding a target immune cell population of an initial population of immune cells.

A culture medium can comprise a base culture medium and an IL-2Rβγc agonist peptide.

A base culture medium can comprise any suitable culture medium for culturing immune cells such as T cells.

A base culture media refers to any suitable starting media that is supplemented with one or more stimulants of immune cell proliferation. For example, a base culture media can comprise a balanced salt solution such as PBS, DPBS, HBSS, EBSS, Dulbecco's Modified Eagle's Medium (DMEM), Click's medium, Minimal Essential Medium (MEM), Basal Medium Eagle (BME), F-10, F-12, RPMI 1640, Glasgow Minimal Essential Medium (GMEM), alpha Minimal Essential Medium (alpha MEM), Iscove's Modified Dulbecco's Medium (IMDM), M199, OPTMIZER™ Pro, OPTMIZER™ CTS™ T-Cell Expansion Basal Medium (ThermoFisher), OPTMIZER™, OPTMIZER™ Complete, IMMUNOCULT™ XF (STEMCELL™ Technologies), AIM VIM, TEXMACS™ medium, PRIME-XV® T cell CDM, X-VIVO™ 15 (Lonza), TRANSACT™ TIL expansion medium, or any combination thereof. A base culture medium can be serum-free. A base culture media can comprise PRIME-XV® T cell CDM. A base culture medium can comprise OPTMIZER™. A base culture media can comprise OPTMIZER™ Pro. A base culture medium further comprises immune cell serum replacement (ICSR).

A culture media provided by the present disclosure can be used for culturing immune cells such as, for example, expanding a target population of immune cells such as T cells.

A target immune cell population can comprise immune cells for which the growth of the target immune cells is stimulated relative to other immune cells in the culture medium upon incubation with an IL-2Rαγc agonist peptide.

For example, a target immune cell population can comprise a target T cell population.

For example, a target T cell population can comprise CD4+ T cells and CD8+ T cells.

A culture medium can comprise a concentration of an IL-2Rαγc agonist peptide, for example, greater than 1 nM, greater than 10 nM, greater than 100 nM, greater than 200 nM, greater than 500 nM, greater than 1,000 nM, or greater than 2,000 nM.

A culture medium can comprise a concentration of an IL-2Rαγc agonist peptide], for example, less than 5,000 nM, less than 2,000 nM, less than 1,000 nM, less than 500 nM, less than 200 nM, less than 100 nM, less than 50 nM, or less than 10 nM.

A culture medium can comprise a concentration of an IL-2Rαγc agonist peptide, for example, from 1 nM to 5,000 nM, from 10 nM to 1,000 nM, or from 50 nM to 500 nM.

A culture medium can comprise a concentration of an IL-2Rβγc agonist peptide sufficient to stimulate the proliferation of a target immune cell population, such as a population of CD4+ T cells and CD8+ T cells.

A culture medium can comprise a second stimulant of the proliferation of the target immune cell population in addition to an IL-2Rβγc agonist peptide.

For example, a second stimulant for the proliferation of the target immune cell population can comprise, for example, an IL-7Rαγc agonist peptide, an IL-15Rβγc agonist peptide, or a combination thereof.

An IL-7Rαγc agonist peptide refers to a peptide comprising an IL-7Rα ligand and an Rγc ligand, wherein the IL-7Rαγc agonist peptide has an EC50 for STAT5 phosphorylation of TF-1-7a cells of less than 100 μM, less than 10 μM, less than 1 μM, less than 100 pM, less than 10 pM, or less than 1 pM.

An IL-7Rα ligand can bind to the hu-IL-7Rα subunit with an IC₅₀ of less than 100 μM, less than 1 μM, less than 0.1 μM, or less than 0.01 μM.

Examples of suitable IL-7Rα ligands are disclosed, for example, in U.S. Pat. No. 11,254,729 and in U.S. Application Publication No. 2021/0253670 A1, each of which is incorporated by reference in its entirety.

In an IL-7Rαγc agonist peptide, an Rγc ligand can be an Rγc ligand as disclosed herein and can be the same or different Rγc ligand as in the IL-2Rαγc agonist peptide.

An IL-7Rαγc agonist peptide can comprise an IL-7Rα ligand bonded to a Rγc ligand through an IL-7Rαγc ligand linker.

The IL-7Rα ligand and the Rγc ligand can be bonded in an N—C, C—C, C—N, or N—N orientation.

An IL-7Rαγc ligand linker can be a non-peptidyl ligand linker or a peptidyl ligand linker.

A peptidyl ligand linker can be a flexible ligand linker or a rigid ligand linker.

Examples of flexible ligand linkers comprise an amino acid sequence of any one of SEQ ID NO: 1-28.

Examples of rigid ligand linkers comprise an amino acid sequence of any one of SEQ ID NO: 31-30.

An IL-7Rαγc agonist peptide provided by the present disclosure can comprise an amino acid sequence of any one of SEQ ID NO: 853-878 and 880-930.

| | |
|---|---|
| SEQ ID NO: 853 | VHRIPWCTLDPGGLQCAWLRQMGGGGSGGVVCQDWEGVELCWQ |
| SEQ ID NO: 854 | VHRIPWCTLDPGGLQCAWLRQGGGGSGGVVCQDWEGVELCWQ |
| SEQ ID NO: 855 | VHRIPWCTLDPGGLQCAWLRGGGGSGGVVCQDWEGVELCWQ |
| SEQ ID NO: 856 | HRIPWCTLDPGGLQCAWLRQMGGGGSGGVVCQDWEGVELCWQ |
| SEQ ID NO: 857 | HRIPWCTLDPGGLQCAWLRQGGGGSGGVVCQDWEGVELCWQ |
| SEQ ID NO: 858 | HRIPWCTLDPGGLQCAWLRGGGGSGGVVCQDWEGVELCWQ |
| SEQ ID NO: 859 | RIPWCTLDPGGLQCAWLRQMGGGGSGGVVCQDWEGVELCWQ |
| SEQ ID NO: 860 | RIPWCTLDPGGLQCAWLRQGGGGSGGVVCQDWEGVELCWQ |
| SEQ ID NO: 861 | RIPWCTLDPGGLQCAWLRGGGGSGGVVCQDWEGVELCWQ |
| SEQ ID NO: 862 | IPWCTLDPGGLQCAWLRQMGGGGSGGVVCQDWEGVELCWQ |
| SEQ ID NO: 863 | IPWCTLDPGGLQCAWLRQGGGGSGGVVCQDWEGVELCWQ |
| SEQ ID NO: 864 | IPWCTLDPGGLQCAWLRGGGGSGGVVCQDWEGVELCWQ |
| SEQ ID NO: 865 | IEGRGGQCIHWDIETLLSCVGGGGSGGVVCQDWEGVELCWQ |
| SEQ ID NO: 866 | IEGRGGVPWCTLDPGSLQCAWFGGGGSGGVVCQDWEGVELCWQ |
| SEQ ID NO: 867 | IEGRGGRYECADLPGGLHCEFRGGGGSGGVVCQDWEGVELCWQ |
| SEQ ID NO: 868 | RHFDDIIPWCTLDPGSLQCAYLGGGGSGGVVCQDWEGVELCWQ |
| SEQ ID NO: 869 | VHRIPWCTLDPGGLQCAWLRQMGGGGSGGVVCQDWEGVELCWQPPA |
| SEQ ID NO: 870 | VHRIPWCTLDPGGLQCAWLRQGGGGSGGVVCQDWEGVELCWQPPA |
| SEQ ID NO: 871 | VHRIPWCTLDPGGLQCAWLRGGGGSGGVVCQDWEGVELCWQPPA |
| SEQ ID NO: 872 | GWGIPWCTLDPGSLQCAWLGKHGGGGSGGVVCQDWEGVELCWQPPA |
| SEQ ID NO: 873 | VHRIPWCTLDPGGLQCAWLRQGGGGGSGGVVCQDWEGVELCWQPPA |
| SEQ ID NO: 874 | VHRIPWCTLDPGGLQCAWLRGGGGGGSGGVVCQDWEGVELCWQPPA |
| SEQ ID NO: 875 | VHRIPWCTLDPGGLQCAWLRQGGGGSGGVVCQDWEGVELCWQGG |
| SEQ ID NO: 876 | VHRIPWCTLDPGGLQCAWLRGGGGSGGVVCQDWEGVELCWQGG |
| SEQ ID NO: 877 | GWGIPWCTLDPGSLQCAWLGKHGGGGSGGVVCQDWEGVELCWQGG |
| SEQ ID NO: 878 | VHRIPWCTLDPGGLQCAWLRQM(PA)₈GVVCQDWEGVELCWQGG |
| SEQ ID NO: 880 | HLGVPWCTLDPGSIQCAWLAKHGGGGSGGVVCQDWEGVELCWQ |
| SEQ ID NO: 881 | VVCQDWEGVELCWQGGGGSGGRHFDDIIPWCTLDPGSLQCAYL |
| SEQ ID NO: 882 | VVCQDWEGVELCWQGGGGSGGHLGVPWCTLDPGSIQCAWLAKH |
| SEQ ID NO: 883 | HCKHWDLESLLLCVGGGGSGGVVCQDWEGVELCWQ |
| SEQ ID NO: 884 | QCVHWDLDTLFGCIREQLELGGGGSGGVVCQDWEGVELCWQ |
| SEQ ID NO: 885 | VVCQDWEGVELCWQGGGGSGGQCVHWDLDTLFGCIREQLEL |
| SEQ ID NO: 886 | IRSCLWQPGALHCTWWAEEEPVGGGGSGGVVCQDWEGVELCWQ |
| SEQ ID NO: 887 | VVCQDWEGVELCWQGGGGSGGIRSCLWQPGALHCTWWAEEEPV |
| SEQ ID NO: 888 | IPWCLLDPGGLQCVWLGGGGSGGVVCQDWEGVELCWQ |
| SEQ ID NO: 889 | VVCQDWEGVELCWQGGGGSGGIPWCLLDPGGLQCVWL |
| SEQ ID NO: 890 | VHRIPWCTLDPGGLQCAWLRQMGGGGSGGVVCQDWEGVELCWQG |
| SEQ ID NO: 891 | VHRIPWCTLDPGGLQCAWLRQMGGGGSGGVVCQDWEGVELCWQGG |
| SEQ ID NO: 892 | GVHRIPWCTLDPGGLQCAWLRQMGGGGSGGVVCQDWEGVELCWQGG |
| SEQ ID NO: 893 | GGVHRIPWCTLDPGGLQCAWLRQMGGGGSGGVVCQDWEGVELCWQGG |
| SEQ ID NO: 894 | VHRIPWCTLDPGGLQCAWLRGGGGSGGVVCQDWEGVELCWQ |
| SEQ ID NO: 895 | WGIPWCTLDPGSLQCAWLGKHGGGGSGGVVCQDWEGVELCWQ |
| SEQ ID NO: 896 | VHRIPWCTLDPGGLQCAWLRQGGGGSGGVVCQDWEGVELCWQ |
| SEQ ID NO: 897 | VHRIPWCTLDPGGLQCAWLRMGGGGSGGVVCQDWEGVELCWQ |
| SEQ ID NO: 898 | VHRIPWCTLDPGGLQCAWIRQMGGGGSGGVVCQDWEGVELCWQ |
| SEQ ID NO: 899 | VHRIPWCTLDPGGLQCAWVRQMGGGGSGGVVCQDWEGVELCWQ |
| SEQ ID NO: 900 | VHRIPWCTLDPGGLQCAWARQMGGGGSGGVVCQDWEGVELCWQ |
| SEQ ID NO: 901 | GVHRIPWCTLDPGGLQCAWLRQMGGGGSGGVVCQDWEGVELCWQG |
| SEQ ID NO: 902 | VHRIPWCTLDPGGLQCAWLRQGGGGSGGVVCQDWEGVELCWQ |
| SEQ ID NO: 903 | VHRIPWCTLDPGGLQCAWLRGGGGSGGVVCQDWEGVELCWQ |
| SEQ ID NO: 904 | VHRIPWCTLDPGGLQCAWLGKHGGGGSGGVVCQDWEGVELCWQ |
| SEQ ID NO: 905 | VHRIPWCTLDPGGLQCAWLRMGGGGSGGVVCQDWEGVELCWQ |
| SEQ ID NO: 906 | GWGIPWCTLDPGGLQCAWLRQMGGGGSGGVVCQDWEGVELCWQ |
| SEQ ID NO: 907 | GWGIPWCTLDPGGLQCAWLRQGGGGSGGVVCQDWEGVELCWQ |
| SEQ ID NO: 908 | GWGIPWCTLDPGGLQCAWLRGGGGSGGVVCQDWEGVELCWQ |
| SEQ ID NO: 909 | GWGIPWCTLDPGGLQCAWLGKHGGGGSGGVVCQDWEGVELCWQ |
| SEQ ID NO: 910 | GWGIPWCTLDPGGLQCAWLRMGGGGSGGVVCQDWEGVELCWQ |
| SEQ ID NO: 911 | IPWCTLDPGGLQCAWLRQMGGGGSGGVVCQDWEGVELCWQ |
| SEQ ID NO: 912 | IPWCTLDPGGLQCAWLRQGGGGSGGVVCQDWEGVELCWQ |
| SEQ ID NO: 913 | IPWCTLDPGGLQCAWLRGGGGSGGVVCQDWEGVELCWQ |
| SEQ ID NO: 914 | IPWCTLDPGGLQCAWLGKHGGGGSGGVVCQDWEGVELCWQ |
| SEQ ID NO: 915 | IPWCTLDPGGLQCAWLRMGGGGSGGVVCQDWEGVELCWQ |
| SEQ ID NO: 916 | VHRIPWCTLDPGGLQCAWLRQM-X⁴⁰⁰-VVCQDWEGVELCWQ |
| SEQ ID NO: 917 | VHRIPWCTLDPGGLQCAWLRQ-X⁴⁰⁰-VVCQDWEGVELCWQ |
| SEQ ID NO: 918 | VHRIPWCTLDPGGLQCAWLR-X⁴⁰⁰-VVCQDWEGVELCWQ |
| SEQ ID NO: 919 | VHRIPWCTLDPGGLQCAWLGKH-X⁴⁰⁰-VVCQDWEGVELCWQ |
| SEQ ID NO: 920 | VHRIPWCTLDPGGLQCAWLRM-X⁴⁰⁰-VVCQDWEGVELCWQ |
| SEQ ID NO: 921 | GWGIPWCTLDPGGLQCAWLRQM-X⁴⁰⁰-VVCQDWEGVELCWQ |
| SEQ ID NO: 922 | GWGIPWCTLDPGGLQCAWLRQ-X⁴⁰⁰-VVCQDWEGVELCWQ |
| SEQ ID NO: 923 | GWGIPWCTLDPGGLQCAWLR-X⁴⁰⁰-VVCQDWEGVELCWQ |
| SEQ ID NO: 924 | GWGIPWCTLDPGGLQCAWLGKH-X⁴⁰⁰-VVCQDWEGVELCWQ |
| SEQ ID NO: 925 | GWGIPWCTLDPGGLQCAWLRM-X⁴⁰⁰-VVCQDWEGVELCWQ |
| SEQ ID NO: 926 | IPWCTLDPGGLQCAWLRQM-X⁴⁰⁰-VVCQDWEGVELCWQ |
| SEQ ID NO: 927 | IPWCTLDPGGLQCAWLRQ-X⁴⁰⁰-VVCQDWEGVELCWQ |
| SEQ ID NO: 928 | IPWCTLDPGGLQCAWLR-X⁴⁰⁰-VVCQDWEGVELCWQ |
| SEQ ID NO: 929 | IPWCTLDPGGLQCAWLGKH-X⁴⁰⁰-VVCQDWEGVELCWQ |
| SEQ ID NO: 930 | IPWCTLDPGGLQCAWLRM-X⁴⁰⁰-VVCQDWEGVELCWQ |

In an IL-7Rαγc agonist peptide of any one of SEQ ID NO: 916-930, X⁴⁰⁰ can be selected from, for example, (G)ₙ (SEQ ID NO: 11), (GS)ₙ (SEQ ID NO: 12), (GGS)ₙ (SEQ ID NO: 13), (GGGS)ₙ (SEQ ID NO: 14), (GGGGS)ₙ (SEQ ID NO: 15) or a combination of any of the foregoing, where n can independently be an integer from 1 to 5.

In an IL-7Rαγc agonist peptide of any one of SEQ ID NO: 916-930, X⁴⁰⁰ can be -GGGGSGG- (SEQ ID NO: 25).

In an IL-7Rαγc agonist peptide of any one of SEQ ID NO: 853-878 and 880-930, the agonist peptide can comprise flanking glycines on each terminus, such as two flanking glycines on one or both termini.

An IL-7Rαγc agonist peptide provided by the present disclosure can comprise a truncated amino acid sequence of any one of SEQ ID NO: 853-878 and 880-930.

An IL-7Rαγc agonist peptide provided by the present disclosure can have a sequence similarity greater than 60%, greater than 70%, greater than 80%, or greater than 90% to the amino acid sequence of any one of SEQ ID NO: 853-878 and 880-930.

An IL-7Rαγc agonist peptide provided by the present disclosure can comprise an amino acid sequence selected from any one of SEQ ID NO: 853-878 and 880-930 or a truncated amino acid sequence of any one of SEQ ID NO: 916-930, wherein the amino acid sequence can independently comprise from 1 to 4 glycines (G) (SEQ ID NO: 30) on the N-terminus, on the C-terminus, or on both the N- and C-termini.

An IL-7Rαγc agonist peptide provided by the present disclosure can comprise an amino acid sequence selected from any one of SEQ ID NO: 853-878 and 880-930 or a truncated amino acid sequence of any one of SEQ ID NO: 853-878 and 880-930, wherein the amino acid sequence comprises one or more amino acid substitutions such as from 1 to 5 amino acid substitutions. An amino acid substitution can be a conservative amino acid substitution.

An IL-7Rαγc agonist peptide provided by the present disclosure can comprise:
an amino acid sequence of any one of SEQ ID NO: 853-878 and 880-930;
a truncated amino acid sequence of any one of SEQ ID NO: 853-878 and 880-930;
an amino acid sequence of any one of SEQ ID NO: 853-878 and 880-930 comprising from 1 to 5 amino acid substitutions;
an amino acid sequence of any one of SEQ ID NO: 853-878 and 880-930 comprising from 1 to 5 flanking glycines on the N-terminus, the C-terminus, or both the N-terminus and the C-terminus; and/or
an amino acid sequence having greater than 60% sequence similarity to an amino acid sequence of any one of SEQ ID NO: 853-878 and 880-930.

An IL-7Rαγc agonist peptide can comprise an amino acid sequence of any one of SEQ ID NO: 853, 869, 878, 890-893, 901, and 916.

An IL-7Rαγc agonist peptide can comprise an amino acid sequence of any one of SEQ ID NO: 890-900.

An IL-7Rαγc agonist peptide can comprise an amino acid sequence of any one of SEQ ID NO: 891.

An IL-7Rαγc agonist peptide can bind to each of the IL-7Rα subunit and to the IL-7Rγc subunit, such as each of the hu-IL-7Rα subunit and to the hu-IL-7Rγc subunit with an IC₅₀ of less than 100 μm, less than 10 μm, less than 1 μm, less than 100 pM, less than 10 pM, or less than 1 pM.

IL-7Rαγc agonist peptides can be full agonists or partial agonists of IL-7R.

Examples of suitable IL-7Rαγc agonist peptides are disclosed, for example, in U.S. Publication No. 2021/0253670 A1, which is incorporated by reference in its entirety.

A culture medium can comprise a concentration of an IL-2Rβγc agonist peptide, for example, of greater than 1 nM, greater than 10 nM, greater than 100 nM, greater than 200 nM, greater than 500 nM, greater than 1,000 nM, or greater than 2,000 nM.

A culture medium can comprise a concentration of an IL-2Rβγc agonist peptide, for example, of less than 5,000 nM, less than 2,000 nM, less than 1,000 nM, less than 500 nM, less than 200 nM, less than 100 nM, less than 50 nM, or less than 10 nM.

A culture medium can comprise a concentration of an IL-2Rβγc agonist peptide, for example, of from 1 nM to 5,000 nM, from 10 nM, to 1,000 nM, or from 50 nM to 500 nM.

A culture medium can comprise a concentration of an IL-2Rβγc agonist peptide sufficient to stimulate the proliferation of a target immune cell population, such as the same target immune cell population being proliferated by incubation with the IL-7Rαγc agonist peptide.

A culture medium provided by the present disclosure can comprise an antibody directed to the target immune cell population. The antibody can be a stimulant for the proliferation of the target immune cell population. The antibody can be a monoclonal antibody.

For example, for a culture medium comprising a target T cell population, the antibody can be directed to the target immune cell population, such as a target T cell population.

For example, when the target T cell population comprises CD4+ T cells and CD8+ T cells, the antibody can comprise an anti-CD3 antibody and anti-CD28 antibody.

A culture medium can comprise a concentration of an antibody, for example, of greater than 0.1 ng/mL, greater than 1 ng/mL, greater than 5 ng/mL, greater than 10 ng/mL, greater than 50 ng/mL, greater than 100 ng/mL, or greater than 500 ng/mL.

A culture medium can comprise a concentration of an antibody, for example, of less than 500 ng/mL, less than 100 ng/mL, less than 50 ng/mL, less than 10 ng/mL, less than 5 ng/mL, less than 1 ng/mL, or less than 0.5 ng/mL.

A culture medium can comprise a concentration of an antibody for example, of from 0.1 ng/mL to 500 ng/mL, from 1 ng/mL to 100 ng/mL, or from 5 ng/mL to 50 ng/mL.

A culture medium can comprise a concentration of an antibody sufficient to augment the proliferation of the target immune cell population.

Methods of expanding a target population of T cells provided by the present disclosure can comprise a desirable or intended phenotype of immune cells such as T cells.

For example, methods provided by the present disclosure can provide a population of T cells enriched in Tcm cells and Tscm cells.

An immune cell growth medium comprising a first growth stimulant such as an IL-2Rβγc agonist peptide, a second growth stimulant such as an IL-7Rαγc agonist peptide, and optionally one or more antibodies can produce a subpopulation of T cells or a combination of subpopulations of T cells enriched in Tcm and Tscm cells.

For example, a population of T cells enriched in Tcm and/or Tscm cells can comprise greater than 60%, 65%, 70%, 75%, or 80% combined Tcm cells and Tscm cells, wherein percent is based on the total number of T cells in the sample.

A population of T cells (e.g., genetically modified T cells) enriched in Tcm and Tscm cells can comprise 60% to 90% of combined Tcm cells and Tscm cells.

A population of T cells enriched in Tcm and/or Tscm cells can comprise greater than 20%, 25%, 30%, 35%, 40%, 45%, 50%, 55%, or 60% Tscm cells, where percent is based on the total number of T cells.

Methods provided by the present disclosure comprise expanding a target immune cell population of an initial immune cell population by incubating the initial immune cell population in the presence of an IL-2Rβγc agonist peptide.

A target immune cell population can comprise immune cells for which cell growth is stimulated in the presence of an IL-2Rαγc agonist peptide, either alone or in combination with another growth stimulant for the target immune cell population.

For example, a target immune cell population can comprise a target T cell population.

A target immune cell population can comprise, for example, a target Tcm cell population, a target Tscm cell population, or a target Tcm cell population and a target Tscm cell population.

A target immune cell population can comprise, for example, a population of CD4+ cells, a population of CD8+ cells, or a population of CD4+ and CD8+ cells.

A target immune cell population can comprise a population of engineered immune cells.

A population of engineered immune cells can be engineered to express one or more chimeric antigen receptors.

An initial immune cell population can be derived from a sample obtained from a patient.

An initial immune cell population can be derived from a sample obtained from a donor.

An initial immune cell population can be derived from a suitable tissue.

An initial immune cell population can comprise allogeneic immune cells.

An initial immune cell population can comprise autologous immune cells.

Incubating the initial immune cell population to expand the target immune cell population can comprise incubating the immune cells in a culture medium provided by the present disclosure.

Following incubation in the presence of an IL-2Rβγc agonist peptide, the concentration of the target immune cell population can be expanded, for example, by greater than 10-times, greater than 100-times, greater than 1,000-times, greater than 10,000-times, greater than 100,000-times, or greater than 1,000,000-times, compared to the concentration of the target immune cell population before incubation with the IL-2Rβγc agonist peptide.

The expansion of the target immune cell population can be determined, for example, at greater than 3 days, greater than 5 days, greater than 7 days, greater than 10 days, greater than 20 days, or greater than 30 days, after the beginning of incubation.

The expansion of the target immune cell population can be determined, for example, from 3 days to 50 days, from 3 days to 40 days, from 3 days to 30 days, or from 3 days to 20 days, after the beginning of incubation.

Incubating can comprise incubating the target immune cell population with a second growth stimulant for the target immune cell population. For example, a second growth stimulant can comprise an IL-7Rαγc agonist peptide, an IL-15Rβγc agonist peptide, or a combination thereof.

The target immune cell population can be expanded by incubating in a medium comprising both the IL-2Rβγc agonist peptide and a second growth stimulant such as an IL-7Rαγc agonist peptide.

The target immune cell population can be expanded by incubating in a medium comprising the IL-2Rβγc agonist peptide without a second growth stimulant and subsequently incubating in a medium comprising the second growth stimulant without the IL-2Rβγc agonist peptide. Thus, the target immune cell population can be expanded by incubating the target immune cell population in different growth media during the course of expansion, where at least one of the growth media comprises an IL-2Rβγc agonist peptide.

An immune cell subpopulation can be expanded by incubating an immune cell population in the presence of an IL-2Rβγc agonist peptide.

The immune cell population can be expanded by incubating an immune cell population in the presence of an IL-2Rβγc agonist peptide provided by the present disclosure as the only stimulant of cell proliferation.

The immune cell population can be expanded by incubating an immune cell population in the presence of an IL-2Rβγc agonist peptide in combination with a second stimulant of cell growth proliferation.

The second stimulant of cell growth proliferation can be, for example, an agent that stimulates a CD3 TCR complex; an anti-CD3 antibody or antigen-binding fragment thereof, an anti CD28 antibody or antigen-binding fragment thereof, an anti-CD2 antibody or antigen-binding fragment thereof a protein kinase C activator, a growth factor such as a T cell growth factor, a peptide such as an IL-7Rαγc agonist peptide or an IL-15αγc agonist peptide, or a combination of any of the foregoing.

For example, a population of T cells can be incubated in the presence of an anti-CD3 antibody and an anti-CD28 antibody under conditions appropriate for stimulating the proliferation of the T cells. An anti-CD3 antibody and an anti-CD28 antibody can be bonded to or immobilized on a bead, plate or other substrates.

A second stimulant of cell growth stimulation can be an IL-7Rαγc agonist peptide.

A suitable combination of second stimulants of cell growth proliferation can comprise, for example, an anti-CD3 antibody and an anti-CD28 antibody.

A suitable combination of second stimulants of cell growth proliferation can comprise, for example, an anti-CD3 antibody, an anti-CD28 antibody, and an IL-7Rαγc agonist peptide.

Immune cells can be incubated with an IL-2Rβγc agonist peptide provided by the present disclosure wherein the concentration of the IL-2Rβγc agonist peptide is greater than 1 nM, greater than 10 nM, greater than 100 nM, greater than 250 nM, greater than 500 nM, greater than 1,000 nM, or greater than 5,000 nM.

A concentration of an IL-2Rβγc agonist peptide can be, for example, greater than 0.1 μg/mL, greater than 0.5 μg/mL, greater than 1 μg/mL, greater than 5 μg/mL, or greater than 10 μg/mL. A concentration of an IL-2Rβγc agonist peptide in the cell growth medium can be, for example, from 0.01 μg/mL to 100 μg/mL, from 0.1 μg/mL to 10 μg/mL, or from 0.5 μg/mL to 5 μg/mL.

A concentration of an antibody such as an anti-CD3 antibody or an anti-CD28 antibody in the cell growth media can be, for example, greater than 0.1 ng/mL, greater than 0.5 ng/mL, greater than 1 ng/mL, greater than 5 ng/mL, greater than 10 ng/mL, greater than 50 ng/mL, greater than 100 ng/mL, or greater than 500 ng/mL.

A concentration of an antibody such as an anti-CD3 antibody or an anti-CD28 antibody in the cell growth media can be, for example, from 0.1 ng/mL to 1,000 ng/mL, from 1 ng/mL to 100 ng/mL, or from 5 ng/mL to 50 ng/mL.

During cell proliferation, fresh media containing the stimulants of cell proliferation can be provided, for example, every day, every two days, every three days, every four days, every five days, every six days, every week, every two weeks, every three weeks or every four weeks.

The immune cells, such as T cells, can be activated and expanded, either prior to or after genetic modification of the immune cells. Immune cells can be activated and expanded prior to genetic modification of the immune cells. Immune cells can be activated and expanded after genetic modification of the immune cells, such as engineered immune cells

Immune cell expansion can be accomplished by incubating the targeted immune cells in any suitable culture medium.

An immune-cell expansion medium provided by the present disclosure can comprise an IL-2Rβγc agonist peptide provided by the present disclosure.

For example, a targeted immune cell population, such as a targeted T cell population, can be stimulated in vitro by contact with, for example, an anti-CD3 antibody and/or anti-CD28 antibody, or antigen-binding fragment thereof. For co-stimulation of an accessory molecule on the surface of the immune cells, a ligand that binds the accessory molecule can be used. For example, a population of immune cells can be contacted with an anti-CD3 antibody and an anti-CD28 antibody, under conditions appropriate for stimulating the proliferation of the immune cells. The anti-CD3 antibody and an anti-CD28 antibody can be immobilized. For example, the antibody can be displayed on a bead, matrix, plate, or other substrates.

Methods provided by the present disclosure can comprise incubating a population of immune cells such as T cells with a second proliferation stimulant, such as anti-CD3 and anti-CD28 antibodies, and a cytokine such as IL-7 and/or IL-15. The second proliferation stimulant can be an IL-7Rαγc agonist peptide or an IL-15Rβγc agonist peptide. The antibodies can be immobilized on a substrate. For example, anti-CD3 and anti-CD28 antibodies can be attached to a bead such as a Dynabead® system, a CD3/CD28 activator/stimulator system for physiological activation of human immune cells such as human T cells. Immune cells can be activated and stimulated to proliferate with suitable antibodies and other cytokines, in addition to IL-7 and IL-15

In methods provided by the present disclosure, a targeted immune cell population, such as a targeted T cell population, can be expanded by activating a target immune cell population by incubating a population of primary cells with a suitable antibody and expanding the activated target immune cell population by incubating the activated target immune cell population in the presence of an IL-2Rβγc agonist peptide.

Methods provided by the present disclosure comprise culturing immune cells such as T cells using a cell growth medium comprising an IL-2Rβγc agonist peptide provided by the present disclosure. In addition to an IL-2Rβγc agonist peptide, the cells can be cultured with a second stimulant of immune cell proliferation such as an IL-7Rαγc agonist peptide, an IL-15Rβγc agonist peptide, or a combination thereof. The cells can be cultured for a duration, for example, of less than 1 week, for one week, for two weeks, for three weeks, for weeks, or for five weeks. Cells can be cultured, for example, for from 1 day to 7 days, from 1 day to 5 days, or from 1 day to 3 days. Cells can be cultured, for example, for 1 day, 2 days, 3 days, 4 days, 5 days, 6 days, or 7 days.

Cells can be cultured for a suitable duration to provide a desired enrichment of a target subpopulation of immune cells. For example, a suitable enrichment of a target subpopulation of immune cells such as T cells can increase the relative number of immune cells by greater than 10 times, greater than 100 times, greater than 200 times, greater than 400 times, greater than 600 times, greater than 800 times, or greater than 1,000 times.

Cells useful in the methods provided by the present disclosure can be derived from primary cells such as primary human cells. The primary cells can be derived from a biological sample such as, for example, tissue, fluid, and other samples taken directly from a subject or patient, as well as samples resulting from one or more processing steps, such as separation, centrifugation, genetic engineering such as transduction with a viral vector, washing, and/or incubation. The biological sample can be a sample obtained directly from a biological source or a biological sample that is processed. Biological samples include, for example, body fluids, such as blood, plasma, serum, cerebrospinal fluid, synovial fluid, urine and sweat, tissue and organ samples, including processed samples derived from any of the foregoing. Biological samples can be obtained, for example, from peripheral blood mononuclear cells, bone marrow, lymph node tissue, cord blood, thymus tissue, stem cell- or iPSC-derived immune cells, tissue from a site of infection, ascites, pleural effusion, spleen tissue, and tumors.

Cells suitable for culturing using the media and methods provided by the present disclosure include immune cells.

Cells can be derived from a healthy donor, from a patient diagnosed with a disease such as cancer, an autoimmune disease, or a viral disease, or from a patient diagnosed with an infection. Cells can be part of a mixed population of cells that present different phenotypic characteristics.

Cells can be obtained from a subject or patient who will ultimately receive the expanded immune cells. Cells can be obtained from a donor, who is a different individual from the subject who will receive the expanded immune cells.

Immune cells can comprise T cells. T cells can be obtained from a number of sources, including peripheral blood mononuclear cells (PBMCs), bone marrow, lymph nodes tissue, cord blood, thymus tissue, stem cell- or iPSC-derived T cells, tissue from a site of infection, ascites, pleural effusion, spleen tissue, and tumors. T cells can be obtained from a volume of blood collected from a subject using any number of techniques known to the skilled person.

Cells can be obtained from the circulating blood of an individual by apheresis. An apheresis product can include lymphocytes, including T cells, monocytes, granulocytes, B cells, other nucleated white blood cells, red blood cells, and platelets. Cells collected by apheresis can be washed to remove the plasma fraction and placed in an appropriate buffer or media for subsequent processing.

PBMCs can be used directly for genetic modification with the immune cells, such as CARS or TCRs, using methods provided by the present disclosure. After isolating the PBMCs, T lymphocytes can be further isolated and both cytotoxic and helper T lymphocytes can be sorted into naïve, memory, and effector T cell subpopulations either before or after genetic modification and/or expansion.

PBMCs can be isolated from whole blood by centrifugation through a density gradient to remove red blood cells and granulocytes. A specific subpopulation of T cells, such as CCR7+, CD95+, CD122+, CD27+, CD69+, CD127+, CD28+, CD3+, CD4+, CD8+, CD+, CD25+, CD62L+, CD45RA+, and CD45RO+ T cells can be further isolated by positive or negative selection techniques. A specific subpopulation of T cells can comprise CD4+ cells and CD8+ cells.

For example, enrichment of an immune cell population such as a T cell population by negative selection can be accomplished with a combination of antibodies directed to surface markers unique to the negatively selected cells. Flow cytometry and cell sorting can also be used to isolate cell populations of interest for use in the present disclosure.

In some embodiments, a population of T cells is enriched for CD4+ cells, CD8+ cells, or a mixture of both CD4+ and CD8+ cells.

T cells can be further sorted, for example, naïve, central memory, and effector cells by identifying cell surface antigens that are associated with each of these types of T cells. For example, CD4+ T cells and CD8+ T cells can be sorted into naive, central memory, and effector cells by identifying cell populations with cell surface antigens.

Cell growth media and methods provided by the present disclosure can be useful for in vitro expansion of immune cells such as engineered immune cells such as CAR-T cells.

Engineered immune cells can be allogeneic or autologous immune cells.

An engineered immune cell can be a T cell such as an inflammatory a T-lymphocyte, a cytotoxic T-lymphocyte, a regulatory T-lymphocyte, a helper T-lymphocyte, a tumor-infiltrating lymphocyte (TIL), an NK cell, an NK-T-cell, a gamma delta T cell, a TCR-expressing cell, a dendritic cell, a killer dendritic cell, a mast cell, a B-cell, or a combination of any of the foregoing. An engineered immune cell can be derived, for example, from a CD4+T-lymphocyte and/or CD8+T-lymphocytes. An engineered immune cell can be a T cell.

An engineered immune cell can be derived, for example, from a stem cell. Stem cells can be adult stem cells, non-human embryonic stem cells, more particularly non-human stem cells, cord blood stem cells, progenitor cells, bone marrow stem cells, induced pluripotent stem cells, totipotent stem cells, hematopoietic stem cells, or combinations of any of the foregoing.

Cells can be obtained or prepared from peripheral blood. For example, cells can be obtained or prepared from peripheral blood mononuclear cells (PBMCs), from bone marrow, or from umbilical cord blood. Cells can be human cells. Cells can be transfected or transduced by a nucleic acid vector using methods, such as electroporation, sonoporation, biolistics, lipid transfection, polymer transfection, nanoparticles, or polyplexes.

Engineered immune cells can comprise an antigen binding agent such as an antigen binding domain, an antibody, or an antibody fragment.

An engineered immune cell can comprise a population of CARs where each CAR comprises an extracellular antigen-binding domain. An engineered immune cell can comprise a population of CARS where each CAR comprises the same extracellular antigen-binding domains.

A chimeric antigen receptor (CAR) refers to a protein that specifically recognizes target antigen, such as target antigen on cancer cells. When bound to the target antigen, the CAR can activate an immune cell to attack and destroy the cell expressing the target antigen.

A CAR can incorporate costimulatory or signaling domains to increase its potency.

A targeted immune cell population can be engineered to express an antigen binding moiety.

An antigen binding moiety can be directed to any specific antigen such as, for example, an antigen expressed on the surface of a tumor cell or in a tumor cell environment, an antigen expressed on an immune cell, an antigen expressed on the surface of a cell expressing predominantly the IL-2Rβ and IL-2Rγc subunits of IL-2R such as CD4+ T-cells and CD8+ T-cells, or NK cells.

Examples of suitable antigen targets expressed on tumor cells include fibroblast activation protein (FAP), the A1 domain of tenascin-C (TnC A1), the A2 domain of tenascin-C (TnC A2), the extra-cellular domain B of fibronectin (EDB), carcinoembryonic antigen (CEA), and the melanoma-associated chondroitin sulfate proteoglycan (MCSP).

Other examples of suitable tumor antigens that can be used for targeting include MAGE, MART-1/Melan-A, gplOO, Dipeptidyl peptidase 4 (DPP-4), adenosine deaminase-binding protein (ADAbp), cyclophilin b, Colorectal associated antigen (CRC)-0017-A/GA733, Carcinoembryonic Antigen (CEA) and its immunogenic epitopes CAP-1 and CAP-2, etv6, amll, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-AS, MAGE-A6, MAGE-A7, MAGE-AS, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-CS), GAGE-family of tumor antigens such as GAGE-I, GAGE-2, GAGE-3, GAGE-4, GAGE-5, GAGE-6, GAGE-7, GAGE-8, GAGE-9, BAGE, RAGE, LAGE-I, NAG, GnT-V, MUM-1, CDK4, tyrosinase, p53, MUC family, HER2/neu, p21ras, RCAS1, a-fetoprotein, E-cadherin, a-catenin, -catenin and y-catenin, p120ctn, gplOO Pme1117, PRAME, NY-ES0-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, lmp-1, PIA, EBY-encoded nuclear antigen (EBNA)-1, brain glycogen phosphorylase, SSX-1, SSX-2 (HOM-MEL-40), SSX-1, SSX-4, SSX-5, SCP-1 and CT-7, and cerbB-2.

Examples of viral antigens include influenza virus hemagglutinin, Epstein-Barr virus LMP-1, hepatitis C virus E2 glycoprotein, HIV gp160, and HIV gp120.

Examples of ECM antigens include syndecan, heparanase, integrins, osteopontin, link proteins, cadherins, laminin, laminin type EGF, lectin, fibronectin, notch, tenascin, and matrixin.

Targeted IL-2Rβγc agonist peptides can be configured to bind, for example, to a cell surface antigen selected from FAP, Her2, EGFR, IGF-1R, CD2 (T-cell surface antigen), CD3 (heteromultimer associated with the TCR), CD22 (B-cell receptor), CD23 (low affinity IgE receptor), CD30 (cytokine receptor), CD33 (myeloid cell surface antigen), CD40 (tumor necrosis factor receptor), IL-6R (IL6 receptor), CD20, MCSP, and PDGFR (platelet-derived growth factor receptor).

A targeted IL-2Rβγc agonist peptide can comprise an antigen binding moiety capable of binding to an antigen or to a receptor expressed on the surface of a cell that also expresses IL-2R. Examples of cells expressing IL-2R include, for example, naïve T-cells, memory T-cells, and activated T-cells in CD8+ T-cells, CD4+ T-cells.

A targeted IL-2Rβγc agonist peptide\w can comprise an antigen binding moiety capable of binding to an antigen or a receptor expressed by a cell that expresses the IL-2Rβ and IL-2Rγc subunits of IL-2R. Examples of cells expressing the IL-2Rβ and Rγc subunits of IL-2R include, for example, naïve T-cells, memory T-cells, and activated T-cells in CD4+ T-cells and CD8+ T-cells.

Examples of antigens expressed on the surface of naïve CD4+ T-cells include CD4−, CD45RA, CCR7, CD27, and CD28.

Examples of antigens expressed on the surface of naïve CD8+ T-cells include CD8, CD45RA, CCR7, CD27, and CD28.

Examples of antigens expressed on the surface of CD4+ T-cells include Th1 cell markers such as CD4, CXCR3, CCR5, and IL12Rβ2; Th2 cell markers such as CD4, CCR4, and IL4Ra; Th9 cell markers such as CD4, CCR3, and CCR5; Th17 cell markers such as CD4, CCR6, CCR4, and IL23R; Th22 cell markers such as CD4, CCR10, CCR4, and CCR6; Treg cell markers such as CD4, CD127, CD25, and CTLA-4; and Tth cell markers such as CD4, CXCR5, CD40L, PD-1, and ICOS.

Examples of antigens expressed on the surface of cytotoxic CD8+ T cells include CD8.

Examples of memory T-cell antigens include CCR5, CCR7, CD11a, CD27, CD28, CD45RA, CD45RO, CD57, CD95, and CD62L.

Examples of naive T-cell antigens include CD45RA, CCR7, CD62L, CD27, CD28, CD127, and CD132.

A targeted IL-2Rβγc agonist peptide can comprise an antigen binding moiety capable of binding to an antigen or receptor expressed on the surface of cells, having a role in regulating the immune response.

Examples of antigens expressed by cells associated with regulating the immune response include PD-1, CTLA-4, CD20, and CD30.

Immune cells can be genetically modified prior to expansion.

The process for manufacturing allogeneic CAR-T therapy involves first harvesting healthy, selected, screened, and tested immune cells such as T cells from healthy donors.

Immune cells such as T cells are engineered to express CARs, which recognize certain cell surface proteins that are expressed in hematologic or solid tumors. Allogeneic T cells can be gene edited to reduce the risk of graft versus host disease (GvHD) and to prevent allogeneic rejection. For example, a T cell receptor gene such as TCRa or TCR˜ can be knocked out to avoid GvHD. The CD52 gene can be knocked out to render the CAR T product resistant to anti-CD52 antibody treatment. Anti-CD52 antibody treatment can be used to suppress the host immune system and to allow the CAR T to stay engrafted to achieve full therapeutic impact. After gene editing, the engineered T cells then undergo a purification step and then cryopreserved in vials for delivery to patients.

Autologous chimeric antigen receptor (CAR) T cell therapy involves collecting T cells from a patient and genetically engineering the T cells to express CARS that recognize target expressed on the cell surface of one or more specific cancer cells of the patient. The engineered T cells can be cryopreserved and subsequently administered to the patient.

Methods of manufacturing an immune cell population comprise activating a target immune cell population from primary cells to provide an activated target immune cell population; and expanding the activated target immune cell population, wherein expanding comprises incubating the activated target immune cell population in the presence of an IL-2Rbγc agonist peptide to provide an expanded target immune cell population.

The target immune cell population can comprise, for example, T cells and/or NK cells.

The population of primary cells can comprise any suitable populations in primary cells that comprise immune cells.

A population of primary cells can be obtained from any suitable source such as disclosed herein.

A population of primary cells can comprise human primary cells.

A population of human primary cells can comprise mononuclear cells.

A mononuclear cell refers to a cell found in blood that has a single, round nucleus such as a lymphocyte or a monocyte. Mononuclear cells can be obtained from body fluids such as blood and including, for example, peripheral blood, bone marrow and cord blood by known methods such as centrifugation, magnetic beads and flow cytometry. Mononuclear cells may be those derived from stem cells such as iPS cells, ES cells and somatic stem cells. A mononuclear cell can be a peripheral blood mononuclear cell (PBMC).

Mononuclear cells can comprise peripheral blood mononuclear cells (PBMCs) obtained from a patient or comprise peripheral blood mononuclear cells (PBMCs) obtained from a donor.

A biological sample from which immune cells are derived or isolated can be blood or a blood-derived sample or can be derived from an apheresis or leukapheresis product. Suitable examples from which immune cells can be derived include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organs.

Cell samples can also be obtained in the context of cell therapy such as adoptive cell therapy, from autologous and allogeneic sources.

Immune cells for use in partial reprogramming methods can be derived from cell lines such as T cell lines. Immune cells can be obtained from a xenogeneic source such as, for example, from mouse, rat, non-human primate, and pig.

In partial reprogramming methods, T cells can be isolated from a source and partially reprogrammed to ameliorate aging and improve the function of the T cells. Examples of suitable source cells include, but are not limited to, peripheral blood mononuclear cells (PBMCs). T cells for use in the methods herein may include but are not limited to cultured T cells, e.g., primary T cells or T cells from a suitable cultured T cell line, or T cells obtained from a mammal. If obtained from a mammal, the source cells can be obtained from numerous sources, including but not limited to blood, bone marrow, lymph node, tumor, thymus, spleen, or other tissues or fluids. Source cells can also be enriched for or purified. The T cells can be any type of T cells and can be of any developmental stage, including for example, CD4+ helper T cells, e.g., Th1 and Th2 cells, CD8+ cytotoxic T cells, a mixture of CD4+ and CD8+T cells, peripheral blood mononuclear cells (PBMCs), peripheral blood leukocytes (PBLs), tumor-infiltrating cells (TILs), memory T cells, and naive T cells.

T cells can be isolated from tumors, such as tumor-infiltrating lymphocytes such as T cells for use in a partial reprogramming method.

A tumor-infiltrating lymphocytes or TILs refers to a population of cells originally obtained as cells that have left the bloodstream of a subject and migrated into a tumor. TILs include, for example, CD8+ cytotoxic T cells (lymphocytes), CD4+T helper cells, and natural killer cells. TILs include both primary and secondary TILs. “Primary TILs” refer to TILs obtained from patient tissue samples. TILs can be categorized as expressing a biomarker such as CD4, CD8, TCRa, CD27, CD28, CD56, CCR7, CD45RA, CD45RO, CD95, PD-1, CD25, or a combination of any of the foregoing.

A source cell can have a naive T cell (Tn) phenotype, a central memory T cell (Tcm) phenotype, an effector memory T cell (Tem) phenotype, or a stem-like T cell (Tscm).

The phenotypes of Tn, Tcm and Tem cells are known in the art and are described elsewhere herein. For example, CCR7 and CD62L are expressed by Tn and Tcm cells but are not expressed by Tem cells. The transcription factors LEF1, FOXPl and KLF7 are expressed by Tn and Tcm cells but are not expressed by Tem cells. CD45RO and KLRG1 are not expressed by Tn cells but are expressed by Tem cells. Alternatively, or additionally, Tn and Tcm cells may be characterized by longer telomeres as compared to those of Tem cells.

A specific subpopulation of T cells such as, for example, CD3+, CD45+, CD137+, CD25+, CD28+, CD4+, CD8+, CD45RA+, GITR+, and/or CD45RO+ T cells, can be isolated by positive or negative selection techniques such as using fluorescence-based or magnetic-based cell sorting. For example, T cells can be isolated by incubation with any of a variety of commercially available antibody-conjugated beads, such as Dynabeads®, CELLection™, DETACHaBEAD™ (Thermo Fisher Scientific) or MACS® cell separation products (Miltenyi Biotec), for a duration sufficient for positive selection of the desired T cells or negative selection for removal of unwanted cells.

In methods provided by the present disclosure, a target immune cell population can be any suitable immune cell population for which proliferation is stimulated by incubation with an IL-2Rβγc agonist peptide.

For example, a target immune cell population can comprise CD4+ T cells and CD8+ T cells.

In methods of the present disclosure, activating the target immune cell population comprises incubating the target immune cell population in the presence of an IL-2Rβγc agonist peptide.

The IL-2Rβγc agonist peptide can be immobilized on a substrate such as, for example, a bead, matrix, or plate.

For example, when the target immune cell population comprises CD4+ T cells and CD8+ T cells, the CD4+ T cells and CD8+ T cells can be incubated in the presence of an anti-CD3 monoclonal antibody and an anti-CD28 monoclonal antibody.

Activating a target immune cell population can comprise incubating a population of primary cells comprising the target immune cell population in the presence of an antibody to the target immune cell population.

The antibody can be immobilized on a substrate including any of the substrates disclosed herein.

Expansion of the activated target immune cell population can comprise incubating the activated target immune cell population in the presence of a second growth stimulant for the proliferation of the activated target immune cell population.

The second growth stimulant can be, for example, a suitable agonist peptide such an IL-7Rαγc agonist peptide, an IL-15Rβγc agonist peptide, a cytokine such as a natural cytokine or an engineered cytokine, an antibody, or a combination of any of the foregoing.

The second stimulant can be an agonist peptide. For example, the second stimulant can be an IL-7Rαγc agonist peptide, an IL-15Rβγc agonist peptide, or a combination thereof.

Expanding an activated target immune cell population can comprise incubating the activated target immune cell population in the presence of an IL-2Rβγc agonist peptide together with a second growth stimulant such as an IL-7Rαγc agonist peptide and/or an IL-15Rβγc agonist peptide.

Expanding an activated target immune cell population can comprise incubating the activated target immune cell population in the presence of an IL-2Rβγc agonist peptide in the absence of a second growth stimulant such as an IL-7Rαγc agonist peptide and/or an IL-15Rβγc agonist peptide.

Expanding an activated target immune cell population can comprise incubating the activated target immune cell population in the presence of an IL-2Rβγc agonist peptide. Expanding an activated target immune cell population can comprise incubating the activated target immune cell population in the presence of an IL-2Rβγc agonist peptide for a period of time, and then incubating the activated target immune cell population in the presence of a second growth stimulant and in the absence of the IL-2Rβγc agonist peptide for a period of time.

Methods of manufacturing provided by the present disclosure can comprise, for example, before activating the target immune cell population, enriching the population of primary cells with the target immune cell population.

Enriching the target immune cell population can comprise, for example, increasing the number of target immune cell population by 10 times, 10² times, 10³ times, 10⁴ times, 10⁵ times, or 10⁶ times compared to the initial target immune cell population in the sample, where the number of target immune cells is based on the number of cells per volume.

For example, for in vitro sorting of a population of immune cells, where a subset of the population of immune cells comprises engineered immune cells expressing an antigen-specific CAR comprising epitopes specific for monoclonal antibodies, can comprise contacting the population of immune cells with a monoclonal antibody specific for the epitopes and selecting the immune cells that bind to the monoclonal antibody to obtain a population of cells enriched in engineered immune cells expressing an antigen-specific CAR-T.

A monoclonal antibody specific for an epitope can be conjugated to a fluorophore. In this method, the step of selecting the cells that bind to the monoclonal antibody can be done by Fluorescence Activated Cell Sorting (FACS).

A monoclonal antibody specific for an epitope can be conjugated to a magnetic particle. In this method, the step of selecting the cells that bind to the monoclonal antibody can be done by Magnetic Activated Cell Sorting (MACS).

Methods of manufacturing provided by the present disclosure can comprise, for example, after activating the target immune cell population, engineering the activated target immune cell population to express a cell surface receptor.

Engineering can comprise, for example, transducing the activated target immune cell population with a viral vector to provide a transduced activated target immune cell population.

A viral vector can comprise, for example, a viral vector expressing a T cell receptor (TCR) or a chimeric antigen receptor (CAR).

A TCR or CAR-T can recognize a specific antigenic moiety on a surface of a target cell.

An antigenic moiety can be, for example, an MHC class I dependent antigenic moiety, an MHC class II dependent antigenic moiety, or a combination thereof.

Methods of manufacturing provided by the present disclosure can comprise, for example, before activating the target immune cell population, enriching the population of primary cells with the target immune cell population.

For example, the target immune cell population can be enriched, for example, by greater than 10 times, greater than 10² times, or greater than 10³ times, greater than 10⁴ times, greater than 10⁵ times, greater than 10⁶ times, or greater than 10⁷ times. Enrichment refers to the increased concentration of the target immune cell population in the enriched media compared to the concentration of the target immune cell population before incubation according to methods provided by the present disclosure. For example, for an initial concentration of the target immune cell population of 10³ cells/mL, and an enriched concentration of the target immune cell population of 10⁶ cells/mL, the concentration of the target immune cell population is increased or enriched by 10³ times.

In methods of manufacturing where the target immune cell population comprises CD4+ T cells and CD8+ T cells, the population of primary cells can be enriched for CD4+ T cells and CD8+ T cells.

In methods of manufacturing provided by the present disclosure, enriching can comprise removing or increasing the concentration of a specific cell population of the population of primary cells.

For example, enriching can comprise removing CD57+ T cells from the population of primary cells to provide a depleted population of CD57+ cells.

The production and manufacture of engineered immune cells can comprise harvesting immune cells from a patient followed by in vitro expansion of a target population of immune cells using methods provided by the present disclosure to provide a desired enrichment of the target population of immune cells having the desired cell phenotype or combination of phenotypes.

For example, T cell expansion can result in a desired combination of T cells that includes subsets of naive T cells (Tn), memory T cells including stem-cell like memory (Tscm), central memory (Tcm), and effector memory (Tem) T cells), and effector (Teff) T cells. The varying amounts of different T cell subsets can affect the therapeutic profile and efficacy of the resulting engineered T cells. In particular, T cell expansion methods that result in enriched amounts of T cells in early differentiation stages can be therapeutically useful.

Cell growth media provided by the present disclosure can be used for T-cell expansion in which the proportion of Tscm and/or Tcm is enriched. Tscm cells are the least-differentiated type of memory T cells and, for adoptive T-cell therapy, can be useful in promoting prolonged in vivo T-cell proliferation following administration of the engineered cells to a patient. Such cell culture media and immune cells prepared using culture media provided by the present disclosure can result in more potent adoptive cell transfer therapies including, for example, CAR-T therapies.

Cell growth media comprising combinations of different stimulants of cell proliferation in combination with an extracellular modulator of cell metabolism can result in proliferation/expansion of a desired immune cell population having a specific cell phenotype or a combination of cells having different phenotypes.

A population of immune cells provided by the present disclosure can comprise a population of immune cells that are enriched in a target immune cell population compared to that of the initial population of primary cells.

For example, an enriched population of immune cells can have a concentration of a target population of immune cells that is greater than 10 times, greater than 10² times, greater than 10³ times, or greater than 10⁴ times that of the concentration of the initial population of primary cells.

An enriched population of immune cells can comprise, for example, an enriched population of T cells.

For example, an enriched population of immune cells provided by the present disclosure can comprise an enriched population of CD4+ T cells and CD8+ T cells.

An enriched population of immune cells provided by the present disclosure can be prepared using a culture medium provided by the present disclosure.

An enriched population of immune cells provided by the present disclosure can be prepared using a method of expanding a target immune cell population provided by the present disclosure.

An enriched population of immune cells provided by the present disclosure can be prepared using a method of cell manufacturing provided by the present disclosure.

An enriched population of T cells can be harvested from the culture medium, washed, concentrated, and combined with pharmaceutically acceptable excipients to provide a pharmaceutical composition suitable for administration to a patient. A pharmaceutical composition can be formulated for infusion. Examples of suitable infusion media include, for example, an isotonic medium formulation, such as normal saline, Normosol™ R (Abbott), or Plasma-Lyte™ A (Baxter), but also 5% dextrose in water or Ringer's lactate can be utilized. The infusion medium can be supplemented with human serum albumin.

A therapeutically effective number of T cells can be, for example, greater than 10⁴ cells, greater than 10⁶ cells, greater than 10⁸ cells, greater than 10¹⁰ cells, or greater than 10¹² cells.

A therapeutically effective concentration of T cells for administering to a patient can be, for example greater than 10⁴ cells/mL, greater than 10⁶ cells/mL, or greater than 10⁸ cells/mL.

A therapeutically effective number of cells can range, for example, from 10⁵ to 10¹² cells per kg patient weight.

CAR-T treatments can be administered multiple times at dosages within these ranges. The cells can be autologous, allogeneic, or heterologous to the patient undergoing therapy.

A pharmaceutical composition provided by the present disclosure can comprise, for example, an enriched population of immune cells provided by the present disclosure, such as an enriched population of T cells such as an enriched population of CD4+ T cells and CD8+ T cells.

A pharmaceutical composition provided by the present disclosure can comprise a therapeutically effective concentration of an enriched population of immune cells provided by the present disclosure for treating a disease in a patient.

A pharmaceutical composition provided by the present disclosure can comprise an enriched population of immune cells provided by the present disclosure in combination with one or more pharmaceutically acceptable excipients.

A pharmaceutical composition provided by the present disclosure can be an intravenous formulation.

Aspects of the present invention include immobilized IL-2Rβγc agonist peptides.

An immobilized IL-2Rβγc agonist peptide comprises an IL-2Rβγc agonist peptide such as an IL-2Rβγc agonist peptide provided by the present disclosure bound to a substrate.

A substrate can comprise any suitable substrate that can be added to a culture medium. The substrate prevents or minimizes the IL-2Rβγc agonist peptides from being internalized into cells.

Examples of suitable substrates include beads, matrices, and plates.

An IL-2Rβγc agonist peptide such as an IL-2Rβγc agonist provided by the present disclosure can be bound to a substrate through a substrate linker.

A substrate linker can comprise, for example, a suitable peptidyl linker and/or a synthetic linker as disclosed herein.

Kits provided by the present disclosure can comprise an IL-2Rβγc agonist peptide provided by the present disclosure. An IL-2Rβγc agonist peptide can be provided, for example, as a lyophilizate or as a stable solution. A kit can comprise a culture medium containing an IL-2Rβγc agonist peptide provided by the present disclosure. An IL-2Rβγc agonist peptide can be provided as free peptide or can be bound to a substrate such as bead or matrix.

Kits can further comprise, for example, a second stimulant of immune cell proliferation such as an IL-7Rαγc agonist peptide, an IL-15βγc agonist peptide, or a combination thereof.

A kit can comprise a monoclonal antibody for activating a target immune cell population. For example, an antibody can comprise an anti-CD3 monoclonal antibody, and anti-CD28 monoclonal antibody, or a combination thereof.

An enriched or expanded immune cell population provided by the present disclosure, can be incorporated into pharmaceutical compositions to be administered to a patient by any appropriate route of administration including intradermal, intramuscular, intraperitoneal, intravenous, subcutaneous, intranasal, epidural, oral, peroral, sublingual, intracerebral, intravaginal, transdermal, rectal, inhalation, or topical. A pharmaceutical composition provided by the present disclosure can be an injectable formulation. Pharmaceutical compositions provided by the present disclosure can be injectable intravenous formulations. Pharmaceutical compositions provided by the present disclosure can be oral formulations. Oral formulations may be oral dosage forms. A pharmaceutical composition may be formulated for intravenous administration or for subcutaneous administration.

Pharmaceutical compositions provided by the present disclosure may comprise a therapeutically effective amount of an enriched immune cell population together with a suitable amount of one or more pharmaceutically acceptable vehicles so as to provide a composition for proper administration to a patient. Suitable pharmaceutical vehicles and methods of preparing pharmaceutical compositions are described in the art.

Accordingly, it is within the capability of those of skill in the art to assay and use IL-2Rβγc agonist peptides and/or pharmaceutical compositions thereof for therapy.

Enriched immune cell populations, and/or pharmaceutical composition thereof can generally be used in an amount effective to achieve the intended purpose. For use to treat a disease such as cancer, an autoimmune disease or an inflammatory disease, an enriched immune cell population and/or pharmaceutical composition thereof, may be administered or applied in a therapeutically effective amount.

The amount of an enriched immune cell population and/or pharmaceutical composition of any of the foregoing that will be effective in the treatment of a particular disorder or condition disclosed herein will depend in part on the nature of the disorder or condition, and can be determined by standard clinical techniques known in the art. In addition, in vitro or in vivo assays may optionally be employed to help identify optimal dosage ranges. The amount of an enriched immune cell population and/or pharmaceutical composition of any of the foregoing administered will depend on, among other factors, the patient being treated, the weight of the patient, the severity of the affliction, the manner of administration and the judgment of the prescribing physician.

An enriched immune cell population can be assayed in vitro and in vivo, for the desired therapeutic activity, prior to use in humans. For example, in vitro assays may be used to determine whether the administration of a specific compound or a combination of compounds is preferred. The compounds can also be demonstrated to be effective and safe using animal model systems.

In certain embodiments, a therapeutically effective dose of an enriched immune cell population and/or pharmaceutical composition of any of the foregoing will provide therapeutic benefit without causing substantial toxicity. The toxicity of an enriched immune cell population and/or pharmaceutical compositions of any of the foregoing may be determined using standard pharmaceutical procedures and may be readily ascertained by the skilled artisan. The dose ratio between toxic and therapeutic effect is the therapeutic index. An enriched immune cell population and/or pharmaceutical composition of any of the foregoing exhibits a particularly high therapeutic index in treating disease and disorders. A dose of an enriched immune cell population and/or pharmaceutical composition of any of the foregoing will be within a range of circulating concentrations that include an effective dose with minimal toxicity.

An enriched immune cell population provided by the present disclosure, or a pharmaceutical composition thereof may be included in a kit that may be used to administer the compound to a patient for therapeutic purposes. A kit may include a pharmaceutical composition comprising an enriched immune cell population provided by the present disclosure suitable for administration to a patient and instructions for administering the pharmaceutical composition to the patient. The kit can be a kit for treating cancer, for treating an autoimmune disease, or for treating an inflammatory disease. A kit for use in treating cancer in a patient can comprise an enriched immune cell population provided by the present disclosure, a pharmaceutically acceptable vehicle for administering the compound, and instructions for administering the compound to a patient.

The pharmaceutical compositions can be included in a container, pack, or dispenser together with instructions for administration.

Instructions supplied with a kit may be printed and/or supplied, for example, as an electronic-readable medium, a video cassette, an audiotape, a flash memory device, or may be published on an internet web site or distributed to a patient and/or health care provider as electronic communication.

A method of treating a disease in a patient provided by the present disclosure can comprise administering to a patient in need of such treatment a therapeutically effective amount of a pharmaceutical composition provided by the present disclosure.

Enriched immune cell populations provided by the present disclosure can be used, for example, to treat diseases such as cancer, an autoimmune disease, or an infectious disease, including a viral disease such as COVID-19.

An enriched immune cell population provided by the present disclosure and pharmaceutical compositions of any of the foregoing may be administered to a patient to treat an organ transplant.

An enriched immune cell population provided by the present disclosure and pharmaceutical compositions of any of the foregoing may be administered to a patient together with another compound for treating an autoimmune disease in the subject. The at least one other therapeutic agent may be an enriched immune cell population provided by the present disclosure. An enriched immune cell population and the at least one other therapeutic agent may act additively or synergistically. The at least one additional therapeutic agent may be included in the same pharmaceutical composition or vehicle comprising the enriched immune cell population or may be in a separate pharmaceutical composition or vehicle. Accordingly, methods provided by the present disclosure further include, in addition to administering an enriched immune cell population, administering one or more therapeutic agents effective for treating an autoimmune disease or a different disease, disorder, or condition than an autoimmune disease. Methods provided by the present disclosure include administration of an enriched immune cell population and one or more other therapeutic agents, provided that the combined administration does not inhibit the therapeutic efficacy of an enriched immune cell population and/or does not produce adverse combination effects.

Enriched immune cell populations provided by the present disclosure may be used for treating cancer in a patient. The cancer can be, for example, a solid tumor or a metastasis.

Enriched immune cell populations provided by the present disclosure or pharmaceutical compositions thereof can be used to treat, for example, one or more of the following cancers: acute lymphoblastic leukemia, acute myeloid leukemia, adrenocortical carcinoma, appendix cancer, astrocytoma, atypical teratoid/rhabdoid tumor, basal cell carcinoma (nonmelanoma), B-cell lymphoma, bladder cancer, bone cancer, brain and spinal cord tumors, brain stem cancer, brain tumor, breast cancer, bronchial tumors, Burkitt lymphoma, carcinoid tumor, carcinoma of head and neck, central nervous system embryonal tumors, cerebellar astrocytoma, cerebral astrocytoma/malignant glioma, cervical cancer, chordoma, chronic lymphocytic leukemia, chronic myelogenous leukemia, colorectal cancer, craniopharyngioma, cutaneous T-cell lymphoma, desmoplastic small round cell tumor, ductal carcinoma, dye cancer, endocrine pancreas tumors (islet cell tumors), endometrial cancer, ependymoblastoma, esophageal cancer, esthesioneuroblastoma, Ewing family of tumors, extracranial germ cell tumor, extrahepatic bile duct cancer, gallbladder cancer, gastric cancer, gastrointestinal carcinoid tumor, gastrointestinal stromal tumor, gestational trophoblastic tumor, glioblastoma, glioma, hairy cell leukemia, head and neck cancer, heart cancer, hematopoetic tumors of the lymphoid lineage, hepatocellular cancer, Hodgkin lymphoma, hypopharyngeal cancer, hypothalamic and visual pathway glioma, IDs-related lymphoma, intraocular melanoma, islet cell tumors, Kaposi sarcoma, kidney cancer, Langerhans cell histiocytosis, laryngeal cancer, leukemia, lip and oral cavity cancer, male breast cancer, malignant fibrous histiocytoma, malignant germ cell tumors, malignant mesothelioma, medulloblastoma, melanoma, Merkel cell carcinoma, mesothelioma, mouth cancer, multiple endocrine neoplasia syndrome, multiple myeloma, mycosis fungoides, myelodysplastic, myeloproliferative neoplasms, nasal cavity and paranasal sinus cancer, nasopharyngeal cancer, neuroblastoma, non-Hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pineal parenchymal tumors, pineoblastoma and supratentorial primitive neuroectodermal tumors, pituitary tumor, plasma cell neoplasm/multiple myeloma, pleuropulmonary blastoma, pregnancy and breast cancer, primary central nervous system lymphoma, primary liver cancer, primary metastatic squamous neck cancer with occult, prostate cancer, rectal cancer, renal cell cancer, renal pelvis and ureter, respiratory tract carcinoma, retinoblastoma, rhabdomyosarcoma, salivary gland cancer, sarcoma, Sezary syndrome, skin cancer, small intestine cancer, soft tissue sarcoma, squamous cell carcinoma (nonmelanoma), stomach cancer, supratentorial primitive neuroectodermal tumors, T-cell lymphoma, testicular cancer, throat cancer, thymoma and thymic carcinoma, thyroid cancer, transitional cell cancer, urethral cancer, uterine sarcoma, vaginal cancer, visual pathway and hypothalamic glioma, vulvar cancer, Waldenström macroglobulinemia, Wilms tumor, and systemic and central metastases of any of the foregoing.

A cancer can be a solid tumor, a metastatic cancer, and/or a blood malignancy such as lymphoma or leukemia.

Enriched immune cell populations provided by the present disclosure or pharmaceutical compositions thereof can be used to treat solid tumors.

Enriched immune cell populations provided by the present disclosure or pharmaceutical compositions thereof can be used to treat tumor metastases.

Enriched immune cell populations provided by the present disclosure or pharmaceutical compositions thereof can be used to treat circulating tumor cells.

Enriched immune cell populations provided by the present disclosure or pharmaceutical compositions thereof can be used to treat, for example, a cancer selected from primary adult and childhood brain and CNS cancers including glioblastoma (GBM) and astrocytoma, skin cancers including melanoma, lung cancers including small cell lung cancers, non-small cell lung cancers (NSCLC), and large cell lung cancers, breast cancers including triple-negative breast cancer (TNBC), blood cancers including myelodysplastic syndrome (MDS), multiple myeloma (MM), and acute myeloid leukemia (AML), prostate cancer including castrate-resistant prostate cancer (CRPC), liver cancers including hepatocellular carcinoma (HCC), esophageal and gastric cancers, and any systemic and central metastases of any of the foregoing.

The amount of an enriched immune cell population provided by the present disclosure, or pharmaceutical composition thereof that will be effective in the treatment of a cancer can depend, at least in part, on the nature of the disease and may be determined by standard clinical techniques known in the art. In addition, in vitro or in vivo assays may be employed to help identify optimal dosing ranges. Dosing regimens and dosing intervals may also be determined by methods known to those skilled in the art. The amount of an enriched immune cell population provided by the present disclosure administered may depend on, among other factors, the patient being treated, the weight of the patient, the severity of the disease, the route of administration, and the judgment of the prescribing physician.

For systemic administration, a therapeutically effective dose may be estimated initially from in vitro assays. Initial doses may also be estimated from in vivo data, e.g., animal models, using techniques that are known in the art. Such information may be used to more accurately determine useful doses in humans. One having ordinary skill in the art may optimize administration to humans based on animal data.

A dose of an enriched immune cell population provided by the present disclosure and appropriate dosing intervals may be selected to maintain a sustained therapeutically effective concentration of the enriched immune cell population provided by the present disclosure in the blood of a patient, and in certain embodiments, without exceeding a minimum adverse concentration.

A pharmaceutical composition comprising an enriched immune cell population provided by the present disclosure may be administered, for example, once per week, every 2 weeks, every 3 weeks, every 4 weeks, every 5 weeks, or every 6 weeks. Dosing may be provided alone or in combination with other drugs and may continue as long as required for effective treatment of the disease. Dosing may also be undertaken using continuous or semi-continuous administration over a period of time. Dosing includes administering a pharmaceutical composition to a mammal, such as a human, in a fed or fasted state.

A pharmaceutical composition may be administered in a single dosage form or in multiple dosage forms or as a continuous or an accumulated dose over a period of time. When multiple dosage forms are used the amount of an enriched immune cell population provided by the present disclosure contained within each of the multiple dosage forms may be the same or different.

Pharmaceutical compositions comprising an enriched immune cell population may be administered to treat a disease in a patient so as to provide a therapeutically effective concentration of the enriched immune cell population in the blood of a patient for an extended period of time such as, for example, for at least 1 day, for at least 1 week, at least 2 weeks, at least 4 weeks, at least 5 weeks, or at least 6 weeks.

The amount of an enriched immune cell population administered may vary during a treatment regimen.

Pharmaceutical compositions provided by the present disclosure may further comprise one or more pharmaceutically active compounds in addition to an enriched immune cell population provided by the present disclosure. Such compounds may be provided, for example, to treat the cancer being treated with the enriched immune cell population or to treat a disease, disorder, or condition other than the cancer being treated with the enriched immune cell population, to treat a side-effect caused by administering the enriched immune cell population, to augment the efficacy of the enriched immune cell population, and/or to modulate the activity of the enriched immune cell population.

An enriched immune cell population provided by the present disclosure may be used in combination with at least one other therapeutic agent. An enriched immune cell population may be administered to a patient together with another compound for treating cancer in the patient. The at least one other therapeutic agent can be a second, different enriched immune cell population. An enriched immune cell population and the at least one other therapeutic agent may act additively or, and in certain embodiments, synergistically with another enriched immune cell population. The at least one additional therapeutic agent may be included in the same pharmaceutical composition or vehicle comprising the enriched immune cell population or may be in a separate pharmaceutical composition or vehicle. Accordingly, methods provided by the present disclosure further include, in addition to administering an enriched immune cell population, administering one or more therapeutic agents effective for treating cancer or a different disease, disorder or condition than cancer. Methods provided by the present disclosure include administration of an enriched immune cell population and one or more other therapeutic agents provided that the combined administration does not inhibit the therapeutic efficacy of the enriched immune cell population and/or does not produce adverse combination effects.

A pharmaceutical composition comprising an enriched immune cell population may be administered concurrently with the administration of another therapeutic agent, which may be part of the same pharmaceutical composition, or in a different pharmaceutical composition, as that comprising an enriched immune cell population. An enriched immune cell population may be administered prior or subsequent to administration of another therapeutic agent. In certain combination therapies, the combination therapy may comprise alternating between administering an enriched immune cell population and a composition comprising another therapeutic agent, e.g., to minimize adverse drug effects associated with a particular drug. When an enriched immune cell population is administered concurrently with another therapeutic agent that potentially may produce an adverse drug effect including, for example, toxicity, the other therapeutic agent may be administered at a dose that falls below the threshold at which the adverse drug reaction is elicited.

A pharmaceutical composition comprising an enriched immune cell population provided by the present disclosure may be administered with one or more substances, for example, to enhance, modulate and/or control release, bioavailability, therapeutic efficacy, therapeutic potency, and/or stability, of the enriched immune cell population. For example, a pharmaceutical composition comprising an enriched immune cell population can be co-administered with an active agent having pharmacological effects that enhance the therapeutic efficacy of the enriched immune cell population.

An enriched immune cell population provided by the present disclosure, or a pharmaceutical composition thereof, may be administered in conjunction with an agent known or believed to be effective in treating a disease such as cancer, an autoimmune disease, or an inflammatory disease in a patient, such as the same disease being treated with the enriched immune cell population.

An enriched immune cell population, or a pharmaceutical composition thereof may be administered in conjunction with an agent known or believed to interfere with cell proliferation.

An enriched immune cell population, or a pharmaceutical composition thereof, may be administered in conjunction with an agent known or believed to interfere with cellular metabolism, to be an anti-metabolite, to interfere with RNA transcription, to interfere with RNA translation, to interfere with cellular protein synthesis, to interfere with synthesis of precursors for DNA synthesis and replication, to interfere with purine synthesis, to interfere with nucleoside synthesis, to interact with mTOR, to be an mTOR inhibitor, or to interfere with cell cycle checkpoints.

An enriched immune cell population or a pharmaceutical composition thereof may be administered in conjunction with a checkpoint inhibitor including a CTLA-4 inhibitor such as ipilimumab, a PD-1 inhibitor such as pembrolizumab and nivolumab, and/or a PD-LI inhibitor such as atezolizumab, avelumab, and durvalumab. An enriched immune cell population or a pharmaceutical composition thereof may be administered in conjunction with an immunomodulator such as CD137/4-1BB, CD27, GITR, and/or CD40.

An enriched immune cell population or a pharmaceutical composition thereof may be administered in conjunction with an agent known or believed to be cytotoxic, to cause DNA damage, to cause cell cycle arrest, or to cause mitotic catastrophe.

An enriched immune cell population or a pharmaceutical composition thereof may be administered in conjunction with an agent known or believed to modulate glutathione concentration, to modulate glutathione concentration within cells, to decrease glutathione concentration within cells, to reduce glutathione uptake into cells, to reduce glutathione synthesis, or to reduce glutathione synthesis within cells.

An enriched immune cell population or a pharmaceutical composition thereof may be administered in conjunction with an agent known or believed to interfere with neovascularization, to reduce neovascularization, or to promote neovascularization.

An enriched immune cell population or a pharmaceutical composition thereof may be administered in conjunction with an agent known or believed to interfere with hormone homeostasis, to interfere with hormone synthesis, to interfere with hormone receptor binding, or to interfere with hormone signal transduction.

An enriched immune cell population or a pharmaceutical composition thereof may be administered in conjunction with an agent known or believed to interfere with growth factor homeostasis, to interfere with growth factor receptor expression, to interfere with growth factor binding to growth factor receptors, to interfere with growth factor receptor signal transduction, to interfere with the Hedgehog (Hh) signaling, to inhibit the Hedgehog pathway signaling, to inhibit ALK (anaplastic lymphoma kinase) pathway signaling, or to inhibit the non-homologous end joining (NHEJ) pathway.

An enriched immune cell population or a pharmaceutical composition thereof may be administered in conjunction with one or more agents known or believed to be a VEGFR (vascular endothelial growth factor receptor) inhibitor, a RTK (receptor tyrosine kinase) inhibitor, a sodium channel current blocker, a FAK (focal adhesion kinase) inhibitor, a GLI (glioma-associated oncogene) inhibitor, a GLI1 inhibitor, a GLI2 inhibitor, a GLI3 inhibitor, a MAPK (mitogen-activated protein kinase) inhibitor, a MAPK/ERK pathway (also known as Ras-Raf-MEK-ERK pathways) inhibitor, a MEK1 inhibitor, a MEK2 inhibitor, a MEK5 inhibitor, a MEK5/ERK5 inhibitor, aRTA (renal tubular acidosis) inhibitor, a ALK (anaplastic lymphoma kinase) inhibitor, Aa LK kinase inhibitor, a nuclear translocation inhibitor, a PORCN (porcupine) inhibitor, a 5-ARI (5α-reductase inhibitor), topoisomerase inhibitor, a Ras (rat sarcoma) inhibitor, a K-ras inhibitor, a CERK (ceramide kinase) inhibitor, a PKB (protein kinase B, also known as AKT) inhibitor, a AKT1 inhibitor, EZH2 (enhancer of zeste homolog 2) inhibitor, a BET (bromodomain and extraterminal domain motif) inhibitor, a SYK (spleen tyrosine kinase) inhibitor, JAK (Janus kinase) inhibitors, a SYK/JAK inhibitor, a IDO (indoleamine-pyrrole 2,3-dioxygenase) inhibitor, a IDO1 inhibitor, a RXR (retinoic X receptors) activating agent, a selective RXR activating agent, a p-glycoprotein inhibitor, a ERK inhibitor, a PI3K (phosphatidylinositol-4,5-bisphosphate 3-kinase) inhibitor, a BRD (bromodomain-containing protein) inhibitor, a BRD2 inhibitor, a BRD3 inhibitor, a BRD4 inhibitor, a BRDT (bromodomain testis-specific protein) inhibitor, a reverse transcriptase inhibitor, a NRT (nucleoside analog reverse-transcriptase) inhibitor, a PIM (proviral integrations of moloney virus) inhibitor, a EGFR (epidermal growth factor receptor) inhibitor, a photosensitizer, a radiosensitizer, a ROS (proto-oncogene, receptor tyrosine kinase) inhibitor, a ROS 1 (ROS proto-oncogene 1) inhibitor, a CK (casein kinase) inhibitor, a CK2 inhibitor, a Bcr-Abl (breakpoint cluster region—Abelson proto-oncogene) tyrosine-kinase inhibitor such as dasatinib, a microtubule stabilizing agent, a microtubule depolymerization/disassembly inhibitor, a DNA intercalator, an androgen receptor antagonist, a chemoprotective agents, a HDAC (histone deacetylase) inhibitor, a DPP (dipeptidyl peptidase) inhibitor, a DPP-4 inhibitor, BTK (Bruton's tyrosine kinase) inhibitor, a kinase inhibitor such as imatinib, a tyrosine kinase inhibitor such as nilotinib, a ARP (poly (ADP-ribose) polymerase) inhibitor, a CDK (cyclin-dependent kinase) inhibitor, a CDK4 inhibitor, a CDK6 inhibitor, a CDK4/6 inhibitor, a HIF1α (hypoxia-inducible factor 1-α) inhibitor, a DNA ligase inhibitor, a DNA ligase IV inhibitor, a NHEJ (non-homologous end joining) inhibitor, a DNA ligase IV, a NHEJ inhibitor and a RAF inhibitor, a TKI and a RAF inhibitor, a TKI and RAF inhibitor such as sorafenib, a PDT (photodynamic therapy) sensitizer, an ATR (ataxia telangiectasia- and Rad3-related protein kinase) inhibitor, or a combination of any of the foregoing.

An enriched immune cell population or a pharmaceutical composition thereof may be administered in conjunction with one or more chemotherapeutic agents, such as, for example, a VEGFR inhibitor such as fruquintinib, motesanib/AMG-706, vatalanib; a RTK inhibitor such as ponatinib; a sodium channel blocker such as GS967; a FAK inhibitor such as TAE226; a GLI1 and GLI2 inhibitor such as GANT61, a MEK inhibitor such as binimetinib; a RTA inhibitor such as linifanib; an ALK inhibitor such as brigstinib; bromopyruvic acid; a DNA alkylating agent such as thiotepa; nuclear translocations factors such as JSH-23; a PORCn inhibitor such as Wnt-059; a 5α-reductase inhibitor such as dutasteride; a topoisomerase inhibitor such as carubicin; a RAS inhibitor such as Kobe0065; a CerK inhibitor such as NVP-231; an AKT inhibitor such as uprosertib; a EZH2 inhibitor such as GSK-503; a BET bromodomain inhibitor such as OTX015; a MEK5/ERK5 inhibitor such as BIX02189; a Syl/JAK inhibitor such as cerdulatinib; an IDO1 inhibitor such as NLG919; a retinoic X receptor activating agent such as bexsrotene; a PGP inhibitor such as acotiamide or actotiamide HCl; an Erk inhibitor such SCH772984; a PI3K inhibitor such as gedatolisib; a JAK inhibitor such as ruxolitinib; an AKT inhibitor such as afuresertib or afuresertib HCl; an ALK1 inhibitor such as ceritinib; an HDAC inhibitor such as abexinostat; a DPP inhibitor such as oamarigliptin; an EGFR inhibitor such as gefittinib; an EZH2 inhibitor such as GSK126; a BTK inhibitor such as ibrutinib; a kinase inhibitor such as imatinin HCl; an IDO inhibitor such as INCB024360; a DNA crosslinker such as mitomycin C; a tyrosine kinase inhibitor such as nilotinib, a PARP inhibitor such as olaparib; a tubulin stabilization promoter such as paclitaxel; a CDK4/6 inhibitor such as palbociclib; a RTK inhibitor such as sunitinib; a PDT sensitizer such as tslsporfin; a p-glycoprotein inhibitor such as tariquidar; an ATR inhibitor such as VE-822; an HDAC inhibitor such as PCI-24781; a DPP inhibitor such as omarigliptin; an EGFR inhibitor such as gefinib; an EZH2 inhibitor such as GSK126; a BTK inhibitor such as irbrutinib; an IDO inhibitor such as INCB024360; or a combination of any of the foregoing.

For example, an enriched immune cell population or a pharmaceutical composition thereof may be administered in conjunction with another chemotherapeutic agent, such as, for example, N-acetyl cysteine (NAC), adriamycin, alemtuzumab, amifostine, arsenic trioxide, ascorbic acid, bendamustine, bevacizumab, bortezomib, busulfan, buthionine sulfoxime, carfilzomib, carmustine, clofarabine, cyclophosphamide, cyclosporine, cytarabine, dasatinib, datinomycin, defibrotide, dexamethasone, docetaxel, doxorubicin, etoposide, filgrastim, floxuridine, fludarabine, gemcitabine, interferon alpha, ipilimumab, lenalidomide, leucovorin, melphalan, mycofenolate mofetil, paclitaxel, palifermin, panobinostat, pegfilrastim, prednisolone, prednisone, revlimid, rituximab, sirolimus, sodium 2-mercaptoethane sulfonate (MESNA), sodium thiosulfate, tacrolimus, temozolomide, thalidomide, thioguanine, thiotepa, topotecan, velcade, or a combination of any of the foregoing.

An enriched immune cell population or a pharmaceutical compositions thereof can be used in combination therapy with other chemotherapeutic agents including one or more antimetabolites such as folic acid analogs; pyrimidine analogs such as fluorouracil, floxuridine, and cytosine arabinoside; purine analogs such as mercaptopurine, thiogunaine, and pentostatin; natural products such as vinblastine, vincristine, etoposide, tertiposide, dactinomycin, daunorubicin, doxurubicin, bleomycin, mithamycin, mitomycin C, L-asparaginase, and interferon alpha; platinum coordination complexes such as cis-platinum, and carboplatin; mitoxantrone; hydroxyurea; procarbazine; hormones and antagonists such as prednisone, hydroxyprogesterone caproate, medroxyprogesterone acetate, megestrol acetate, diethylstilbestrol, ethinyl estradiol, tamoxifen, testosterone propionate, fluoxymesterone, flutamide, and leuprolide, anti-angiogenesis agents or inhibitors such as angiostatin, retinoic acids, paclitaxel, estradiol derivatives, and thiazolopyrimidine derivatives; apoptosis prevention agents; triptolide; colchicine; luliconazole; and radiation therapy.

An enriched immune cell population or a pharmaceutical composition thereof may be co-administered with a compound that inhibits DNA repair such as, for example, O6-benzylguanine (O6-BG).

An enriched immune cell population or a pharmaceutical composition thereof may be administered in conjunction with one or more chemotherapeutic agents, such as, for example, abarelix, abiraterone, abiraterone acetate, n-acetyl cysteine, aclarubicin hydrochloride, adriamycin, adenine, afatinib, afatinib dimaleate, alemtuzumab, alendronate sodium, alitretinoin, allopurinol sodium, altretamine, amifostine, aminoglutethimide, aminolevulinic acid, amrubicin, amsacrine, anastrozole, angiostatin, apremilast, aprepitant, arsenic trioxide, ascorbic acid, l-asparaginase, azacitidine, azathioprine sodium, bazedoxifene (serm), belinostat, bendamustine hcl, O6-benzylguanine, bevacizumab, bexarotene, bicalutamide, biricodar, bleomycin sulfate, bortezomib, bosutinib, brivudine, buserelin, busulfan, buthionine sulfoxime, cabazitaxel, cabozantinib, capecitabine, carboplatin, carboquone, carfilzomib, carmofur, carmustine, ceritinib, chlorambucil, cisplatin, cladribine, clodronate disodium, clofarabine, crizotinib, cyclophosphamide, cyclosporine, cytarabine, cytosine arabinoside, dabrafenib, dacarbazine, dactinomycin, dasatinib, datinomycin, daunorubicin, decitabine, defribrotide, degarelix acetate, dexamethasone, dexrazoxane hydrochloride, diaziquone, diethyl stilbestrol, docetaxel, doxifluridine, doxorubicin hydrochloride, doxorubicin free base, dromostanolone propionate, dutasteride, eltrombopag, enzalutamide, epirubicin hydrochloride, eribulin mesylate, erlotinib hydrochloride, estramustine phosphate sodium, ethinyl estradiol, etoposide phosphate, etoposide, everolimus, exemestane, fentanyl, filgrastim, fingolimod, floxuridine, fludarabine phosphate, fluorouracil, fluoxymesterone, flutamide, formestane, formylmelphalan, fosaprepitant, fotemustine, fulvestrant, gefitinib, gemcitabine hydrochloride, gemcitabine free base, glutathione, glyciphosphoramide, glyfosfin, goserelin acetate, granisetron hydrochloride, heptaplatin, hexyl 5-aminolevulinate, histrelin acetate, hydroxyprogesterone caproate, hydroxyurea, ibandronate sodium, ibrutinib, icotinib, idarubicin HCl, idelalisib, idoxuridine, ifosfamide, interferon alpha, imatinib mesylate, imiquimod, ingenol mebutate, ipilimumab, irinotecan hydrochloride, ixabepilone, lanreotide acetate, lapatinib free base, lapatinib ditosylate, lasofoxifene, lenalidomide, letrozole, leucovorin calcium, leuprolide acetate, levamisole hydrochloride, levoleucovorin calcium, iobenguane, lobaplatin, lomustine, maropitant, masoprocol, mechlorethamine hydrochloride, megestrol acetate, medroxyprogesterone acetate, melphalan hydrochloride, mercaptopurine, mercaptoethane sulfonate sodium, methotrexate, methoxsalen, methyl aminolevulinate, methylene blue, methylisoindigotin, mifamurtide, miltefosine, miriplatin, mithamycin, mitobronitol, mitomycin C, mitotane, mitoxantrone hydrochloride, mycophenolate mofetil, nabiximols, nafarelin, nandrolone, nedaplatin, nelarabine, netupitant, nilotinib, nilutamide, nimustine, nintedanib, nocodazole, octreotide, olaparib, omacetaxine mepesuccinate, ondansetron hydrochloride, oxaliplatin, paclitaxel, palbociclib, palifermin, palonosetron hydrochloride, pamidronate disodium, panobinostat, pasireotide, pazopanib hydrochloride, pegfilrastim, pemetrexed disodium, pentostatin, peplomycin, pipobroman, pirarubicin, plerixafor, plicamycin, pomalidomide, ponatinib, porfimer sodium, porfiromycin, pralatrexate, prednimustine, prednisolone, prednisone, procarbazine hydrochloride, quinagolide hydrochloride, raloxifene, raltitrexed, radotinib, ranimustine, retinoic acids, revlimide, rituxinab, romidepsin, ruxolitinib, ruxolitinib phosphate, semustine, sirolimus, sodium thiosulfate, sorafenib free base, sorafenib tosylate, streptozocin, sufentanil, sunitinib, tacrolimus, talaporfin sodium, tamibarotene, tamoxifen citrate, tapentadol, temoporfin, temozolomide, temsirolimus, teniposide, teriflunomide, tertiposide, testolactone, testosterone propionate, thalidomide, thioguanine, thiotepa, thymalfasin, toceranib phosphate, topotecan hydrochloride, toremifene citrate, trabectedin, trametinib, tretinoin, trilostane, triptorelin, tropisetron, uramustine, valrubicin, vandetanib, vedotin, vemurafenib, verteporfin, vinblastine, vincristine sulfate, vincristine free base, vindesine, vinorelbine tartrate, vorinostat, and zoledronic acid.

An enriched immune cell population or a pharmaceutical composition thereof may be administered in conjunction with one or more chemotherapeutic agents such as, for example, abemaciclib, abiraterone acetate, ABVD, ABVE, ABVE-PC, AC, acalabrutinib, AC-T, ADE, ado-trastuzumab emtansine, afatinib dimaleate, aldesleukin, alectinib, alemtuzumab, alpelisib, amifostine, aminolevulinic acid hydrochloride, anastrozole, apalutamide, aprepitant, arsenic trioxide, asparaginase Erwinia chrysanthemi, atezolizumab, avelumab, axicabtagene ciloleucel, axitinib, azacitidine, BEACOPP, belinostat, bendamustine hydrochloride, BEP, bevacizumab, bexarotene, bicalutamide, binimetinib, bleomycin sulfate, blinatumomab, bortezomib, bosutinib, brentuximab vedotin, brigatinib, BuMel, busulfan, cabazitaxel, cabozantinib-s-malate, CAF, calaspargase pegol-mknl, capecitabine, caplacizumab-yhdp, CAPDX, carboplatin, carboplatin-taxol, carfilzomib, carmustine, carmustine implant, CEM, cemiplimab-rwlc, ceritinib, cetuximab, CEV, chlorambucil, chlorambucil-prednisone, CHOP, cisplatin, cladribine, clofarabine, CMF, cobimetinib, copanlisib hydrochloride, COPDAC, COPP, COPP-ABV, crizotinib, CVP, cyclophosphamide, cytarabine, cytarabine liposome, dabrafenib mesylate, dacarbazine, dacomitinib, dactinomycin, daratumumab, darbepoetin a, dasatinib, daunorubicin hydrochloride, daunorubicin hydrochloride and cytarabine liposome, decitabine, defibrotide sodium, degarelix, denileukin diftitox, denosumab, dexamethasone, dexrazoxane hydrochloride, dinutuximab, docetaxel, doxorubicin hydrochloride, doxorubicin hydrochloride liposome, durvalumab, duvelisib, elotuzumab, eltrombopag olamine, emapalumab-lzsg, enasidenib mesylate, encorafenib, enzalutamide, epirubicin hydrochloride, EPOCH, epoetin alfa, erdafitinib, eribulin mesylate, erlotinib hydrochloride, etoposide, etoposide phosphate, everolimus, exemestane, fec, filgrastim, fludarabine phosphate, fluorouracil injection, fluorouracil—topical, flutamide, folfiri, folfiri-bevacizumab, folfiri-cetuximab, folfirinox, folfox, fostamatinib disodium, FU-LV, fulvestrant, gefitinib, gemcitabine hydrochloride, gemcitabine-cisplatin, gemcitabine-oxaliplatin, gemtuzumab ozogamicin, gilteritinib fumarate, glasdegib maleate, glucarpidase, goserelin acetate, granisetron, HPV bivalent vaccine, HPV bivalent vaccine, recombinant HPV nonavalent vaccine, HPV nonavalent vaccine, recombinant, HPV quadrivalent vaccine, HPV uadrivalent vaccine recombinant, hydroxyurea, hyper-CVAD, ibritumomab tiuxetan, ibrutinib, ICE, idarubicin hydrochloride, idelalisib, ifosfamide, imatinib mesylate, imiquimod, inotuzumab ozogamicin, interferon α-2b recombinant, iobenguane I¹³¹ ipilimumab, irinotecan hydrochloride, irinotecan hydrochloride liposome, ivosidenib, ixabepilone, ixazomib citrate, JEB, lanreotide acetate, lapatinib ditosylate, larotrectinib sulfate, lenalidomide, lenvatinib mesylate, letrozole, leucovorin calcium, leuprolide acetate, lomustine, lorlatinib, lutetium Lu 177-dotatate, mechlorethamine hydrochloride, megestrol acetate, melphalan, melphalan hydrochloride, mercaptopurine, mesna, methotrexate, methylnaltrexone bromide, midostaurin, mitomycin c, mitoxantrone hydrochloride, mogamulizumab-kpkc, moxetumomab pasudotox-tdfk, MVAC, necitumumab, nelarabine, neratinib maleate, netupitant and palonosetron hydrochloride, nilotinib, nilutamide, niraparib tosylate monohydrate, nivolumab, obinutuzumab, OEPA, ofatumumab, OFF, olaparib, olaratumab, omacetaxine mepesuccinate, ondansetron hydrochloride, OPPA, osimertinib mesylate, oxaliplatin, paclitaxel, paclitaxel albumin-stabilized nanoparticle formulation, PAD, palbociclib, palifermin, palonosetron hydrochloride, palonosetron hydrochloride and netupitant, pamidronate disodium, panitumumab, panobinostat, pazopanib hydrochloride, PCV, PEB, pegaspargase, pegfilgrastim, peginterferon α-2b, pembrolizumab, pemetrexed disodium, pertuzumab, plerixafor, polatuzumab vedotin-piiq, pomalidomide, ponatinib hydrochloride, pralatrexate, prednisone, procarbazine hydrochloride, propranolol hydrochloride, radium 223 dichloride, raloxifene hydrochloride, ramucirumab, rasburicase, ravulizumab-cwvz, R-CHOP, R-CVP, recombinant HPV bivalent vaccine, recombinant HPV nonavalent vaccine, recombinant HPV quadrivalent vaccine, recombinant interferon α-2b, regorafenib, R-EPOCH, ribociclib, R-ICE, rituximab, rituximab and hyaluronidase human, rolapitant hydrochloride, romidepsin, romiplostim, rucaparib camsylate, ruxolitinib phosphate, siltuximab, sipuleucel-t, sonidegib, sorafenib tosylate, STANFORD V, sunitinib malate, TAC, tagraxofusp-erzs, talazoparib tosylate, talc, talimogene laherparepvec, tamoxifen citrate, temozolomide, temsirolimus, thalidomide, thioguanine, thiotepa, tisagenlecleucel, tocilizumab, topotecan hydrochloride, toremifene, TPF, trabectedin, trametinib, trastuzumab, trastuzumab and hyaluronidase-oysk, trifluridine and tipiracil hydrochloride, uridine triacetate, VAC, Valrubicin, VAMP, vandetanib, VeIP, vemurafenib, venetoclax, vinblastine sulfate, vincristine sulfate liposome, vinorelbine tartrate, vip, vismodegib, vorinostat, XELIRI, XELOX, Ziv-aflibercept, zoledronic acid, and combinations of any of the foregoing.

The efficacy of an enriched immune cell population or a pharmaceutical composition thereof for treating cancer may be assessed using in vitro and animal studies and in clinical trials.

The suitability of an enriched immune cell population or a pharmaceutical composition thereof in treating cancer may be determined by methods described in the art.

An enriched immune cell population or a pharmaceutical composition thereof may be administered to a patient in need of such treatment to treat an autoimmune disease.

Examples of autoimmune diseases include Addison's disease, agammaglobulinemia, alopecia areata, amyloidosis, ankylosing spondylitis, anti-GBM/anti-TBN nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune pancreatitis, autoimmune retinopathy, autoimmune urticaria, axonal and neuronal neuropathy, Balo's disease, Bechet's disease, benign mucosal pemphigoid, bullous pemphigoid, Castleman disease, celiac disease, Chagas disease, chronic inflammatory demyelinating polyneuropathy, chronic recurrent multifocal osteomyelitis, Churg-Strauss, cicatricial pemphigoid, Cogan's syndrome, cold agglutinin disease, congenital heart block, Coxsackie myocarditis, CREST syndrome, Crohn's disease, dermatitis herpetiformis, dermatomyositis, Devic's disease, discoid lupus, Dressler's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis, giant cell myocarditis, glomerulonephritis, Goodpasture's syndrome, granulomatosis with polyangiitis, Graves' disease, Guillain-Barre syndrome, Hashimoto's thyroiditis, hemolytic anemia, Henoch-Schonlein purpura, herpes gestationis or pemphigoid gestationis, hypogammaglobulinemia, IgA nephropathy, IgG4-related sclerosing disease, immune thrombocytopenic purpura, inclusion body myositis, interstitial cystitis, juvenile arthritis, juvenile diabetes, juvenile myositis, Kawasaki disease, Lambert-Eaton syndrome, leukocytoclastic vasculitis, lichen planus, lichen sclerosis, ligneous conjunctivitis, linear IgA disease, lupus, Lyme disease chronic, Meniere's diseases, microscopic polyangiitis, mixed connective tissue disease, Mooren's ulcer, Mucha-Habermann disease, multiple sclerosis, myasthenia gravis, myositis, narcolepsy, neuromyelitis, optica, neutropenia, ocular cicatricial pemphigoid, optic neuritis, palindromic rheumatism, PANDAS, paraneoplastic cerebellar degeneration, paroxysmal nocturnal hemoglobinuria, Parry Romberg syndrome, pars planitis, Parsonage-Turner syndrome, pemphigus, peripheral neuropathy, perivenous encephalomyelitis, pernicious anemia, POEMS syndrome, polyarteritis nodosa, polyglandular syndromes, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjogren's syndrome, sperm and testicular autoimmunity, stiff person syndrome, subacute bacterial endocarditis, Susac's syndrome, sympathetic ophthalmia, Takayasu's arteritis, temporal arteritis, thrombocytopenic purpura, Tolosa-Hunt syndrome, transverse myelitis, type 1 diabetes, ulcerative colitis, undifferentiated connective tissue disease, uveitis, vasculitis, vitiligo, and Wegener's granulomatosis.

An enriched immune cell population or a pharmaceutical composition thereof can be used to treat autoimmune disorders such as, for example, lupus, graft-versus-host disease, hepatitis C-induced vasculitis, Type I diabetes, multiple sclerosis, spontaneous loss of pregnancy, atopic diseases, and inflammatory bowel diseases.

An enriched immune cell population can be administered with one or more additional therapeutic agents for treating an autoimmune disease. An IL-2Rβγc agonist peptide or a pharmaceutical composition thereof may be administered in conjunction with one or more immunosuppressants including, for example, corticosteroids such as prednisone, budesonide, and prednisolone; Janus kinase inhibitors such as tofacitinib; calcineurin inhibitors such as cyclosporine and tacrolimus; mTOR inhibitors such as sirolimus and everolimus; IMDH inhibitors such as azathioprine, leflunomide, and mycophenolate; biologics such as abatacept adalimumab, anakinra, certolizumab, etanercept, golimumab, infliximab, ixekizumab, natalizumab, rituximab, secukinumab, tocilizumab, ustekinumab, and vedolizumab; and monoclonal antibodies such as basiliximab and daclizumab.

An enriched immune cell population or a pharmaceutical composition thereof may be administered to a patient to treat a disease associated with the activation, proliferation, metabolism, and/or differentiation of T-cells.

An enriched immune cell population or a pharmaceutical composition thereof may be administered to a patient to treat an organ transplant.

An enriched immune cell population or a pharmaceutical composition thereof may be administered in conjunction with an agent known or believed to interfere with proliferation, to interfere with mitosis, to interfere with DNA replication, or to interfere with DNA repair.

An enriched immune cell population or a pharmaceutical composition thereof may be administered to a patient to treat an immune deficiency disease.

Examples of primary immune deficiency diseases include autoimmune lymphoproliferative syndrome, autoimmune polyglandular syndrome type 1, BENTA disease, caspase eight deficiency state, CARD9 deficiency, chronic granulomatous disease, common variable immunodeficiency, congenital neutropenia syndromes, CTLA4 deficiency, DOCK8 deficiency, GATA2 deficiency, glycosylation disorders, hyper-immunoglobulin E syndromes, hyper-immunoglobulin M syndromes, interferon γ, interleukin 12 and interleukin 23 deficiency, leukocyte adhesion deficiency, LRBA deficiency, PI2 kinase disease, PLCG2-associated antibody deficiency and immune dysregulation, severe combined immunodeficiency, STAT3 dominant-negative disease, STAT3 gain-of-function disease, warts, hypogammaglobulinemia, infections, and myelokathexis syndrome, Wiskott-Aldrich syndrome, X-linked agammaglobulinemia, X-linked lymphoproliferative disease, and XMEN disease.

Secondary immune deficiency disease occurs when the immune system is compromised due to an environmental factor such as infection, chemotherapy, severe burns, or malnutrition. Example of secondary immune deficiency diseases include newborn immunodeficiencies such as immature lymphoid organs, absent memory immunity, low maternal IgG levels, decreased neutrophil storage pool, decreased neutrophil function, and decreased natural killer cell activity; advanced age related immunodeficiencies such as decreased antigen-specific cellular immunity, T-cell oligoconality, and restricted B-cell repertoire; malnutrition related immunodeficiencies such as decreased cellular immune response and weekend mucosal barriers; diabetes mellitus related immunodeficiencies such as decreased mitogen-induced lymphoproliferation, defective phagocytosis, and decreased chemotaxis; chronic uremia related immunodeficiencies such as decreased cellular immune response, decreased generation of memory antibody responses, and decreased chemotaxis; genetic syndromes such as defective phagocytosis, defective chemotaxis, and variable defects of antigen-specific immune responses; and anti-inflammatory, immunomodulatory, and immuno-suppressive drug therapy related immune deficiencies such as lymphopenia, decreased cellular immune response and anergy, decreased proinflammatory cytokines, decreased phagocytosis, decreased chemotaxis, neutropenia, and weakened mucosal barriers; environmental conditions such as increased lymphocyte apoptosis, increased secretion of tolerogenic cytokines, cytopenia, decreased cellular immunity and anergy, and stress-induced nonspecific immune activation; and infectious diseases such as T-cell lymphopenia, decreased cellular immune response and anergy, and defective antigen-specific antibody responses.

An enriched immune cell population or a pharmaceutical composition thereof may be administered to a patient to increase the immune response in immuno-compromised patients.

An enriched immune cell population or a pharmaceutical composition thereof may be administered to a patient to increase the immune response in elderly patients.

An enriched immune cell population or a pharmaceutical composition thereof may be administered to a patient to treat an infectious disease such as a viral disease, where a viral disease refers to a disease caused by a virus.

A viral disease can be caused, for example, by a virus selected from herpes viruses, pox viruses, hepadnaviruses, papilloma viruses, adenoviruses, coronaviruses, orthomyxoviruses, paramyxoviruses, flaviviruses, and caliciviruses. For example, a viral disease can be caused by a virus selected from respiratory syncytial virus (RSV), influenza virus, herpes simplex virus, Epstein-Barr virus, varicella virus, cytomegalovirus, hepatitis A virus, hepatitis B virus, hepatitis C virus, human immunodeficiency virus (HIV), human T-lymphotropic virus, calicivirus, adenovirus, Arena virus, and COVID-19.

Examples of viral diseases include influenza, pneumonia, herpes, hepatitis, hepatitis A, hepatitis B, hepatitis C, chronic fatigue syndrome, sudden acute respiratory syndrome (SARS), gastroenteritis, enteritis, carditis, encephalitis, bronchiolitis, respiratory papillomatosis, meningitis, HIV/AIDS, and mononucleosis, and severe acute respiratory syndrome coronavirus 2 (SARS-CoV-2).

Examples of infectious diseases include Acinetobacter infections, actinomycosis, African sleeping sickness (African trypanosomiasis), AIDS (acquired immunodeficiency syndrome), amoebiasis, anaplasmosis, angiostrongyliasis, anisakiasis, anthrax, Arcanobacterium haemolyticum infection, Argentine hemorrhagic fever, ascariasis, aspergillosis, astrovirus infection, babesiosis, Bacillus cereus infection, bacterial meningitis, bacterial pneumonia, bacterial vaginosis, Bacteroides infection, balantidiasis, bartonellosis, Baylisascaris infection, Bejel, syphilis, yaws, BK virus infection, black piedra, blastocystosis, blastomycosis, Bolivian hemorrhagic fever, botulism (and Infant botulism), Brazilian hemorrhagic fever, brucellosis, bubonic plague, Burkholderia infection, buruli ulcer, calicivirus infection (Norovirus and Sapovirus), campylobacteriosis, candidiasis (Moniliasis; Thrush), capillariasis, carrion's disease, cat-scratch disease, cellulitis, Chagas disease (American trypanosomiasis), chancroid, chickenpox, chikungunya, Chlamydia, Chlamydophila pneumoniae infection (Taiwan acute respiratory agent or TWAR), cholera, chromoblastomycosis, Chytridiomycosis, clonorchiasis, Clostridium difficile colitis, coccidioidomycosis, Colorado tick fever (CTF), common cold (acute viral rhinopharyngitis; Acute coryza, Coronavirus disease 2019 (COVID-19), Creutzfeldt-Jakob disease (CJD), Crimean-Congo hemorrhagic fever (CCHF), cryptococcosis, cryptosporidiosis, cutaneous larva migrans (CLM), cyclosporiasis, cysticercosis, cytomegalovirus infection, Dengue fever, desmodesmus infection, dientamoebiasis, diphtheria, diphyllobothriasis, dracunculiasis, Ebola hemorrhagic fever, echinococcosis, Ehrlichiosis, enterobiasis (pinworm infection), Enterococcus infection, enterovirus infection, epidemic typhus, Epstein-Barr virus infectious mononucleosis (Mono), erythema infectiosum (Fifth disease), fxanthem subitum (Sixth disease), fasciolosis, fasciolopsiasis, fatal familial insomnia (FFI), filariasis, food poisoning by Clostridium perfringens, free-living amebic infection, Fusobacterium infection, gas gangrene (Clostridial myonecrosis), geotrichosis, Gerstmann-Straussler-Scheinker syndrome (GSS), giardiasis, glanders, gnathostomiasis, gonorrhea, granuloma inguinale (Donovanosis), Group A streptococcal infection, Group B streptococcal infection, Haemophilus influenzae infection, hand, foot and mouth disease (HFMD), Hantavirus Pulmonary Syndrome (HPS), Heartland virus disease, Helicobacter pylori infection, hemolytic-uremic syndrome (HUS), hemorrhagic fever with renal syndrome (HFRS), Hendra virus infection, Hepatitis A, Hepatitis B, Hepatitis C, Hepatitis D, Hepatitis E, Herpes simplex, histoplasmosis, hookworm infection, human bocavirus infection, human ewingii ehrlichiosis, human granulocytic anaplasmosis (HGA), human metapneumovirus infection, human monocytic ehrlichiosis, human papillomavirus (HPV) infection, human parainfluenza virus infection, hymenolepiasis, influenza (flu), isosporiasis, Kawasaki disease, keratitis, Kingella kingae infection, Kuru, Lassa fever, Legionellosis (Legionnaires' disease), leishmaniasis, leprosy, leptospirosis, listeriosis, Lyme disease (Lyme borreliosis), lymphatic filariasis (elephantiasis), lymphocytic choriomeningitis, malaria, Marburg hemorrhagic fever (MHF), measles, melioidosis (Whitmore's disease), meningitis, meningococcal disease, metagonimiasis, microsporidiosis, Middle East respiratory syndrome (MERS), molluscum contagiosum (MC), monkeypox, mumps, murine typhus (Endemic typhus), mycetoma, Mycoplasma genitalium infection, Mycoplasma pneumonia, myiasis, neonatal conjunctivitis (Ophthalmia neonatorum), Nipah virus infection, nocardiosis, Norovirus (children and babies), onchocerciasis (River blindness), opisthorchiasis, paracoccidioidomycosis (South American blastomycosis), paragonimiasis, pasteurellosis, pediculosis capitis (Head lice), pediculosis corporis (Body lice), pediculosis pubis (pubic lice, crab lice), pelvic inflammatory disease (PID), pertussis (whooping cough), plague, pneumococcal infection, Pneumocystis pneumonia (PCP), pneumonia, poliomyelitis, Pontiac fever, Prevotella infection, primary amoebic meningoencephalitis (PAM), progressive multifocal leukoencephalopathy, psittacosis, Q fever, rabies, relapsing fever, respiratory syncytial virus infection, rhinosporidiosis, rhinovirus infection, Rickettsia) infection, rickettsialpox, Rift Valley fever (RVF), Rocky Mountain spotted fever (RMSF), rotavirus infection, rubella, salmonellosis, SARS (severe acute respiratory syndrome), scabies, scarlet fever, schistosomiasis, sepsis, shigellosis (bacillary dysentery), shingles (Herpes zoster), smallpox (variola), sporotrichosis, staphylococcal food poisoning, staphylococcal infection, strongyloidiasis, subacute sclerosing panencephalitis, taeniasis, tetanus (lockjaw), Tinea barbae (barber's itch), Tinea capitis (ringworm of the scalp), Tinea corporis (ringworm of the body), Tinea cruris (Jock itch), Tinea manum (ringworm of the hand), Tinea nigra, Tinea pedis (athlete's foot), Tinea unguium (onychomycosis), Tinea versicolor (Pityriasis versicolor), toxocariasis (ocular larva migrans (OLM), toxocariasis (visceral larva migrans (VLM), toxoplasmosis, trachoma, trichinosis, trichomoniasis, trichuriasis (whipworm infection), tuberculosis, tularemia, typhoid fever, typhus fever, Ureaplasma urealyticum infection, valley fever, Venezuelan equine encephalitis, Venezuelan hemorrhagic fever, Vibrio parahaemolyticus enteritis, Vibrio vulnificus infection, viral pneumonia, West Nile fever, white piedra (Tinea blanca), yellow fever, Yersinia pseudotuberculosis infection, yersiniosis, zeaspora, Zika fever, and zygomycosis.

An enriched immune cell population provided by the present disclosure can be used, either alone or in combination, to treat diseases including acute myeloid leukemia, B-cell lymphoma, chronic myelogenous leukemia, depression, gingival recession, hepatitis C, HIV infections, human papillomavirus, idiopathic CD4 lymphopenia, immunodeficiency secondary to organ transplantation, lipodystrophy, Kaposi sarcoma lymphoma, lymphopenia, mantle cell lymphoma, multiple sclerosis, myelodysplastic syndrome, non-Hodgkin lymphoma, recurrent adult diffuse large cell lymphoma, recurrent follicular lymphoma, rheumatoid arthritis, sepsis, and Type 2 diabetes.

An enriched immune cell population provided by the present disclosure can be used to treat cancers such as metastatic breast cancer, breast cancer, colon cancer, bladder cancer, metastatic prostate cancer, stage IV prostate cancer, castration-resistant prostate carcinoma, neuroblastoma, melanoma, kidney cancer, myeloproliferative neoplasm, sarcoma, and neurodermal tumors.

An enriched immune cell population provided by the present disclosure can be used in combination with temozolomide to treat glioblastoma, with atezolizumab to treat skin cancers such as MCC, C5CC and melanoma, with pembrolizumab to treat triple negative breast cancer, and in combination with CAR-T therapy to treat pediatric acute lymphoblastic leukemia.

Pharmaceutical compositions comprising an enriched immune cell population can be administered concurrently with the administration of another therapeutic agent, which may be part of the same pharmaceutical composition as, or in a different pharmaceutical composition than, that comprising an enriched immune cell population. An enriched immune cell population may be administered prior or subsequent to administration of another therapeutic agent. In combination therapy, the combination therapy may comprise alternating between administering an enriched immune cell population and a composition comprising another therapeutic agent, e.g., to minimize adverse drug effects associated with a particular drug. When an enriched immune cell population is administered concurrently with another therapeutic agent that potentially may produce an adverse drug effect including, for example, toxicity, the other therapeutic agent may be administered at a dose that falls below the threshold at which the adverse drug reaction is elicited.

Pharmaceutical compositions comprising an enriched immune cell population may be administered with one or more substances to enhance, modulate and/or control release, bioavailability, therapeutic efficacy, therapeutic potency, stability, and the like of a compound of an enriched immune cell population. For example, to enhance the therapeutic efficacy of an enriched immune cell population, metabolite thereof, or a pharmaceutical composition of any of the foregoing may be co-administered with one or more active agents to increase the absorption or diffusion of the enriched immune cell population from the gastrointestinal tract to the systemic circulation, or to inhibit degradation of the enriched immune cell population in the blood of a subject. A pharmaceutical composition comprising an enriched immune cell population may be co-administered with an active agent having pharmacological effects that enhance the therapeutic efficacy of the enriched immune cell population.

An enriched immune cell population, or a pharmaceutical composition comprising any of the foregoing may be administered in conjunction with an agent known or believed to be effective in treating an inflammatory disease or an autoimmune disease in a patient.

An enriched immune cell population, or a pharmaceutical composition comprising any of the foregoing may be administered in conjunction with an agent known or believed to interfere with proliferation. An enriched immune cell population, or a pharmaceutical composition comprising any of the foregoing may be administered in conjunction with an agent known or believed to interfere with mitosis. An enriched immune cell population, or a pharmaceutical composition comprising any of the foregoing may be administered in conjunction with an agent known or believed to interfere with DNA replication. An enriched immune cell population, or a pharmaceutical composition comprising an enriched immune cell population may be administered in conjunction with an agent known or believed to interfere with DNA repair.

An enriched immune cell population or a pharmaceutical composition thereof may be administered to a patient together with another compound for treating an inflammatory disease or an autoimmune disease in the patient. The at least one other therapeutic agent may be a different enriched immune cell population provided by the present disclosure. An enriched immune cell population and the at least one other therapeutic agent may act additively or synergistically. The at least one additional therapeutic agent may be included in the same pharmaceutical composition or vehicle comprising the enriched immune cell population or may be in a separate pharmaceutical composition or vehicle. Accordingly, methods provided by the present disclosure further include, in addition to administering an enriched immune cell population, administering one or more therapeutic agents effective for treating an inflammatory disease or an autoimmune disease or a different disease, disorder or condition than an inflammatory disease or an autoimmune disease. Methods provided by the present disclosure include administering an enriched immune cell population and one or more other therapeutic agents provided that the combined administration does not inhibit the therapeutic efficacy of the enriched immune cell population and/or does not produce adverse combination effects.

Enriched immune cell populations provided by the present disclosure can be administered either alone, or as a pharmaceutical composition in combination with diluents and/or with other therapeutic agents including cytokines such as IL-2, IL-7, and IL-15, immune cell populations, and/or compounds comprising IL-7Rαγc agonist peptides, IL-2Rβγc agonist peptides, or IL-15Rβγc agonist peptides.

Methods for treatment provided by the present disclosure comprise administering to a patient in need thereof an effective amount of at least one CAR, or immune cell comprising a CAR, wherein the CAR-T or immune cell is produced using methods or providing an enriched immune cell population provided by the present disclosure.

In certain methods of treatment provided by the present disclosure an immune cell-mediated immune response can be induced in a patient by administering an effective amount of an engineered immune cell population to the patient. The immune cell-mediated immune response can be directed against a target cell or population of cells. An engineered immune cell can comprise a chimeric antigen receptor (CAR). A target cell can be a tumor cell. A method for treating or preventing a malignancy can comprise administering to a patient an effective amount of at least one isolated antigen binding domain. A method for treating or preventing a malignancy can comprise administering to a patient an effective amount an enriched immune cell population, wherein the enriched immune cell population comprises at least one chimeric antigen receptor, a T cell receptor, and/or isolated antigen binding domain. CAR-containing immune cells can be used to treat malignancies involving the aberrant expression of biomarkers.

CAR-containing immune cells can be used to treat, for example, small cell lung cancer, melanoma, low grade gliomas, glioblastoma, medullary thyroid cancer, carcinoids, dispersed neuroendocrine tumors in the pancreas, bladder and prostate, testicular cancer, and lung adenocarcinomas with neuroendocrine features.

Methods of treatment provided by the present disclosure include methods for reducing the size of a tumor in a patient comprising administering to a patient an enriched engineered immune cell to the patient, wherein the enriched engineered immune cell comprises a chimeric antigen receptor comprising an antigen binding domain and binds to an antigen on the tumor.

An engineered immune cell can be delivered to a tumor bed.

An engineered immune cell provided by the present disclosure can be an autologous immune cell such as an autologous T cell.

An engineered immune cell provided by the present disclosure can be an allogeneic immune cell such as an allogeneic T cell.

An engineered immune cell provided by the present disclosure can be a heterologous immune cell such as a heterologous T cell.

An engineered immune cell provided by the present disclosure can be transfected or transduced in vivo.

An engineered immune cell can be transfected or transduced ex vivo.

### Aspects

The invention is further defined by one or more of the following aspects.

Aspect 1. A culture medium for expanding a target immune cell population comprising a first stimulant of proliferation for the target immune cell population proliferation, wherein the first stimulant comprises an IL-2Rβγc agonist peptide.

Aspect 2. The culture medium of aspect 1, wherein the IL-2Rβγc agonist peptide comprises:
(a) an IL-2Rβ ligand, wherein the IL-2Rβ ligand comprises:
   an amino acid sequence of any one of SEQ ID NO: 74-172, 173-253, 330-560, 561-587, 588-608, 609-618, and 770-785;
   a truncated amino acid sequence of any one of SEQ ID NO: 74-172, 588-608, 173-253, 330-560, 561-587, 609-618, and 770-785;
   an amino acid sequence of any one of SEQ ID NO: 74-172, 173-253, 330-560, 561-587, 588-608, 609-618, and 770-785 having from 1 to 5 amino acid substitutions;
   an amino acid sequence of any one of SEQ ID NO: 74-172, 173-253, 330-560, 561-587, 588-608, 609-618, and 770-785 having from 1 to 5 flanking glycines on the N-terminus, the C-terminus, or both the N-terminus and the C-terminus; and/or
   an amino acid sequence having greater than 60% sequence similarity to an amino acid sequence of any one of SEQ ID NO: 74-172, 173-253, 330-560, 561-587, 588-608, 609-618, or 770-785; and
(b) an IL-2Rγc ligand, wherein the Rγc ligand comprises:
   an amino acid sequence of any one of SEQ ID NO: 631-715, 723-749, 786-795, and 797-800;
   a truncated amino acid sequence of any one of SEQ ID NO: 631-715, 723-749, 786-795, and 797-800;
   an amino acid sequence of any one of SEQ ID NO: 631-715, 723-749, 786-795, and 797-800 having from 1 to 5 amino acid substitutions;
   an amino acid sequence of any one of SEQ ID NO: 631-715, 723-749, 786-795, and 797-800 having from 1 to 5 flanking glycines on the N-terminus, the C-terminus, or both the N-terminus and the C-terminus; and/or
   an amino acid sequence having greater than 60% sequence similarity to an amino acid sequence of any one of SEQ ID NO: 631-715, 723-749, 786-795, and 797-800.

Aspect 3. The culture medium of aspect 2, wherein the IL-2Rβ ligand comprises an amino acid sequence of any one of SEQ ID NO: 74-172, 173-253, 330-560, 561-587, 588-608, 609-618, and 770-785.

Aspect 4. The culture medium of aspect 2, wherein the IL-2Rβ ligand comprises an amino acid sequence of any one of SEQ ID NO: 393-410, 420-495, 500-556, 786-795, 134, and 797-800 having from 1 to 5 amino acid substitutions.

Aspect 5. The culture medium of aspect 2, wherein the IL-2Rβ ligand comprises an amino acid sequence having greater than 60% sequence similarity to the amino acid sequence of any one of SEQ ID NO: 74-172, 173-253, 330-560, 561-587, 588-608, 609-618, or 770-785.

Aspect 6. The culture medium of any one of aspects 2 to 3, wherein the IL-2Rβ ligand comprises an amino acid sequence of any one of SEQ ID NO: 330-560, 561-587, 609-618, or 770-785.

Aspect 7. The culture medium of aspect 2, wherein the IL-2Rβ ligand comprises an amino acid sequence of any one of SEQ ID NO: 527.

Aspect 8. The culture medium of any one of aspect 2 to 6, wherein the IL-2Rγc ligand comprises an amino acid sequence of any one of SEQ ID NO: 631-718, 723-749, 786-795, 652, and 797-800.

Aspect 9. The culture medium of any one of aspect 2 to 6, wherein the IL-2Rγc ligand comprises an amino acid sequence of any one of SEQ ID NO: 631-718, 723-749, 786-795, 652, and 797-800, having from 1 to 5 amino acid substitutions.

Aspect 10. The culture medium of any one of aspect 2 to 6, wherein the IL-2Rγc ligand comprises having greater than 60% sequence similarity to the amino acid sequence of any one of SEQ ID NO: 631-718, 723-749, 786-795, 652, and 797-800.

Aspect 11. The culture medium of any one of aspects 2 to 5, wherein the IL-2Rγc ligand comprises an amino acid sequence of any one of SEQ ID NO: 786-795, 652, and 797-800.

Aspect 12. The culture medium of any one of aspect 2 to 6, wherein the IL-2Rγc ligand comprises an amino acid sequence of SEQ ID NO: 652.

Aspect 13. The culture medium of any one of aspects 2 to 12, wherein the IL-2Rβ ligand is bound to the IL-2Rγc ligand through a ligand linker.

Aspect 14. The culture medium of aspect 13, wherein the ligand linker comprises a peptidyl ligand linker.

Aspect 15. The culture medium of aspect 1, wherein the IL-2Rβγc agonist peptide comprises:
an amino acid sequence of any one of SEQ ID NO: 801-804, 809-812 and 813-852;
a truncated amino acid sequence of any one of SEQ ID NO: 801-804 and 809-812, and 813-852;
an amino acid sequence of any one of SEQ ID NO: 801-804, 809-812, and 813-852 having from 1 to 10 amino acid substitutions;
an amino acid sequence of any one of SEQ ID NO: 801-804, 809-812, and 813-852 having from 1 to 5 flanking glycines on the N-terminus, the C-terminus, or both the N-terminus and the C-terminus; an/or
an amino acid sequence having greater than 60% sequence similarity to an amino acid sequence of any one of SEQ ID NO: 801-804, 809-812, and 813-852.

Aspect 16. The culture medium of aspect 1, wherein the IL-2Rβγc agonist peptide comprises an amino acid sequence of any one of SEQ ID NO: 801-804, 809-812, and 813-852.

Aspect 17. The culture medium of aspect 1, wherein the IL-2Rβγc agonist peptide comprises an amino acid sequence of any one of SEQ ID NO: 801-804, 809-812, and 813-852 having from 1 to 10 amino acid substitutions.

Aspect 18. The culture medium of aspect 1, wherein the IL-2Rβγc agonist peptide comprises an amino acid sequence having greater than 60% sequence similarity to the amino acid sequence of any one of SEQ ID NO: 801-804, 809-812, and 813-852.

Aspect 19. The culture medium of aspect 1, wherein the IL-2Rβγc agonist peptide comprises an amino acid sequence of any one of SEQ ID NO: 801-804, 809-812, and 813-852.

Aspect 20. The culture medium of aspect 1, wherein the IL-2Rβγc agonist peptide comprises an amino acid sequence of any one of SEQ ID NO: 851 and 852.

Aspect 21. The culture medium of any one of aspects 1 to 20, wherein the IL-2Rβγc agonist peptide comprises an immobilized IL-2Rβγc agonist peptide.

Aspect 22. The culture medium any one of aspects 1 to 21, wherein the culture medium comprises a second stimulant of proliferation for the target immune cell population.

Aspect 23. The culture medium of aspect 22, wherein the second stimulant comprises an IL-7Rαγc agonist peptide.

Aspect 24. The culture medium of aspect 23, wherein the IL-7Rαγc agonist peptide comprises an amino acid sequence of any one of SEQ ID NO: 853-878 and 880-930.

Aspect 25. The culture medium of aspect 23, wherein the IL-7Rαγc agonist peptide comprises an amino acid sequence of any one of SEQ ID NO: 853, 869, 878, 890-893, 901, and 916.

Aspect 26. The culture medium of any one of aspects 22 to 25, wherein the second stimulant comprises an IL-15Rβγc agonist peptide.

Aspect 27. The culture medium of any one of aspects 1 to 26, wherein the culture medium comprises an antibody directed to the target immune cell population.

Aspect 28. The culture medium of any one of aspects 1 to 27, wherein the target immune cell population comprises a target T cell population.

Aspect 29. The culture medium of aspect 28, wherein the target T cell population comprises CD4+ T cells and CD8+ T cells.

Aspect 30. The culture medium of any one of aspects 28 to 29, wherein the culture medium comprises an antibody directed to the target T cell population.

Aspect 31. The culture medium of aspect 30, wherein the antibody directed to the target T cell population comprises an anti-CD3 antibody, an anti-CD28 antibody, or a combination thereof.

Aspect 32. A method of expanding a target immune cell population of an initial immune cell population, comprising incubating an initial immune cell population in the culture medium of any one of aspects 1 to 31 to provide an expanded target immune cell population.

Aspect 33. The method of aspect 32, wherein the target immune cell population comprises a target T cell population.

Aspect 34. The method of aspect 33, wherein the expanded target T cell population comprises Tcm cells, Tscm cells, or both Tcm cells and Tscm cells.

Aspect 35. The method of aspect 34, wherein the expanded target T cell population comprises CD4+ T cells and CD8+ T cells.

Aspect 36. The method of any one of aspects 32 to 35, wherein, a number of cells per milliliter of blood of the target immune cell population is expanded by greater than 10 times compared to an initial immune cell population; and percent is based on the number of cells per milliliter of blood of the target immune cell population prior to expansion.

Aspect 37. The method of any one of aspects 32 to 36, wherein the expanded target immune cell population is enriched by greater than 100 times as measured over a period from 10 days to 30 days compared to the initial population of target immune cells.

Aspect 38. The method of any one of aspects 32 to 37, wherein the target immune cell population comprises a population of engineered immune cells.

Aspect 39. The method of aspect 38, wherein the population of engineered immune cells express one or more chimeric antigen receptors.

Aspect 40. The method of any one of aspects 32 to 39, wherein the initial immune cell population comprises allogeneic immune cells.

Aspect 41. The method of any one of aspects 32 to 40, wherein the initial immune cell population comprises autologous immune cells.

Aspect 42. The method of any one of aspects 32 to 41, wherein incubating comprises incubating in the presence of an IL-7Rαγc agonist peptide.

Aspect 43. The method of aspect 42, wherein incubating in the presence of an IL-7Rαγc agonist peptide comprises incubating together with the IL-2Rβγc agonist peptide.

Aspect 44. The method of aspect 42, wherein incubating in the presence of an IL-7Rαγc agonist peptide comprises incubating in a culture medium that does not comprise an IL-2Rβγc agonist peptide.

Aspect 45. A method of manufacturing an immune cell population, comprising activating a target immune cell population of a population of primary cells to provide an activated target immune cell population; and expanding the activated target immune cell population, wherein expanding comprises incubating the activated target immune cell population in the culture medium of any one of aspects 1 to 31 to provide an expanded target immune cell population.

Aspect 46. The method of aspect 45, wherein the population of primary cells comprise human primary cells.

Aspect 47. The method of aspect 46, wherein the human primary cells comprise mononuclear cells.

Aspect 48. The method of aspect 47, wherein the mononuclear cells comprise peripheral blood mononuclear cells (PBMCs) from a patient.

Aspect 49. The method of aspect 47, wherein the mononuclear cells comprise peripheral blood mononuclear cells (PBMCs) from a donor.

Aspect 50. The method of any one of aspects 45 to 49, comprising, before activating the target immune cell population, enriching the target immune cell population in the population of primary cells.

Aspect 51. The method of any one of aspects 45 to 50, wherein, after activating the target immune cell population, causing the activated target immune cell population to express a cell surface receptor.

Aspect 52. The method of aspect 51, wherein engineering comprises transducing the activated target immune cell population with a viral vector to provide a transduced activated target immune cell population.

Aspect 53. The method of aspect 52, wherein the viral vector comprises a viral vector expressing a T cell receptor (TCR) or a chimeric antigen receptor (CAR).

Aspect 54. The method of aspect 53, wherein the TCR or CAR recognizes a specific antigenic moiety on a surface of a target cell.

Aspect 55. The method of aspect 54, wherein the antigenic moiety is MHC class I and/or MHC class II dependent.

Aspect 56. The method of any one of aspects 45 to 45, wherein activating comprises incubating the target immune cell population in the presence of an antibody to the target immune cell population.

Aspect 57. The method of aspect 56, wherein the antibody is immobilized on a substrate.

Aspect 58. The method of any one of aspects 45 to 57, wherein the IL-2Rβγc agonist peptide is immobilized on a substrate.

Aspect 59. The method of any one of aspects 45 to 58, wherein expanding comprises incubating the activated target immune cell population in the presence of an IL-7Rαγc agonist peptide.

Aspect 60. The method of aspect 59, wherein incubating the activated target immune cell population in the presence of an IL-7Rαγc agonist peptide comprises incubating the activated target immune cell population together with the IL-2Rβγc agonist peptide.

Aspect 61. The method of aspect 59, wherein incubating the activated target immune cell population in the presence of an IL-7Rαγc agonist peptide comprises incubating the activated target immune cell population in a culture medium that does not comprise an IL-2Rβγc agonist peptide.

Aspect 62. The method of any one of aspects 45 to 61, wherein the target immune cell population comprises CD4+ T cells and CD8+ T cells.

Aspect 63. The method of aspect 62, comprising, before activating the CD4+ T cells and CD8+ T cells, enriching the CD4+ T cells and CD8+ T cells in the population of primary cells.

Aspect 64. The method of aspect 63, wherein enriching comprises removing CD57+ T cells from the population of primary cells to provide a depleted population of CD57+ cells.

Aspect 65. The method of any one of aspects 62 to 64, wherein activating comprises incubating the CD4+ T cells and CD8+ T cells in the presence of anti-CD3 monoclonal antibody and an anti-CD28 monoclonal antibody.

Aspect 66. The method of any one of aspects 45 to 65, comprising, before activating, enriching the target immune cell population in the population of primary cells.

Aspect 67. An enriched population of immune cells prepared using the culture medium of any one of aspects 1 to 31.

Aspect 68. An enriched population of immune cells prepared using the method of any one of aspects 32 to 44.

Aspect 69. An enriched population of immune cells prepared using the method of any one of aspects 45 to 66.

Aspect 70. The enriched population of immune cells of any one of aspects 67 to 69, wherein the enriched population of immune cells comprises T cells.

Aspect 71. The enriched population of immune cells of aspect 70, wherein the enriched population of T cells comprises CD4+ T cells and CD8+ T cells.

Aspect 72. A pharmaceutical composition comprising the enriched population of immune cells of any one of aspects 67 to 71.

Aspect 73. A method of treating a disease in a patient comprising administering to a patient in need of such treatment a therapeutically effective amount of the pharmaceutical composition of aspect 72.

Aspect 74. The method of aspect 73, wherein the disease is cancer, a viral disease, or an autoimmune disease.

Aspect 75. The method of aspect 74, wherein the cancer is selected from primary adult and childhood brain and CNS cancers including glioblastoma (GBM) and astrocytoma, skin cancers including melanoma, lung cancers including small cell lung cancers, non-small cell lung cancers (NSCLC), and large cell lung cancers, breast cancers including triple negative breast cancer (TNBC), blood cancers including myelodysplastic syndrome (MDS), multiple myeloma (MM), and acute myeloid leukemia (AML), prostate cancer including castrate resistant prostate cancer (CRPC), liver cancers including hepatocellular carcinoma (HCC), esophageal and gastric cancers, and any systemic and central metastases of any of the foregoing.

Aspect 76. The method of aspect 73, wherein the viral disease is selected from influenza, pneumonia, herpes, hepatitis, hepatitis A, hepatitis B, hepatitis C, chronic fatigue syndrome, sudden acute respiratory syndrome (SARS), gastroenteritis, enteritis, carditis, encephalitis, bronchiolitis, respiratory papillomatosis, meningitis, HIV/AIDS, and mononucleosis, and severe acute respiratory syndrome coronavirus 2 (SARS-CoV-2).

Aspect 77. The method of aspect 73, wherein the autoimmune disease is selected from lupus, graft-versus-host disease, hepatitis C-induced vasculitis, Type I diabetes, multiple sclerosis, spontaneous loss of pregnancy, atopic diseases, and inflammatory bowel diseases.

Aspect 78. An article of manufacture comprising the culture medium of any one of aspects 1 to 31.

Aspect 79. An article of manufacture comprising the enriched population of immune cells of any one of aspects 67 to 71.

Aspect 80. An article of manufacture comprising the pharmaceutical composition of aspect 72.

Aspect 81. An immobilized IL-2Rβγc agonist peptide, comprising an IL-2Rβγc agonist peptide bound to a substrate.

Aspect 82. The immobilized IL-2Rβγc agonist peptide of aspect 81, wherein the IL-2Rβγc agonist peptide comprises:
(a) an IL-2Rβ ligand, wherein the IL-2Rβ ligand comprises:
   an amino acid sequence of any one of SEQ ID NO: 74-172, 173-253, 330-560, 561-587, 588-608, 609-618, and 770-785;
   a truncated amino acid sequence of any one of SEQ ID NO: 74-172, 173-253, 330-560, 561-587, 588-608, 609-618, and 770-785;
   an amino acid sequence of any one of SEQ ID NO: 74-172, 173-253, 330-560, 561-587, 588-608, 609-618, and 770-785 having from 1 to 5 amino acid substitutions;
   an amino acid sequence of any one of SEQ ID NO: 74-172, 173-253, 330-560, 561-587, 588-608, 609-618, and 770-785 having from 1 to 5 flanking glycines on the N-terminus, the C-terminus, or both the N-terminus and the C-terminus; and/or
   an amino acid sequence having greater than 60% sequence similarity to an amino acid sequence of any one of SEQ ID NO: 74-172, 173-253, 330-560, 561-587, 588-608, 609-618, and 770-785; and
(b) an Rγc ligand, wherein the Rγc ligand comprises:
   an amino acid sequence of any one of SEQ ID NO: 631-715, 723-749, 786-795, and 797-800;
   a truncated amino acid sequence of any one of SEQ ID NO: 631-715, 723-749, 786-795, and 797-800;
   an amino acid sequence of any one of SEQ ID NO: 631-715, 723-749, 786-795, and 797-800 having from 1 to 5 amino acid substitutions;
   an amino acid sequence of any one of SEQ ID NO: 631-715, 723-749, 786-795, and 797-800 having from 1 to 5 flanking glycines on the N-terminus, the C-terminus, or both the N-terminus and the C-terminus; and/or
   an amino acid sequence having greater than 60% sequence similarity to an amino acid sequence of any one of SEQ ID NO: 631-715, 723-749, 786-795, and 797-800.

Aspect 83. The immobilized IL-2Rβγc agonist peptide of any one of aspects 81 to 82, wherein the IL-2Rβγc agonist peptide comprises an IL-2Rβ ligand comprising an amino acid sequence of any one of SEQ ID NO: 74-172, 173-253, 330-560, 561-587, 588-608, 609-618, and 770-785.

Aspect 84. The immobilized IL-2Rβγc agonist peptide of any one of aspects 81 to 82, wherein the IL-2Rβ ligand comprises an amino acid sequence of any one of SEQ ID NO: 591, 598, 755, 756, 760, 761, 765, 766.

Aspect 85. The immobilized IL-2Rβγc agonist peptide of any one of aspects 81 to 82, wherein the IL-2Rβ ligand comprises an amino acid sequence of any one of SEQ ID NO: 134 and 527.

Aspect 86. The immobilized IL-2Rβγc agonist peptide of any one of aspects 81 to 85, wherein the IL-2Rβγc agonist peptide comprises an Rγc ligand comprising an amino acid sequence of any one of SEQ ID NO: 631-715, 723-749, 786-795, and 797-800.

Aspect 87. The immobilized IL-2Rβγc agonist peptide of any one of aspects 81 to 85 wherein the Rγc ligand comprises an amino acid sequence of any one of SEQ ID NO: 652, 786-788, 791, 792, and 795.

Aspect 88. The immobilized IL-2Rβγc agonist peptide of any one of aspects 81 to 85 wherein the Rγc ligand comprises an amino acid sequence of any one of SEQ ID NO: 652.

Aspect 89. The immobilized IL-2Rβγc agonist peptide of any one of aspects 82 to 88, wherein the IL-2Rβ ligand is bound to the Rγc ligand through a ligand linker.

Aspect 90. The immobilized IL-2Rβγc agonist peptide of aspect 89, wherein the ligand linker comprises a peptidyl ligand linker.

Aspect 91. The immobilized IL-2Rβγc agonist peptide of aspect 81, wherein the IL-2Rβγc agonist peptide comprises:
an amino acid sequence of any one of SEQ ID NO: 801-804, 809-812, and 813-852;
a truncated amino acid sequence of any one of SEQ ID NO: 801-804, 809-812, and 813-852.
an amino acid sequence of any one of SEQ ID NO: 801-804, 809-812, and 813-852 having from 1 to 5 amino acid substitutions;
an amino acid sequence of any one of SEQ ID NO: 801-804, 809-812, and 813-852 having from 1 to 5 flanking glycines on the N-terminus, the C-terminus, or both the N-terminus and the C-terminus; and/or
an amino acid sequence having greater than 60% sequence similarity to an amino acid sequence of any one of SEQ ID NO: 801-804, 809-812, and 813-852.

Aspect 92. The immobilized IL-2Rβγc agonist peptide of aspect 81, wherein the IL-2Rβγc agonist peptide comprises:
an amino acid sequence of any one of SEQ ID NO: 834-852 and 801-833;
an amino acid sequence of any one of SEQ ID NO: 834-852 and 801-833 having from 1 to 10 amino acid substitutions; and/or
an amino acid sequence having greater than 60% sequence similarity to the amino acid sequence of any one of SEQ ID NO: 834-852 and 801-833.

Aspect 93. The immobilized IL-2Rβγc agonist peptide of aspect 81, wherein the IL-2Rβγc agonist peptide comprises:
an amino acid sequence of any one of SEQ ID NO: 801, 802, 851, and 852;
an amino acid sequence of any one of SEQ ID NO: 801, 802, 851, and 852 having from 1 to 10 amino acid substitutions; and/or
an amino acid sequence having greater than 60% sequence similarity to the amino acid sequence of any one of SEQ ID NO: 801, 802, 851, and 852.

Aspect 94. The immobilized IL-2Rβγc agonist peptide of any one of aspects 81 to 93, wherein the substrate comprises a bead.

Aspect 95. The immobilized IL-2Rβγc agonist peptide of any one of aspects 81 to 93, wherein the substrate comprises a bead, a matrix, or a plate.

Aspect 96. The immobilized IL-2Rβγc agonist peptide of any one of aspects 81 to 95, wherein the IL-2Rβγc agonist peptide is bound to the substrate through a substrate linker.

### EXAMPLES

The following examples describe in detail culture media comprising an IL-2Rβγc agonist peptide, methods of expanding a targeted immune cell population, and methods of manufacturing a targeted immune cell population.

It will be apparent to those skilled in the art that many modifications, both to materials and methods, may be practiced without departing from the scope of the invention.

### Example 1

### pSTAT5 Flow Cytometry

For resting conditions, frozen PBMCs were cultured 1 day. The cells were stained with a viability dye for 30 minutes at 37° C., followed by incubation with surface marker antibodies for 30 minutes on ice, and then treated with a serially diluted IL-7Rαγc agonist peptide (SEQ ID NO: 891) or a serially diluted IL-2Rβγc agonist peptide (SEQ ID NO: 851) in T cell medium for 30 minutes. After incubation, the cells were washed, fixed, permeabilized according to the manufacturer's instructions, and stained with pSTAT5 and intracellular antibodies for 50 minutes on ice. The antibody-stained cells were analyzed immediately by flow cytometry using a NovoCyte® Advanteon™ flow cytometer (Agilent), and the data were analyzed using FlowJo™ software (FlowJo LLC). Fluorescence minus one (FMO) controls were used to draw gates. For measuring pSTAT5 in activated PBMCs, the overnight rested cells were incubated with CD3/CD28 Dynabeads™ (ThermoFisher Scientific) at 1 bead to 1 cell ratio for 3 days and then rested for 2 days in the fresh medium without the Dynabeads™ before testing the compounds.

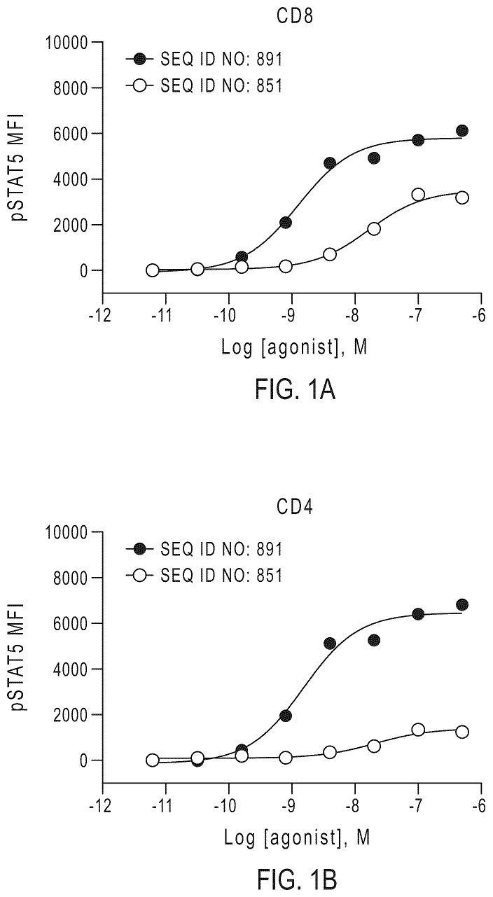

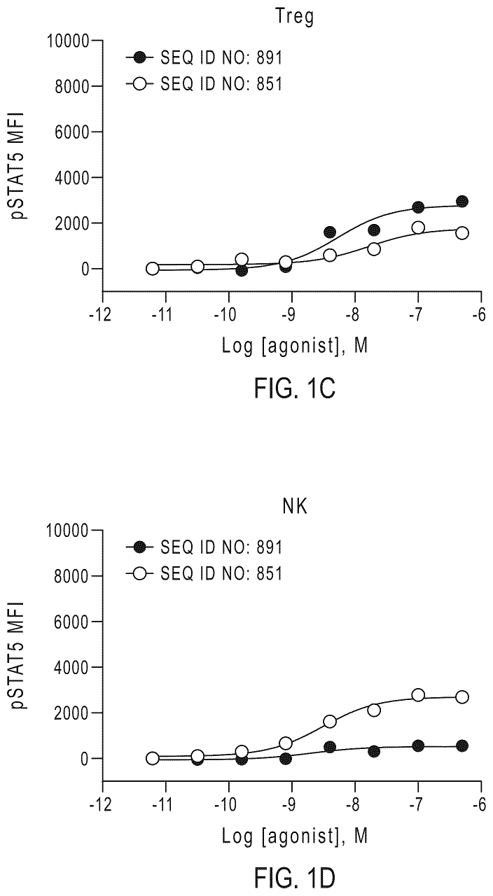

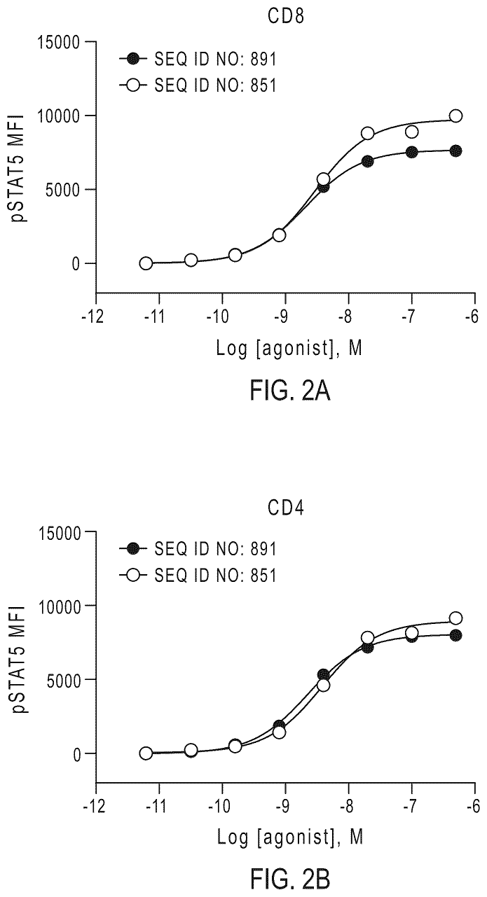

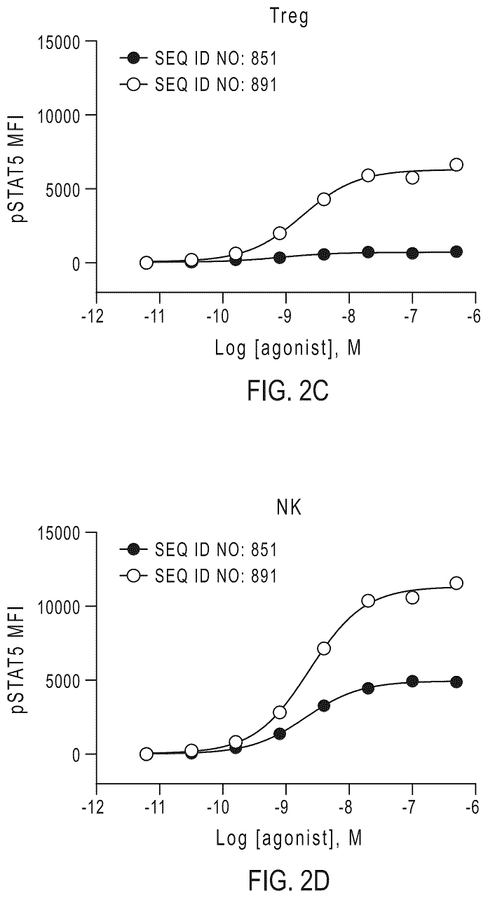

The results for resting PBMCs are shown in FIGS. 1A-1D and for PMBCs activated with anti-CD3 and anti-CD28 monoclonal antibodies are shown in FIGS. 2A-2D.

### Example 2

### Immune Cell Proliferation

Frozen human PBMCs from 5 healthy donors (Stem Cell Technologies, cat no. 70025.2) were thawed and rested overnight at 37° C., 5% CO₂ in CTS™ OpTmizer™ T Cell Expansion SFM (ThermoFisher Scientific No. A1048501). Four million cells were cultured in a plate coated with or without 10 ng/mL of CD3 antibody (Clone SP34-2, Fisher Scientific) and in the presence or absence of 250 nM of an IL-7Rαγc agonist peptide (SEQ ID NO: 891) or an IL-2Rβγc agonist peptide (SEQ ID NO: 852). Fresh media containing the agonist peptides were provided every 2-3 days. Cell aliquots were taken weekly, and cell surface staining was performed using antibodies, followed by fixable viability dye staining and analyzed using a NovoCyte® Advanteon™ flow cytometer (Agilent). The data were analyzed using FlowJo™ software (FloJo LLC). Fluorescent minus one (FMO) controls were used to draw gates.

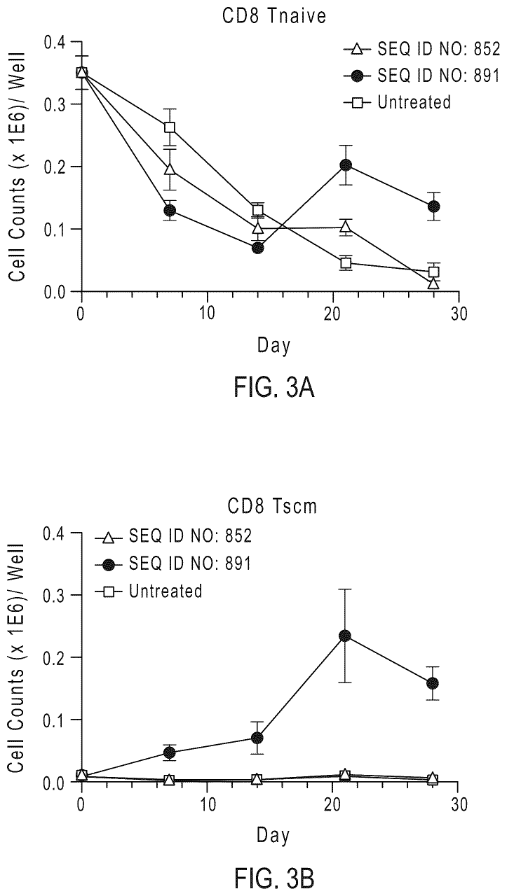

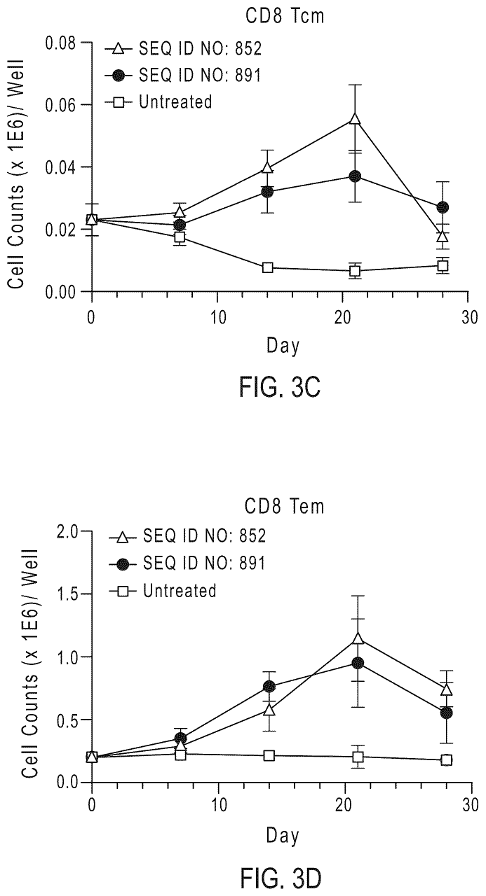

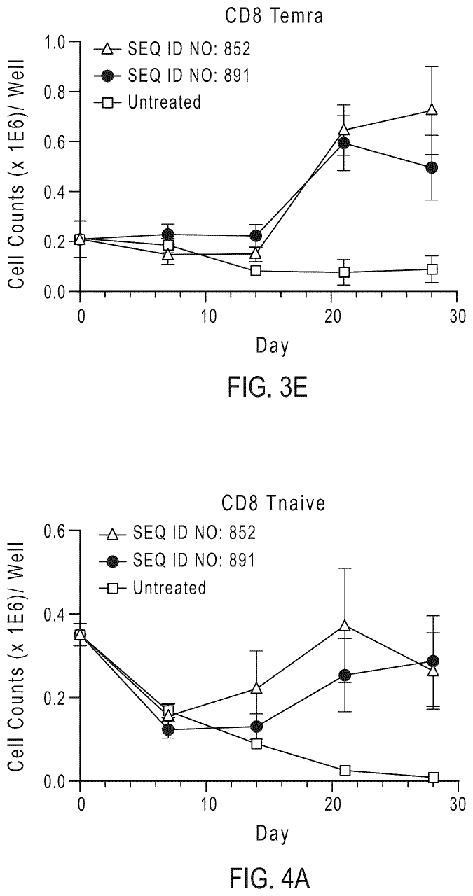

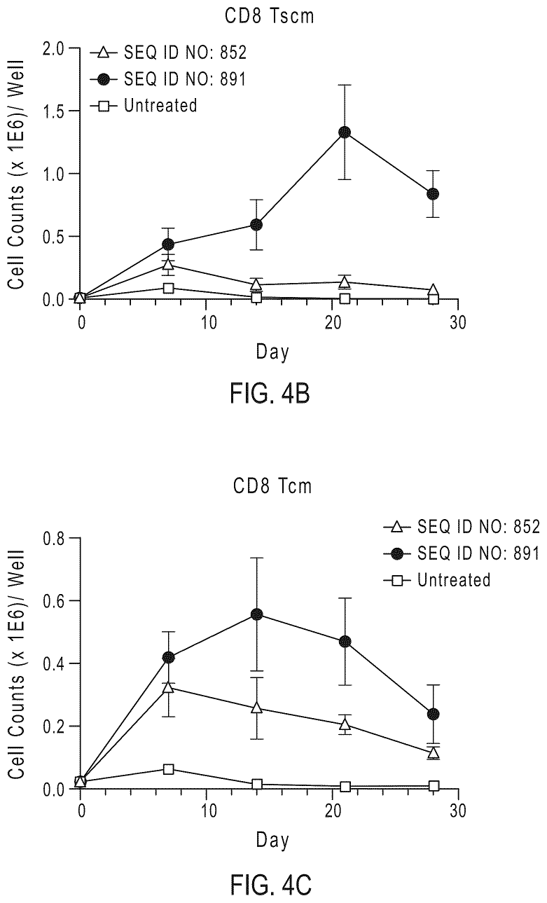

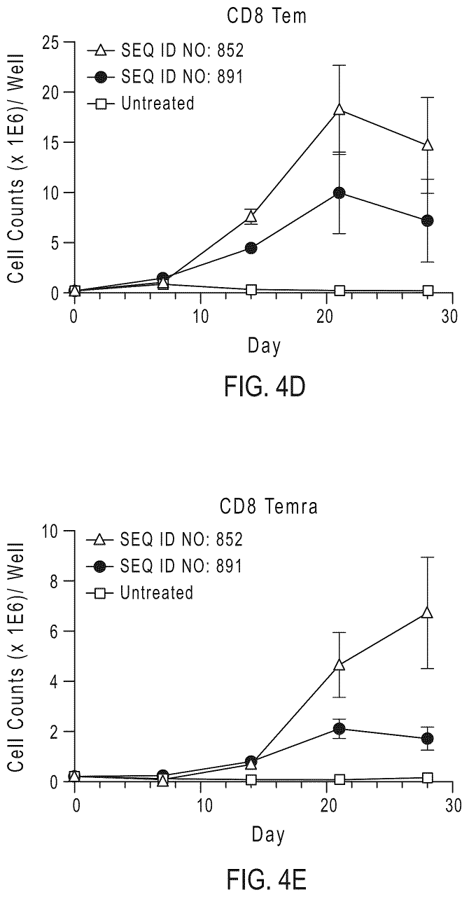

The cell counts for T cell subpopulations during incubation of resting PBMCs are shown in FIGS. 3A-3E and for PMBCs activated with 10 ng/mL anti-CD3 monoclonal antibody are shown in FIGS. 4A-4E.

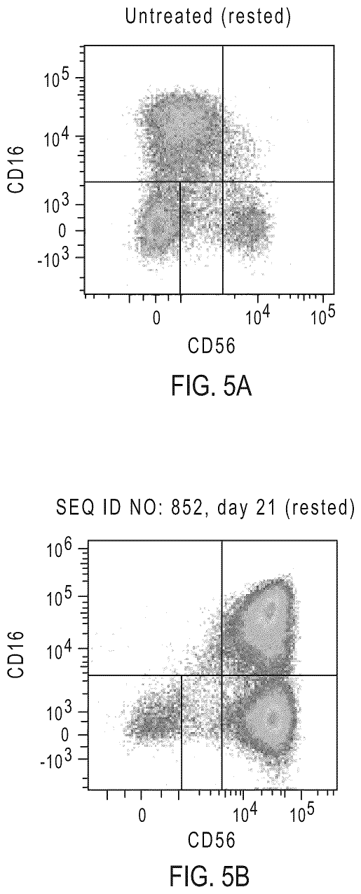

NK cell subpopulation profiles following incubation of resting and activated PBMCs with an IL-2Rβγc agonist peptide (SEQ ID NO: 852) for 21 days are shown in FIGS. 5A and 5B, respectively.

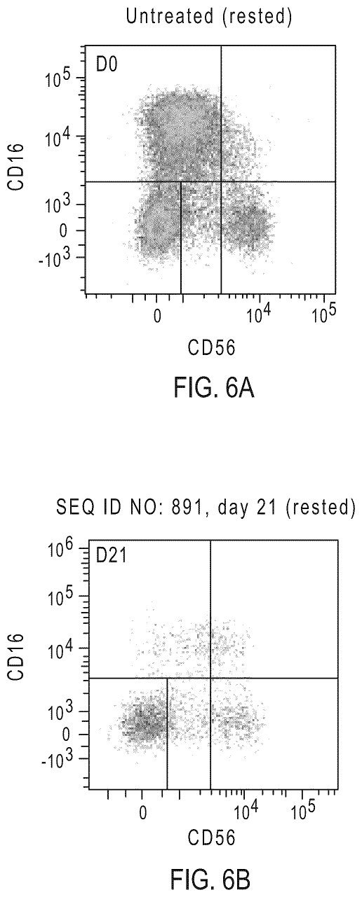

NK cell subpopulation profiles following incubation of resting and activated PBMCs with an IL-7Rαγc agonist peptide (SEQ ID NO: 891) for 21 days are shown in FIGS. 6A and 6B, respectively.

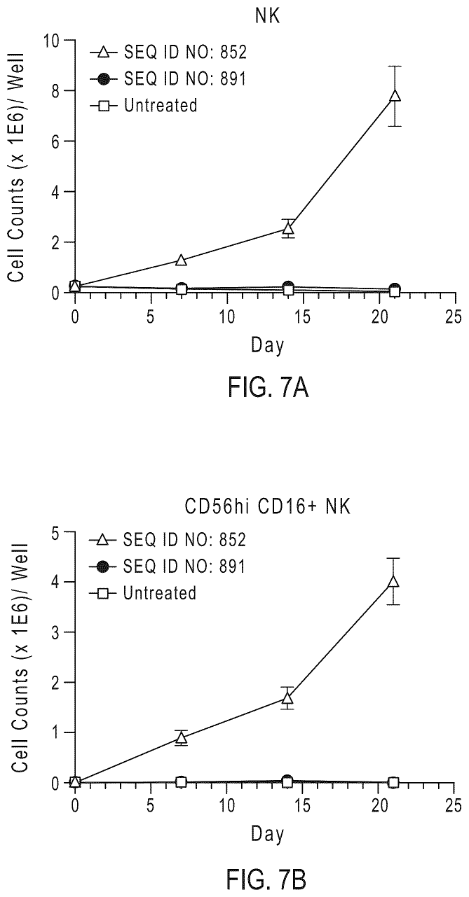

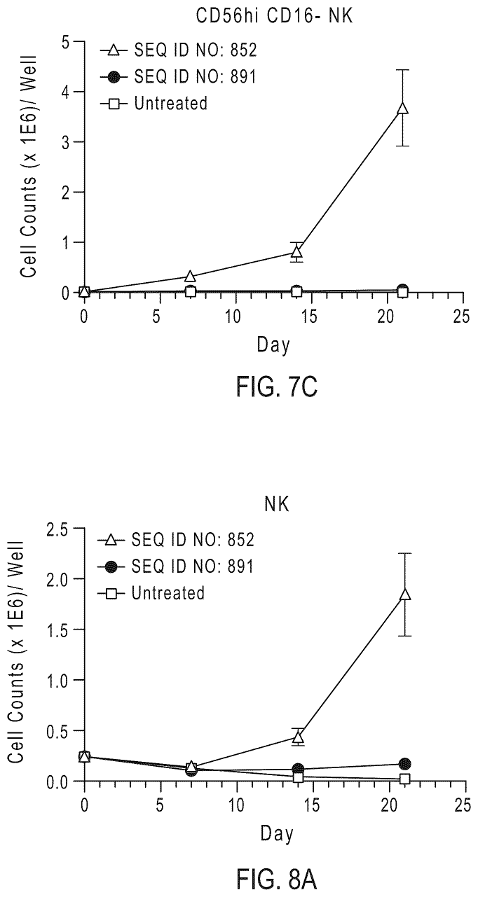

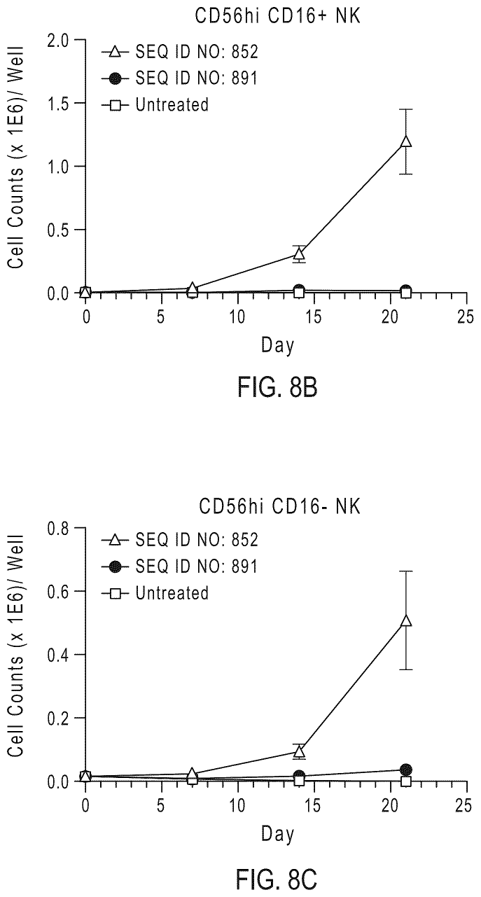

The cell counts for NK cell subpopulations during incubation of resting PBMCs without a agonist peptide, with an IL-2Rβγc agonist peptide (SEQ ID NO: 852), or with an IL-2Rβγc agonist peptide (SEQ ID NO: 852) are shown in FIGS. 7A-7C and for PMBCs activated with 10 ng/mL anti-CD3 monoclonal antibody are shown in FIGS. 8A-8C.

### Example 3

### Cell Counting

Cell cultures were initially plated at 4×10⁶ cells in 2 mL per well. At each assay time point, 50 μL/well of cells were taken from each culture and incubated with 200 μL of the viability dye solution. The Novocyte® Advanteon™ flow cytometer was set up to analyze 50 μL (12.5 μL original culture volume), which allowed the calculation of the absolute live cell counts. Because the culture volumes change over time due to splitting and removal, dilution factors were considered when calculating the total number of cells.

### Example 4

### Proliferation of NK-92 Cells with IL-2 and an IL-2Rβγc Agonist Peptide

NK-92 cell proliferation in response to IL-2Rβγc agonist peptide (SEQ ID NO: 801 acylated) or IL-2 was measured using Ki67 staining. The nuclear protein Ki67 was present during all active phases of the cell cycle but was absent in resting cells. NK-92 cells were resuspended in starvation medium and plated at 2×10⁵ cells/well in a 96-well plate. Three-fold serial dilution of the test compound was then added to the cells for 48 h. Following the incubation period, cells were treated with Live/Dead® Fixable Aqua Dye (ThermoFisher No. L34957) for 30 min to stain for viable cells. The cells were then washed with PBS and then fixed and permeabilized for 1 h at 25° C. with Foxp3 Transcription Factor Fix/Perm® buffer (eBioscience No. 00-5523). Cells were washed and then stained with anti-Ki67 PE antibody (ThermoFisher No. 12-5698-82). After a final wash the cells were analyzed by flow cytometry on an LSR II instrument (Becton Dickinson). Data analysis was performed using FlowJo™ software. The median fluorescence intensity of Ki67+ cells was plotted as a function of test compound concentration.

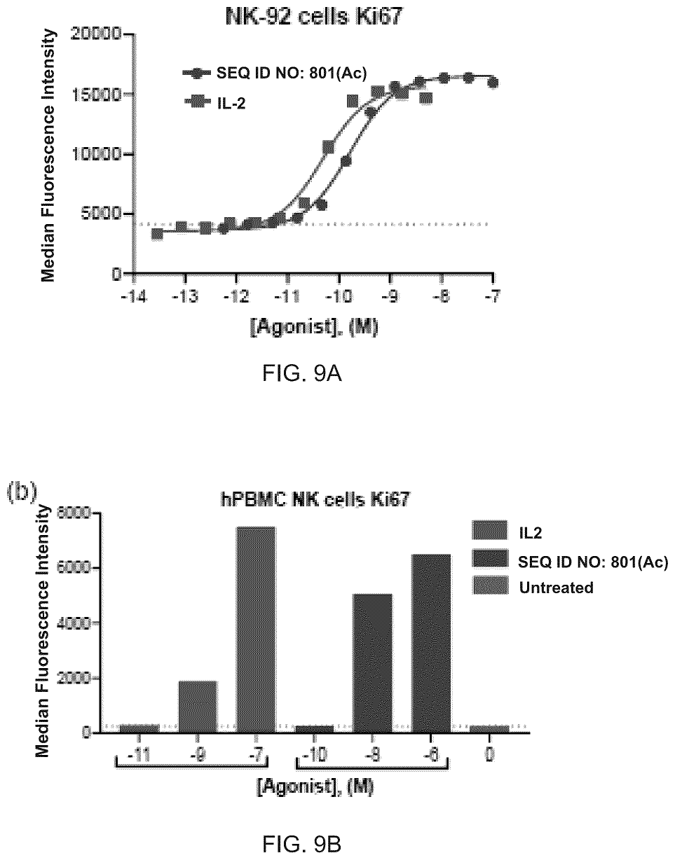

The results are presented in FIGS. 9A and 9B.

### Example 5

### Proliferation in NK Cells from Human PBMCs

Human PBMCs were isolated from a buffy coat by density gradient centrifugation (Lymphoprep®, Stemcell Technologies No. 07811) and cultured overnight in T-cell medium (CTS OpTmizer®, ThermoFisher #A1048501) at 3×10⁶ cells/mL in a T75 flask. The following day, cells were resuspended in fresh medium and plated at 5×10⁵ cells/well in a 96-well cell culture plate. Three-fold serial dilutions of either IL-2 or an IL-2Rβγc ligand (SEQ ID NO: 802 acylated) were added to the cells and incubated for 3 days at 37° C. After the treatment, cells were incubated in viability dye (Live/Dead® Fixable Aqua Cell Stain Kit, ThermoFisher #L349650) for 30 min at 37° C., after which surface antibody staining was then performed in PBS+2% FBS for 30 min on ice. Cells were fixed and permeabilized with Fixation/Permeabilization Buffer (eBioscience Foxp3/Transcription Staining Buffer Set, ThermoFisher #00-5523-00) for 30 min on ice. Intracellular (Ki-67) staining was performed in Permeabilization Buffer for 30 min on ice and the treated cells resuspended in PBS+2% FBS prior to FACS analysis. NK cells were identified as CD56+ and/or CD159a+ cells from CD3− and CD20− (non-T, non-B cell) populations. Antibody conjugates used for cell surface and intracellular staining are shown in Table 1.

TABLE 1
Antibody conjugates used for cell surface and intracellular staining.

| Marker | CD3 | Ki-67 | CD56 | CD20 | CD45RA | CD8 | CD159a | Live/Dead |
|---|---|---|---|---|---|---|---|---|
| Fluor | FITC | PE | PerCP- | PE-Cy7 | APC | BV421 | BV650 | Aqua |
| | | | eFluor710 | | | | | |
| Clone | UCHT1 | SolA15 | CMSSB | 2H7 | HI100 | SK1 | 13411 | — |
| Supplier | Invitrogen | Invitrogen | Invitrogen | BioLegend | BD | BioLegend | BD | Invitrogen |
| Cat. No. | CD300 | 12-2698-82 | 46-0567-42 | 302312 | 550855 | 344748 | 747920 | L349650 |

Finally, it should be noted that there are alternative ways of implementing the embodiments disclosed herein. Accordingly, the present embodiments are to be considered as illustrative and not restrictive, and the claims are not to be limited to the details given herein but may be modified within the scope and equivalents thereof.

What is claimed is:

1. A culture medium for expanding a target immune cell population comprising a first stimulant of proliferation for the target immune cell population proliferation, wherein the first stimulant comprises an IL-2Rβγc agonist peptide, wherein the IL-2Rβγc agonist peptide comprises:
(a) an IL-2Rβ ligand, wherein the IL-2Rβ ligand comprises:
an amino acid sequence of any one of SEQ ID NO: 134, 527, 591, 598, 755, 756, 760, 761, 765, 766, 770, 776, and 777; or
an amino acid sequence having greater than 80% sequence similarity to any one of SEQ ID NO: 134, 527, 591, 598, 755, 756, 760, 761, 765, 766, 770, 776, and 777; and
(b) an IL-2Rγc ligand, wherein the IL-2Rγc ligand comprises:
an amino acid sequence of any one of SEQ ID NO: 652, 786-788, 791, 792, and 795; or
an amino acid sequence having greater than 80% sequence similarity to any one of SEQ ID NO: 652, 786-788, 791, 792, and 795.

2. The culture medium of claim 1, wherein the IL-2Rβ ligand comprises SEQ ID NO: 134.

3. The culture medium of claim 1, wherein the IL-2Rβ ligand comprises an amino acid sequence having greater than 80% sequence similarity to SEQ ID NO: 134.

4. The culture medium of claim 1, wherein the IL-2Rβ ligand comprises SEQ ID NO: 527.

5. The culture medium of claim 1, wherein the IL-2Rβ ligand comprises an amino acid sequence having greater than 80% sequence similarity to SEQ ID NO: 527.

6. The culture medium of claim 1, wherein the IL-2Rγc ligand comprises SEQ ID NO: 652.

7. The culture medium of claim 1, wherein the IL-2Rγc ligand comprises an amino acid sequence having greater than 80% sequence similarity to SEQ ID NO: 652.

8. The culture medium of claim 1, wherein the IL-2Rβ ligand is bound to the IL-2Rγc ligand through a ligand linker.

9. The culture medium of claim 8, wherein the ligand linker comprises a peptidyl ligand linker.

10. The culture medium of claim 1, wherein the IL-2Rβγc agonist peptide comprises any one of SEQ ID NO: 801-804 and 809-812, 829-830, 833, 834, 839, and 844-852, wherein X³⁰⁰ is selected from any one of SEQ ID NO: 1-39.

11. The culture medium of claim 1, wherein the IL-2Rβγc agonist peptide comprises any one of SEQ ID NO: 801-804.

12. The culture medium of claim 1, wherein the IL-2Rβγc agonist peptide comprises an amino acid sequence having greater than 80% sequence similarity to any one of SEQ ID NO: 801-804, 829-830, 833, 834, 839, and 844-852, wherein X³⁰⁰ is selected from any one of SEQ ID NO: 1-39.

13. The culture medium of claim 1, wherein the IL-2Rβγc agonist peptide comprises any one of SEQ ID NO: 801 and 802.

14. The culture medium of claim 1, wherein the IL-2Rβγc agonist peptide comprises any one of SEQ ID NO: 834, 839, and 844-852, wherein X⁴⁰⁰ is selected from any one of SEQ ID NO: 1-39.

15. The culture medium of claim 1, wherein the IL-2Rβγc agonist peptide comprises an amino acid sequence having greater than 80% sequence similarity to any one of SEQ ID NO: 834, 839, and 844-852, wherein X⁴⁰⁰ is selected from any one of SEQ ID NO: 1-39.

16. The culture medium of claim 1, wherein the IL-2Rβγc agonist peptide comprises any one of SEQ ID NO: 851 and 852.

17. The culture medium of claim 1, wherein the IL-2Rβγc agonist peptide is immobilized.

18. The culture medium of claim 1, wherein the culture medium comprises a second stimulant of proliferation for the target immune cell population.

19. The culture medium of claim 18, wherein the second stimulant comprises an IL-7Rαγc agonist peptide, an IL-15Rβγc agonist peptide, or a combination thereof.

20. The culture medium of claim 1, wherein the culture medium comprises an antibody directed to the target immune cell population.

21. An enriched population of immune cells prepared using the culture medium of claim 1.

22. A method of expanding a target immune cell population of an initial immune cell population, comprising incubating an initial immune cell population in the culture medium of claim 1 to provide an expanded target immune cell population.

23. An expanded population of immune cells prepared using the method of claim 22.

24. A pharmaceutical composition comprising the expanded population of immune cells of claim 23.

25. A method of treating a disease in a patient comprising administering to a patient in need of such treatment a therapeutically effective amount of the pharmaceutical composition of claim 24.
